(19) United States
(12) Patent Application Publication
(10) Pub. No.: US 2026/0284073 A1
(43) Pub. Date: Sep. 24, 2026

(54) Azepino Analogs and Methods of Making and Using the Same

(71) Applicant: Kuleon LLC, Seattle, WA (US)

(72) Inventors: Jeremy Forest, Richmond Beach, WA (US); David Gilles, Tacoma, WA (US)

(73) Assignee: Kuleon LLC, Seattle, WA (US)

(21) Appl. No.: 19/691,267

(22) Filed: May 28, 2026

Related U.S. Application Data
(63) Continuation of application No. PCT/US24/57374, filed on Nov. 26, 2024.
(60) Provisional application No. 63/605,223, filed on Dec. 1, 2023.
(60) Provisional application No. 63/633,821, filed on Apr. 14, 2024.
(60) Provisional application No. 63/639,691, filed on Apr. 28, 2024.
(60) Provisional application No. 63/640,885, filed on May 1, 2024.
(60) Provisional application No. 63/647,743, filed on May 15, 2024.

(51) Int. Cl. A61K 31/55; C07D 471/22

(52) U.S. Cl. CPC A61K 31/55; C07D 471/22

(57) ABSTRACT

The present application relates to azepino compounds analogs, as well as methods of using them for treating and preventing a variety of conditions.

### CROSS-REFERENCE TO RELATED APPLICATIONS

This application is a continuation of International Application No. PCT/US2024/057374, filed Nov. 26, 2024, which claims priority to U.S. Provisional Application No. 63/647,743, filed May 15, 2024, U.S. Provisional Application No. 63/640,885, filed May 1, 2024, U.S. Provisional Application No. 63/639,691, filed Apr. 28, 2024, U.S. Provisional Application No. 63/633,821, filed Apr. 14, 2024, and U.S. Provisional Application No. 63/605,223, filed Dec. 1, 2023, the entire contents of which are incorporated by reference herein for all purposes.

### TECHNICAL FIELD

This disclosure relates to serotonin and nicotinic receptor modulators, including azepino analogs of Formula I, II, III, IV, V, VI, VII, VIIIa, VIIIb, IXa, IXb, Xa, Xb, XIa or XIb, and methods of using them for treating and preventing a variety of conditions.

### BACKGROUND

Presently, there is no cure for drug addiction. Indeed, the overwhelming majority (up to 85%) of patients undergoing modern day drug rehabilitation relapse back into compulsive drug taking. This relapse is motivated by the intense craving for the drug that is experienced by the drug-withdrawn person suffering from addiction even after years of being drug free. Psychosocial therapy is widely employed for the long-term treatment of drug addiction, but there remains an exceptionally high incidence of relapse to drug taking in the drug-withdrawn addict.

Psychostimulant and opioid drugs exhibit extremely high abuse liability. These drugs are often abused in a relatively drawn out “binge and crash” pattern known as a “run”. This typically lasts for several days, during which time the user foregoes food and sleep. Long-term, heavy use can initiate violent rages, induce anxiety, confusion and insomnia, and evoke a number of psychotic features, including intense paranoia, hallucinations, and delusions that endure for years after drug use has ceased.

To date, very little work has been done in developing novel drugs that target the impulsive features of drug-taking behavior, drug reward, and drug relapse. Accordingly, there continues to be a need to develop safe and effective drugs that target these features of addiction.

### SUMMARY

Disclosed herein are compounds of Formula I:

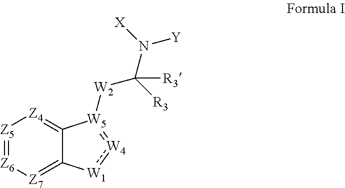

wherein
[symbol] is a single or double bond as needed to satisfy atom valences;
X and Y are each independently selected from hydrogen, deuterium, optionally substituted C₁-C₈ alkyl, optionally substituted C₂-C₈ alkenyl, and optionally substituted C₂-C₈ alkynyl, or Y is taken together with X and the nitrogen atom therebetween to form a 3- to 7-membered heterocyclic ring optionally including 1 to 2 additional ring heteromoieties selected from O, S, S(O), SO₂, and NR₉;
W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond;
W₂ is selected from —C(R₃₀)(R₃₁)— and —(C(R₃₀)(R₃₁))₂—;
W₄ is selected from C(R₂)(R_{2′}) and NR_{1c} when a [symbol] to which W₄ is attached is a single bond, and W₄ is selected from C(R₂) and N when a [symbol] to which W₄ is attached is a double bond;
W₅ is selected from C(R₅₁) and N when the [symbol] to which W₅ is attached is a single bond, and W₅ is C when the [symbol] to which W₅ is attached is a double bond; Z₄ is selected from N and CR₄;
Z₅ is selected from N and CR₅;
Z₆ is selected from N and CR₆;
Z₇ is selected from N and CR₇;
R₁ₐ and R_{1c} are each independently selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈ or Y is absent and R₁, is taken together with the nitrogen to which it is attached and the nitrogen atom to which X is attached to form an optionally substituted 5- to 9-membered heterocyclic ring optionally including 1 to 2 additional ring heteromoieties selected from O, S, S(O), SO₂, and NR₉;
R₂, R_{2′}, R₃, R_{3′}, R₄, R₅, R₆, R₇, R₃₀, R₃₁, R₅₀, R₅₁, and R_{51′} are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl, and —OSO₂R₈, or Y is absent and R₃ is taken together with the carbon to which it is attached and the nitrogen atom to which X is attached to form an optionally substituted 3- to 9-membered heterocyclic ring optionally including 1 to 2 additional ring heteromoieties selected from O, S, S(O), SO₂, and NR₉, or Y is absent and R₂ is taken together with the carbon to which it is attached and the nitrogen atom to which X is attached to form an optionally substituted 5- to 9-membered heterocyclic ring optionally including 1 to 2 additional ring heteromoieties selected from O, S, S(O), SO₂, and NR₉;
R₈ is, independently for each occurrence, selected from hydrogen, deuterium, —N(R₉)₂, optionally substituted C₁-C₈ alkyl, optionally substituted C₂-C₈ alkenyl, optionally substituted C₂-C₈ alkynyl, optionally substituted heteroaryl, and optionally substituted aryl; and
R₉ is, independently for each occurrence, is selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl;
and salts, solvates, hydrates, enantiomers, polymorphs and prodrugs thereof.

Disclosed herein are also compounds of Formula II:

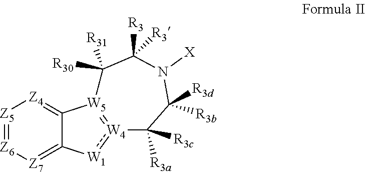

wherein
X is selected from hydrogen, deuterium, optionally substituted C₁-C₈ alkyl, optionally substituted C₂-C₈ alkenyl, and optionally substituted C₂-C₈ alkynyl, or X is absent and R₃, together with the carbon to which it is attached and the nitrogen associated with the absent X, and the atoms therebetween, are taken to form an optionally substituted 3- to 9-membered heterocyclic ring optionally including 1 to 2 additional ring heteromoieties selected from O, S, S(O), SO₂, and NR₉;
[symbol] is a single or double bond as needed to satisfy atom valences;
W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond;
W₄ is selected from CR₂ and N when a [symbol] to which W₄ is attached is a single bond, and W₄ is C when a [symbol] to which W₄ is attached is a double bond;
W₅ is selected from CR_{51′} and N when the [symbol] to which W₅ is attached is a single bond, and W₅ is C when the [symbol] to which W₅ is attached is a double bond;
Z₄ is selected from N and CR₄;
Z₅ is selected from N and CR₅;
Z₆ is selected from N and CR₆;
Z₇ is selected from N and CR₇;
R₁ₐ is selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈;
R₂, R₃, R_{3′}, R₃ₐ, R_{3b}, R_{3c}, R_{3d}, R₄, R₅, R₆, R₇, R₃₀, R₃₁, R₅₀, R₅₁, and R_{51′} are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈alkyl, optionally substituted —C₁-C₈ alkoxy, optionally substituted C₂-C₈ alkenyl, optionally substituted C₂-C₈ alkynyl, and —OSO₂R₈ or X is absent and the nitrogen associated with the absent X is taken together with R₃ₐ, R_{3b}, R_{3c}, or R_{3d} and the carbons to which they are attached, and any atoms therebetween, to form an optionally substituted 3- to 9-membered heterocyclic ring optionally including 1 to 2 additional ring heteromoieties selected from O, S, S(O), SO₂, and NR₉, or R₃ₐ or R_{3c} is taken together with R_{3d} or R_{3b}, and the carbons to which they are attached, to form an optionally substituted 3- to 8-membered cycloalkyl or heterocyclic ring optionally including 1 to 2 additional ring heteromoieties selected from O, S, S(O), SO₂, and NR₉;
R₈ is, independently for each occurrence, selected from hydrogen, deuterium, —N(R₉)₂, optionally substituted C₁-C₈ alkyl, optionally substituted C₂-C₈ alkenyl, optionally substituted C₂-C₈ alkynyl, optionally substituted heteroaryl, and optionally substituted aryl; and
R₉ is, independently for each occurrence, is selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl;
and salts, solvates, hydrates, enantiomers, polymorphs and prodrugs thereof.

Disclosed here are also compounds of Formula III:

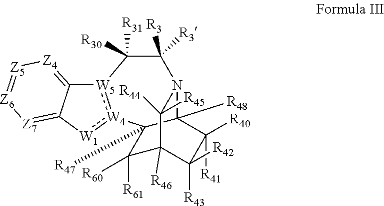

wherein
[symbol] is a single or double bond as needed to satisfy atom valences;
W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond;
W₄ is selected from CR₂ and N when a [symbol] to which W₄ is attached is a single bond, and W₄ is C when a [symbol] to which W₄ is attached is a double bond;
W₅ is selected from CR₅₁ and N when the [symbol] to which W₅ is attached is a single bond, and W₅ is C when the [symbol] to which W₅ is attached is a double bond;
Z₄ is selected from N and CR₄;
Z₅ is selected from N and CR₅;
Z₆ is selected from N and CR₆;
Z₇ is selected from N and CR₇;
R₁ₐ is selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈;
R₂, R₃, R_{3′}, R₄, R₅, R₆, R₇, R₃₀, R₃₁, R₄₄, R₄₅, R₄₆, R₄₈, R₅₀, R₅₁, R_{51′}, R₆₀ and R₆₁ are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl and —OSO₂R₈;
R₄₀, R₄₁, R₄₂, R₄₃, and R₄₇ are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl, optionally substituted heteroaryl, optionally substituted aryl, optionally substituted cycloalkyl and optionally substituted heterocyclyl, or R₄₀ is taken together with R₄₁ or R₄₂, together with the carbons to which they are attached, to form an optionally substituted 3- to 9-membered heterocyclic ring optionally including 1 to 2 additional ring heteromoieties selected from O, S, S(O), SO₂, and NR₉, or R₄₁ is taken together with R₄₃, together with the carbons to which they are attached, to form an optionally substituted 3- to 9-membered heterocyclic ring optionally including 1 to 2 additional ring heteromoieties selected from O, S, S(O), SO₂, and NR₉;
R₈ is, independently for each occurrence, selected from hydrogen, deuterium, —N(R₉)₂, optionally substituted C₁-C₈ alkyl, optionally substituted C₂-C₈ alkenyl, optionally substituted C₂-C₈ alkynyl, optionally substituted heteroaryl, and optionally substituted aryl;
R₉ is, independently for each occurrence, selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl;
and salts, solvates, hydrates, enantiomers, polymorphs and prodrugs thereof.

The disclosure also relates to compositions comprising, consisting of, or consisting essentially of a compound of Formula I, II, III, IV, V, VI, VII, VIIIa, VIIIb, IXa, IXb, Xa, Xb, XIa or XIb and an excipient. The disclosure further relates to pharmaceutical compositions comprising a therapeutically effective amount of a compound of Formula I, II, III, IV, V, VI, VII, VIIIa, VIIIb, IXa, IXb, Xa, Xb, XIa or XIb, wherein the excipient is a pharmaceutically acceptable carrier.

The present disclosure further relates to a method of preventing or treating a psychological disorder comprising the step of administering to a subject in need thereof a therapeutically effective amount of a compound of Formula I, II, III, IV, V, VI, VII, VIIIa, VIIIb, IXa, IXb, Xa, Xb, XIa or XIb, or a pharmaceutical composition containing the same.

Embodiments of the disclosure also relate to a composition comprising, consisting of, or consisting essentially of a first compound selected from compounds of Formula I, II, III, IV, V, VI, VII, VIIIa, VIIIb, IXa, IXb, Xa, Xb, XIa or XIb; and a second active compound. In certain embodiments, the second active compound comprises a serotonergic compound.

Also described herein are methods of preventing or treating inflammation and/or pain comprising administering to a subject in need thereof a therapeutically effective amount of a compound of Formula I, II, III, IV, V, VI, VII, VIIIa, VIIIb, IXa, IXb, Xa, Xb, XIa or XIb, or a composition (e.g., a pharmaceutically-acceptable composition) containing said compound of Formula I, II, III, IV, V, VI, VII, VIIIa, VIIIb, IXa, IXb, Xa, Xb, XIa or XIb.

Unless context indicates differently, reference to a compound of Formula I, II, III, IV, V, VI, VII, VIIIa, VIIIb, IXa, IXb, Xa, Xb, XIa or XIb includes all subgenera of any one of those Formulae (e.g., Formulae II(a), II(b), etc.).

### DETAILED DESCRIPTION

### Compounds

Disclosed herein are compounds of Formula I:

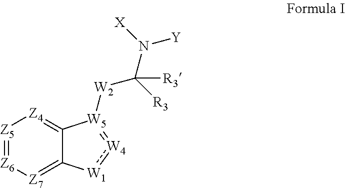

wherein
[symbol] is a single or double bond as needed to satisfy atom valences;
X and Y are each independently selected from hydrogen, deuterium, optionally substituted C₁-C₈ alkyl, optionally substituted C₂-C₈ alkenyl, and optionally substituted C₂-C₈alkynyl, or Y is taken together with X and the nitrogen atom therebetween to form a 3- to 7-membered heterocyclic ring optionally including 1 to 2 additional ring heteromoieties selected from O, S, S(O), SO₂, and NR₉;
W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond;
W₂ is selected from —C(R₃₀)(R₃₁)— and —(C(R₃₀)(R₃₁))₂—;
W₄ is selected from C(R₂)(R_{2′}) and NR₁, when a [symbol] to which W₄ is attached is a single bond, and W₄ is selected from C(R₂) and N when a [symbol] to which W₄ is attached is a double bond;
W₅ is selected from C(R₅₁) and N when the [symbol] to which W₅ is attached is a single bond, and W₅ is C when the [symbol] to which W₅ is attached is a double bond;
Z₄ is selected from N and CR₄;
Z₅ is selected from N and CR₅;
Z₆ is selected from N and CR₆;
Z₇ is selected from N and CR₇;
R₁ₐ and R_{1c} are each independently selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈ or Y is absent and R_{1c} is taken together with the nitrogen to which it is attached and the nitrogen atom to which X is attached to form an optionally substituted 5- to 9-membered heterocyclic ring optionally including 1 to 2 additional ring heteromoieties selected from O, S, S(O), SO₂, and NR₉;
R₂, R_{2′}, R₃, R_{3′}, R₄, R₅, R₆, R₇, R₃₀, R₃₁, R₅₀, R₅₁, and R₅₁ are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl, and —OSO₂R₈ or Y is absent and R₃ is taken together with the carbon to which it is attached and the nitrogen atom to which X is attached to form an optionally substituted 3- to 9-membered heterocyclic ring optionally including 1 to 2 additional ring heteromoieties selected from O, S, S(O), SO₂, and NR₉, or Y is absent and R₂ is taken together with the carbon to which it is attached and the nitrogen atom to which X is attached to form an optionally substituted 5- to 9-membered heterocyclic ring optionally including 1 to 2 additional ring heteromoieties selected from O, S, S(O), SO₂, and NR₉;
R₈ is, independently for each occurrence, selected from hydrogen, deuterium, —N(R₉)₂, optionally substituted C₁-C₈ alkyl, optionally substituted C₂-C₈ alkenyl, optionally substituted C₂-C₈ alkynyl, optionally substituted heteroaryl, and optionally substituted aryl; and
R₉ is, independently for each occurrence, is selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl;
and salts, solvates, hydrates, enantiomers, polymorphs and prodrugs thereof.

Also disclosed herein are compounds of Formula II, III, IV, V, VI, VII, VIIIa, VIIIb, IXa, IXb, Xa, Xb, XIa and XIb, as described herein.

As used herein, the term “alkyl” refers to straight, branched or cyclic saturated hydrocarbon group. As used herein, alkyl has 1 to 20 carbon atoms, 1 to 10 carbon atoms, 1 to 8 carbon atoms, 1 to 6 carbon atoms or 1 to 3 carbon atoms. Examples of alkyl groups include methyl, ethyl, propyl, isopropyl, n-butyl, isobutyl, sec-butyl, tert-butyl, pentyl, isopentyl, hexyl, heptyl, octyl, cyclopropyl, cyclobutyl, cyclopentyl, cyclohexyl, cycloheptyl, cyclooctyl, cyclononyl, and cyclodecyl. When an alkyl residue having a specific number of carbons is named by chemical name or identified by molecular formula, all positional isomers having that number of carbons may be encompassed; thus, for example, “butyl” includes n-butyl, isobutyl, sec-butyl, and tert-butyl; and “propyl” includes n-propyl and isopropyl. In some embodiments, a deuterium atom may be a replacement for a hydrogen atom.

When the alkyl groups described herein are said to be “substituted,” they may be substituted with any substituent or substituents as those found in the exemplary compounds and embodiments disclosed herein, as well as deuterium, alkyl (including, e.g., cycloalkyl), keto (═O), aryl, heteroaryl, hydroxy, alkoxy, alkyl sulfonamido, aryl sulfonamido, and halo. In certain embodiments, the optional substituents themselves may be optionally substituted with any of the substituents set forth in the exemplary compounds and embodiments set forth herein. For example, any of the alkyl groups set forth herein may be substituted with an aryl and/or heteroaryl group, wherein the aryl and heteroaryl groups themselves are optionally substituted with one or more groups selected from hydroxyl, halo, or C₁-C₈ alkoxy. In certain embodiments, at least one hydrogen in a compound of Formula I, II, III, IV, V, VI, VII, VIIIa, VIIIb, IXa, IXb, Xa, Xb, XIa or XIb is replaced with a deuterium.

As used herein, the term “alkenyl” refers to an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. When the alkenyl and alkynyl groups described herein are said to be “substituted,” they may be substituted with any substituent or substituents found in the exemplary compounds and embodiments disclosed herein, as well as deuterium, aryl, heteroaryl, hydroxy, —C(O)R₈, —C(O)OR₈, alkoxy, alkyl sulfonamido, aryl sulfonamido, and halo.

As used herein, the term “cycloalkenyl” refers to a cyclic alkyl group that is partially unsaturated. Unless context dictates otherwise, the term “cycloalkyl” shall include “cycloalkenyl” embodiments.

As used herein, the term “alkoxy” refers to —O-(alkyl), wherein alkyl is as defined above.

As used herein, the term “aryl” refers to an aromatic carbocyclic group of from 6 to 14 carbon atoms having a single ring (e.g., phenyl) or multiple condensed rings (e.g., naphthyl or anthryl). In some embodiments, aryl groups contain 6-14 carbons and in others from 6 to 12 or even 6 to 10 carbon atoms in the ring portions of the groups. Particular aryls include phenyl, biphenyl, naphthyl and the like. The phrase “aryl groups” also includes groups containing fused rings, such as fused aromatic-aliphatic ring systems (e.g., indanyl, tetrahydronaphthyl, and the like). When the aryl groups described herein are said to be “substituted,” they may be substituted with any substituent or substituents found in the exemplary compounds and embodiments disclosed herein, as well as deuterium, aryl, alkyl, heteroaryl, hydroxyl, and halo.

As used herein, the term “heteroaryl” refers to an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic.

As used herein, the term “heterocyclic ring” or “heterocyclyl” or “heterocycloalkyl” refers to a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. When the heterocyclyl groups described herein are said to be “substituted,” they may be substituted with any substituent or substituents found in the exemplary compounds and embodiments disclosed herein, as well as deuterium, alkyl, alkenyl, aryl, heteroaryl, hydroxy, alkoxy, —C(O)R₈, —C(O)OR₈, alkyl sulfonamido, aryl sulfonamido, and halo. Heterocyclyl groups encompass saturated and partially saturated ring systems. Further, the terms “heterocyclyl” and the like are intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom.

As used herein, the term “heteromoieties” refers to any groups containing a heteroatom, for example, amino, O, Se, Se(O), SeO₂, S, S(O), and SO₂.

As used herein, the term “halo” or “halogen” refers to a fluorine, chlorine, bromine or iodine atom.

As used herein, the term “hydroxyl” refers to —OH group.

As used herein, the term “alkyl sulfonamido” refers to a moiety containing —S(═O)₂—N(R)₂, wherein each R group is chosen from an optionally substituted alkyl and H.

As used herein, the term “aryl sulfonamido” refers to a moiety containing —S(═O)₂—N(R)₂, wherein each R group is chosen from an optionally substituted aryl and H.

In some embodiments, the compound of Formula I, II, III, IV, V, VI, VII, VIIIa, VIIIb, IXa, IXb, Xa, Xb, XIa or XIb contains one or more stereocenters. In some circumstances, the compound of Formula I, II, III, IV, V, VI, VII, VIIIa, VIIIb, IXa, IXb, Xa, Xb, XIa or XIb comprises a racemic mixture. In some embodiments, the compound of Formula I, II, III, IV, V, VI, VII, VIIIa, VIIIb, IXa, IXb, Xa, Xb, XIa or XIb comprises the (S) enantiomer. In some embodiments, the compound of Formula I, II, III, IV, V, VI, VII, VIIIa, VIIIb, IXa, IXb, Xa, Xb, XIa or XIb comprises the (R) enantiomer. In some embodiments, the (S) and (R) designations refer to the absolute stereochemistry of a compound having more than one stereocenter. In such cases, the conformation of one of those stereocenters may be referred to in terms of its relative (D) or (L) configuration.

In some embodiments, X and Y are independently selected from hydrogen, deuterium, and optionally substituted C₁-C₈ alkyl, wherein the alkyl group comprises a cycloalkyl moiety (e.g., cyclopropyl, cyclobutyl, etc.).

In some embodiments, R₂, R_{2′}, R₃, R_{3′}, R₄, R₅, R₆, R₇, R₃₀, R₃₁, R₅₀, R₅₁, R_{51′}, R₆₀ and R₆₁ are each independently selected from hydrogen, deuterium, —N(R₉)₂, —C(O)R₃, —OC(O)OR₃, —C(O)OR₃, —OC(O)R₃, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl and optionally substituted C₂-C₈ alkynyl.

In some embodiments, Y is absent and R₃ is taken together with the carbon to which it is attached and the nitrogen atom to which X is attached to form an optionally substituted 3- to 9-membered heterocyclic ring optionally including 1 to 2 additional ring heteromoieties selected from O, S, S(O), SO₂, and NR₉.

In some embodiments, Y is absent and W₄ is C(R₂)(R_{2′}), C(R₂) or NR_{1c} wherein R_{1c} or R₂ is taken together with the nitrogen or carbon to which it is attached, respectively, and the nitrogen atom to which X is attached to form an optionally substituted 5- to 9-membered heterocyclic ring optionally including 1 to 2 additional ring heteromoieties selected from O, S, S(O), SO₂, and NR₉. Exemplary compounds of such embodiments include, but are not limited to, those compounds of Formula II.

In some embodiments, R₄ and R₅ are each independently selected from hydrogen, deuterium, —N(R₉)₂, —SR₉, optionally substituted C₁-C₈ alkyl, optionally substituted C₂-C₈ alkenyl, optionally substituted C₂-C₈ alkynyl, halo, hydroxyl, —C₁-C₈ alkoxy, —C(O)R₈, —OC(O)R₈, —OC(O)OR₈, —OP(O)(OR₉)₂, and —OSO₂R₈. In some embodiments, R₄ and R₅ are each independently selected from hydrogen, deuterium, optionally substituted C₁-C₈ alkyl, optionally substituted C₂-C₈ alkenyl, halo, hydroxyl, —C₁-C₈ alkoxy, —OC(O)R₈, —OC(O)OR₈, —OP(O)(OR₉)₂, and —OSO₂R₈. In certain embodiments, R₅ is hydroxyl. In certain embodiments, R₅ is —OC(O)R₈. In certain embodiments, R₅ is hydrogen. In certain embodiments, at least one of R₄ or R₅ is deuterium.

In certain embodiments, at least one of R₄ and R₅ is selected from C₁-C₅ alkoxy group, or in some embodiments, a C₂-C₄ alkoxy group, wherein the alkoxy group may be a straight chain or branched, for example a straight chain, and may be methoxy or ethoxy. In some embodiments, R₅ is C₁-C₅ alkoxy. In some embodiments, R₄ is selected from hydrogen and fluorine, and R₅ is C₁-C₅ alkoxy. In some embodiments, at least one of R₄ and R₅ is selected from C₁-C₅ alkyl or C₁-C₄ alkyl, for example a straight chain C₁-C₄ alkyl. In some embodiments, R₅ is selected from methyl, ethyl, n-propyl or n-butyl, and for example methyl or ethyl. In some embodiments, at least one of R₄ and R₅ is halo. In some embodiments, R₄ is fluoro. In some embodiments, R₄ is fluoro and R₅ is selected from hydrogen and C₁-C₅ alkoxy. In some embodiments, at least one of R₄ or R₅ is —OC(O)R₈. In some embodiments, R₄ is selected from —OC(O)R₈ and R₅ is hydrogen or fluoro.

In some embodiments, R₈ is selected from optionally substituted C₁-C₈ alkyl, optionally substituted C₂-C₈ alkenyl, optionally substituted C₂-C₈ alkynyl, optionally substituted aryl, and optionally substituted heteroaryl. In some embodiments, R₉ is selected from hydrogen, deuterium, optionally substituted C₁-C₈ alkyl, optionally substituted C₂-C₈ alkenyl, optionally substituted C₂-C₈ alkynyl, optionally substituted heteroaryl and optionally substituted aryl. In some embodiments, R₈ is selected from methyl, ethyl, propyl, and isopropyl. In some embodiments, R₉ is selected from methyl, ethyl, propyl, and isopropyl.

Exemplary halo residues for compounds of Formula I, II, III, IV, V, VI, VII, VIIIa, VIIIb, IXa, IXb, Xa, Xb, XIa or XIb include chloro, bromo, fluoro, and iodo. In certain embodiments, the compounds of Formula I, II, III, IV, V, VI, VII, VIIIa, VIIIb, IXa, IXb, Xa, Xb, XIa or XIb comprise at least one fluoro residue.

In some embodiments, W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond.

In some embodiments, W₁ is selected from NR₁a, O, Se, Se(O), SeO₂, S, S(O), and SO₂. In some embodiments, W₁ is NR₁a. In some embodiments, W₁ is 0. In some embodiments, W₁ is S. In some embodiments, W₁ is Se. In some embodiments, the [symbol] to which W₁ is attached is a double bond and W₁ is N. In some embodiments, the [symbol] to which W₁ is attached is a single bond and W₁ is NR₁a. In some embodiments, the [symbol] to which W₁ is attached is a double bond and W₁ is CR₅₀. In some embodiments, the [symbol] to which W₁ is attached is a single bond and W₁ is C(R₅₀)(R₅₁).

In some embodiments, Z₆ is selected from N and CR₆; and Z₇ is selected from N and CR₇. In some embodiments, Z₆ is N. In some embodiments, Z₇ is N.

In some embodiments, R₁ₐ and R_{1c} are each independently selected from hydrogen, deuterium, optionally substituted C₁-C₈ alkyl, optionally substituted C₂-C₈ alkenyl, optionally substituted C₂-C₈ alkynyl, —C(O)R₃, —C(O)OR₃, —P(O)(OR₉)₂, —C(O)N(R₉)₂, —SOR₃, and —SO₂R₃.

In some embodiments, R₁ₐ is hydrogen. In some embodiments, R₁ₐ is optionally substituted C₁-C₈alkyl. In some embodiments, R₁ₐ is optionally substituted C₂-C₄ alkyl. In some embodiments, R₁ₐ is methyl. In some embodiments, R₁ₐ is ethyl. In some embodiments, R₁ₐ is isopropyl.

In some embodiments, W₂ is selected from —C(R₃₀)(R₃₁)— and —(C(R₃₀)(R₃₁))₂—. In some embodiments, R₃₀ and R₃₁, independently for each occurrence, are selected from hydrogen and deuterium. Thus, in some embodiments, W₂ is selected from —CD₂—, —CDH—, —(CD₂)₂—, —CH₂—, and —(CH₂)₂—. In some embodiments, W₂ is —CH₂—. In some embodiments, W₂ is —(CH₂)₂—. In some embodiments, W₂ is —CD₂—. In some embodiments, W₂ is —(CD₂)₂—. In some embodiments, W₂ is —CDH—. In some embodiments, when W₂ is —CDH—, W₂ represents a stereocenter in the (R) or (S) conformation.

In certain embodiments, W₄ is selected from C(R₂)(R_{2′}) and NR_{1c} when a [symbol] to which W₄ is attached is a single bond, and W₄ is selected from C(R₂) and N when a [symbol] to which W₄ is attached is a double bond. In certain embodiments, W₅ is selected from C(R_{51′}) and N when the [symbol] to which W₅ is attached is a single bond, and W₅ is C when the [symbol] to which W₅ is attached is a double bond. In certain embodiments, the [symbol] between W₄ and W₅ is a double bond, wherein W₅ is C and W₄ is N or C(R₂).

In certain embodiments, the alkyl groups of the compounds of Formula I, II, III, IV, V, VI, VII, VIIIa, VIIIb, IXa, IXb, Xa, Xb, XIa or XIb are selected from C₁-C₈ alkyl, C₂-C₈ alkyl, C₃-C₈alkyl, and C₄-C₈ alkyl, or methyl, ethyl, propyl, isopropyl, n-butyl, isobutyl, sec-butyl, tert-butyl pentyl, isopentyl, hexyl, heptyl, octyl, etc. In certain embodiments, the alkenyl groups of the compounds of Formula I, II, III, IV, V, VI, VII, VIIIa, VIIIb, IXa, IXb, Xa, Xb, XIa or XIb are selected from C₂-C₈ alkenyl, C₃-C₈ alkenyl, and C₄-C₈ alkenyl, or ethenyl, propenyl, butenyl, pentenyl, hexenyl, heptenyl, octenyl, etc.

In certain embodiments, the alkyl and alkenyl groups of the compounds of Formula I, II, III, IV, V, VI, VII, VIIIa, VIIIb, IXa, IXb, Xa, Xb, XIa or XIb may be unsubstituted or substituted with one or more groups selected from deuterium, aryl, heteroaryl, hydroxy, alkoxy, alkyl sulfonamido, aryl sulfonamido, and halo.

In certain embodiments, the cycloalkyl and heterocyclyl groups of the compounds of Formula I, II, III, IV, V, VI, VII, VIIIa, VIIIb, IXa, IXb, Xa, Xb, XIa or XIb may be unsubstituted or substituted with one or more groups selected from deuterium, alkyl, alkenyl, aryl, heteroaryl, hydroxy, alkoxy, alkyl sulfonamido, aryl sulfonamido, and halo.

In certain embodiments, the aryl and heteroaryl groups of the compounds of Formula I, II, III, IV, V, VI, VII, VIIIa, VIIIb, IXa, IXb, Xa, Xb, XIa or XIb may be unsubstituted or substituted with one or more groups selected from deuterium, aryl, alkyl, heteroaryl, hydroxyl, and halo.

In certain embodiments, the alkoxy groups of the compounds of Formula I, II, III, IV, V, VI, VII, VIIIa, VIIIb, IXa, IXb, Xa, Xb, XIa or XIb may be unsubstituted or substituted with one or more groups selected from deuterium, aryl, alkyl, heteroaryl, hydroxyl, and halo.

In some embodiments, X and/or Y may be a straight chain C₁-C₄ alkyl, or a C₂-C₄ alkenyl. In some embodiments, X and Y are each methyl, X and Y are each ethyl, or X is methyl and Y is ethyl. In certain embodiments, X and/or Y are an C₁-C₈ alkyl or C₂-C₈ alkenyl optionally substituted with at least one halo group, such as fluorine. In certain embodiments, at least one of X or Y comprises a group selected from —CF₃, —CHF₂, —CH₂F, —CH₂CF₃, —CH₂CHF₂, and —CH₂CH₂F. In certain embodiments, at least one of X or Y comprises a group selected from —CD₃, —CH₂CD₃, —CD₂CH₃, and —CD2CD₃.

In some embodiments, X is unsubstituted C₁-C₈ alkyl. In some embodiments, X is methyl. In some embodiments, X is ethyl. In some embodiments, X is n-propyl. In some embodiments, X is isopropyl. In some embodiments, X is cyclopropyl. In some embodiments, Y is hydrogen. In some embodiments, Y is unsubstituted C₁-C₈ alkyl. In some embodiments, Y is methyl. In some embodiments, Y is ethyl. In some embodiments, Y is n-propyl. In some embodiments, Y is isopropyl. In some embodiments, Y is cyclopropyl.

In some embodiments, R₂, R₃, R_{3′}, R₆ and R₇ are each independently selected from hydrogen, deuterium, halo, or C₁-C₄ alkyl, for example a straight chain C₁-C₄ alkyl. In some embodiments, R₂, R₃, R_{3′}, R₆ and R₇ are each independently selected from hydrogen, deuterium, halo, methyl, ethyl, n-propyl, isopropyl, n-butyl and isobutyl. In other embodiments, R₂, R₃, R_{3′}, R₆ and R₇ are each independently selected from hydrogen, deuterium, methyl, and ethyl.

In some embodiments, R₂ is hydrogen. In some embodiments, R₃ and R_{3′}, are each independently selected from hydrogen, methyl, and ethyl. In some embodiments, R₃ is hydrogen. In some embodiments, R₃ is methyl and R₃ is hydrogen. In some embodiments, R₃ and R_{3′}, are both hydrogen. In some embodiments, R₃ and R_{3′}, are both deuterium. In some embodiments, R₃ is hydrogen and R₃ is deuterium.

In some embodiments, when R₃ and R₃, are not the same, it represents a stereocenter wherein the compound of Formula I, II, III, IV, V, VI, VII, VIIIa, VIIIb, IXa, IXb, Xa, Xb, XIa or XIb comprises a racemic mixture. In some embodiments, when R₃ and R₃, are not the same, it represents a stereocenter wherein the compound of Formula I, II, III, IV, V, VI, VII, VIIIa, VIIIb, IXa, IXb, Xa, Xb, XIa or XIb comprises the (S) enantiomer. In some embodiments, when R₃ and R₃, are not the same, it represents a stereocenter wherein the compound of Formula I, II, III, IV, V, VI, VII, VIIIa, VIIIb, IXa, IXb, Xa, Xb, XIa or XIb comprises the (R) enantiomer. In some embodiments, a racemic mixture can be resolved to provide a pure enantiomer or a mixture enhanced with either the (R) or (S) enantiomer. In some embodiments, the compounds described herein exhibit an enantiomeric excess (EE) of greater than about 50%, such as at least about 60%, at least about 75%, at least about 80%, at least about 90%, at least about 95%, at least about 96%, at least about 97%, at least about 98%, or at least about 99%. Thus, in some embodiments the compounds described herein exhibit an enantiomeric purity of about 98%, about 99% or even about 100%.

In some embodiments, R₆ and R₇ are each independently selected hydrogen, halo, methyl, ethyl, n-propyl, isopropyl, n-butyl and isobutyl. In some embodiments, R₆ is selected from hydrogen and halo. In some embodiments, R₆ is selected from hydrogen and fluorine. In some embodiments, R₆ is fluorine. In some embodiments, R₆ is selected from CrC alkoxy and halo. In some embodiments, R₆ is selected from methoxy, chloro and fluoro. In some embodiments, R₆ is fluorine. In some embodiments, R₆ is methoxy. In some embodiments, R₇ is selected from hydrogen and optionally substituted C₁-C₄ alkyl. In some embodiments, R₇ is selected from hydrogen, methyl and ethyl. In some embodiments, R₇ is optionally substituted C₁-C₄ alkyl. In some embodiments, when R₆ is fluoro, then R₇ is selected from hydrogen and optionally substituted C₁-C₄ alkyl.

In some embodiments, R₄ is hydrogen and R₅ is selected from optionally substituted C₁-C₈ alkyl, optionally substituted C₂-C₈ alkenyl, halo, hydroxyl, —C₁-C₈ alkoxy, —OC(O)R₈, —OC(O)OR₈, —OP(O)(OR₉)₂, and —OSO₂R₈. In some embodiments, R₅ is selected from unsubstituted C₁-C₈ alkyl, hydroxyl, —C₁-C₈ alkoxy, —OC(O)R₈, —OC(O)OR₈, —OP(O)(OR₉)₂, and —OSO₂R₈. In some embodiments, R₅ is selected from unsubstituted C₂-C₈ alkyl, hydroxyl, —C₁-C₈ alkoxy, —OC(O)R₈, —OC(O)OR₈, —OP(O)(OR₉)₂, and —OSO₂R₈. In some embodiments, R₅ is hydroxy. In some embodiments, R₅ is —OC(O)R₈. In some embodiments, R₈ is unsubstituted C₁-C₄ alkyl. In some embodiments, R₈ is methyl.

In some embodiments, R₅ is hydrogen and R₄ is selected from optionally substituted C₁-C₈ alkyl, optionally substituted C₂-C₈ alkenyl, halo, hydroxyl, —C₁-C₈ alkoxy, —OC(O)R₈, —OC(O)OR₈, —OP(O)(OR₉)₂, and —OSO₂R₈. In some embodiments, R₄ is selected from unsubstituted C₁-C₈ alkyl, hydroxyl, —C₁-C₈ alkoxy, —OC(O)R₈, —OC(O)OR₈, —OP(O)(OR₉)₂, and —OSO₂R₈. In some embodiments, R₄ is selected from unsubstituted C₁-C₈ alkyl, —C₂-C₈ alkoxy, —OC(O)R₈, —OC(O)OR₈, —OP(O)(OR₉)₂, and —OSO₂R₈. In some embodiments, R₄ is —OC(O)R₈. In some embodiments, R₈ is unsubstituted C₁-C₄ alkyl. In some embodiments, R₈ is methyl.

In certain embodiments, one or more hydrogen atoms on compounds of Formula I, II, III, IV, V, VI, VII, VIIIa, VIIIb, IXa, IXb, Xa, Xb, XIa or XIb may be replaced with one or more deuterium atoms. For example, in certain embodiments, R₆ may comprise a deuterium atom as a replacement for a hydrogen, or when R₇ is a —CH₃, each hydrogen atom may be replaced to form a —CD₃ residue. Similarly, another non-limiting example includes when X and/or Y is a —CH₃, each hydrogen atom may be replaced to form a —CD₃ residue.

In some embodiments, the compounds disclosed herein are of Formula II:

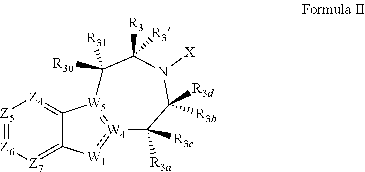

X is selected from hydrogen, deuterium, optionally substituted C₁-C₈ alkyl, optionally substituted C₂-C₈ alkenyl, and optionally substituted C₂-C₈ alkynyl, or X is absent and R_{3c} together with the carbon to which it is attached and the nitrogen associated with the residue N—X, and the atoms therebetween, are taken to form an optionally substituted 3- to 9-membered heterocyclic ring optionally including 1 to 2 additional ring heteromoieties selected from O, S, S(O), SO₂, and NR₉;
[symbol] is a single or double bond as needed to satisfy atom valences;
W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond;
W₄ is selected from CR₂ and N when a [symbol] to which W₄ is attached is a single bond, and W₄ is C when a [symbol] to which W₄ is attached is a double bond;
W₅ is selected from CR_{51′} and N when the [symbol] to which W₅ is attached is a single bond, and W₅ is C when the [symbol] to which W₅ is attached is a double bond;
Z₄ is selected from N and CR₄;
Z₅ is selected from N and CR₅;
Z₆ is selected from N and CR₆;
Z₇ is selected from N and CR₇;
R₁ₐ is selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈;
R₂, R₃, R_{3′}, R₃ₐ, R_{3b}, R_{3c}, R_{3d}, R₄, R₅, R₆, R₇, R₃₀, R₃₁, R₅₀, R₅₁, and R_{51′} are each independently selected from hydrogen, deuterium, —N(R₉)₂, —C(O)R₈, —OC(O)OR₈, —C(O)OR₈, —OC(O)R₃, —SR₉, —P(O)(OR₉)₂, —C(O)N(R₉)₂, halo, hydroxyl, optionally substituted C₁-C₈alkyl, optionally substituted —C₁-C₈ alkoxy, optionally substituted C₂-C₈ alkenyl, optionally substituted C₂-C₈ alkynyl, and —OSO₂R₈, or X together with the nitrogen to which is attached is taken with R₃ₐ, R_{3b}, R₃, or R_{3d} and the carbons to which they are attached, and any atoms therebetween, to form an optionally substituted 3- to 9-membered heterocyclic ring optionally including 1 to 2 additional ring heteromoieties selected from O, S, S(O), SO₂, and NR₉, or R₃ₐ or R_{3c} is taken together with R_{3d} or R_{3b}, and the carbons to which they are attached, to form an optionally substituted 3- to 8-membered cycloalkyl or heterocyclic ring optionally including 1 to 2 additional ring heteromoieties selected from O, S, S(O), SO₂, and NR₉;
R₈ is, independently for each occurrence, selected from hydrogen, deuterium, —N(R₉)₂, optionally substituted C₁-C₈ alkyl, optionally substituted C₂-C₈ alkenyl, optionally substituted C₂-C₈ alkynyl, optionally substituted heteroaryl, and optionally substituted aryl; and
R₉ is, independently for each occurrence, is selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl;
and salts, solvates, hydrates, enantiomers, polymorphs and prodrugs thereof.

In some embodiments, X is absent and R_{3c} together with the carbon to which it is attached and the nitrogen associated with the absent X, and the atoms therebetween, are taken to form an optionally substituted 3- to 9-membered heterocyclic ring optionally including 1 to 2 additional ring heteromoieties selected from O, S, S(O), SO₂, and NR₉.

Exemplary compounds of the present disclosure include:

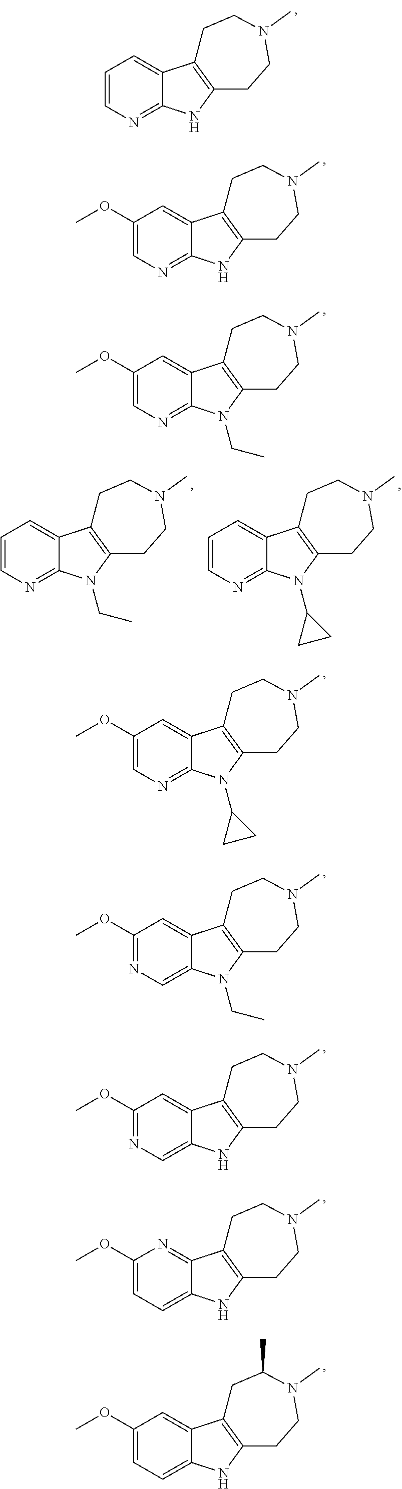

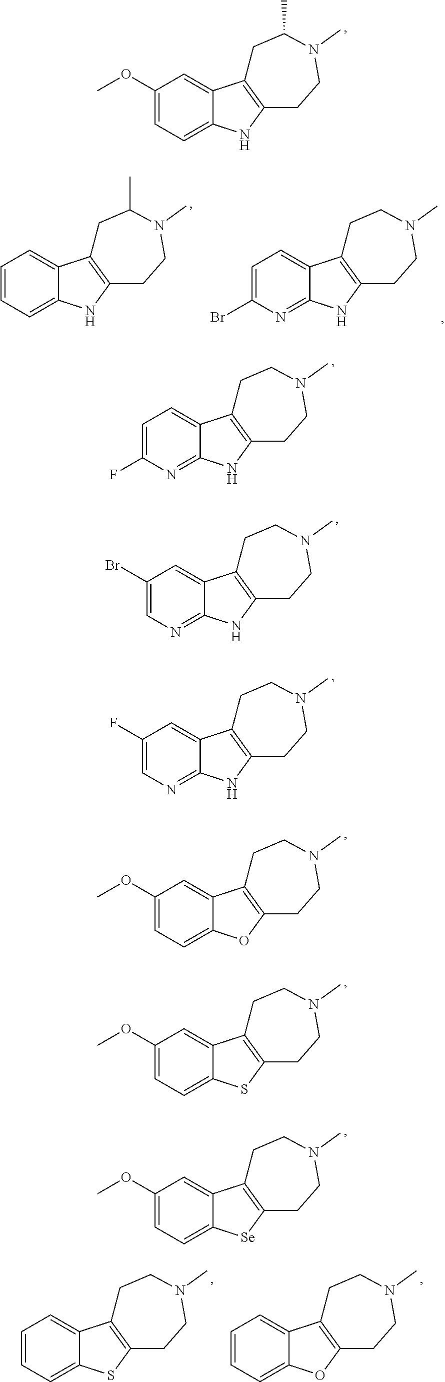

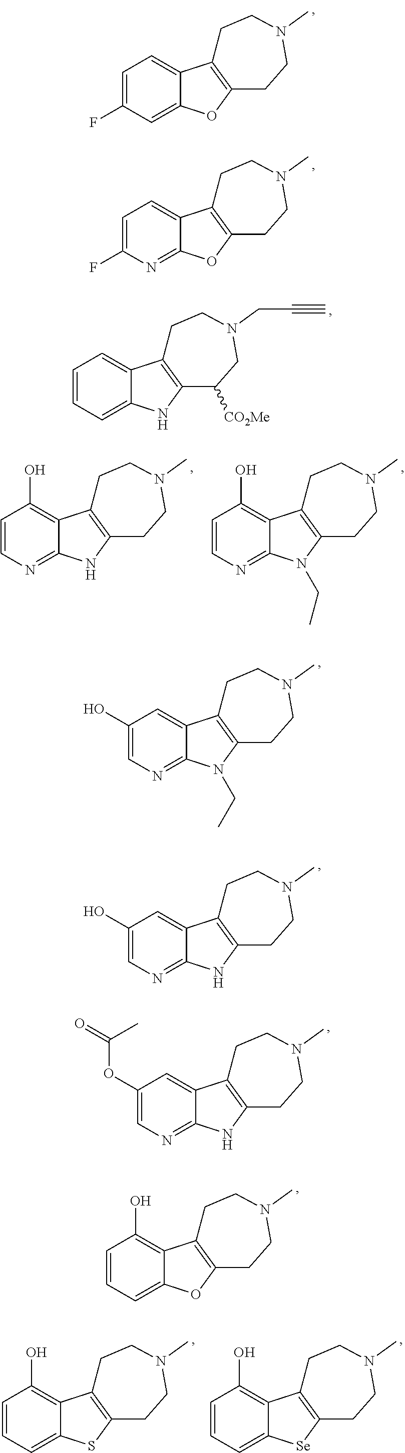

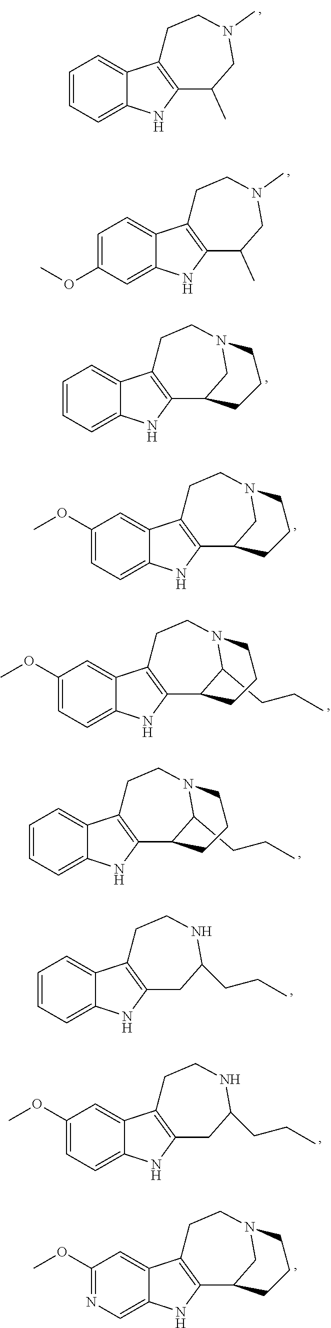

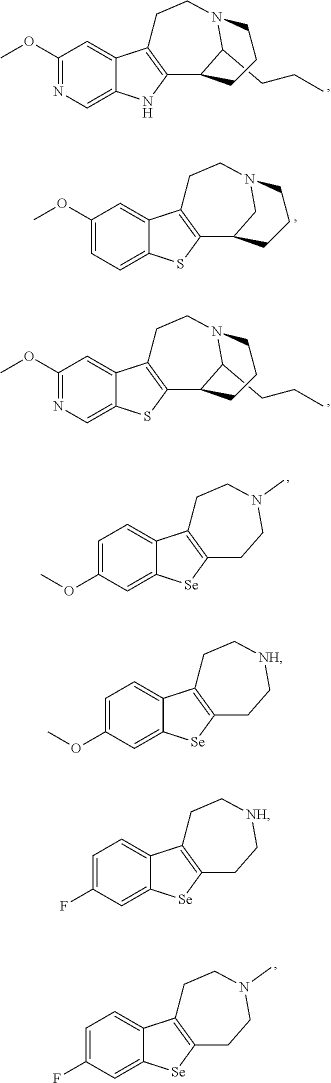

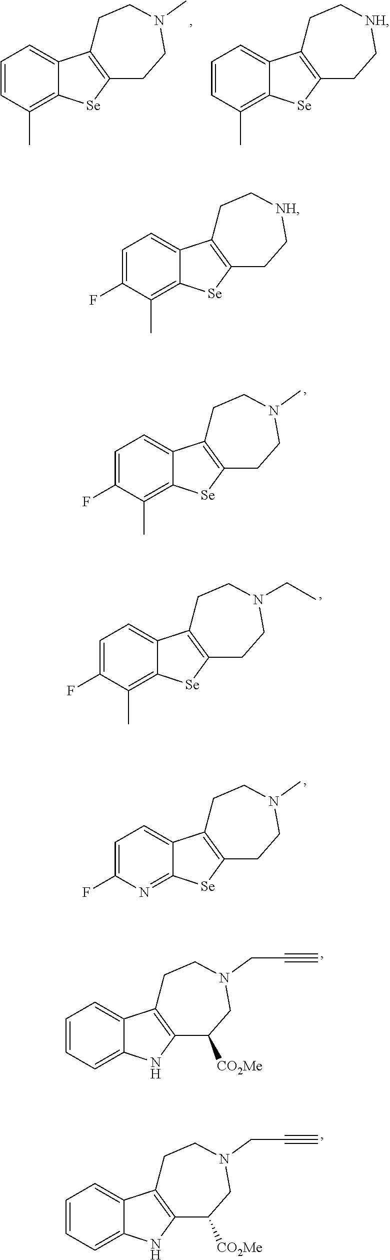

and salts, solvates, hydrates, enantiomers, polymorphs and prodrugs of any of the foregoing compounds.

Other exemplary compounds include compounds of Formula III:

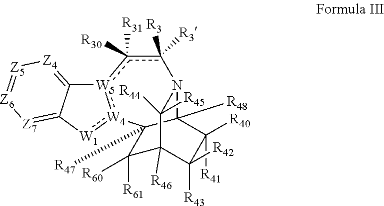

wherein
[symbol] is a single or double bond as needed to satisfy atom valences;
W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond;
W₄ is selected from CR₂ and N when a [symbol] to which W₄ is attached is a single bond, and W₄ is C when a [symbol] to which W₄ is attached is a double bond;
W₅ is selected from CR_{51′} and N when both of the [symbol] groups to which W₅ is attached are single bonds, and W₅ is C when one of the [symbol] groups to which W₅ is attached is a double bond;
Z₄ is selected from N and CR₄;
Z₅ is selected from N and CR₅;
Z₆ is selected from N and CR₆;
Z₇ is selected from N and CR₇;
R₁ₐ is selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈;
R₂, R₃, R_{3′}, R₄, R₅, R₆, R₇, R₃₀, R₃₁, R₄₄, R₄₅, R₄₆, R₄₈, R₅₀, R₅₁, R_{51′}, R₆₀ and R₆₁ are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl and —OSO₂R₈;
R₄₀, R₄₁, R₄₂, R₄₃, and R₄₇ are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, —OSO₂R₈, halo, hydroxyl, optionally substituted C₁-C₈ alkyl, optionally substituted —C₁-C₈ alkoxy, optionally substituted C₂-C₈ alkenyl and optionally substituted C₂-C₈ alkynyl, or R₄₀ is taken together with R₄₁ or R₄₂, together with the carbons to which they are attached, to form an optionally substituted 3- to 9-membered heterocyclic ring optionally including 1 to 2 additional ring heteromoieties selected from O, S, S(O), SO₂, and NR₉, or R₄₁ is taken together with R₄₃, together with the carbons to which they are attached, to form an optionally substituted 3- to 9-membered heterocyclic ring optionally including 1 to 2 additional ring heteromoieties selected from O, S, S(O), SO₂, and NR₉;
R₈ is, independently for each occurrence, selected from hydrogen, deuterium, —N(R₉)₂, optionally substituted C₁-C₈ alkyl, optionally substituted C₂-C₈ alkenyl, optionally substituted C₂-C₈ alkynyl, optionally substituted heteroaryl, and optionally substituted aryl;
R₉ is, independently for each occurrence, selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl,
wherein when the [symbol] adjacent to R₃ is a double bond, R₃₁ and R_{3′} are absent and the [symbol] adjacent to R₃₀ and W₅ is a single bond, and when the [symbol] adjacent to R₃₀ and W₅ is a double bond, then W₅ is C and the [symbol] between W₅ and W₄ is a single bond;
and salts, solvates, hydrates, enantiomers, polymorphs and prodrugs thereof.

In certain embodiments, R₄₀, R₄₁, R₄₂, R₄₃, and R₄₇ are each independently selected from hydrogen, deuterium, hydroxyl, —N(R₉)₂, —SR₉, hydroxyl, halo, optionally substituted C₁-C₈alkyl, —C₁-C₈ alkoxy, optionally substituted C₂-C₈ alkenyl, —C(O)R₈, —OC(O)R₈, —OC(O)OR₈, optionally substituted aryl, optionally substituted heteroaryl, optionally substituted cycloalkyl and optionally substituted heterocyclyl, wherein the optional substituents are selected from —SR₉, hydroxyl, halo, and —C(O)R₈.

Exemplary compounds of Formula III include, but are not limited to:

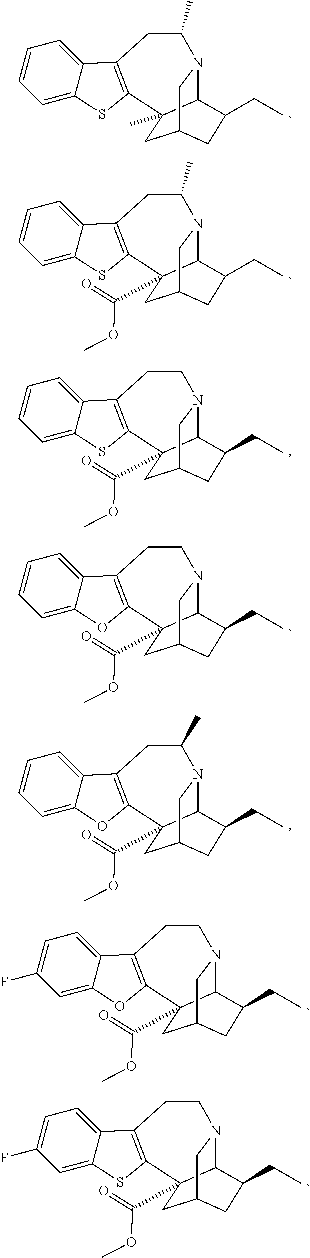

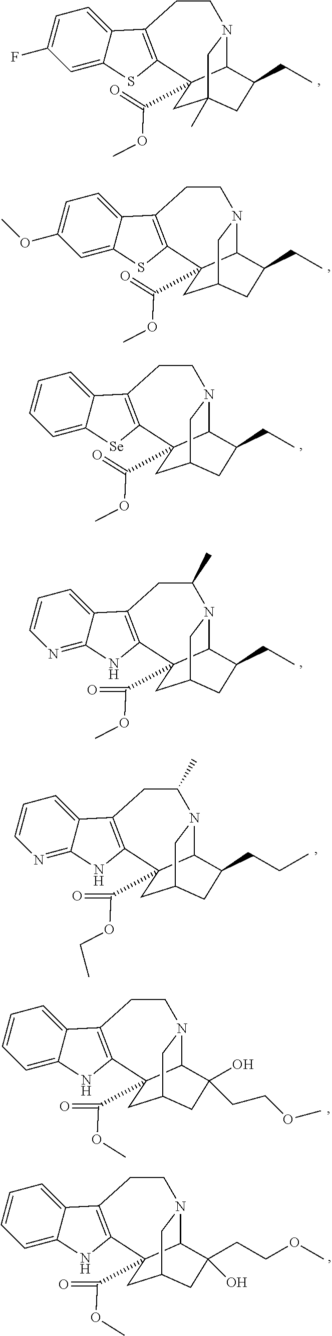

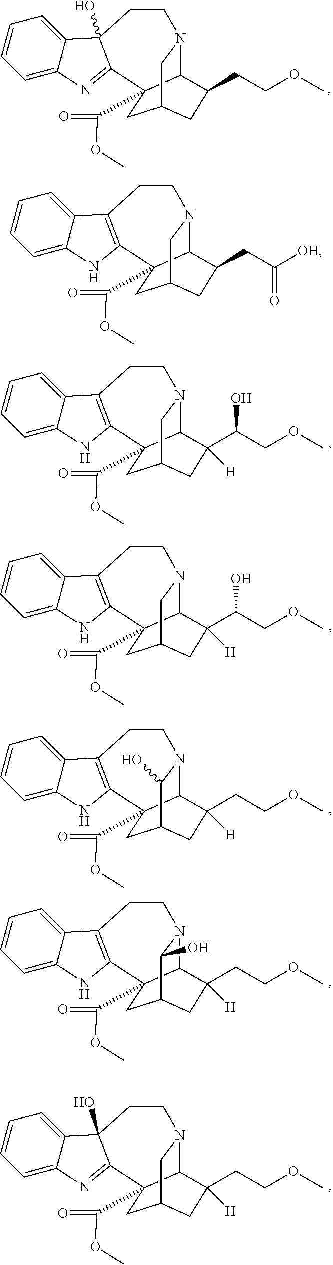

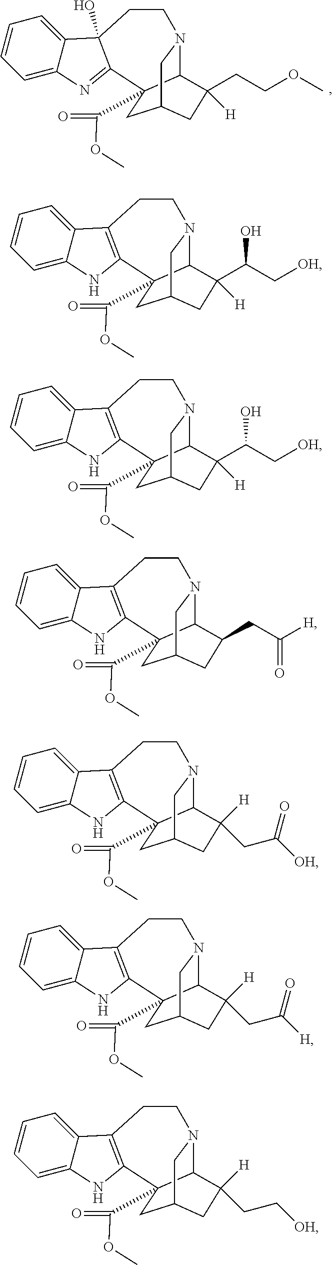

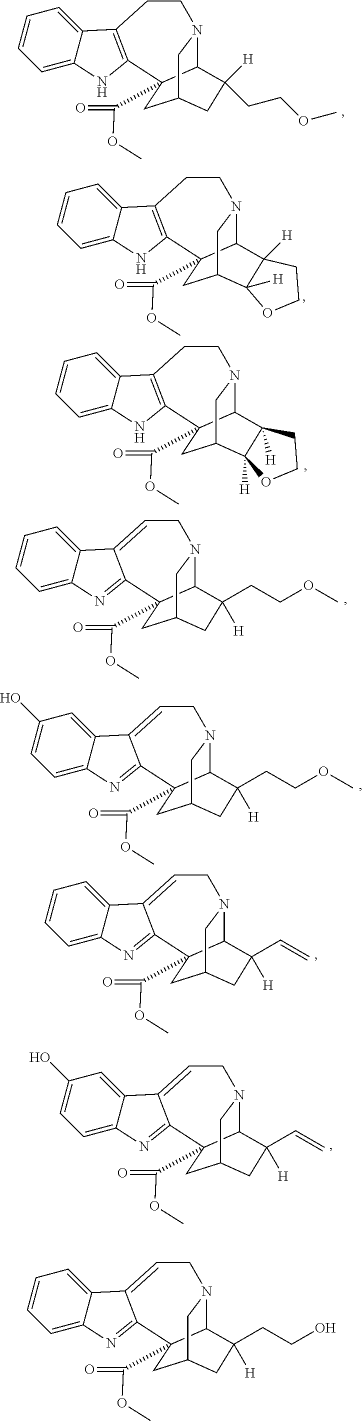

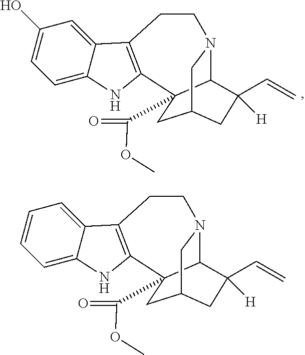

and salts, solvates, hydrates, enantiomers, polymorphs and prodrugs of any of the foregoing compounds.

Other exemplary compounds include compounds of Formula IV:

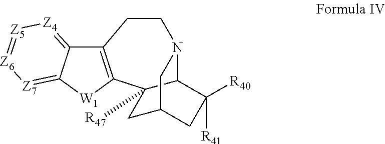

wherein
W₁ is selected from NR₁ₐ, O, S, S(O), SO₂, Se, Se(O), and SeO₂;
Z₄ is selected from N and CR₄;
Z₅ is selected from N and CR₅;
Z₆ is selected from N and CR₆;
Z₇ is selected from N and CR₇;
R₁ₐ is selected from hydrogen, deuterium, optionally substituted C₁-C₈ alkyl, optionally substituted C₂-C₈ alkenyl, —C(O)R₈, —C(O)OR₈, —P(O)(OR₉)₂, —C(O)N(R₉)₂, —SOR₈, and —SO₂R₈;
R₄₀, R₄₁, and R₄₇ are each independently selected from hydrogen, deuterium, —N(R₉)₂, —SR₉, hydroxyl, halo, optionally substituted C₁-C₈ alkyl, —C₁-C₈ alkoxy, optionally substituted C₂-C₈ alkenyl, —C(O)R₈, —OC(O)R₈, —C(O)OR₈, —OC(O)OR₈, optionally substituted aryl, optionally substituted heteroaryl, optionally substituted cycloalkyl and optionally substituted heterocyclyl;
R₄, R₅, R₆ and R₇ are each independently selected from hydrogen, deuterium, optionally substituted C₁-C₈ alkyl, optionally substituted C₂-C₈ alkenyl, halo, hydroxyl, —N(R₉)₂, —SR₉, —C₁-C₈alkoxy, —OC(O)R₃, —OC(O)OR₃, —OP(O)(OR₉)₂, and —OSO₂R₈;
R₈ is selected from hydrogen, deuterium, —N(R₉)₂, optionally substituted C₁-C₈alkyl, optionally substituted C₂-C₈ alkenyl, and optionally substituted aryl;
R₉ is independently selected from hydrogen, deuterium, optionally substituted C₁-C₈alkyl, optionally substituted C₂-C₈ alkenyl, and optionally substituted aryl;
and salts, solvates, hydrates, enantiomers, polymorphs and prodrugs thereof.

Further exemplary embodiments of the disclosure include, but are not limited to:

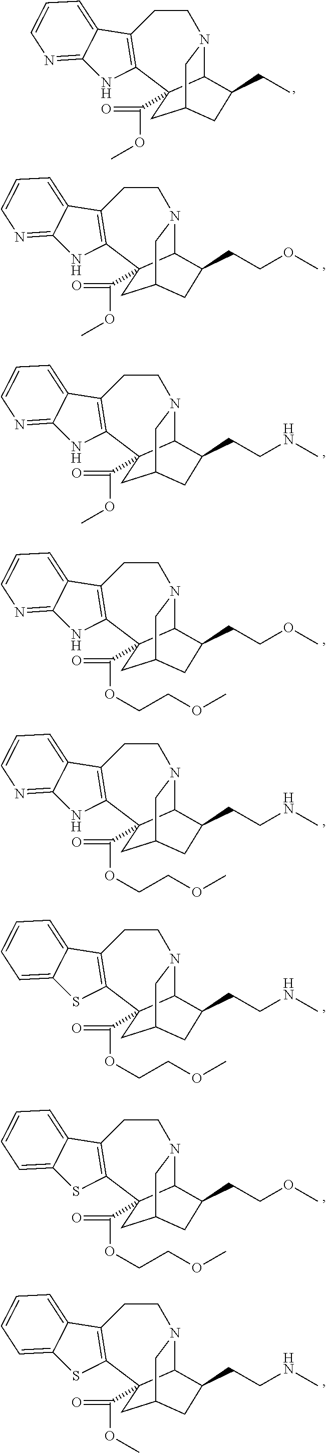

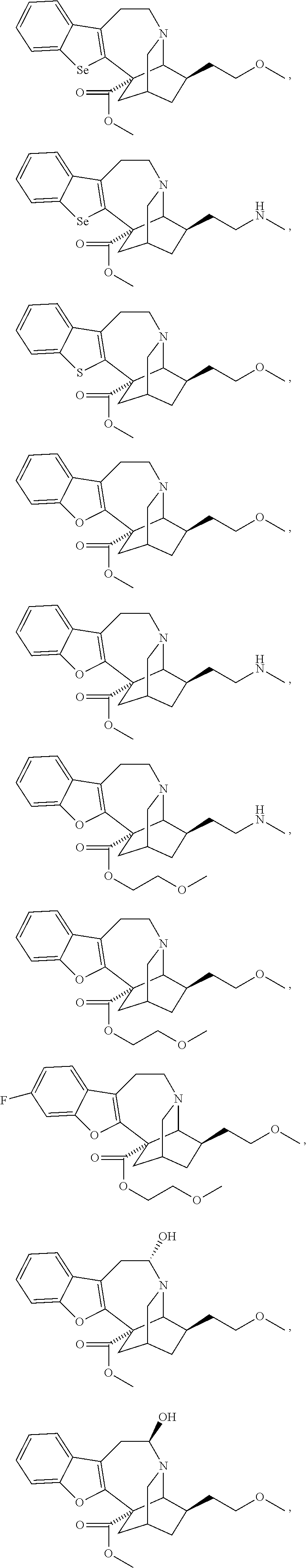

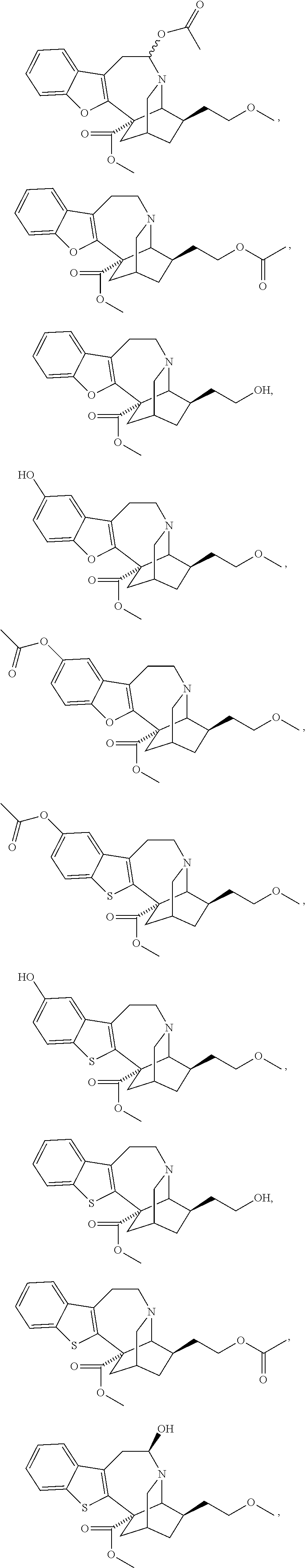

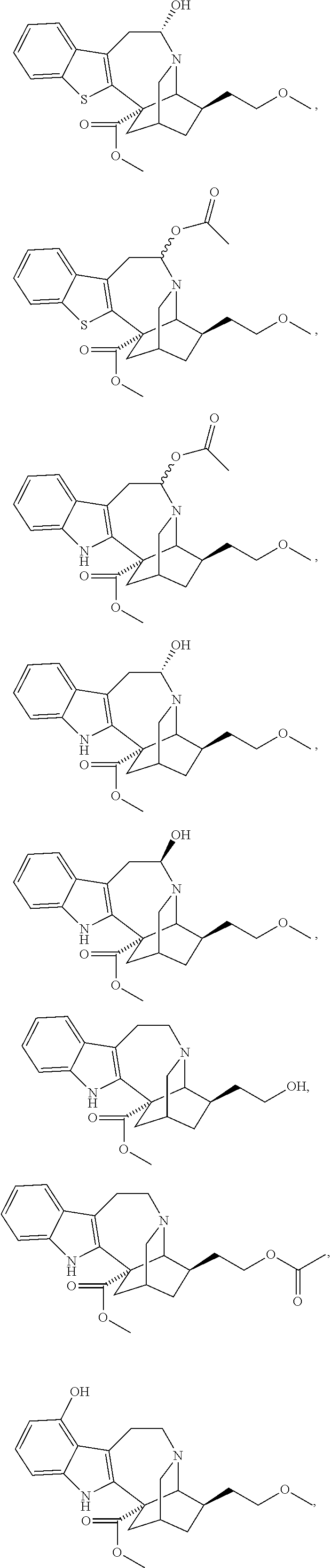

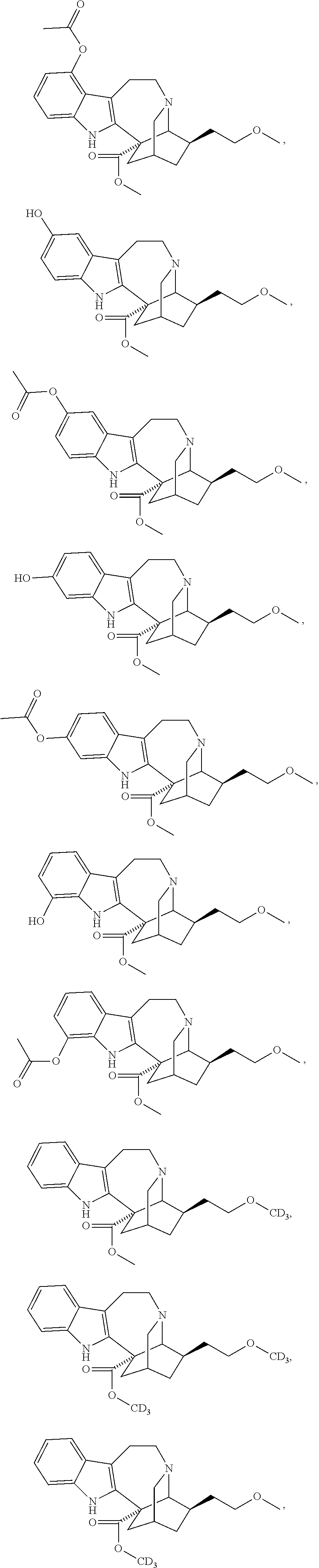

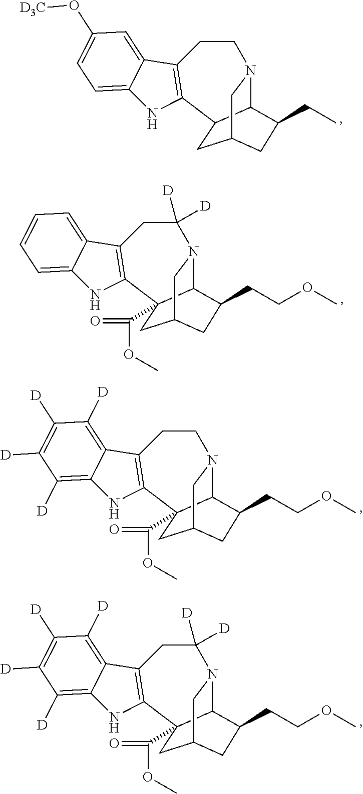

and salts, solvates, hydrates, enantiomers, polymorphs and prodrugs of any of the foregoing compounds.

In some embodiments, the compounds of Formula I, II, III, IV, V, VI, VII, VIIIa, VIIIb, IXa, IXb, Xa, Xb, XIa or XIb comprise salts. In some embodiments, the compounds of Formula I, II, III, IV, V, VI, VII, VIIIa, VIIIb, IXa, IXb, Xa, Xb, XIa or XIb comprise pharmaceutically acceptable salts. Exemplary salts include, but are not limited to, HCl, HI, HBr, HF, ascorbate, hydrofumarate, fumarate, oxalate, maleate, and the like. In certain embodiments, the compound of Formula I, II, III, IV, V, VI, VII, VIIIa, VIIIb, IXa, IXb, Xa, Xb, XIa or XIb is in its free-base form. In some embodiments, the compound of Formula I, II, III, IV, V, VI, VII, VIIIa, VIIIb, IXa, IXb, Xa, Xb, XIa or XIb comprises a salt, such as a [1:1] salt (e.g., HCl, hydrofumarate) or a [2:1] salt (e.g., oxalate, fumarate). For the [1:1] salts, one ammonium cation of one compound of Formula I, II, III, IV, V, VI, VII, VIIIa, VIIIb, IXa, IXb, Xa, Xb, XIa or XIb is balanced by a single anion (Cl—, I—, etc.). For the [2:1] salts, two ammonium cations of two molecules of Formula I, II, III, IV, V, VI, VII, VIIIa, VIIIb, IXa, IXb, Xa, Xb, XIa or XIb are balanced by a dianionic species, such as a dianion derived from di-acids such as oxalic acid and fumaric acid. Other exemplary salts include zwitterionic forms of compounds of Formula I, II, III, IV, V, VI, VII, VIIIa, VIIIb, IXa, IXb, Xa, Xb, XIa or XIb, such as when R₄ is —P(O)(OH)₂, wherein deprotonation of an —OH on R₄ may result in intramolecular coordination of the resulting —O⁻ with the quaternary ethylammonium residue (e.g., —(CH₂)₂N⁺H(CH₃)₂).

In certain embodiments, the compounds described herein are enantiomers of the compounds of Formula I, II, III and IV. For example, in certain embodiments are described compounds of Formula V:

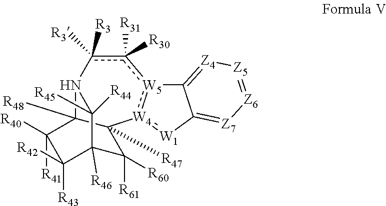

wherein the variables are previously defined herein in Formula III.

Exemplary compounds of Formula V include, but are not limited to:

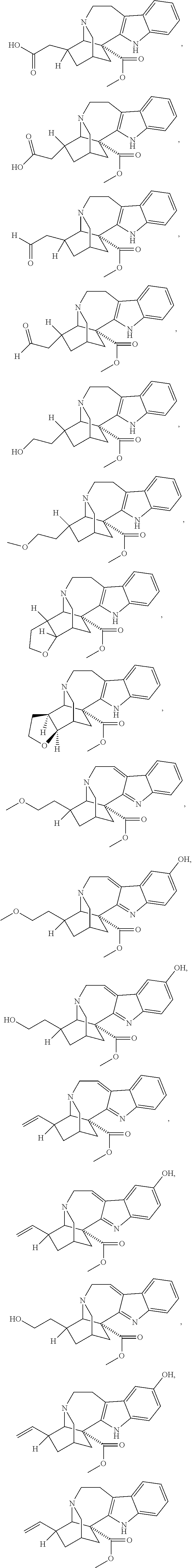

and salts, solvates, hydrates, enantiomers, polymorphs and prodrugs of any of the foregoing compounds.

In some embodiments, the compounds described herein comprise a racemic mixture of compounds, such as a mixture of a compound selected from Formula III and its enantiomer represented by Formula V.

In some embodiments are described compounds of Formula VI:

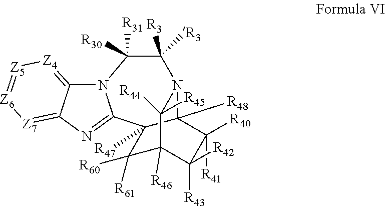

wherein the variables are previously defined herein.

In some embodiments are described compounds of Formula VII:

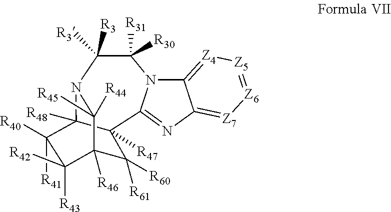

wherein the variables are previously defined herein.

Exemplary compounds of Formula VI include, but are not limited to:

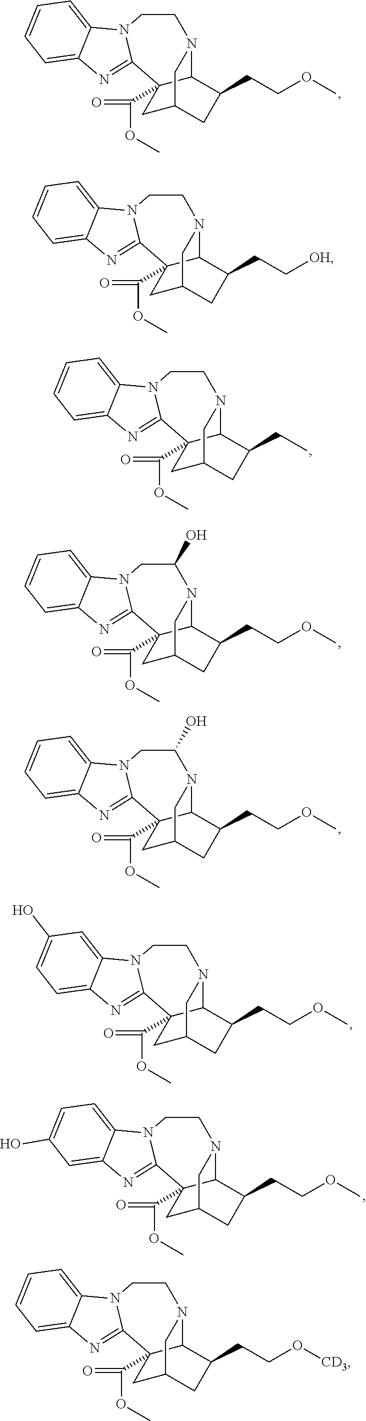

and salts, solvates, hydrates, enantiomers, polymorphs and prodrugs of any of the foregoing compounds.

In some embodiments are described compounds of Formula VIIIa and Formula VIIIb:

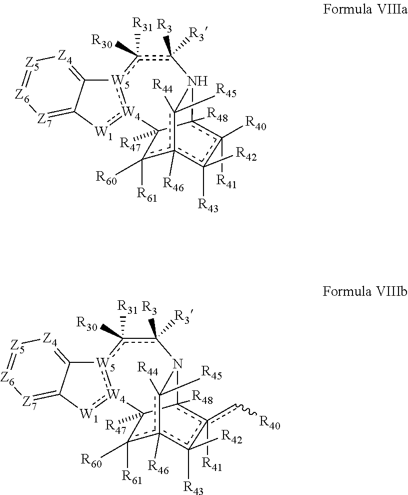

wherein the variables are as previously described herein,
and further wherein when the [symbol] adjacent to R₃ is a double bond, R₃₁ and R_{3′}, are absent and the [symbol] adjacent to R₃₀ and W₅ is a single bond; when the [symbol] adjacent to R₄₄ is a double bond, R₄₅ and R₄₆ are absent; when the [symbol] adjacent to R₆₀ is a double bond, R₆₁ and R₄₆ are absent; when the [symbol] adjacent to R₄₂ and R₄₆ is a double bond, R₄₆ and R₄₃ are absent; when the [symbol] adjacent to R₄₁ and R₄₃ is a double bond, R₄₁ and R₄₃ are absent; when the [symbol] adjacent to R₄₂ and R₄₁ is a double bond, R₄₁ and R₄₃ are absent; when the [symbol] adjacent to R₄₀ in Formula VIIIb is a double bond, R₄₁ is absent; and when the [symbol] adjacent to R₃₀ and W₅ is a double bond, then W₅ is C and the [symbol] between W₅ and W₄ is a single bond.

In certain embodiments of compounds of Formula VIIIa, R₄₀ is not ethyl, vinyl, 2-bromoethyl, 2-hydroxyethyl, 2-fluoroethyl, 2-cyanoethyl, or 2-N₃-ethyl. In certain embodiments for compounds of Formula VIIIa, R₄₀ is not ethyl, vinyl, 2-bromoethyl, 2-hydroxyethyl, 2-fluoroethyl, 2-cyanoethyl, or 2-N₃-ethyl when R₄₇ is —C(═O)OMe.

In certain embodiments for compounds of Formula VIIIb, R₄₀ is not methyl. In certain embodiments for compounds of Formula VIIIb, R₄₀ is not methyl when R₄₇ is —C(═O)OMe. In certain embodiments for compounds of Formula VIIIa or VIIlb, R₄₀ is selected from optionally substituted C₂-C₈ alkyl, optionally substituted C₂-C₈ alkenyl, or optionally substituted C₂-C₈ alkynyl.

Exemplary compounds of Formula VIIIa and VIIIb include, but are not limited to:

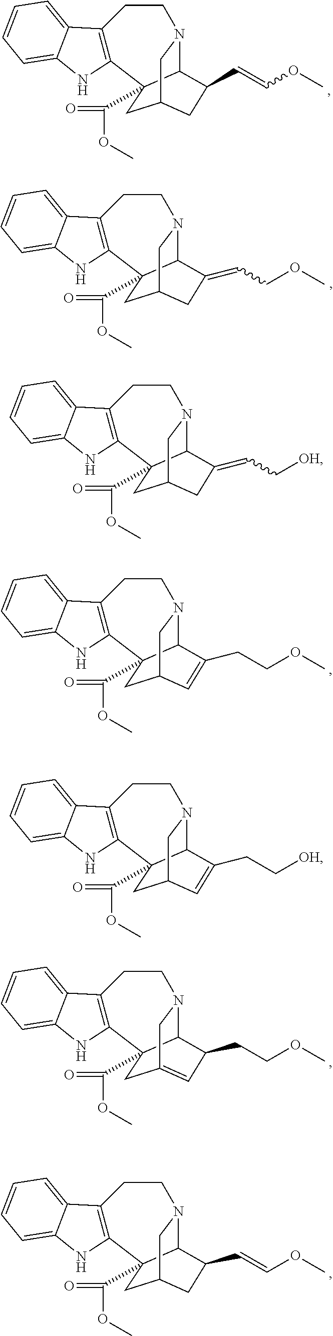

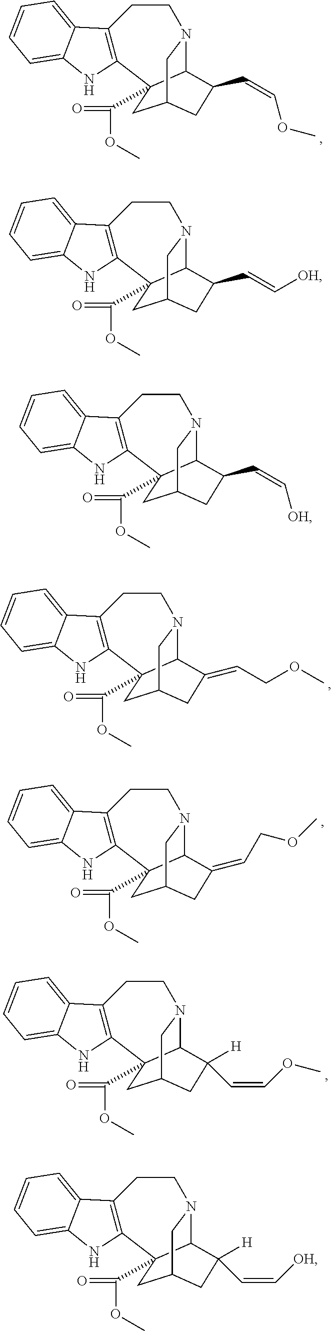

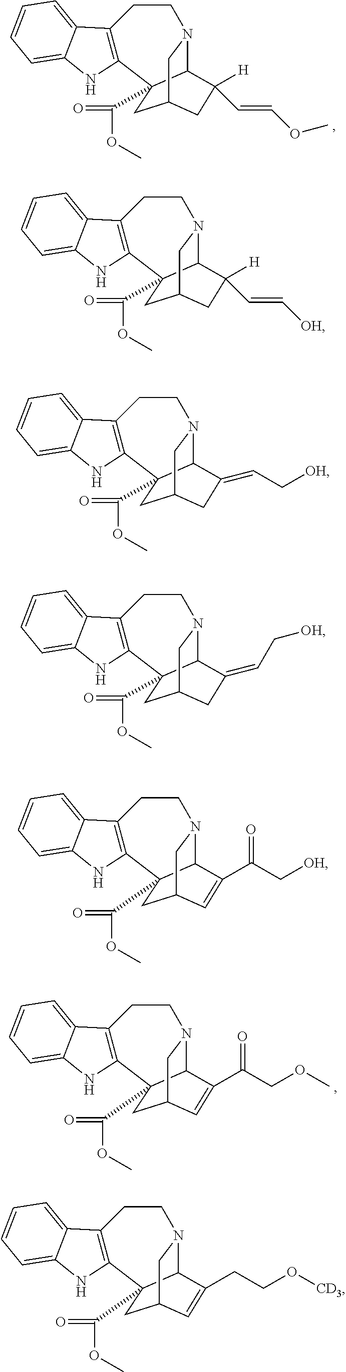

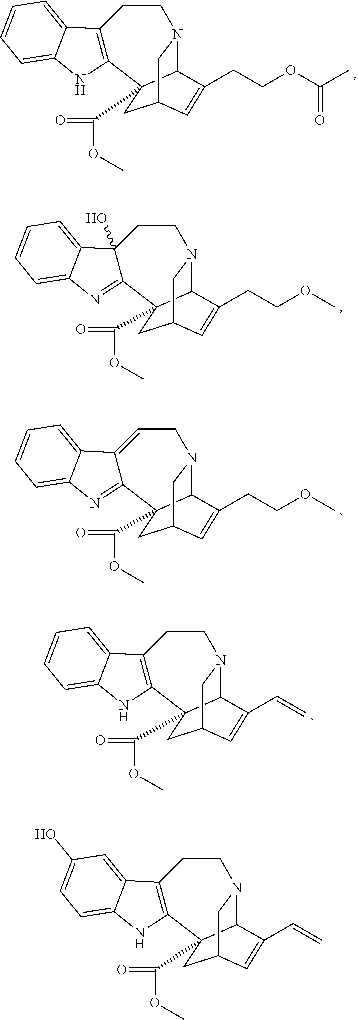 [SMILES: COC(=O)[C@@]12CC3C=C(CCOC(C)=O)C1N(CCc1c2[nH]c2ccccc12)C3 ; COCCC1=CC2CN3CCC4(O)C(=Nc5ccccc54)[C@](C(=O)OC)(C2)C13 ; COCCC1=CC2CN3CC=C4C(=Nc5ccccc54)[C@](C(=O)OC)(C2)C13 ; C=CC1=CC2CN3CCc4c([nH]c5ccccc45)[C@](C(=O)OC)(C2)C13 ; C=CC1=CC2CN3CCc4c([nH]c5ccc(O)cc45)[C@](C(=O)OC)(C2)C13]

and salts, solvates, hydrates, enantiomers, polymorphs and prodrugs of any of the foregoing compounds.

In some embodiments are described compounds of Formula IXa and Formula IXb:

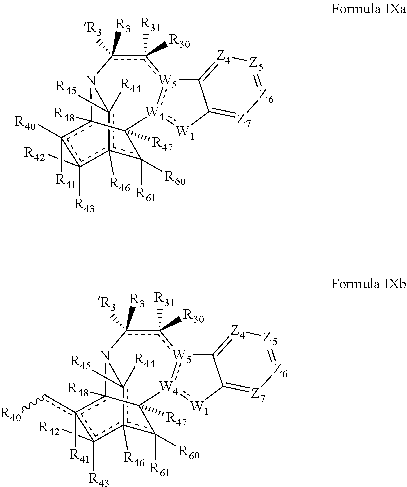

wherein the variables are as previously described herein,
and further wherein when the [symbol] adjacent to R₃ is a double bond, R₃₁ and R_{3′} are absent and the [symbol] adjacent to R₃₀ and W₅ is a single bond; when the [symbol] adjacent to R₄₄ is a double bond, R₄₅ and R₄₆ are absent; when the [symbol] adjacent to R₆₀ is a double bond, R₆₁ and R₄₆ are absent; when the [symbol] adjacent to R₄₂ and R₄₆ is a double bond, R₄₆ and R₄₃ are absent; when the [symbol] adjacent to R₄₁ and R₄₈ is a double bond, R₄₁ and R₄₈ are absent; when the [symbol] adjacent to R₄₂ and R₄₁ is a double bond, R₄₁ and R₄₃ are absent; when the [symbol] adjacent to R₄₀ in Formula IXb is a double bond, R₄₁ is absent; and when the [symbol] adjacent to R₃₀ and W₅ is a double bond, then W₅ is C and the [symbol] between W₅ and W₄ is a single bond.

In certain embodiments of compounds of Formula IXa, R₄₀ is not ethyl, vinyl, 2-bromoethyl, 2-hydroxyethyl, 2-fluoroethyl, 2-cyanoethyl, or 2-N₃-ethyl. In certain embodiments for compounds of Formula IXa, R₄₀ is not ethyl, vinyl, 2-bromoethyl, 2-hydroxyethyl, 2-fluoroethyl, 2-cyanoethyl, or 2-N₃-ethyl when R₄₇ is —C(═O)OMe.

In certain embodiments for compounds of Formula IXb, R₄₀ is not methyl. In certain embodiments for compounds of Formula IXb, R₄₀ is not methyl when R₄₇ is —C(═O)OMe. In certain embodiments for compounds of Formula IXa or IXb, R₄₀ is selected from optionally substituted C₂-C₈ alkyl, optionally substituted C₂-C₈ alkenyl, or optionally substituted C₂-C₈ alkynyl.

Exemplary compounds of Formula IXa and IXb include, but are not limited to:

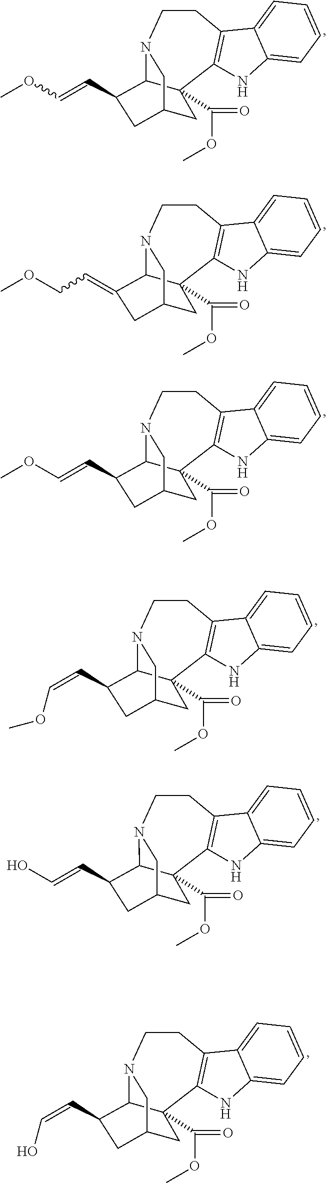

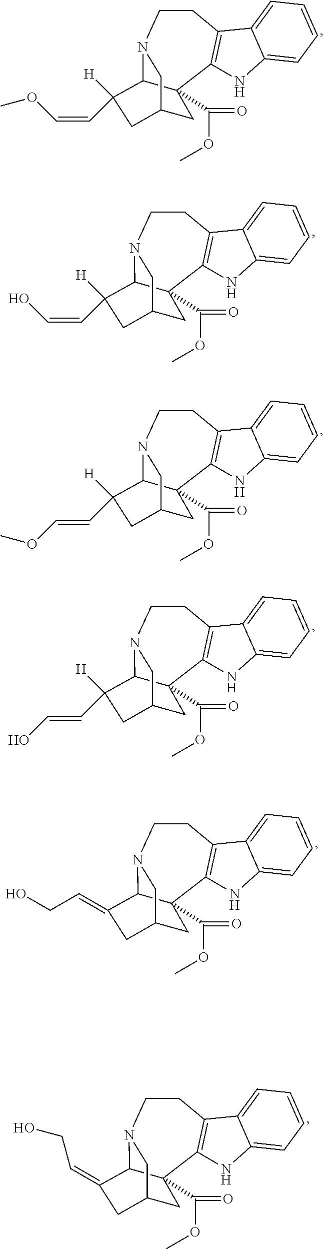

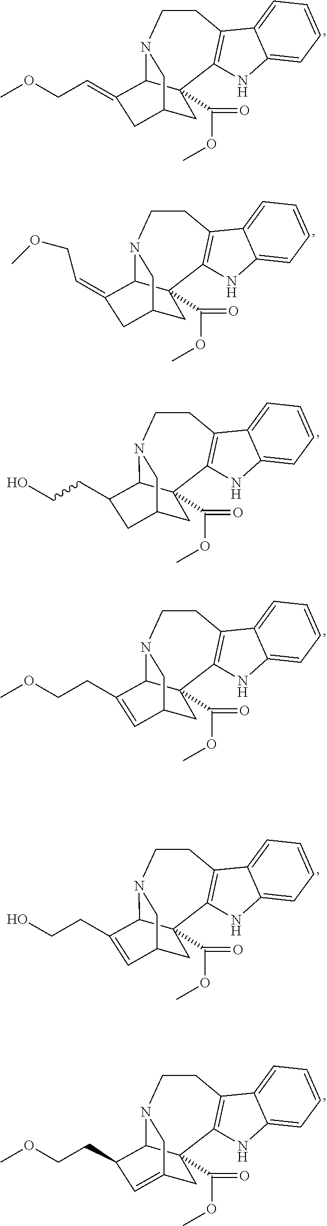 [SMILES: COC/C=C1\CC2CN3CCc4c([nH]c5ccccc45)[C@@](C(=O)OC)(C2)C13 ; COC/C=C1/CC2CN3CCc4c([nH]c5ccccc45)[C@@](C(=O)OC)(C2)C13 ; COC(=O)[C@]12CC3CC(CCO)C1N(CCc1c2[nH]c2ccccc12)C3 ; COCCC1=CC2CN3CCc4c([nH]c5ccccc45)[C@@](C(=O)OC)(C2)C13 ; COC(=O)[C@]12CC3C=C(CCO)C1N(CCc1c2[nH]c2ccccc12)C3 ; COCC[C@@H]1C=C2CN3CCc4c([nH]c5ccccc45)[C@@](C(=O)OC)(C2)C13]

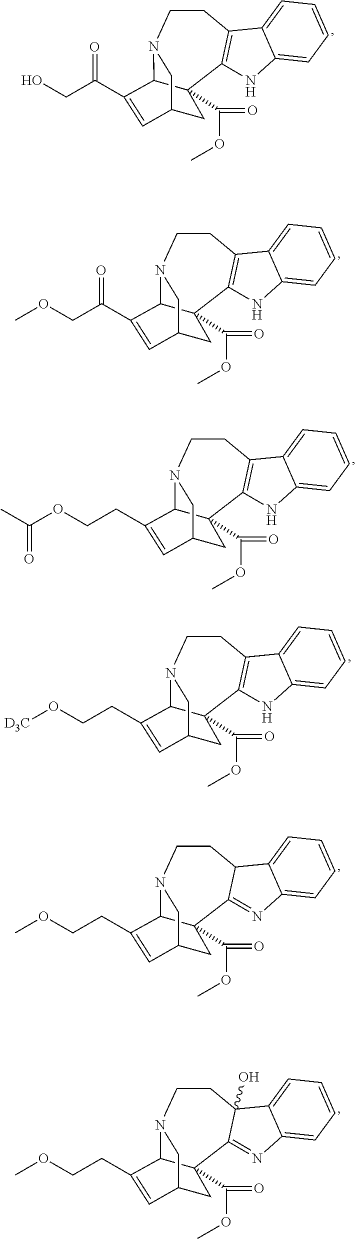 [SMILES: COC(=O)[C@]12CC3C=C(C(=O)CO)C1N(CCc1c2[nH]c2ccccc12)C3 ; COCC(=O)C1=CC2CN3CCc4c([nH]c5ccccc45)[C@@](C(=O)OC)(C2)C13 ; COC(=O)[C@]12CC3C=C(CCOC(C)=O)C1N(CCc1c2[nH]c2ccccc12)C3 ; [2H]C([2H])([2H])OCCC1=CC2CN3CCc4c([nH]c5ccccc45)[C@@](C(=O)OC)(C2)C13 ; COCCC1=CC2CN3CCC4C(=Nc5ccccc54)[C@@](C(=O)OC)(C2)C13 ; COCCC1=CC2CN3CCC4(O)C(=Nc5ccccc54)[C@@](C(=O)OC)(C2)C13]

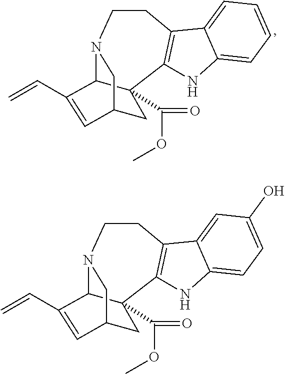 [SMILES: C=CC1=CC2CN3CCc4c([nH]c5ccccc45)[C@@](C(=O)OC)(C2)C13 ; C=CC1=CC2CN3CCc4c([nH]c5ccc(O)cc45)[C@@](C(=O)OC)(C2)C13]

and salts, solvates, hydrates, enantiomers, polymorphs and prodrugs of any of the foregoing compounds.

Consistent with the foregoing embodiments, in some embodiments the compounds are selected from Formula Xa or Xb:

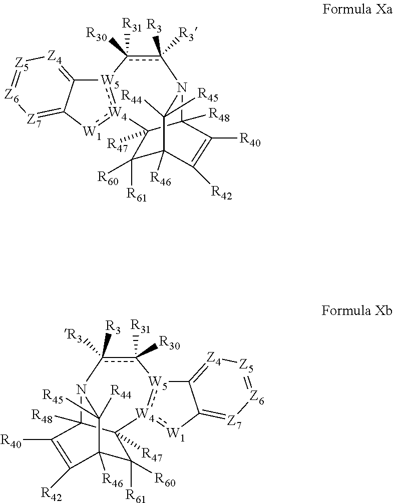

wherein the variables are as previously described herein.

In certain embodiments for compounds of Formula Xa or Xb, R₄₀ is not ethyl, vinyl, 2-bromoethyl, 2-hydroxyethyl, 2-fluoroethyl, 2-cyanoethyl, or 2-N₃-ethyl. In certain embodiments for compounds of Formula Xa or Xb, R₄₀ is not ethyl, vinyl, 2-bromoethyl, 2-hydroxyethyl, 2-fluoroethyl, 2-cyanoethyl, or 2-N₃-ethyl when R₄₇ is —C(═O)OMe.

Consistent with the foregoing embodiments, in some embodiments the compounds are selected from Formula XIa or XIb:

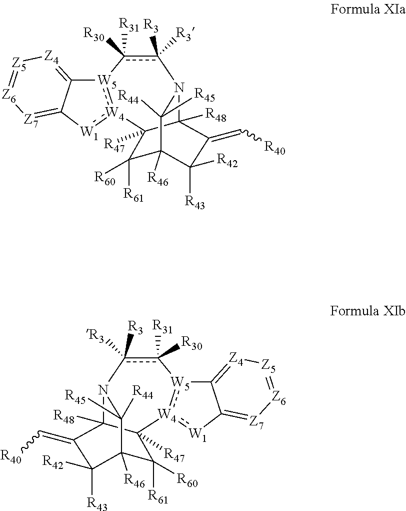

wherein the variables are as previously described herein.

In certain embodiments for compounds of Formula XIa or XIb, R₄₀ is not methyl. In certain embodiments for compounds of Formula XIa or XIb, R₄₀ is not methyl when R₄₇ is —C(═O)OMe. In certain embodiments for compounds of Formula XIa or XIb, R₄₀ is selected from optionally substituted C₂-C₈ alkyl, optionally substituted C₂-C₈ alkenyl, or optionally substituted C₂-C₈ alkynyl.

Exemplary compounds include compounds of Formula II(a) set forth below in Table 1:

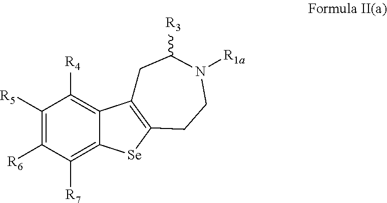

TABLE 1

| Ref. | R₁ₐ | R₃ | R₄ | R₅ | R₆ | R₇ |
|---|---|---|---|---|---|---|
| 1 | —CH₃ | H | —OCH₃ | H | H | H |
| 2 | —CH₃ | —CH₃ | —OCH₃ | H | H | H |
| 3 | —CH₃ | —CH₂CH₃ | —OCH₃ | H | H | H |
| 4 | —CH₃ | H | —OCH₃ | —CH₃ | H | H |
| 5 | —CH₃ | H | —OCH₃ | H | F | H |
| 6 | —CH₃ | H | —OCH₃ | H | H | —CH₃ |
| 7 | —CH₂CH₃ | —CH₃ | —OCH₃ | H | H | H |
| 8 | —CH₂CH₃ | —CH₂CH₃ | —OCH₃ | H | H | H |
| 9 | —CH₂CH₃ | H | —OCH₃ | —CH₃ | H | H |
| 10 | —CH₂CH₃ | H | —OCH₃ | H | F | H |
| 11 | —CH₂CH₃ | H | —OCH₃ | H | H | —CH₃ |
| 12 | —(CH₂)₂CH₃ | —CH₃ | —OCH₃ | H | H | H |
| 13 | —(CH₂)₂CH₃ | —CH₂CH₃ | —OCH₃ | H | H | H |
| 14 | —(CH₂)₂CH₃ | H | —OCH₃ | H | F | H |
| 15 | —(CH₂)₂CH₃ | H | —OCH₃ | H | H | —CH₃ |
| 16 | —CH(CH₃)₂ | —CH₃ | —OCH₃ | H | H | H |
| 17 | —CH(CH₃)₂ | —CH₂CH₃ | —OCH₃ | H | H | H |
| 18 | —CH(CH₃)₂ | H | —OCH₃ | H | F | H |
| 19 | —(CH₂)₃CH₃ | H | —OCH₃ | H | H | H |
| 20 | —(CH₂)₃CH₃ | —CH₃ | —OCH₃ | H | H | H |
| 21 | —CH₂—HC═CH₂ | H | —OCH₃ | H | H | H |
| 22 | —CH₃ | H | —OH | H | H | H |
| 23 | —CH₃ | —CH₃ | —OH | H | H | H |
| 24 | —CH₃ | —CH₂CH₃ | —OH | H | H | H |
| 25 | —CH₃ | H | —OH | H | F | H |
| 26 | —CH₃ | H | —OH | H | H | —CH₃ |
| 27 | —CH₂CH₃ | H | —OH | H | H | H |
| 28 | —CH₂CH₃ | —CH₃ | —OH | H | H | H |
| 29 | —CH₂CH₃ | —CH₂CH₃ | —OH | H | H | H |
| 30 | —CH₂CH₃ | H | —OH | H | F | H |
| 31 | —CH₂CH₃ | H | —OH | H | H | —CH₃ |
| 32 | —(CH₂)₂CH₃ | H | —OH | H | H | H |
| 33 | —(CH₂)₂CH₃ | —CH₃ | —OH | H | H | H |
| 34 | —(CH₂)₂CH₃ | —CH₂CH₃ | —OH | H | H | H |
| 35 | —(CH₂)₂CH₃ | H | —OH | H | F | H |
| 36 | —CH(CH₃)₂ | H | —OH | H | H | H |
| 37 | —CH(CH₃)₂ | —CH₃ | —OH | H | H | H |
| 38 | —CH(CH₃)₂ | —CH₂CH₃ | —OH | H | H | H |
| 39 | —(CH₂)₃CH₃ | H | —OH | H | H | H |
| 40 | —(CH₂)₃CH₃ | H | —OH | H | H | H |
| 41 | —CH₂—HC═CH₂ | H | —OH | H | H | H |
| 42 | —CH₃ | H | —OC(O)CH₃ | H | H | H |
| 43 | —CH₃ | —CH₃ | —OC(O)CH₃ | H | H | H |
| 44 | —CH₃ | —CH₂CH₃ | —OC(O)CH₃ | H | H | H |
| 45 | —CH₃ | H | —OC(O)CH₃ | —CH₃ | H | H |
| 46 | —CH₃ | H | —OC(O)CH₃ | H | F | H |
| 47 | —CH₃ | H | —OC(O)CH₃ | H | H | —CH₃ |
| 48 | —CH₂CH₃ | H | —OC(O)CH₃ | H | H | H |
| 49 | —CH₂CH₃ | —CH₃ | —OC(O)CH₃ | H | H | H |
| 50 | —CH₂CH₃ | —CH₂CH₃ | —OC(O)CH₃ | H | H | H |
| 51 | —CH₂CH₃ | H | —OC(O)CH₃ | H | F | H |
| 52 | —CH₂CH₃ | H | —OC(O)CH₃ | H | H | —CH₃ |
| 53 | —(CH₂)₂CH₃ | H | —OC(O)CH₃ | H | H | H |
| 54 | —(CH₂)₂CH₃ | —CH₃ | —OC(O)CH₃ | H | H | H |
| 55 | —(CH₂)₂CH₃ | —CH₂CH₃ | —OC(O)CH₃ | H | H | H |
| 56 | —(CH₂)₂CH₃ | H | —OC(O)CH₃ | H | F | H |
| 57 | —CH(CH₃)₂ | H | —OC(O)CH₃ | H | H | H |
| 58 | —CH(CH₃)₂ | —CH₃ | —OC(O)CH₃ | H | H | H |
| 59 | —CH(CH₃)₂ | —CH₂CH₃ | —OC(O)CH₃ | H | H | H |
| 60 | —(CH₂)₃CH₃ | H | —OC(O)CH₃ | H | H | H |
| 61 | —(CH₂)₃CH₃ | —CH₃ | —OC(O)CH₃ | H | H | H |
| 62 | —CH₂—HC═CH₂ | H | —OC(O)CH₃ | H | H | H |
| 63 | —CH₃ | H | H | —OCH₃ | H | H |
| 64 | —CH₃ | —CH₃ | H | —OCH₃ | H | H |
| 65 | —CH₃ | —CH₂CH₃ | H | —OCH₃ | H | H |
| 66 | —CH₃ | H | H | —OCH₃ | H | H |
| 67 | —CH₃ | H | H | —OCH₃ | F | H |
| 68 | —CH₃ | H | H | —OCH₃ | H | —CH₃ |
| 69 | —CH₂CH₃ | H | H | —OCH₃ | H | H |
| 70 | —CH₂CH₃ | —CH₃ | H | —OCH₃ | H | H |
| 71 | —CH₂CH₃ | —CH₂CH₃ | H | —OCH₃ | H | H |
| 72 | —CH₂CH₃ | H | H | —OCH₃ | F | H |
| 73 | —CH₂CH₃ | H | H | —OCH₃ | H | —CH₃ |
| 74 | —(CH₂)₂CH₃ | H | H | —OCH₃ | H | H |
| 75 | —(CH₂)₂CH₃ | —CH₃ | H | —OCH₃ | H | H |
| 76 | —(CH₂)₂CH₃ | —CH₂CH₃ | H | —OCH₃ | H | H |
| 77 | —(CH₂)₂CH₃ | H | H | —OCH₃ | F | H |
| 78 | —CH(CH₃)₂ | H | H | —OCH₃ | H | H |
| 79 | —CH(CH₃)₂ | —CH₃ | H | —OCH₃ | H | H |
| 80 | —CH(CH₃)₂ | —CH₂CH₃ | H | —OCH₃ | H | H |
| 81 | —(CH₂)₃CH₃ | H | H | —OCH₃ | H | H |
| 82 | —(CH₂)₃CH₃ | —CH₃ | H | —OCH₃ | H | H |
| 83 | —CH₂—HC═CH₂ | H | H | —OCH₃ | H | H |
| 84 | —CH₃ | H | H | —OH | H | H |
| 85 | —CH₃ | —CH₃ | H | —OH | H | H |
| 86 | —CH₃ | —CH₂CH₃ | H | —OH | H | H |
| 87 | —CH₃ | H | H | —OH | F | H |
| 88 | —CH₃ | H | H | —OH | H | —CH₃ |
| 89 | —CH₂CH₃ | H | H | —OH | H | H |
| 90 | —CH₂CH₃ | —CH₃ | H | —OH | H | H |
| 91 | —CH₂CH₃ | —CH₂CH₃ | H | —OH | H | H |
| 92 | —CH₂CH₃ | H | H | —OH | F | H |
| 93 | —CH₂CH₃ | H | H | —OH | H | —CH₃ |
| 94 | —(CH₂)₂CH₃ | H | H | —OH | H | H |
| 95 | —(CH₂)₂CH₃ | —CH₃ | H | —OH | H | H |
| 96 | —(CH₂)₂CH₃ | —CH₂CH₃ | H | —OH | H | H |
| 97 | —(CH₂)₂CH₃ | H | H | —OH | F | H |
| 98 | —(CH₂)₂CH₃ | H | H | —OH | H | H |
| 99 | —CH(CH₃)₂ | —CH₃ | H | —OH | H | H |
| 100 | —CH(CH₃)₂ | —CH₂CH₃ | H | —OH | H | H |
| 101 | —CH(CH₃)₂ | H | H | —OH | H | H |
| 102 | —(CH₂)₃CH₃ | —CH₃ | H | —OH | H | H |
| 103 | —(CH₂)₃CH₃ | H | H | —OH | H | H |
| 104 | —CH₃ | H | H | —OC(O)CH₃ | H | H |
| 105 | —CH₃ | —CH₃ | H | —OC(O)CH₃ | H | H |
| 106 | —CH₃ | —CH₂CH₃ | H | —OC(O)CH₃ | H | H |
| 107 | —CH₃ | H | —CH₃ | —OC(O)CH₃ | H | H |
| 108 | —CH₃ | H | H | —OC(O)CH₃ | F | H |
| 109 | —CH₃ | H | H | —OC(O)CH₃ | H | —CH₃ |
| 110 | —CH₂CH₃ | H | H | —OC(O)CH₃ | H | H |
| 111 | —CH₂CH₃ | —CH₃ | H | —OC(O)CH₃ | H | H |
| 112 | —CH₂CH₃ | —CH₂CH₃ | H | —OC(O)CH₃ | H | H |
| 113 | —CH₂CH₃ | H | H | —OC(O)CH₃ | F | H |
| 114 | —CH₂CH₃ | H | H | —OC(O)CH₃ | H | —CH₃ |
| 115 | —(CH₂)₂CH₃ | H | H | —OC(O)CH₃ | H | H |
| 116 | —(CH₂)₂CH₃ | —CH₃ | H | —OC(O)CH₃ | H | H |
| 117 | —(CH₂)₂CH₃ | —CH₂CH₃ | H | —OC(O)CH₃ | H | H |
| 118 | —(CH₂)₂CH₃ | H | H | —OC(O)CH₃ | F | H |
| 119 | —CH(CH₃)₂ | H | H | —OC(O)CH₃ | H | H |
| 120 | —CH(CH₃)₂ | —CH₃ | H | —OC(O)CH₃ | H | H |
| 121 | —CH(CH₃)₂ | —CH₂CH₃ | H | —OC(O)CH₃ | H | H |
| 122 | —(CH₂)₃CH₃ | H | H | —OC(O)CH₃ | H | H |
| 123 | —(CH₂)₃CH₃ | —CH₃ | H | —OC(O)CH₃ | H | H |
| 124 | —CH₂—HC═CH₂ | H | H | —OC(O)CH₃ | H | H |
| 125 | —CH₃ | H | Br | H | H | H |
| 126 | —CH₃ | —CH₃ | Br | H | H | H |
| 127 | —CH₃ | —CH₂CH₃ | Br | H | H | H |
| 128 | —CH₃ | H | Br | —CH₃ | H | H |
| 129 | —CH₃ | H | Br | H | F | H |
| 130 | —CH₃ | H | Br | H | H | —CH₃ |
| 131 | —CH₂CH₃ | H | Br | H | H | H |
| 132 | —CH₂CH₃ | —CH₃ | Br | H | H | H |
| 133 | —CH₂CH₃ | —CH₂CH₃ | Br | H | H | H |
| 134 | —CH₂CH₃ | H | Br | H | F | H |
| 135 | —CH₂CH₃ | H | Br | H | H | —CH₃ |
| 135 | —(CH₂)₂CH₃ | H | Br | H | H | H |
| 137 | —(CH₂)₂CH₃ | —CH₃ | Br | H | H | H |
| 138 | —(CH₂)₂CH₃ | —CH₂CH₃ | Br | H | H | H |
| 139 | —(CH₂)₂CH₃ | H | Br | H | F | H |
| 140 | —CH(CH₃)₂ | H | Br | H | H | H |
| 141 | —CH(CH₃)₂ | —CH₃ | Br | H | H | H |
| 142 | —CH(CH₃)₂ | —CH₂CH₃ | Br | H | H | H |
| 143 | —(CH₂)₃CH₃ | H | Br | H | H | H |
| 144 | —(CH₂)₃CH₃ | —CH₃ | Br | H | H | H |
| 145 | —CH₂—HC═CH₂ | H | Br | H | H | H |
| 146 | —CH₃ | H | Cl | H | H | H |
| 147 | —CH₃ | —CH₃ | Cl | H | H | H |
| 148 | —CH₃ | —CH₂CH₃ | Cl | H | H | H |
| 149 | —CH₃ | H | Cl | H | F | H |
| 150 | —CH₃ | H | Cl | H | H | —CH₃ |
| 151 | —CH₂CH₃ | H | Cl | H | H | H |
| 152 | —CH₂CH₃ | —CH₃ | Cl | H | H | H |
| 153 | —CH₂CH₃ | —CH₂CH₃ | Cl | H | H | H |
| 154 | —CH₂CH₃ | H | Cl | H | F | H |
| 155 | —CH₂CH₃ | H | Cl | H | H | —CH₃ |
| 156 | —(CH₂)₂CH₃ | H | Cl | H | H | H |
| 157 | —(CH₂)₂CH₃ | —CH₃ | Cl | H | H | H |
| 158 | —(CH₂)₂CH₃ | —CH₂CH₃ | Cl | H | H | H |
| 159 | —(CH₂)₂CH₃ | H | Cl | H | F | H |
| 160 | —CH(CH₃)₂ | H | Cl | H | H | H |
| 161 | —CH(CH₃)₂ | —CH₃ | Cl | H | H | H |
| 162 | —CH(CH₃)₂ | —CH₂CH₃ | Cl | H | H | H |
| 163 | —(CH₂)₃CH₃ | H | Cl | H | H | H |
| 164 | —(CH₂)₃CH₃ | —CH₃ | Cl | H | H | H |
| 165 | —CH₂—HC═CH₂ | H | Cl | H | H | H |
| 166 | —CH₃ | H | F | H | H | H |
| 167 | —CH₃ | —CH₃ | F | H | H | H |
| 168 | —CH₃ | —CH₂CH₃ | F | H | H | H |
| 169 | —CH₃ | H | F | —CH₃ | H | H |
| 170 | —CH₃ | H | F | H | F | H |
| 171 | —CH₃ | H | F | H | H | —CH₃ |
| 172 | —CH₂CH₃ | H | F | H | H | H |
| 173 | —CH₂CH₃ | —CH₃ | F | H | H | H |
| 174 | —CH₂CH₃ | —CH₂CH₃ | F | H | H | H |
| 175 | —CH₂CH₃ | H | F | H | F | H |
| 176 | —CH₂CH₃ | H | F | H | H | —CH₃ |
| 177 | —(CH₂)₂CH₃ | H | F | H | H | H |
| 178 | —(CH₂)₂CH₃ | —CH₃ | F | H | H | H |
| 179 | —(CH₂)₂CH₃ | —CH₂CH₃ | F | H | H | H |
| 180 | —(CH₂)₂CH₃ | H | F | H | F | H |
| 181 | —CH(CH₃)₂ | H | F | H | H | H |
| 182 | —CH(CH₃)₂ | —CH₃ | F | H | H | H |
| 183 | —CH(CH₃)₂ | —CH₂CH₃ | F | H | H | H |
| 184 | —(CH₂)₃CH₃ | H | F | H | H | H |
| 185 | —(CH₂)₃CH₃ | —CH₃ | F | H | H | H |
| 186 | —CH₂—HC═CH₂ | H | F | H | H | H |
| 187 | —CH₃ | H | H | Br | H | H |
| 188 | —CH₃ | H | H | Br | H | H |
| 189 | —CH₃ | —CH₃ | H | Br | H | H |
| 190 | —CH₃ | —CH₂CH₃ | H | Br | H | H |
| 191 | —CH₃ | H | H | Br | F | H |
| 192 | —CH₃ | H | H | Br | H | —CH₃ |
| 193 | —CH₂CH₃ | H | H | Br | H | H |
| 194 | —CH₂CH₃ | —CH₃ | H | Br | H | H |
| 195 | —CH₂CH₃ | —CH₂CH₃ | H | Br | H | H |
| 196 | —CH₂CH₃ | H | H | Br | F | H |
| 197 | —CH₂CH₃ | H | H | Br | H | —CH₃ |
| 198 | —(CH₂)₂CH₃ | H | H | Br | H | H |
| 199 | —(CH₂)₂CH₃ | —CH₃ | H | Br | H | H |
| 200 | —(CH₂)₂CH₃ | —CH₂CH₃ | H | Br | H | H |
| 201 | —(CH₂)₂CH₃ | H | H | Br | F | H |
| 202 | —CH(CH₃)₂ | H | H | Br | H | H |
| 203 | —CH(CH₃)₂ | —CH₃ | H | Br | H | H |
| 204 | —CH(CH₃)₂ | —CH₂CH₃ | H | Br | H | H |
| 205 | —(CH₂)₃CH₃ | H | H | Br | H | H |
| 206 | —(CH₂)₃CH₃ | —CH₃ | H | Br | H | H |
| 207 | —CH₂—HC═CH₂ | H | H | Br | H | H |
| 208 | —CH₃ | H | H | Cl | H | H |
| 209 | —CH₃ | —CH₃ | H | Cl | H | H |
| 210 | —CH₃ | —CH₂CH₃ | H | Cl | H | H |
| 211 | —CH₃ | H | H | Cl | F | H |
| 212 | —CH₃ | H | H | Cl | H | —CH₃ |
| 213 | —CH₂CH₃ | H | H | Cl | H | H |
| 214 | —CH₂CH₃ | —CH₃ | H | Cl | H | H |
| 215 | —CH₂CH₃ | —CH₂CH₃ | H | Cl | H | H |
| 216 | —CH₂CH₃ | H | H | Cl | F | H |
| 217 | —CH₂CH₃ | H | H | Cl | H | —CH₃ |
| 218 | —(CH₂)₂CH₃ | H | H | Cl | H | H |
| 219 | —(CH₂)₂CH₃ | —CH₃ | H | Cl | H | H |
| 220 | —(CH₂)₂CH₃ | —CH₂CH₃ | H | Cl | H | H |
| 221 | —(CH₂)₂CH₃ | H | H | Cl | F | H |
| 222 | —CH(CH₃)₂ | H | H | Cl | H | H |
| 223 | —CH(CH₃)₂ | —CH₃ | H | Cl | H | H |
| 224 | —CH(CH₃)₂ | —CH₂CH₃ | H | Cl | H | H |
| 225 | —(CH₂)₃CH₃ | H | H | Cl | H | H |
| 226 | —(CH₂)₃CH₃ | —CH₃ | H | Cl | H | H |
| 227 | —CH₂—HC═CH₂ | H | H | Cl | H | H |
| 228 | —CH₃ | H | H | F | H | H |
| 229 | —CH₃ | —CH₃ | H | F | H | H |
| 230 | —CH₃ | —CH₂CH₃ | H | F | H | H |
| 231 | —CH₃ | H | H | F | F | —CH₃ |
| 232 | —CH₃ | H | H | F | F | H |
| 233 | —CH₃ | H | H | F | H | —CH₃ |
| 234 | —CH₂CH₃ | H | H | F | H | H |
| 235 | —CH₂CH₃ | —CH₃ | H | F | H | H |
| 236 | —CH₂CH₃ | —CH₂CH₃ | H | F | H | H |
| 237 | —CH₂CH₃ | H | H | F | F | H |
| 238 | —CH₂CH₃ | H | H | F | H | —CH₃ |
| 239 | —(CH₂)₂CH₃ | H | H | F | H | H |
| 240 | —(CH₂)₂CH₃ | —CH₃ | H | F | H | H |
| 241 | —(CH₂)₂CH₃ | —CH₂CH₃ | H | F | H | H |
| 242 | —(CH₂)₂CH₃ | H | H | F | F | H |
| 243 | —CH(CH₃)₂ | H | H | F | H | H |
| 244 | —CH(CH₃)₂ | —CH₃ | H | F | H | H |
| 245 | —CH(CH₃)₂ | —CH₂CH₃ | H | F | H | H |
| 246 | —(CH₂)₃CH₃ | H | H | F | H | H |
| 247 | —(CH₂)₃CH₃ | —CH₃ | H | F | H | H |
| 248 | —CH₂—HC═CH₂ | H | H | F | H | H |
| 249 | —CH₃ | H | H | H | H | H |
| 250 | —CH₃ | H | —OH | H | H | H |
| 251 | —CH₃ | H | —OC(O)CH₃ | H | H | H |
| 252 | —CH₃ | H | —OCH₃ | H | H | H |
| 253 | —CH₃ | H | Br | H | H | H |
| 254 | —CH₃ | H | Cl | H | H | H |
| 255 | —CH₃ | H | F | H | H | H |
| 256 | —CH₃ | —CH₃ | H | H | H | H |
| 257 | —CH₃ | —CH₃ | —OH | H | H | H |
| 258 | —CH₃ | —CH₃ | —OC(O)CH₃ | H | H | H |
| 259 | —CH₃ | —CH₃ | —OCH₃ | H | H | H |
| 260 | —CH₃ | —CH₃ | Br | H | H | H |
| 261 | —CH₃ | —CH₃ | Cl | H | H | H |
| 262 | —CH₃ | —CH₃ | F | H | H | H |
| 263 | —CH₃ | H | H | H | F | H |
| 264 | —CH₃ | H | —OH | H | F | H |
| 265 | —CH₃ | H | —OC(O)CH₃ | H | F | H |
| 266 | —CH₃ | H | —OCH₃ | H | F | H |
| 267 | —CH₃ | H | Br | H | F | H |
| 268 | —CH₃ | H | Cl | H | F | H |
| 269 | —CH₃ | H | F | H | F | H |
| 270 | —CH₃ | H | H | H | H | —CH₃ |
| 271 | —CH₃ | H | —OH | H | H | —CH₃ |
| 272 | —CH₃ | H | —OC(O)CH₃ | H | H | —CH₃ |
| 273 | —CH₃ | H | —OCH₃ | H | H | —CH₃ |
| 274 | —CH₃ | H | Br | H | H | —CH₃ |
| 275 | —CH₃ | H | Cl | H | H | —CH₃ |
| 276 | —CH₃ | H | F | H | H | —CH₃ |
| 277 | —CH₃ | H | H | —OH | H | H |
| 278 | —CH₃ | H | H | —OC(O)CH₃ | H | H |
| 279 | —CH₃ | H | H | —OCH₃ | H | H |
| 280 | —CH₃ | H | H | Br | H | H |
| 281 | —CH₃ | H | H | Cl | H | H |
| 282 | —CH₃ | H | H | F | H | H |
| 283 | —CH₃ | —CH₃ | H | H | H | H |
| 284 | —CH₃ | —CH₃ | H | —OH | H | H |
| 285 | —CH₃ | —CH₃ | H | —OC(O)CH₃ | H | H |
| 286 | —CH₃ | —CH₃ | H | —OCH₃ | H | H |
| 287 | —CH₃ | —CH₃ | H | Br | H | H |
| 288 | —CH₃ | —CH₃ | H | Cl | H | H |
| 289 | —CH₃ | —CH₃ | H | F | H | H |
| 290 | —CH₃ | H | H | H | F | H |
| 291 | —CH₃ | H | H | —OH | F | H |
| 292 | —CH₃ | H | H | —OC(O)CH₃ | F | H |
| 293 | —CH₃ | H | H | —OCH₃ | F | H |
| 294 | —CH₃ | H | H | Br | F | H |
| 295 | —CH₃ | H | H | Cl | F | H |
| 296 | —CH₃ | H | H | F | F | H |
| 297 | —CH₃ | H | H | H | H | —CH₃ |
| 298 | —CH₃ | H | H | —OH | H | —CH₃ |
| 299 | —CH₃ | H | H | —OC(O)CH₃ | H | —CH₃ |
| 300 | —CH₃ | H | H | —OCH₃ | H | —CH₃ |
| 301 | —CH₃ | H | H | Br | H | —CH₃ |
| 302 | —CH₃ | H | H | Cl | H | —CH₃ |
| 303 | —CH₃ | H | H | F | H | —CH₃ |
| 304 | H | H | —OH | H | H | H |
| 305 | H | —CH₃ | —OH | H | H | H |
| 306 | H | —CH₂CH₃ | —OH | H | H | H |
| 307 | H | H | —OH | —CH₃ | H | H |
| 308 | H | H | —OH | H | F | H |
| 309 | H | H | —OH | H | H | —CH₃ |
| 310 | H | H | H | H | H | H |
| 311 | H | —CH₃ | H | H | H | H |
| 312 | H | —CH₂CH₃ | H | H | H | H |
| 313 | H | H | H | —CH₃ | H | H |
| 314 | H | H | H | H | F | H |
| 315 | H | H | H | H | H | —CH₃ |
| 316 | H | H | H | H | —OCH₃ | H |
| 317 | H | H | H | H | F | —CH₃ |
| 318 | —CH₃ | H | H | H | —OCH₃ | H |
| 319 | —CH₃ | H | H | H | F | —CH₃ |

Other exemplary compounds include compounds of Formula II(b) set forth below in Table 2:

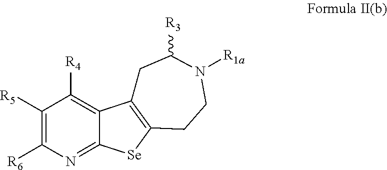

TABLE 2

| Ref. | R₁ₐ | R₃ | R₄ | R₅ | R₆ |
|---|---|---|---|---|---|
| 1 | —CH₃ | H | —OCH₃ | H | H |
| 2 | —CH₃ | —CH₃ | —OCH₃ | H | H |
| 3 | —CH₃ | —CH₂CH₃ | —OCH₃ | H | H |
| 4 | —CH₃ | H | —OCH₃ | CH₃ | H |
| 5 | —CH₃ | H | —OCH₃ | H | F |
| 6 | —CH₃ | H | —OCH₃ | H | —OCH₃ |
| 7 | —CH₂CH₃ | —CH₃ | —OCH₃ | H | H |
| 8 | —CH₂CH₃ | —CH₂CH₃ | —OCH₃ | H | H |
| 9 | —CH₂CH₃ | H | —OCH₃ | —CH₃ | H |
| 10 | —CH₂CH₃ | H | —OCH₃ | H | F |
| 11 | —CH₂CH₃ | H | —OCH₃ | H | —OCH₃ |
| 12 | —(CH₂)₂CH₃ | —CH₃ | —OCH₃ | H | H |
| 13 | —(CH₂)₂CH₃ | —CH₂CH₃ | —OCH₃ | H | H |
| 14 | —(CH₂)₂CH₃ | H | —OCH₃ | H | F |
| 15 | —(CH₂)₂CH₃ | H | —OCH₃ | H | —OCH₃ |
| 16 | —CH(CH₃)₂ | —CH₃ | —OCH₃ | H | H |
| 17 | —CH(CH₃)₂ | —CH₂CH₃ | —OCH₃ | H | H |
| 18 | —CH(CH₃)₂ | H | —OCH₃ | H | F |
| 19 | —(CH₂)₃CH₃ | H | —OCH₃ | H | H |
| 20 | —(CH₂)₃CH₃ | —CH₃ | —OCH₃ | H | H |
| 21 | —CH₂—HC═CH₂ | H | —OCH₃ | H | H |
| 22 | —CH₃ | H | —OH | H | H |
| 23 | —CH₃ | —CH₃ | —OH | H | H |
| 24 | —CH₃ | —CH₂CH₃ | —OH | H | H |
| 25 | —CH₃ | H | —OH | H | F |
| 26 | —CH₃ | H | —OH | H | —OCH₃ |
| 27 | —CH₂CH₃ | H | —OH | H | H |
| 28 | —CH₂CH₃ | —CH₃ | —OH | H | H |
| 29 | —CH₂CH₃ | —CH₂CH₃ | —OH | H | H |
| 30 | —CH₂CH₃ | H | —OH | H | F |
| 31 | —CH₂CH₃ | H | —OH | H | —OCH₃ |
| 32 | —(CH₂)₂CH₃ | H | —OH | H | H |
| 33 | —(CH₂)₂CH₃ | —CH₃ | —OH | H | H |
| 34 | —(CH₂)₂CH₃ | —CH₂CH₃ | —OH | H | H |
| 35 | —(CH₂)₂CH₃ | H | —OH | H | F |
| 36 | —CH(CH₃)₂ | H | —OH | H | H |
| 37 | —CH(CH₃)₂ | —CH₃ | —OH | H | H |
| 38 | —CH(CH₃)₂ | —CH₂CH₃ | —OH | H | H |
| 39 | —(CH₂)₃CH₃ | H | —OH | H | H |
| 40 | —(CH₂)₃CH₃ | H | —OH | H | H |
| 41 | —CH₂—HC═CH₂ | H | —OH | H | H |
| 42 | —CH₃ | H | —OC(O)CH₃ | H | H |
| 43 | —CH₃ | —CH₃ | —OC(O)CH₃ | H | H |
| 44 | —CH₃ | —CH₂CH₃ | —OC(O)CH₃ | H | H |
| 45 | —CH₃ | H | —OC(O)CH₃ | —CH₃ | H |
| 46 | —CH₃ | H | —OC(O)CH₃ | H | F |
| 47 | —CH₃ | H | —OC(O)CH₃ | H | —OCH₃ |
| 48 | —CH₂CH₃ | H | —OC(O)CH₃ | H | H |
| 49 | —CH₂CH₃ | —CH₃ | —OC(O)CH₃ | H | H |
| 50 | —CH₂CH₃ | —CH₂CH₃ | —OC(O)CH₃ | H | H |
| 51 | —CH₂CH₃ | H | —OC(O)CH₃ | H | F |
| 52 | —CH₂CH₃ | H | —OC(O)CH₃ | H | —OCH₃ |
| 53 | —(CH₂)₂CH₃ | H | —OC(O)CH₃ | H | H |
| 54 | —(CH₂)₂CH₃ | —CH₃ | —OC(O)CH₃ | H | H |
| 55 | —(CH₂)₂CH₃ | —CH₂CH₃ | —OC(O)CH₃ | H | H |
| 56 | —(CH₂)₂CH₃ | H | —OC(O)CH₃ | H | F |
| 57 | —CH(CH₃)₂ | H | —OC(O)CH₃ | H | H |
| 58 | —CH(CH₃)₂ | —CH₃ | —OC(O)CH₃ | H | H |
| 59 | —CH(CH₃)₂ | —CH₂CH₃ | —OC(O)CH₃ | H | H |
| 60 | —(CH₂)₃CH₃ | H | —OC(O)CH₃ | H | H |
| 61 | —(CH₂)₃CH₃ | —CH₃ | —OC(O)CH₃ | H | H |
| 62 | —CH₂—HC═CH₂ | H | —OC(O)CH₃ | H | H |
| 63 | —CH₃ | H | H | —OCH₃ | H |
| 64 | —CH₃ | —CH₃ | H | —OCH₃ | H |
| 65 | —CH₃ | —CH₂CH₃ | H | —OCH₃ | H |
| 66 | —CH₃ | H | H | —OCH₃ | H |
| 67 | —CH₃ | H | H | —OCH₃ | F |
| 68 | —CH₃ | H | H | —OCH₃ | —OCH₃ |
| 69 | —CH₂CH₃ | H | H | —OCH₃ | H |
| 70 | —CH₂CH₃ | —CH₃ | H | —OCH₃ | H |
| 71 | —CH₂CH₃ | —CH₂CH₃ | H | —OCH₃ | H |
| 72 | —CH₂CH₃ | H | H | —OCH₃ | F |
| 73 | —CH₂CH₃ | H | H | —OCH₃ | —OCH₃ |
| 74 | —(CH₂)₂CH₃ | H | H | —OCH₃ | H |
| 75 | —(CH₂)₂CH₃ | —CH₃ | H | —OCH₃ | H |
| 76 | —(CH₂)₂CH₃ | —CH₂CH₃ | H | —OCH₃ | H |
| 77 | —(CH₂)₂CH₃ | H | H | —OCH₃ | F |
| 78 | —CH(CH₃)₂ | H | H | —OCH₃ | H |
| 79 | —CH(CH₃)₂ | —CH₃ | H | —OCH₃ | H |
| 80 | —CH(CH₃)₂ | —CH₂CH₃ | H | —OCH₃ | H |
| 81 | —(CH₂)₃CH₃ | H | H | —OCH₃ | H |
| 82 | —(CH₂)₃CH₃ | —CH₃ | H | —OCH₃ | H |
| 83 | —CH₂—HC═CH₂ | H | H | —OCH₃ | H |
| 84 | —CH₃ | H | H | —OH | H |
| 85 | —CH₃ | —CH₃ | H | —OH | H |
| 86 | —CH₃ | —CH₂CH₃ | H | —OH | H |
| 87 | —CH₃ | H | H | —OH | F |
| 88 | —CH₃ | H | H | —OH | —OCH₃ |
| 89 | —CH₂CH₃ | H | H | —OH | H |
| 90 | —CH₂CH₃ | —CH₃ | H | —OH | H |
| 91 | —CH₂CH₃ | —CH₂CH₃ | H | —OH | H |
| 92 | —CH₂CH₃ | H | H | —OH | F |
| 93 | —CH₂CH₃ | H | H | —OH | —OCH₃ |
| 94 | —(CH₂)₂CH₃ | H | H | —OH | H |
| 95 | —(CH₂)₂CH₃ | —CH₃ | H | —OH | H |
| 96 | —(CH₂)₂CH₃ | —CH₂CH₃ | H | —OH | H |
| 97 | —(CH₂)₂CH₃ | H | H | —OH | F |
| 98 | —CH(CH₃)₂ | H | H | —OH | H |
| 99 | —CH(CH₃)₂ | —CH₃ | H | —OH | H |
| 100 | —CH(CH₃)₂ | —CH₂CH₃ | H | —OH | H |
| 101 | —(CH₂)₃CH₃ | H | H | —OH | H |
| 102 | —(CH₂)₃CH₃ | —CH₃ | H | —OH | H |
| 103 | —CH₂—HC═CH₂ | H | H | —OH | H |
| 104 | —CH₃ | H | H | —OC(O)CH₃ | H |
| 105 | —CH₃ | —CH₃ | H | —OC(O)CH₃ | H |
| 106 | —CH₃ | —CH₂CH₃ | H | —OC(O)CH₃ | H |
| 107 | —CH₃ | H | —CH₃ | —OC(O)CH₃ | H |
| 108 | —CH₃ | H | H | —OC(O)CH₃ | F |
| 109 | —CH₃ | H | H | —OC(O)CH₃ | —OCH₃ |
| 110 | —CH₂CH₃ | H | H | —OC(O)CH₃ | H |
| 111 | —CH₂CH₃ | —CH₃ | H | —OC(O)CH₃ | H |
| 112 | —CH₂CH₃ | —CH₂CH₃ | H | —OC(O)CH₃ | H |
| 113 | —CH₂CH₃ | H | H | —OC(O)CH₃ | F |
| 114 | —CH₂CH₃ | H | H | —OC(O)CH₃ | —OCH₃ |
| 115 | —(CH₂)₂CH₃ | H | H | —OC(O)CH₃ | H |
| 116 | —(CH₂)₂CH₃ | —CH₃ | H | —OC(O)CH₃ | H |
| 117 | —(CH₂)₂CH₃ | —CH₂CH₃ | H | —OC(O)CH₃ | H |
| 118 | —(CH₂)₂CH₃ | H | H | —OC(O)CH₃ | F |
| 119 | —CH(CH₃)₂ | H | H | —OC(O)CH₃ | H |
| 120 | —CH(CH₃)₂ | —CH₃ | H | —OC(O)CH₃ | H |
| 121 | —CH(CH₃)₂ | —CH₂CH₃ | H | —OC(O)CH₃ | H |
| 122 | —(CH₂)₃CH₃ | H | H | —OC(O)CH₃ | H |
| 123 | —(CH₂)₃CH₃ | —CH₃ | H | —OC(O)CH₃ | H |
| 124 | —CH₂—HC═CH₂ | H | H | —OC(O)CH₃ | H |
| 125 | —CH₃ | H | Br | H | H |
| 126 | —CH₃ | —CH₃ | Br | H | H |
| 127 | —CH₃ | —CH₂CH₃ | Br | H | H |
| 128 | —CH₃ | H | Br | —CH₃ | H |
| 129 | —CH₃ | H | Br | H | F |
| 130 | —CH₃ | H | Br | H | —OCH₃ |
| 131 | —CH₂CH₃ | H | Br | H | H |
| 132 | —CH₂CH₃ | —CH₃ | Br | H | H |
| 133 | —CH₂CH₃ | —CH₂CH₃ | Br | H | H |
| 134 | —CH₂CH₃ | H | Br | H | F |
| 135 | —CH₂CH₃ | H | Br | H | —OCH₃ |
| 135 | —(CH₂)₂CH₃ | H | Br | H | H |
| 137 | —(CH₂)₂CH₃ | —CH₃ | Br | H | H |
| 138 | —(CH₂)₂CH₃ | —CH₂CH₃ | Br | H | H |
| 139 | —(CH₂)₂CH₃ | H | Br | H | F |
| 140 | —CH(CH₃)₂ | H | Br | H | H |
| 141 | —CH(CH₃)₂ | —CH₃ | Br | H | H |
| 142 | —CH(CH₃)₂ | —CH₂CH₃ | Br | H | H |
| 143 | —(CH₂)₃CH₃ | H | Br | H | H |
| 144 | —(CH₂)₃CH₃ | —CH₃ | Br | H | H |
| 145 | —CH₂—HC═CH₂ | H | Br | H | H |
| 146 | —CH₃ | H | Cl | H | H |
| 147 | —CH₃ | —CH₃ | Cl | H | H |
| 148 | —CH₃ | —CH₂CH₃ | Cl | H | H |
| 149 | —CH₃ | H | Cl | H | F |
| 150 | —CH₃ | H | Cl | H | —OCH₃ |
| 151 | —CH₂CH₃ | H | Cl | H | H |
| 152 | —CH₂CH₃ | —CH₃ | Cl | H | H |
| 153 | —CH₂CH₃ | —CH₂CH₃ | Cl | H | H |
| 154 | —CH₂CH₃ | H | Cl | H | F |
| 155 | —CH₂CH₃ | H | Cl | H | —OCH₃ |
| 156 | —(CH₂)₂CH₃ | H | Cl | H | H |
| 157 | —(CH₂)₂CH₃ | —CH₃ | Cl | H | H |
| 158 | —(CH₂)₂CH₃ | —CH₂CH₃ | Cl | H | H |
| 159 | —(CH₂)₂CH₃ | H | Cl | H | F |
| 160 | —CH(CH₃)₂ | H | Cl | H | H |
| 161 | —CH(CH₃)₂ | —CH₃ | Cl | H | H |
| 162 | —CH(CH₃)₂ | —CH₂CH₃ | Cl | H | H |
| 163 | —(CH₂)₃CH₃ | H | Cl | H | H |
| 164 | —(CH₂)₃CH₃ | —CH₃ | Cl | H | H |
| 165 | —CH₂—HC═CH₂ | H | Cl | H | H |
| 166 | —CH₃ | H | F | H | H |
| 167 | —CH₃ | —CH₃ | F | H | H |
| 168 | —CH₃ | —CH₂CH₃ | F | H | H |
| 169 | —CH₃ | H | F | —CH₃ | H |
| 170 | —CH₃ | H | F | H | F |
| 171 | —CH₃ | H | F | H | —OCH₃ |
| 172 | —CH₂CH₃ | H | F | H | H |
| 173 | —CH₂CH₃ | —CH₃ | F | H | H |
| 174 | —CH₂CH₃ | —CH₂CH₃ | F | H | H |
| 175 | —CH₂CH₃ | H | F | H | F |
| 176 | —CH₂CH₃ | H | F | H | —OCH₃ |
| 177 | —(CH₂)₂CH₃ | H | F | H | H |
| 178 | —(CH₂)₂CH₃ | —CH₃ | F | H | H |
| 179 | —(CH₂)₂CH₃ | —CH₂CH₃ | F | H | H |
| 180 | —(CH₂)₂CH₃ | H | F | H | F |
| 181 | —CH(CH₃)₂ | H | F | H | H |
| 182 | —CH(CH₃)₂ | —CH₃ | F | H | H |
| 183 | —CH(CH₃)₂ | —CH₂CH₃ | F | H | H |
| 184 | —(CH₂)₃CH₃ | H | F | H | H |
| 185 | —(CH₂)₃CH₃ | —CH₃ | F | H | H |
| 186 | —CH₂—HC═CH₂ | H | F | H | H |
| 187 | —CH₃ | H | H | Br | H |
| 188 | —CH₃ | H | H | Br | H |
| 189 | —CH₃ | —CH₃ | H | Br | H |
| 190 | —CH₃ | —CH₂CH₃ | H | Br | H |
| 191 | —CH₃ | H | H | Br | F |
| 192 | —CH₃ | H | H | Br | —OCH₃ |
| 193 | —CH₂CH₃ | H | H | Br | H |
| 194 | —CH₂CH₃ | —CH₃ | H | Br | H |
| 195 | —CH₂CH₃ | —CH₂CH₃ | H | Br | H |
| 196 | —CH₂CH₃ | H | H | Br | F |
| 197 | —CH₂CH₃ | H | H | Br | —OCH₃ |
| 198 | —(CH₂)₂CH₃ | H | H | Br | H |
| 199 | —(CH₂)₂CH₃ | —CH₃ | H | Br | H |
| 200 | —(CH₂)₂CH₃ | —CH₂CH₃ | H | Br | H |
| 201 | —(CH₂)₂CH₃ | H | H | Br | F |
| 202 | —CH(CH₃)₂ | H | H | Br | H |
| 203 | —CH(CH₃)₂ | —CH₃ | H | Br | H |
| 204 | —CH(CH₃)₂ | —CH₂CH₃ | H | Br | H |
| 205 | —(CH₂)₃CH₃ | H | H | Br | H |
| 206 | —(CH₂)₃CH₃ | —CH₃ | H | Br | H |
| 207 | —CH₂—HC═CH₂ | H | H | Br | H |
| 208 | —CH₃ | H | H | Cl | H |
| 209 | —CH₃ | —CH₃ | H | Cl | H |
| 210 | —CH₃ | —CH₂CH₃ | H | Cl | H |
| 211 | —CH₃ | H | H | Cl | F |
| 212 | —CH₃ | H | H | Cl | —OCH₃ |
| 213 | —CH₂CH₃ | H | H | Cl | H |
| 214 | —CH₂CH₃ | —CH₃ | H | Cl | H |
| 215 | —CH₂CH₃ | —CH₂CH₃ | H | Cl | H |
| 216 | —CH₂CH₃ | H | H | Cl | F |
| 217 | —CH₂CH₃ | H | H | Cl | —OCH₃ |
| 218 | —(CH₂)₂CH₃ | H | H | Cl | H |
| 219 | —(CH₂)₂CH₃ | —CH₃ | H | Cl | H |
| 220 | —(CH₂)₂CH₃ | —CH₂CH₃ | H | Cl | H |
| 221 | —(CH₂)₂CH₃ | H | H | Cl | F |
| 222 | —CH(CH₃)₂ | H | H | Cl | H |
| 223 | —CH(CH₃)₂ | —CH₃ | H | Cl | H |
| 224 | —CH(CH₃)₂ | —CH₂CH₃ | H | Cl | H |
| 225 | —(CH₂)₃CH₃ | H | H | Cl | H |
| 226 | —(CH₂)₃CH₃ | —CH₃ | H | Cl | H |
| 227 | —CH₂—HC═CH₂ | H | H | Cl | H |
| 228 | —CH₃ | H | H | F | H |
| 229 | —CH₃ | —CH₃ | H | F | H |
| 230 | —CH₃ | —CH₂CH₃ | H | F | H |
| 231 | —CH₃ | H | H | F | —OCH₃ |
| 232 | —CH₃ | H | H | F | F |
| 233 | —CH₃ | H | H | F | H |
| 234 | —CH₂CH₃ | H | H | F | H |
| 235 | —CH₂CH₃ | —CH₃ | H | F | H |
| 236 | —CH₂CH₃ | —CH₂CH₃ | H | F | H |
| 237 | —CH₂CH₃ | H | H | F | F |
| 238 | —CH₂CH₃ | H | H | F | H |
| 239 | —(CH₂)₂CH₃ | H | H | F | H |
| 240 | —(CH₂)₂CH₃ | —CH₃ | H | F | H |
| 241 | —(CH₂)₂CH₃ | —CH₂CH₃ | H | F | H |
| 242 | —(CH₂)₂CH₃ | H | H | F | F |
| 243 | —CH(CH₃)₂ | H | H | F | H |
| 244 | —CH(CH₃)₂ | —CH₃ | H | F | H |
| 245 | —CH(CH₃)₂ | —CH₂CH₃ | H | F | H |
| 246 | —(CH₂)₃CH₃ | H | H | F | H |
| 247 | —(CH₂)₃CH₃ | —CH₃ | H | F | H |
| 248 | —CH₂—HC═CH₂ | H | H | F | H |
| 249 | —CH₃ | H | H | H | H |
| 250 | —CH₃ | H | —OH | H | H |
| 251 | —CH₃ | H | —OC(O)CH₃ | H | H |
| 252 | —CH₃ | H | —OCH₃ | H | H |
| 253 | —CH₃ | H | Br | H | H |
| 254 | —CH₃ | H | Cl | H | H |
| 255 | —CH₃ | H | F | H | H |
| 256 | —CH₃ | —CH₃ | H | H | H |
| 257 | —CH₃ | —CH₃ | —OH | H | H |
| 258 | —CH₃ | —CH₃ | —OC(O)CH₃ | H | H |
| 259 | —CH₃ | —CH₃ | —OCH₃ | H | H |
| 260 | —CH₃ | —CH₃ | Br | H | H |
| 261 | —CH₃ | —CH₃ | Cl | H | H |
| 262 | —CH₃ | —CH₃ | F | H | H |
| 263 | —CH₃ | H | H | H | F |
| 264 | —CH₃ | H | —OH | H | F |
| 265 | —CH₃ | H | —OC(O)CH₃ | H | F |
| 266 | —CH₃ | H | —OCH₃ | H | F |
| 267 | —CH₃ | H | Br | H | F |
| 268 | —CH₃ | H | Cl | H | F |
| 269 | —CH₃ | H | F | H | F |
| 270 | —CH₃ | H | H | H | —OCH₃ |
| 271 | —CH₃ | H | —OH | H | —OCH₃ |
| 272 | —CH₃ | H | —OC(O)CH₃ | H | —OCH₃ |
| 273 | —CH₃ | H | —OCH₃ | H | —OCH₃ |
| 274 | —CH₃ | H | Br | H | —OCH₃ |
| 275 | —CH₃ | H | Cl | H | —OCH₃ |
| 276 | —CH₃ | H | F | H | —OCH₃ |
| 277 | —CH₃ | H | H | —OH | H |
| 278 | —CH₃ | H | H | —OC(O)CH₃ | H |
| 279 | —CH₃ | H | H | —OCH₃ | H |
| 280 | —CH₃ | H | H | Br | H |
| 281 | —CH₃ | H | H | Cl | H |
| 282 | —CH₃ | H | H | F | H |
| 283 | —CH₃ | —CH₃ | H | H | H |
| 284 | —CH₃ | —CH₃ | H | —OH | H |
| 285 | —CH₃ | —CH₃ | H | —OC(O)CH₃ | H |
| 286 | —CH₃ | —CH₃ | H | —OCH₃ | H |
| 287 | —CH₃ | —CH₃ | H | Br | H |
| 288 | —CH₃ | —CH₃ | H | Cl | H |
| 289 | —CH₃ | —CH₃ | H | F | H |
| 290 | —CH₃ | H | H | H | F |
| 291 | —CH₃ | H | H | —OH | F |
| 292 | —CH₃ | H | H | —OC(O)CH₃ | F |
| 293 | —CH₃ | H | H | —OCH₃ | F |
| 294 | —CH₃ | H | H | Br | F |
| 295 | —CH₃ | H | H | Cl | F |
| 296 | —CH₃ | H | H | F | F |
| 297 | —CH₃ | H | H | H | —OCH₃ |
| 298 | —CH₃ | H | H | —OH | —OCH₃ |
| 299 | —CH₃ | H | H | —OC(O)CH₃ | —OCH₃ |
| 300 | —CH₃ | H | H | —OCH₃ | —OCH₃ |
| 301 | —CH₃ | H | H | Br | —OCH₃ |
| 302 | —CH₃ | H | H | Cl | —OCH₃ |
| 303 | —CH₃ | H | H | F | —OCH₃ |
| 304 | H | H | —OH | H | H |
| 305 | H | —CH₃ | —OH | H | H |
| 306 | H | —CH₂CH₃ | —OH | H | H |
| 307 | H | H | —OH | —CH₃ | H |
| 308 | H | H | —OH | H | F |
| 309 | H | H | —OH | H | —OCH₃ |
| 310 | H | H | H | H | H |
| 311 | H | —CH₃ | H | H | H |
| 312 | H | —CH₂CH₃ | H | H | H |
| 313 | H | H | H | —CH₃ | H |
| 314 | H | H | H | H | F |
| 315 | H | —CH₃ | H | H | —OCH₃ |
| 316 | H | H | H | H | —OCH₃ |
| 317 | H | H | H | H | F |
| 318 | —CH₃ | H | H | H | —OCH₃ |
| 319 | —CH₃ | H | H | H | F |

Other exemplary compounds include compounds of Formula II(c) set forth below in Table 3:

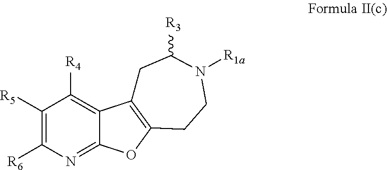

TABLE 3

| Ref. | R₁ₐ | R₃ | R₄ | R₅ | R₆ |
|---|---|---|---|---|---|
| 1 | —CH₃ | H | —OCH₃ | H | H |
| 2 | —CH₃ | —CH₃ | —OCH₃ | H | H |
| 3 | —CH₃ | —CH₂CH₃ | —OCH₃ | H | H |
| 4 | —CH₃ | H | —OCH₃ | —CH₃ | H |
| 5 | —CH₃ | H | —OCH₃ | H | F |
| 6 | —CH₃ | H | —OCH₃ | H | —OCH₃ |
| 7 | —CH₂CH₃ | —CH₃ | —OCH₃ | H | H |
| 8 | —CH₂CH₃ | —CH₂CH₃ | —OCH₃ | H | H |
| 9 | —CH₂CH₃ | H | —OCH₃ | —CH₃ | H |
| 10 | —CH₂CH₃ | H | —OCH₃ | H | F |
| 11 | —CH₂CH₃ | H | —OCH₃ | H | —OCH₃ |
| 12 | —(CH₂)₂CH₃ | —CH₃ | —OCH₃ | H | H |
| 13 | —(CH₂)₂CH₃ | —CH₂CH₃ | —OCH₃ | H | H |
| 14 | —(CH₂)₂CH₃ | H | —OCH₃ | H | F |
| 15 | —(CH₂)₂CH₃ | H | —OCH₃ | H | —OCH₃ |
| 16 | —CH(CH₃)₂ | —CH₃ | —OCH₃ | H | H |
| 17 | —CH(CH₃)₂ | —CH₂CH₃ | —OCH₃ | H | H |
| 18 | —CH(CH₃)₂ | H | —OCH₃ | H | F |
| 19 | —(CH₂)₃CH₃ | H | —OCH₃ | H | H |
| 20 | —(CH₂)₃CH₃ | —CH₃ | —OCH₃ | H | H |
| 21 | —CH₂—HC═CH₂ | H | —OCH₃ | H | H |
| 22 | —CH₃ | H | —OH | H | H |
| 23 | —CH₃ | —CH₃ | —OH | H | H |
| 24 | —CH₃ | —CH₂CH₃ | —OH | H | H |
| 25 | —CH₃ | H | —OH | H | F |
| 26 | —CH₃ | H | —OH | H | —OCH₃ |
| 27 | —CH₂CH₃ | H | —OH | H | H |
| 28 | —CH₂CH₃ | —CH₃ | —OH | H | H |
| 29 | —CH₂CH₃ | —CH₂CH₃ | —OH | H | H |
| 30 | —CH₂CH₃ | H | —OH | H | F |
| 31 | —CH₂CH₃ | H | —OH | H | —OCH₃ |
| 32 | —(CH₂)₂CH₃ | H | —OH | H | H |
| 33 | —(CH₂)₂CH₃ | —CH₃ | —OH | H | H |
| 34 | —(CH₂)₂CH₃ | —CH₂CH₃ | —OH | H | H |
| 35 | —(CH₂)₂CH₃ | H | —OH | H | F |
| 36 | —CH(CH₃)₂ | H | —OH | H | H |
| 37 | —CH(CH₃)₂ | —CH₃ | —OH | H | H |
| 38 | —CH(CH₃)₂ | —CH₂CH₃ | —OH | H | H |
| 39 | —(CH₂)₃CH₃ | H | —OH | H | H |
| 40 | —(CH₂)₃CH₃ | H | —OH | H | H |
| 41 | —CH₂—HC═CH₂ | H | —OH | H | H |
| 42 | —CH₃ | H | —OC(O)CH₃ | H | H |
| 43 | —CH₃ | —CH₃ | —OC(O)CH₃ | H | H |
| 44 | —CH₃ | —CH₂CH₃ | —OC(O)CH₃ | H | H |
| 45 | —CH₃ | H | —OC(O)CH₃ | —CH₃ | H |
| 46 | —CH₃ | H | —OC(O)CH₃ | H | F |
| 47 | —CH₃ | H | —OC(O)CH₃ | H | —OCH₃ |
| 48 | —CH₂CH₃ | H | —OC(O)CH₃ | H | H |
| 49 | —CH₂CH₃ | —CH₃ | —OC(O)CH₃ | H | H |
| 50 | —CH₂CH₃ | —CH₂CH₃ | —OC(O)CH₃ | H | H |
| 51 | —CH₂CH₃ | H | —OC(O)CH₃ | H | F |
| 52 | —CH₂CH₃ | H | —OC(O)CH₃ | H | —OCH₃ |
| 53 | —(CH₂)₂CH₃ | H | —OC(O)CH₃ | H | H |
| 54 | —(CH₂)₂CH₃ | —CH₃ | —OC(O)CH₃ | H | H |
| 55 | —(CH₂)₂CH₃ | —CH₂CH₃ | —OC(O)CH₃ | H | H |
| 56 | —(CH₂)₂CH₃ | H | —OC(O)CH₃ | H | F |
| 57 | —CH(CH₃)₂ | H | —OC(O)CH₃ | H | H |
| 58 | —CH(CH₃)₂ | —CH₃ | —OC(O)CH₃ | H | H |
| 59 | —CH(CH₃)₂ | —CH₂CH₃ | —OC(O)CH₃ | H | H |
| 60 | —(CH₂)₃CH₃ | H | —OC(O)CH₃ | H | H |
| 61 | —(CH₂)₃CH₃ | —CH₃ | —OC(O)CH₃ | H | H |
| 62 | —CH₂—HC═CH₂ | H | —OC(O)CH₃ | H | H |
| 63 | —CH₃ | H | H | —OCH₃ | H |
| 64 | —CH₃ | —CH₃ | H | —OCH₃ | H |
| 65 | —CH₃ | —CH₂CH₃ | H | —OCH₃ | H |
| 66 | —CH₃ | H | H | —OCH₃ | H |
| 67 | —CH₃ | H | H | —OCH₃ | F |
| 68 | —CH₃ | H | H | —OCH₃ | —OCH₃ |
| 69 | —CH₂CH₃ | H | H | —OCH₃ | H |
| 70 | —CH₂CH₃ | —CH₃ | H | —OCH₃ | H |
| 71 | —CH₂CH₃ | —CH₂CH₃ | H | —OCH₃ | H |
| 72 | —CH₂CH₃ | H | H | —OCH₃ | F |
| 73 | —CH₂CH₃ | H | H | —OCH₃ | —OCH₃ |
| 74 | —(CH₂)₂CH₃ | H | H | —OCH₃ | H |
| 75 | —(CH₂)₂CH₃ | —CH₃ | H | —OCH₃ | H |
| 76 | —(CH₂)₂CH₃ | —CH₂CH₃ | H | —OCH₃ | H |
| 77 | —(CH₂)₂CH₃ | H | H | —OCH₃ | F |
| 78 | —CH(CH₃)₂ | H | H | —OCH₃ | H |
| 79 | —CH(CH₃)₂ | —CH₃ | H | —OCH₃ | H |
| 80 | —CH(CH₃)₂ | —CH₂CH₃ | H | —OCH₃ | H |
| 81 | —(CH₂)₃CH₃ | H | H | —OCH₃ | H |
| 82 | —(CH₂)₃CH₃ | —CH₃ | H | —OCH₃ | H |
| 83 | —CH₂—HC═CH₂ | H | H | —OCH₃ | H |
| 84 | —CH₃ | H | H | —OH | H |
| 85 | —CH₃ | —CH₃ | H | —OH | H |
| 86 | —CH₃ | —CH₂CH₃ | H | —OH | H |
| 87 | —CH₃ | H | H | —OH | F |
| 88 | —CH₃ | H | H | —OH | —OCH₃ |
| 89 | —CH₂CH₃ | H | H | —OH | H |
| 90 | —CH₂CH₃ | —CH₃ | H | —OH | H |
| 91 | —CH₂CH₃ | —CH₂CH₃ | H | —OH | H |
| 92 | —CH₂CH₃ | H | H | —OH | F |
| 93 | —CH₂CH₃ | H | H | —OH | —OCH₃ |
| 94 | —(CH₂)₂CH₃ | H | H | —OH | H |
| 95 | —(CH₂)₂CH₃ | —CH₃ | H | —OH | H |
| 96 | —(CH₂)₂CH₃ | —CH₂CH₃ | H | —OH | H |
| 97 | —(CH₂)₂CH₃ | H | H | —OH | F |
| 98 | —CH(CH₃)₂ | H | H | —OH | H |
| 99 | —CH(CH₃)₂ | —CH₃ | H | —OH | H |
| 100 | —CH(CH₃)₂ | —CH₂CH₃ | H | —OH | H |
| 101 | —(CH₂)₃CH₃ | H | H | —OH | H |
| 102 | —(CH₂)₃CH₃ | —CH₃ | H | —OH | H |
| 103 | —CH₂—HC═CH₂ | H | H | —OH | H |
| 104 | —CH₃ | H | H | —OC(O)CH₃ | H |
| 105 | —CH₃ | —CH₃ | H | —OC(O)CH₃ | H |
| 106 | —CH₃ | —CH₂CH₃ | H | —OC(O)CH₃ | H |
| 107 | —CH₃ | H | —CH₃ | —OC(O)CH₃ | H |
| 108 | —CH₃ | H | H | —OC(O)CH₃ | F |
| 109 | —CH₃ | H | H | —OC(O)CH₃ | —OCH₃ |
| 110 | —CH₂CH₃ | H | H | —OC(O)CH₃ | H |
| 111 | —CH₂CH₃ | —CH₃ | H | —OC(O)CH₃ | H |
| 112 | —CH₂CH₃ | —CH₂CH₃ | H | —OC(O)CH₃ | H |
| 113 | —CH₂CH₃ | H | H | —OC(O)CH₃ | F |
| 114 | —CH₂CH₃ | H | H | —OC(O)CH₃ | —OCH₃ |
| 115 | —(CH₂)₂CH₃ | H | H | —OC(O)CH₃ | H |
| 116 | —(CH₂)₂CH₃ | —CH₃ | H | —OC(O)CH₃ | H |
| 117 | —(CH₂)₂CH₃ | —CH₂CH₃ | H | —OC(O)CH₃ | H |
| 118 | —(CH₂)₂CH₃ | H | H | —OC(O)CH₃ | F |
| 119 | —CH(CH₃)₂ | H | H | —OC(O)CH₃ | H |
| 120 | —CH(CH₃)₂ | —CH₃ | H | —OC(O)CH₃ | H |
| 121 | —CH(CH₃)₂ | —CH₂CH₃ | H | —OC(O)CH₃ | H |
| 122 | —(CH₂)₃CH₃ | H | H | —OC(O)CH₃ | H |
| 123 | —(CH₂)₃CH₃ | —CH₃ | H | —OC(O)CH₃ | H |
| 124 | —CH₂—HC═CH₂ | H | H | —OC(O)CH₃ | H |
| 125 | —CH₃ | H | Br | H | H |
| 126 | —CH₃ | —CH₃ | Br | H | H |
| 127 | —CH₃ | —CH₂CH₃ | Br | H | H |
| 128 | —CH₃ | H | Br | —CH₃ | H |
| 129 | —CH₃ | H | Br | H | F |
| 130 | —CH₃ | H | Br | H | —OCH₃ |
| 131 | —CH₂CH₃ | H | Br | H | H |
| 132 | —CH₂CH₃ | —CH₃ | Br | H | H |
| 133 | —CH₂CH₃ | —CH₂CH₃ | Br | H | H |
| 134 | —CH₂CH₃ | H | Br | H | F |
| 135 | —CH₂CH₃ | H | Br | H | —OCH₃ |
| 135 | —(CH₂)₂CH₃ | H | Br | H | H |
| 137 | —(CH₂)₂CH₃ | —CH₃ | Br | H | H |
| 138 | —(CH₂)₂CH₃ | —CH₂CH₃ | Br | H | H |
| 139 | —(CH₂)₂CH₃ | H | Br | H | F |
| 140 | —CH(CH₃)₂ | H | Br | H | H |
| 141 | —CH(CH₃)₂ | —CH₃ | Br | H | H |
| 142 | —CH(CH₃)₂ | —CH₂CH₃ | Br | H | H |
| 143 | —(CH₂)₃CH₃ | H | Br | H | H |
| 144 | —(CH₂)₃CH₃ | —CH₃ | Br | H | H |
| 145 | —CH₂—HC═CH₂ | H | Br | H | H |
| 146 | —CH₃ | H | Cl | H | H |
| 147 | —CH₃ | —CH₃ | Cl | H | H |
| 148 | —CH₃ | —CH₂CH₃ | Cl | H | H |
| 149 | —CH₃ | H | Cl | H | F |
| 150 | —CH₃ | H | Cl | H | —OCH₃ |
| 151 | —CH₂CH₃ | H | Cl | H | H |
| 152 | —CH₂CH₃ | —CH₃ | Cl | H | H |
| 153 | —CH₂CH₃ | —CH₂CH₃ | Cl | H | H |
| 154 | —CH₂CH₃ | H | Cl | H | F |
| 155 | —CH₂CH₃ | H | Cl | H | —OCH₃ |
| 156 | —(CH₂)₂CH₃ | H | Cl | H | H |
| 157 | —(CH₂)₂CH₃ | —CH₃ | Cl | H | H |
| 158 | —(CH₂)₂CH₃ | —CH₂CH₃ | Cl | H | H |
| 159 | —(CH₂)₂CH₃ | H | Cl | H | F |
| 160 | —CH(CH₃)₂ | H | Cl | H | H |
| 161 | —CH(CH₃)₂ | —CH₃ | Cl | H | H |
| 162 | —CH(CH₃)₂ | —CH₂CH₃ | Cl | H | H |
| 163 | —(CH₂)₃CH₃ | H | Cl | H | H |
| 164 | —(CH₂)₃CH₃ | —CH₃ | Cl | H | H |
| 165 | —CH₂—HC═CH₂ | H | Cl | H | H |
| 166 | —CH₃ | H | F | H | H |
| 167 | —CH₃ | —CH₃ | F | H | H |
| 168 | —CH₃ | —CH₂CH₃ | F | H | H |
| 169 | —CH₃ | H | F | —CH₃ | H |
| 170 | —CH₃ | H | F | H | F |
| 171 | —CH₃ | H | F | H | —OCH₃ |
| 172 | —CH₂CH₃ | H | F | H | H |
| 173 | —CH₂CH₃ | —CH₃ | F | H | H |
| 174 | —CH₂CH₃ | —CH₂CH₃ | F | H | H |
| 175 | —CH₂CH₃ | H | F | H | F |
| 176 | —CH₂CH₃ | H | F | H | —OCH₃ |
| 177 | —(CH₂)₂CH₃ | H | F | H | H |
| 178 | —(CH₂)₂CH₃ | —CH₃ | F | H | H |
| 179 | —(CH₂)₂CH₃ | —CH₂CH₃ | F | H | H |
| 180 | —(CH₂)₂CH₃ | H | F | H | F |
| 181 | —CH(CH₃)₂ | H | F | H | H |
| 182 | —CH(CH₃)₂ | —CH₃ | F | H | H |
| 183 | —CH(CH₃)₂ | —CH₂CH₃ | F | H | H |
| 184 | —(CH₂)₃CH₃ | H | F | H | H |
| 185 | —(CH₂)₃CH₃ | —CH₃ | F | H | H |
| 186 | —CH₂—HC═CH₂ | H | F | H | H |
| 187 | —CH₃ | H | H | Br | H |
| 188 | —CH₃ | H | H | Br | H |
| 189 | —CH₃ | —CH₃ | H | Br | H |
| 190 | —CH₃ | —CH₂CH₃ | H | Br | H |
| 191 | —CH₃ | H | H | Br | F |
| 192 | —CH₃ | H | H | Br | —OCH₃ |
| 193 | —CH₂CH₃ | H | H | Br | H |
| 194 | —CH₂CH₃ | —CH₃ | H | Br | H |
| 195 | —CH₂CH₃ | —CH₂CH₃ | H | Br | H |
| 196 | —CH₂CH₃ | H | H | Br | F |
| 197 | —CH₂CH₃ | H | H | Br | —OCH₃ |
| 198 | —(CH₂)₂CH₃ | H | H | Br | H |
| 199 | —(CH₂)₂CH₃ | —CH₃ | H | Br | H |
| 200 | —(CH₂)₂CH₃ | —CH₂CH₃ | H | Br | H |
| 201 | —(CH₂)₂CH₃ | H | H | Br | F |
| 202 | —CH(CH₃)₂ | H | H | Br | H |
| 203 | —CH(CH₃)₂ | —CH₃ | H | Br | H |
| 204 | —CH(CH₃)₂ | —CH₂CH₃ | H | Br | H |
| 205 | —(CH₂)₃CH₃ | H | H | Br | H |
| 206 | —(CH₂)₃CH₃ | —CH₃ | H | Br | H |
| 207 | —CH₂—HC═CH₂ | H | H | Br | H |
| 208 | —CH₃ | H | H | Cl | H |
| 209 | —CH₃ | —CH₃ | H | Cl | H |
| 210 | —CH₃ | —CH₂CH₃ | H | Cl | H |
| 211 | —CH₃ | H | H | Cl | F |
| 212 | —CH₃ | H | H | Cl | —OCH₃ |
| 213 | —CH₂CH₃ | H | H | Cl | H |
| 214 | —CH₂CH₃ | —CH₃ | H | Cl | H |
| 215 | —CH₂CH₃ | —CH₂CH₃ | H | Cl | H |
| 216 | —CH₂CH₃ | H | H | Cl | F |
| 217 | —CH₂CH₃ | H | H | Cl | —OCH₃ |
| 218 | —(CH₂)₂CH₃ | H | H | Cl | H |
| 219 | —(CH₂)₂CH₃ | —CH₃ | H | Cl | H |
| 220 | —(CH₂)₂CH₃ | —CH₂CH₃ | H | Cl | H |
| 221 | —(CH₂)₂CH₃ | H | H | Cl | F |
| 222 | —CH(CH₃)₂ | H | H | Cl | H |
| 223 | —CH(CH₃)₂ | —CH₃ | H | Cl | H |
| 224 | —CH(CH₃)₂ | —CH₂CH₃ | H | Cl | H |
| 225 | —(CH₂)₃CH₃ | H | H | Cl | H |
| 226 | —(CH₂)₃CH₃ | —CH₃ | H | Cl | H |
| 227 | —CH₂—HC═CH₂ | H | H | Cl | H |
| 228 | —CH₃ | H | H | F | H |
| 229 | —CH₃ | —CH₃ | H | F | H |
| 230 | —CH₃ | —CH₂CH₃ | H | F | H |
| 231 | —CH₃ | H | H | F | —OCH₃ |
| 232 | —CH₃ | H | H | F | F |
| 233 | —CH₃ | H | H | F | H |
| 234 | —CH₂CH₃ | H | H | F | H |
| 235 | —CH₂CH₃ | —CH₃ | H | F | H |
| 236 | —CH₂CH₃ | —CH₂CH₃ | H | F | H |
| 237 | —CH₂CH₃ | H | H | F | F |
| 238 | —CH₂CH₃ | H | H | F | H |
| 239 | —(CH₂)₂CH₃ | H | H | F | H |
| 240 | —(CH₂)₂CH₃ | —CH₃ | H | F | H |
| 241 | —(CH₂)₂CH₃ | —CH₂CH₃ | H | F | H |
| 242 | —(CH₂)₂CH₃ | H | H | F | F |
| 243 | —CH(CH₃)₂ | H | H | F | H |
| 244 | —CH(CH₃)₂ | —CH₃ | H | F | H |
| 245 | —CH(CH₃)₂ | —CH₂CH₃ | H | F | H |
| 246 | —(CH₂)₃CH₃ | H | H | F | H |
| 247 | —(CH₂)₃CH₃ | —CH₃ | H | F | H |
| 248 | —CH₂—HC═CH₂ | H | H | F | H |
| 249 | —CH₃ | H | H | H | H |
| 250 | —CH₃ | H | —OH | H | H |
| 251 | —CH₃ | H | —OC(O)CH₃ | H | H |
| 252 | —CH₃ | H | —OCH₃ | H | H |
| 253 | —CH₃ | H | Br | H | H |
| 254 | —CH₃ | H | Cl | H | H |
| 255 | —CH₃ | H | F | H | H |
| 256 | —CH₃ | —CH₃ | H | H | H |
| 257 | —CH₃ | —CH₃ | —OH | H | H |
| 258 | —CH₃ | —CH₃ | —OC(O)CH₃ | H | H |
| 259 | —CH₃ | —CH₃ | —OCH₃ | H | H |
| 260 | —CH₃ | —CH₃ | Br | H | H |
| 261 | —CH₃ | —CH₃ | Cl | H | H |
| 262 | —CH₃ | —CH₃ | F | H | H |
| 263 | —CH₃ | H | H | H | F |
| 264 | —CH₃ | H | —OH | H | F |
| 265 | —CH₃ | H | —OC(O)CH₃ | H | F |
| 266 | —CH₃ | H | —OCH₃ | H | F |
| 267 | —CH₃ | H | Br | H | F |
| 268 | —CH₃ | H | Cl | H | F |
| 269 | —CH₃ | H | F | H | F |
| 270 | —CH₃ | H | H | H | —OCH₃ |
| 271 | —CH₃ | H | —OH | H | —OCH₃ |
| 272 | —CH₃ | H | —OC(O)CH₃ | H | —OCH₃ |
| 273 | —CH₃ | H | —OCH₃ | H | —OCH₃ |
| 274 | —CH₃ | H | Br | H | —OCH₃ |
| 275 | —CH₃ | H | Cl | H | —OCH₃ |
| 276 | —CH₃ | H | F | H | —OCH₃ |
| 277 | —CH₃ | H | H | —OH | H |
| 278 | —CH₃ | H | H | —OC(O)CH₃ | H |
| 279 | —CH₃ | H | H | —OCH₃ | H |
| 280 | —CH₃ | H | H | Br | H |
| 281 | —CH₃ | H | H | Cl | H |
| 282 | —CH₃ | H | H | F | H |
| 283 | —CH₃ | —CH₃ | H | H | H |
| 284 | —CH₃ | —CH₃ | H | —OH | H |
| 285 | —CH₃ | —CH₃ | H | —OC(O)CH₃ | H |
| 286 | —CH₃ | —CH₃ | H | —OCH₃ | H |
| 287 | —CH₃ | —CH₃ | H | Br | H |
| 288 | —CH₃ | —CH₃ | H | Cl | H |
| 289 | —CH₃ | —CH₃ | H | F | H |
| 290 | —CH₃ | H | H | H | F |
| 291 | —CH₃ | H | H | —OH | F |
| 292 | —CH₃ | H | H | —OC(O)CH₃ | F |
| 293 | —CH₃ | H | H | —OCH₃ | F |
| 294 | —CH₃ | H | H | Br | F |
| 295 | —CH₃ | H | H | Cl | F |
| 296 | —CH₃ | H | H | F | F |
| 297 | —CH₃ | H | H | H | —OCH₃ |
| 298 | —CH₃ | H | H | —OH | —OCH₃ |
| 299 | —CH₃ | H | H | —OC(O)CH₃ | —OCH₃ |
| 300 | —CH₃ | H | H | —OCH₃ | —OCH₃ |
| 301 | —CH₃ | H | H | Br | —OCH₃ |
| 302 | —CH₃ | H | H | Cl | —OCH₃ |
| 303 | —CH₃ | H | H | F | —OCH₃ |
| 304 | H | H | —OH | H | H |
| 305 | H | —CH₃ | —OH | H | H |
| 306 | H | —CH₂CH₃ | —OH | H | H |
| 307 | H | H | —OH | —CH₃ | H |
| 308 | H | H | —OH | H | F |
| 309 | H | H | —OH | H | —OCH₃ |
| 310 | H | H | H | H | H |
| 311 | H | —CH₃ | H | H | H |
| 312 | H | —CH₂CH₃ | H | H | H |
| 313 | H | H | H | —CH₃ | H |
| 314 | H | H | H | H | F |
| 315 | H | —CH₃ | H | H | —OCH₃ |
| 316 | H | H | H | H | —OCH₃ |
| 317 | H | H | H | H | F |
| 318 | —CH₃ | H | H | H | —OCH₃ |
| 319 | —CH₃ | H | H | H | F |

Other exemplary compounds include compounds of Formula II(d) set forth below in Table 4:

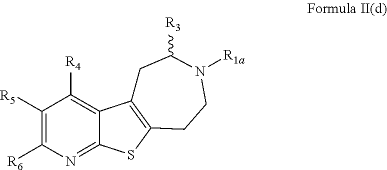

TABLE 4

| Ref. | R₁ₐ | R₃ | R₄ | R₅ | R₆ |
|---|---|---|---|---|---|
| 1 | —CH₃ | H | —OCH₃ | H | H |
| 2 | —CH₃ | —CH₃ | —OCH₃ | H | H |
| 3 | —CH₃ | —CH₂CH₃ | —OCH₃ | H | H |
| 4 | —CH₃ | H | —OCH₃ | —CH₃ | H |
| 5 | —CH₃ | H | —OCH₃ | H | F |
| 6 | —CH₃ | H | —OCH₃ | H | —OCH₃ |
| 7 | —CH₂CH₃ | —CH₃ | —OCH₃ | H | H |
| 8 | —CH₂CH₃ | —CH₂CH₃ | —OCH₃ | H | H |
| 9 | —CH₂CH₃ | H | —OCH₃ | —CH₃ | H |
| 10 | —CH₂CH₃ | H | —OCH₃ | H | F |
| 11 | —CH₂CH₃ | H | —OCH₃ | H | —OCH₃ |
| 12 | —(CH₂)₂CH₃ | —CH₃ | —OCH₃ | H | H |
| 13 | —(CH₂)₂CH₃ | —CH₂CH₃ | —OCH₃ | H | H |
| 14 | —(CH₂)₂CH₃ | H | —OCH₃ | H | F |
| 15 | —(CH₂)₂CH₃ | H | —OCH₃ | H | —OCH₃ |
| 16 | —CH(CH₃)₂ | —CH₃ | —OCH₃ | H | H |
| 17 | —CH(CH₃)₂ | —CH₂CH₃ | —OCH₃ | H | H |
| 18 | —CH(CH₃)₂ | H | —OCH₃ | H | F |
| 19 | —(CH₂)₃CH₃ | H | —OCH₃ | H | H |
| 20 | —(CH₂)₃CH₃ | —CH₃ | —OCH₃ | H | H |
| 21 | —CH₂—HC═CH₂ | H | —OCH₃ | H | H |
| 22 | —CH₃ | H | —OH | H | H |
| 23 | —CH₃ | —CH₃ | —OH | H | H |
| 24 | —CH₃ | —CH₂CH₃ | —OH | H | H |
| 25 | —CH₃ | H | —OH | H | F |
| 26 | —CH₃ | H | —OH | H | —OCH₃ |
| 27 | —CH₂CH₃ | H | —OH | H | H |
| 28 | —CH₂CH₃ | —CH₃ | —OH | H | H |
| 29 | —CH₂CH₃ | —CH₂CH₃ | —OH | H | H |
| 30 | —CH₂CH₃ | H | —OH | H | F |
| 31 | —CH₂CH₃ | H | —OH | H | —OCH₃ |
| 32 | —(CH₂)₂CH₃ | H | —OH | H | H |
| 33 | —(CH₂)₂CH₃ | —CH₃ | —OH | H | H |
| 34 | —(CH₂)₂CH₃ | —CH₂CH₃ | —OH | H | H |
| 35 | —(CH₂)₂CH₃ | H | —OH | H | F |
| 36 | —CH(CH₃)₂ | H | —OH | H | H |
| 37 | —CH(CH₃)₂ | —CH₃ | —OH | H | H |
| 38 | —CH(CH₃)₂ | —CH₂CH₃ | —OH | H | H |
| 39 | —(CH₂)₃CH₃ | H | —OH | H | H |
| 40 | —(CH₂)₃CH₃ | H | —OH | H | H |
| 41 | —CH₂—HC═CH₂ | H | —OH | H | H |
| 42 | —CH₃ | H | —OC(O)CH₃ | H | H |
| 43 | —CH₃ | —CH₃ | —OC(O)CH₃ | H | H |
| 44 | —CH₃ | —CH₂CH₃ | —OC(O)CH₃ | H | H |
| 45 | —CH₃ | H | —OC(O)CH₃ | —CH₃ | H |
| 46 | —CH₃ | H | —OC(O)CH₃ | H | F |
| 47 | —CH₃ | H | —OC(O)CH₃ | H | —OCH₃ |
| 48 | —CH₂CH₃ | H | —OC(O)CH₃ | H | H |
| 49 | —CH₂CH₃ | —CH₃ | —OC(O)CH₃ | H | H |
| 50 | —CH₂CH₃ | CH₂CH₃ | —OC(O)CH₃ | H | H |
| 51 | —CH₂CH₃ | H | —OC(O)CH₃ | H | F |
| 52 | —CH₂CH₃ | H | —OC(O)CH₃ | H | —OCH₃ |
| 53 | —(CH₂)₂CH₃ | H | —OC(O)CH₃ | H | H |
| 54 | —(CH₂)₂CH₃ | —CH₃ | —OC(O)CH₃ | H | H |
| 55 | —(CH₂)₂CH₃ | —CH₂CH₃ | —OC(O)CH₃ | H | H |
| 56 | —(CH₂)₂CH₃ | H | —OC(O)CH₃ | H | F |
| 57 | —CH(CH₃)₂ | H | —OC(O)CH₃ | H | H |
| 58 | —CH(CH₃)₂ | —CH₃ | —OC(O)CH₃ | H | H |
| 59 | —CH(CH₃)₂ | —CH₂CH₃ | —OC(O)CH₃ | H | H |
| 60 | —(CH₂)₃CH₃ | H | —OC(O)CH₃ | H | H |
| 61 | —(CH₂)₃CH₃ | —CH₃ | —OC(O)CH₃ | H | H |
| 62 | —CH₂—HC═CH₂ | H | —OC(O)CH₃ | H | H |
| 63 | —CH₃ | H | H | —OCH₃ | H |
| 64 | —CH₃ | —CH₃ | H | —OCH₃ | H |
| 65 | —CH₃ | —CH₂CH₃ | H | —OCH₃ | H |
| 66 | —CH₃ | H | H | —OCH₃ | H |
| 67 | —CH₃ | H | H | —OCH₃ | F |
| 68 | —CH₃ | H | H | —OCH₃ | —OCH₃ |
| 69 | —CH₂CH₃ | H | H | —OCH₃ | H |
| 70 | —CH₂CH₃ | —CH₃ | H | —OCH₃ | H |
| 71 | —CH₂CH₃ | —CH₂CH₃ | H | —OCH₃ | H |
| 72 | —CH₂CH₃ | H | H | —OCH₃ | F |
| 73 | —CH₂CH₃ | H | H | —OCH₃ | —OCH₃ |
| 74 | —(CH₂)₂CH₃ | H | H | —OCH₃ | H |
| 75 | —(CH₂)₂CH₃ | —CH₃ | H | —OCH₃ | H |
| 76 | —(CH₂)₂CH₃ | —CH₂CH₃ | H | —OCH₃ | H |
| 77 | —(CH₂)₂CH₃ | H | H | —OCH₃ | F |
| 78 | —CH(CH₃)₂ | H | H | —OCH₃ | H |
| 79 | —CH(CH₃)₂ | —CH₃ | H | —OCH₃ | H |
| 80 | —CH(CH₃)₂ | —CH₂CH₃ | H | —OCH₃ | H |
| 81 | —(CH₂)₃CH₃ | H | H | —OCH₃ | H |
| 82 | —(CH₂)₃CH₃ | —CH₃ | H | —OCH₃ | H |
| 83 | —CH₂—HC═CH₂ | H | H | —OCH₃ | H |
| 84 | —CH₃ | H | H | —OH | H |
| 85 | —CH₃ | —CH₃ | H | —OH | H |
| 86 | —CH₃ | —CH₂CH₃ | H | —OH | H |
| 87 | —CH₃ | H | H | —OH | F |
| 88 | —CH₃ | H | H | —OH | —OCH₃ |
| 89 | —CH₂CH₃ | H | H | —OH | H |
| 90 | —CH₂CH₃ | —CH₃ | H | —OH | H |
| 91 | —CH₂CH₃ | —CH₂CH₃ | H | —OH | H |
| 92 | —CH₂CH₃ | H | H | —OH | F |
| 93 | —CH₂CH₃ | H | H | —OH | —OCH₃ |
| 94 | —(CH₂)₂CH₃ | H | H | —OH | H |
| 95 | —(CH₂)₂CH₃ | —CH₃ | H | —OH | H |
| 96 | —(CH₂)₂CH₃ | —CH₂CH₃ | H | —OH | H |
| 97 | —(CH₂)₂CH₃ | H | H | —OH | F |
| 98 | —CH(CH₃)₂ | H | H | —OH | H |
| 99 | —CH(CH₃)₂ | —CH₃ | H | —OH | H |
| 100 | —CH(CH₃)₂ | —CH₂CH₃ | H | —OH | H |
| 101 | —(CH₂)₃CH₃ | H | H | —OH | H |
| 102 | —(CH₂)₃CH₃ | —CH₃ | H | —OH | H |
| 103 | —CH₂—HC═CH₂ | H | H | —OH | H |
| 104 | —CH₃ | H | H | —OC(O)CH₃ | H |
| 105 | —CH₃ | —CH₃ | H | —OC(O)CH₃ | H |
| 106 | —CH₃ | —CH₂CH₃ | H | —OC(O)CH₃ | H |
| 107 | —CH₃ | H | —CH₃ | —OC(O)CH₃ | H |
| 108 | —CH₃ | H | H | —OC(O)CH₃ | F |
| 109 | —CH₃ | H | H | —OC(O)CH₃ | —OCH₃ |
| 110 | —CH₂CH₃ | H | H | —OC(O)CH₃ | H |
| 111 | —CH₂CH₃ | —CH₃ | H | —OC(O)CH₃ | H |
| 112 | —CH₂CH₃ | —CH₂CH₃ | H | —OC(O)CH₃ | H |
| 113 | —CH₂CH₃ | H | H | —OC(O)CH₃ | F |
| 114 | —CH₂CH₃ | H | H | —OC(O)CH₃ | —OCH₃ |
| 115 | —(CH₂)₂CH₃ | H | H | —OC(O)CH₃ | H |
| 116 | —(CH₂)₂CH₃ | —CH₃ | H | —OC(O)CH₃ | H |
| 117 | —(CH₂)₂CH₃ | —CH₂CH₃ | H | —OC(O)CH₃ | H |
| 118 | —(CH₂)₂CH₃ | H | H | —OC(O)CH₃ | F |
| 119 | —CH(CH₃)₂ | H | H | —OC(O)CH₃ | H |
| 120 | —CH(CH₃)₂ | —CH₃ | H | —OC(O)CH₃ | H |
| 121 | —CH(CH₃)₂ | —CH₂CH₃ | H | —OC(O)CH₃ | H |
| 122 | —(CH₂)₃CH₃ | H | H | —OC(O)CH₃ | H |
| 123 | —(CH₂)₃CH₃ | —CH₃ | H | —OC(O)CH₃ | H |
| 124 | —CH₂—HC═CH₂ | H | H | —OC(O)CH₃ | H |
| 125 | —CH₃ | H | Br | H | H |
| 126 | —CH₃ | —CH₃ | Br | H | H |
| 127 | —CH₃ | —CH₂CH₃ | Br | H | H |
| 128 | —CH₃ | H | Br | —CH₃ | H |
| 129 | —CH₃ | H | Br | H | F |
| 130 | —CH₃ | H | Br | H | —OCH₃ |
| 131 | —CH₂CH₃ | H | Br | H | H |
| 132 | —CH₂CH₃ | —CH₃ | Br | H | H |
| 133 | —CH₂CH₃ | —CH₂CH₃ | Br | H | H |
| 134 | —CH₂CH₃ | H | Br | H | F |
| 135 | —CH₂CH₃ | H | Br | H | —OCH₃ |
| 135 | —(CH₂)₂CH₃ | H | Br | H | H |
| 137 | —(CH₂)₂CH₃ | —CH₃ | Br | H | H |
| 138 | —(CH₂)₂CH₃ | —CH₂CH₃ | Br | H | H |
| 139 | —(CH₂)₂CH₃ | H | Br | H | F |
| 140 | —CH(CH₃)₂ | H | Br | H | H |
| 141 | —CH(CH₃)₂ | —CH₃ | Br | H | H |
| 142 | —CH(CH₃)₂ | —CH₂CH₃ | Br | H | H |
| 143 | —(CH₂)₃CH₃ | H | Br | H | H |
| 144 | —(CH₂)₃CH₃ | —CH₃ | Br | H | H |
| 145 | —CH₂—HC═CH₂ | H | Br | H | H |
| 146 | —CH₃ | H | Cl | H | H |
| 147 | —CH₃ | —CH₃ | Cl | H | H |
| 148 | —CH₃ | —CH₂CH₃ | Cl | H | H |
| 149 | —CH₃ | H | Cl | H | F |
| 150 | —CH₃ | H | Cl | H | —OCH₃ |
| 151 | —CH₂CH₃ | H | Cl | H | H |
| 152 | —CH₂CH₃ | —CH₃ | Cl | H | H |
| 153 | —CH₂CH₃ | —CH₂CH₃ | Cl | H | H |
| 154 | —CH₂CH₃ | H | Cl | H | F |
| 155 | —CH₂CH₃ | H | Cl | H | —OCH₃ |
| 156 | —(CH₂)₂CH₃ | H | Cl | H | H |
| 157 | —(CH₂)₂CH₃ | —CH₃ | Cl | H | H |
| 158 | —(CH₂)₂CH₃ | —CH₂CH₃ | Cl | H | H |
| 159 | —(CH₂)₂CH₃ | H | Cl | H | F |
| 160 | —CH(CH₃)₂ | H | Cl | H | H |
| 161 | —CH(CH₃)₂ | —CH₃ | Cl | H | H |
| 162 | —CH(CH₃)₂ | —CH₂CH₃ | Cl | H | H |
| 163 | —(CH₂)₃CH₃ | H | Cl | H | H |
| 164 | —(CH₂)₃CH₃ | CH₃ | Cl | H | H |
| 165 | —CH₂—HC═CH₂ | H | Cl | H | H |
| 166 | —CH₃ | H | F | H | H |
| 167 | —CH₃ | —CH₃ | F | H | H |
| 168 | —CH₃ | —CH₂CH₃ | F | H | H |
| 169 | —CH₃ | H | F | —CH₃ | H |
| 170 | —CH₃ | H | F | H | F |
| 171 | —CH₃ | H | F | H | —OCH₃ |
| 172 | —CH₂CH₃ | H | F | H | H |
| 173 | —CH₂CH₃ | —CH₃ | F | H | H |
| 174 | —CH₂CH₃ | —CH₂CH₃ | F | H | H |
| 175 | —CH₂CH₃ | H | F | H | F |
| 176 | —CH₂CH₃ | H | F | H | —OCH₃ |
| 177 | —(CH₂)₂CH₃ | H | F | H | H |
| 178 | —(CH₂)₂CH₃ | —CH₃ | F | H | H |
| 179 | —(CH₂)₂CH₃ | —CH₂CH₃ | F | H | H |
| 180 | —(CH₂)₂CH₃ | H | F | H | F |
| 181 | —CH(CH₃)₂ | H | F | H | H |
| 182 | —CH(CH₃)₂ | —CH₃ | F | H | H |
| 183 | —CH(CH₃)₂ | —CH₂CH₃ | F | H | H |
| 184 | —(CH₂)₃CH₃ | H | F | H | H |
| 185 | —(CH₂)₃CH₃ | —CH₃ | F | H | H |
| 186 | —CH₂—HC═CH₂ | H | F | H | H |
| 187 | —CH₃ | H | H | Br | H |
| 188 | —CH₃ | H | H | Br | H |
| 189 | —CH₃ | —CH₃ | H | Br | H |
| 190 | —CH₃ | —CH₂CH₃ | H | Br | H |
| 191 | —CH₃ | H | H | Br | F |
| 192 | —CH₃ | H | H | Br | —OCH₃ |
| 193 | —CH₂CH₃ | H | H | Br | H |
| 194 | —CH₂CH₃ | —CH₃ | H | Br | H |
| 195 | —CH₂CH₃ | —CH₂CH₃ | H | Br | H |
| 196 | —CH₂CH₃ | H | H | Br | F |
| 197 | —CH₂CH₃ | H | H | Br | —OCH₃ |
| 198 | —(CH₂)₂CH₃ | H | H | Br | H |
| 199 | —(CH₂)₂CH₃ | —CH₃ | H | Br | H |
| 200 | —(CH₂)₂CH₃ | —CH₂CH₃ | H | Br | H |
| 201 | —(CH₂)₂CH₃ | H | H | Br | F |
| 202 | —CH(CH₃)₂ | H | H | Br | H |
| 203 | —CH(CH₃)₂ | —CH₃ | H | Br | H |
| 204 | —CH(CH₃)₂ | —CH₂CH₃ | H | Br | H |
| 205 | —(CH₂)₃CH₃ | H | H | Br | H |
| 206 | —(CH₂)₃CH₃ | —CH₃ | H | Br | H |
| 207 | —CH₂—HC═CH₂ | H | H | Br | H |
| 208 | —CH₃ | H | H | Cl | H |
| 209 | —CH₃ | —CH₃ | H | Cl | H |
| 210 | —CH₃ | —CH₂CH₃ | H | Cl | H |
| 211 | —CH₃ | H | H | Cl | F |
| 212 | —CH₃ | H | H | Cl | —OCH₃ |
| 213 | —CH₂CH₃ | H | H | Cl | H |
| 214 | —CH₂CH₃ | —CH₃ | H | Cl | H |
| 215 | —CH₂CH₃ | —CH₂CH₃ | H | Cl | H |
| 216 | —CH₂CH₃ | H | H | Cl | F |
| 217 | —CH₂CH₃ | H | H | Cl | —OCH₃ |
| 218 | —(CH₂)₂CH₃ | H | H | Cl | H |
| 219 | —(CH₂)₂CH₃ | —CH₃ | H | Cl | H |
| 220 | —(CH₂)₂CH₃ | —CH₂CH₃ | H | Cl | H |
| 221 | —(CH₂)₂CH₃ | H | H | Cl | F |
| 222 | —CH(CH₃)₂ | H | H | Cl | H |
| 223 | —CH(CH₃)₂ | —CH₃ | H | Cl | H |
| 224 | —CH(CH₃)₂ | —CH₂CH₃ | H | Cl | H |
| 225 | —(CH₂)₃CH₃ | H | H | Cl | H |
| 226 | —(CH₂)₃CH₃ | —CH₃ | H | Cl | H |
| 227 | —CH₂—HC═CH₂ | H | H | Cl | H |
| 228 | —CH₃ | H | H | F | H |
| 229 | —CH₃ | —CH₃ | H | F | H |
| 230 | —CH₃ | —CH₂CH₃ | H | F | H |
| 231 | —CH₃ | H | H | F | —OCH₃ |
| 232 | —CH₃ | H | H | F | F |
| 233 | —CH₃ | H | H | F | H |
| 234 | —CH₂CH₃ | H | H | F | H |
| 235 | —CH₂CH₃ | —CH₃ | H | F | H |
| 236 | —CH₂CH₃ | CH₂CH₃ | H | F | H |
| 237 | —CH₂CH₃ | H | H | F | F |
| 238 | —CH₂CH₃ | H | H | F | H |
| 239 | —(CH₂)₂CH₃ | H | H | F | H |
| 240 | —(CH₂)₂CH₃ | —CH₃ | H | F | H |
| 241 | —(CH₂)₂CH₃ | —CH₂CH₃ | H | F | H |
| 242 | —(CH₂)₂CH₃ | H | H | F | F |
| 243 | —CH(CH₃)₂ | H | H | F | H |
| 244 | —CH(CH₃)₂ | —CH₃ | H | F | H |
| 245 | —CH(CH₃)₂ | —CH₂CH₃ | H | F | H |
| 246 | —(CH₂)₃CH₃ | H | H | F | H |
| 247 | —(CH₂)₃CH₃ | —CH₃ | H | F | H |
| 248 | —CH₂—HC═CH₂ | H | H | F | H |
| 249 | —CH₃ | H | H | H | H |
| 250 | —CH₃ | H | —OH | H | H |
| 251 | —CH₃ | H | —OC(O)CH₃ | H | H |
| 252 | —CH₃ | H | —OCH₃ | H | H |
| 253 | —CH₃ | H | Br | H | H |
| 254 | —CH₃ | H | Cl | H | H |
| 255 | —CH₃ | H | F | H | H |
| 256 | —CH₃ | —CH₃ | H | H | H |
| 257 | —CH₃ | —CH₃ | —OH | H | H |
| 258 | —CH₃ | —CH₃ | —OC(O)CH₃ | H | H |
| 259 | —CH₃ | —CH₃ | —OCH₃ | H | H |
| 260 | —CH₃ | —CH₃ | Br | H | H |
| 261 | —CH₃ | —CH₃ | Cl | H | H |
| 262 | —CH₃ | —CH₃ | F | H | H |
| 263 | —CH₃ | H | H | H | F |
| 264 | —CH₃ | H | —OH | H | F |
| 265 | —CH₃ | H | —OC(O)CH₃ | H | F |
| 266 | —CH₃ | H | —OCH₃ | H | F |
| 267 | —CH₃ | H | Br | H | F |
| 268 | —CH₃ | H | Cl | H | F |
| 269 | —CH₃ | H | F | H | F |
| 270 | —CH₃ | H | H | H | —OCH₃ |
| 271 | —CH₃ | H | —OH | H | —OCH₃ |
| 272 | —CH₃ | H | —OC(O)CH₃ | H | —OCH₃ |
| 273 | —CH₃ | H | —OCH₃ | H | —OCH₃ |
| 274 | —CH₃ | H | Br | H | —OCH₃ |
| 275 | —CH₃ | H | Cl | H | —OCH₃ |
| 276 | —CH₃ | H | F | H | —OCH₃ |
| 277 | —CH₃ | H | H | —OH | H |
| 278 | —CH₃ | H | H | —OC(O)CH₃ | H |
| 279 | —CH₃ | H | H | —OCH₃ | H |
| 280 | —CH₃ | H | H | Br | H |
| 281 | —CH₃ | H | H | Cl | H |
| 282 | —CH₃ | H | H | F | H |
| 283 | —CH₃ | —CH₃ | H | H | H |
| 284 | —CH₃ | —CH₃ | H | —OH | H |
| 285 | —CH₃ | —CH₃ | H | —OC(O)CH₃ | H |
| 286 | —CH₃ | —CH₃ | H | —OCH₃ | H |
| 287 | —CH₃ | —CH₃ | H | Br | H |
| 288 | —CH₃ | —CH₃ | H | Cl | H |
| 289 | —CH₃ | —CH₃ | H | F | H |
| 290 | —CH₃ | H | H | H | F |
| 291 | —CH₃ | H | H | —OH | F |
| 292 | —CH₃ | H | H | —OC(O)CH₃ | F |
| 293 | —CH₃ | H | H | —OCH₃ | F |
| 294 | —CH₃ | H | H | Br | F |
| 295 | —CH₃ | H | H | Cl | F |
| 296 | —CH₃ | H | H | F | F |
| 297 | —CH₃ | H | H | H | —OCH₃ |
| 298 | —CH₃ | H | H | —OH | —OCH₃ |
| 299 | —CH₃ | H | H | —OC(O)CH₃ | —OCH₃ |
| 300 | —CH₃ | H | H | —OCH₃ | —OCH₃ |
| 301 | —CH₃ | H | H | Br | —OCH₃ |
| 302 | —CH₃ | H | H | Cl | —OCH₃ |
| 303 | —CH₃ | H | H | F | —OCH₃ |
| 304 | H | H | —OH | H | H |
| 305 | H | —CH₃ | —OH | H | H |
| 306 | H | —CH₂CH₃ | —OH | H | H |
| 307 | H | H | —OH | —CH₃ | H |
| 308 | H | H | —OH | H | F |
| 309 | H | H | —OH | H | —OCH₃ |
| 310 | H | H | H | H | H |
| 311 | H | —CH₃ | H | H | H |
| 312 | H | —CH₂CH₃ | H | H | H |
| 313 | H | H | H | —CH₃ | H |
| 314 | H | H | H | H | F |
| 315 | H | —CH₃ | H | H | —OCH₃ |
| 316 | H | H | H | H | —OCH₃ |
| 317 | H | H | H | H | F |
| 318 | —CH₃ | H | H | H | —OCH₃ |
| 319 | —CH₃ | H | H | H | F |

### Compositions and Methods

As used herein, the term “5-HT1A” refers to a 5-HT1A receptor. As used herein, the term “5-HT2A” refers to a 5-HT2A receptor.

As used herein, the term “effective amount” in connection with a compound disclosed herein means an amount capable of treating or preventing a disorder, disease or condition, or symptoms thereof, disclosed herein.

As used herein, the term “hallucination” (and related terms such as “hallucinogenic” and “hallucinogen”) refers to a perception in the absence of external stimulus that has qualities of real perception. In some embodiments, hallucinations may be vivid, substantial, and are perceived to be located in external objective space. As used herein, hallucinations may occur in any sensory modality including, but not limited to visual, auditory, olfactory, gustatory, tactile, proprioceptive, equilibrioceptive, nociceptive, thermoceptive and chronoceptive. In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations, chronoceptive hallucinations and any combination thereof. In some embodiments, hallucinations are visual hallucinations.

As used herein, the terms “prevent” or “preventing” refers to means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein, the term “treat” or “treating” refers to an alleviation, in whole or in part, of a disorder, disease or condition, or one or more of the symptoms associated with a disorder, disease, or condition, or slowing or halting of further progression or worsening of those symptoms or alleviating or eradicating the cause(s) of the disorder, disease, or condition itself.

In further embodiments of the present disclosure, described are novel compounds and compositions, as well as methods of administering the same. In some embodiments, a compound provided herein is for use in the methods provided herein. In some embodiments, the disclosure provides the use of a compound provided herein in the preparation of a medicament for treating one or more of the diseases or disorders provided herein.

In certain embodiments, the method comprises administering a serotonin 5-HT1A agonist and a serotonin 5-HT2A agonist. Without being bound to any particular theory, in certain embodiments it has been surprisingly discovered that administering a serotonin 5-HT1A agonist and a serotonin 5-HT2A agonist can be effective in preventing or treating one or more of the conditions described herein. In certain embodiments, it has also been surprisingly discovered that administering a serotonin 5-HT1A agonist and a hallucinogenic 5-HT2A agonist can effectively treat patients without the patients experiencing the hallucinogenic effects of the 5-HT2A agonist. Without intending to be bound by any particular theory, it is believed that the patient can experience a therapeutic effect without experiencing a hallucinogenic manifestation that typically results from the administration of a 5-HT2A agonist because the 5-HT1A agonist can “turn off” the hallucinogenic effects of the of the 5-HT2A agonist without otherwise significantly altering its agonism at a 5-HT2A receptor. In some embodiments, the 5-HT1A agonist is a partial agonist. In some embodiments, the 5-HT1A agonist is a full agonist. In some embodiments, the 5-HT2A agonist is a partial agonist. In some embodiments, the 5-HT2A agonist is a full agonist. In some embodiments, the 5-HT1A and/or 5-HT2A agonists may be selected from compounds of Formula I, II, III, IV, V, VI, VII, VIIIa, VIIIb, IXa, IXb, Xa, Xb, XIa or XIb herein. In some embodiments, the 5-HT1A and the 5-HT2A agonists are the same compound (e.g., a compound of Formula I, II, III, IV, V, VI, VII, VIIIa, VIIIb, IXa, IXb, Xa, Xb, XIa or XIb).

As defined herein, a “full agonist” shall mean an agonist having an Emax % of at least 90% for the relevant serotonin receptor agonist assay (e.g., BRET2, calcium mobilization, beta-arrestin) when compared to an industry-accepted control compound for that particular receptor assay (e.g., serotonin (5-OH-tryptamine)). In some embodiments, a “full agonist” will exhibit an Emax % of at least 90, at least 91, at least 92, at least 93, at least 94, at least 95, at least 96, at least 97, at least 98, or at least 99%. Also defined herein, a “partial agonist” shall mean an agonist having an Emax % of less than 90% for the relevant serotonin receptor when compared to an industry-accepted control compound for that particular receptor (e.g., serotonin (5-OH-tryptamine)). In some embodiments, a “partial agonist” will exhibit an Emax % of less than 85, 80, 75, 70, 65, 60, 55, 50, 45, 40, 35, 30, 25, 20, 15, 10, or even less than 5%. In some embodiments, a partial agonist will exhibit an Emax % of about 0.1 to about 89.9%, such as about 1 to about 89, about 5 to about 85, about 50 to about 88, about 40 to about 85, about 35 to about 75, about 25 to about 65, or about 20 to about 55%.

In some embodiments, the 5-HT1A agonist as used herein is selected from buspirone (8-[4-(4-pyrimidin-2-ylpiperazin-1-yl)butyl]-8-azaspiro[4.5]decane-7,9-dione), 5-OH-buspirone, 6-OH-buspirone, tandospirone ((1R,2R,6S,7S)-4-{4-[4-(pyrimidin-2-yl)piperazin-1-yl]butyl}-4-azatricyclo[5.2.1.02,6]decane-3,5-dione), gepirone (4,4-dimethyl-1-[4-(4-pyrimidin-2-ylpiperazin-1-yl)butyl]piperidine-2,6-dione), alnespirone ((+)-4-dihydro-2H-chromen-3-yl]-propylamino]butyl]-8-azaspiro[4.5]decane-7,9-dione), binospirone (8-[2-(2,3-dihydro-1,4-benzodioxin-2-ylmethylamino)ethyl]-8-azaspiro[4.5]-decane-7,9-dione), ipsapirone (9,9-dioxo-8-[4-(4-pyrimidin-2-ylpiperazin-1-yl)butyl]-9.lamda.6-thia-8-a-zabicyclo[4.3.0]nona-1,3,5-trien-7-one), perospirone (3aR, 7aS)-2-{4-[4-(1,2-benzisothiazol-3-yl)piperazin-1-yl]butyl}hexahydro-1H-isoindole-1,3(2H)-dione, befiradol (F-13,640) (3-chloro-4-fluorophenyl-[4-fluoro-4-([(5-methylpyridin-2-yl)methylamino]methyl)piperidin-1-yl]methanone, repinotan ((R)-(−)-2-[4-[(chroman-2-ylmethyl)-amino]-butyl]-1,1-dioxo-benzo[d]isothiazolone), piclozotan (3-chloro-4-[4-[4-(2-pyridinyl)-1,2,3,6-tetrahydropyridin-1-yl]butyl]-1,4-benzoxazepin-5(4H)-one), osemozotan (5-(3-[((2S)-1,4-benzodioxan-2-ylmethyl)amino]propoxy)-1,3-benzodioxole), flesinoxan (4-fluoro-N-[2-[4-[(3S)-3-(hydroxymethyl)-2,3-dihydro-1,4-benzodioxin-8-yl]piperazin-1-yl]ethyl]benzamide), flibanserin (1-(2-{4-[3-(trifluoromethyl)phenyl]piperazin-1-yl}ethyl)-1,3-dihydro-2H-benzimidazol-2-one), 8-OH-DPAT (7-(Dipropylamino)-5,6,7,8-tetrahydronaphthalen-1-ol), and sarizotan (EMD-128,130) (1-[(2R)-3,4-dihydro-2H-chromen-2-yl]-N-([5-(4-fluorophenyl)pyridin-3-yl]methyl)methanamine), a compound of Formula I, II, III, IV, V, VI, VII, VIIIa, VIIIb, IXa, IXb, Xa, Xb, XIa or XIb, or a prodrug, salt, or derivative thereof.

In some embodiments, the serotonin 5-HT1A agonist and 5-HT2A agonist are administered at the same time. In some embodiments, the serotonin 5-HT1A agonist and 5-HT2A agonist are administered at different times. In some embodiments, the serotonin 5-HT1A agonist and 5-HT2A agonist are administered sequentially. In some embodiments, the serotonin 5-HT1A agonist is administered first, and 5-HT2A agonist is administered second. In some embodiments, the serotonin 5-HT2A agonist is administered about 30 minutes to about 12 hrs after administration of 5-HT1A agonist, such as about 1 hr to about 6 hrs afterwards. In some embodiments, the serotonin 5-HT1A agonist and 5-HT2A agonist are administered at the same time in the same composition. In some embodiments, 5-HT1A agonist is selected from buspirone, 5-OH-buspirone, 6-OH-buspirone, and 8-OH-DPAT. In some embodiments, the 5-HT1A agonist is buspirone. In some embodiments, the 5-HT1A agonist is selected from compounds of Formula I, II, III, IV, V, VI, VII, VIIIa, VIlIb, IXa, IXb, Xa, Xb, XIa or Xb, such as for example compounds of Formula II(a)-II(d). In some embodiments, the 5-HT2A agonist is hallucinogenic. In some embodiments, the 5-HT2A agonist is non-hallucinogenic. In some embodiments, the 5-HT2A agonist is selected from compounds of Formula I, II, III, IV, V, VI, VII, VIIIa, VIIIb, IXa, IXb, Xa, Xb, XIa or XIb, such as for example compounds of Formula II(a)-(d).

In some embodiments, the 5-HT2A agonist and the 5-HT1A agonist may comprise the same compound. In some embodiments, the compounds of Formula I, II, III, IV, V, VI, VII, VIIIa, VIIIb, IXa, IXb, Xa, Xb, XIa or XIb described herein (e.g., compounds of Formula II(a)-II(d)) can act as both 5-HT1A and 5-HT2A receptor agonists. In some embodiments, the compounds described herein are full agonists for both 5-HT1A and 5-HT2A.

In some embodiments, the 5-HT1A agonist and 5-HT2A agonist are full agonists for a 5-HT1A receptor and a 5-HT2A receptor, respectively. In some embodiments, the 5-HT1A agonist exhibits a higher level of molar potency (i.e., lower EC₅₀) for activating a 5-HT1A receptor than the 5-HT2A agonist exhibits for activating the 5-HT2A receptor. Without being bound to any particular scientific theory, in certain embodiments it has been surprisingly discovered that compounds that are agonists for 5-HT1A and 5-HT2A—but which exhibit a higher molar potency for 5-HT1A—may be useful to patients needing/desiring non-hallucinogenic 5-HT2A modulation. In other embodiments, the 5-HT1A agonist is a partial agonist (e.g., buspirone) and 5-HT2A agonist is a full agonist for a 5-HT1A receptor and a 5-HT2A receptors, respectively. In other embodiments, the 5-HT1A agonist is a partial agonist (e.g., buspirone) and 5-HT2A agonist is a partial agonist for a 5-HT1A receptor and a 5-HT2A receptors, respectively.

In certain embodiments are described methods for treating, preventing, ameliorating, or curing a disease or disorder via a non-hallucinogenic therapeutic treatment regimen that includes modulation of a 5-HT1A receptor. In certain embodiments, the method comprises identifying a subject in need of treatment for a disease or condition associated with modulation of a 5-HT1A receptor; selecting a compound of Formula I, II, III, IV, V, VI, VII, VIIIa, VIIIb, IXa, IXb, Xa, Xb, XIa or XIb (e.g., Formula II(a)-II(d)); and administering the compound to the subject in need of treatment, wherein the compound modulates activity at both a 5-HT1A and 5-HT2A receptor. In certain embodiments, the compound of Formula I, II, III, IV, V, VI, VII, VIIIa, VIIIb, IXa, IXb, Xa, Xb, XIa or XIb is a full agonist of a 5-HT1A receptor. In certain embodiments, the compound of Formula I, II, III, IV, V, VI, VII, VIIIa, VIIIb, IXa, IXb, Xa, Xb, XIa or XIb is a full agonist for both 5-HT1A and 5-HT2A receptors. In certain embodiments, the compound of Formula I, II, III, IV, V, VI, VII, VIIIa, VIIIb, IXa, IXb, Xa, Xb, XIa or XIb is a partial agonist for a 5-HT1A receptor and a full agonist for a 5-HT2A receptor. In certain embodiments, the compound of Formula I, II, III, IV, V, VI, VII, VIIIa, VIIIb, IXa, IXb, Xa, Xb, XIa or XIb is a partial agonist for a 5-HT1A receptor and a partial agonist for a 5-HT2A receptor. In certain embodiments, the compound of Formula I, II, III, IV, V, VI, VII, VIIIa, VIIIb, IXa, IXb, Xa, Xb, XIa or XIb exhibits a higher molar potency (lower EC₅₀) for a 5-HT1A receptor when compared to a 5-HT2A receptor.

In certain embodiments, the 5-HT1A agonist has an EC₅₀ for activating a 5-HT1A receptor of less than about 100 nM, such as less than about 75 nm, less than about 50 nm, less than about 25 nm, less than about 15 nm, less than about 10 nm, or less than about 5 nm. In certain embodiments, the 5-HT2A agonist has an EC₅₀ for activating a 5-HT2A receptor of less than about 100 nM, such as less than about 75 nm, less than about 50 nm, less than about 25 nm, less than about 15 nm, less than about 10 nm, or less than about 5 nm. In certain embodiments, the 5-HT1A agonist exhibits an EC₅₀ for activating a 5-HT1A receptor of about 0.01 nM to about 100 nM, such as about 0.05 to about 50 nm, about 0.1 to about 25 nM, or about 0.5 to about 10 nM. In certain embodiments, the 5-HT2A agonist has an EC₅₀ for activating a 5-HT2A receptor of about 0.01 nM to about 100 nM, such as about 0.05 to about 50 nm, about 0.1 to about 25 nM, or about 0.5 to about 10 nM. In certain embodiments, the 5-HT2A agonist has an EC₅₀ for activating a 5-HT2A receptor of about 5 to about 75 nM, such as about 10 to about 60 nm, about 15 to about 50 nM, or about 20 to about 40 nM. In some embodiments, the 5-HT1A agonist/5-HT2A agonist exhibits a 5-HT1A receptor: 5-HT2A receptor EC₅₀ ratio range of about 1:2 to about 1:100, such as about 1:5 to about 1:50 or about 1:10 to about 1:40. In some embodiments, one or more of the compounds of Formula I, II, III, IV, V, VI, VII, VIIIa, VIIIb, IXa, IXb, Xa, Xb, XIa or XIb independently exhibit a 5-HT1A receptor: 5-HT2A receptor EC₅₀ ratio range of about 1:2 to about 1:100, such as about 1:5 to about 1:50 or about 1:10 to about 1:40. Relevant testing parameters to determine full vs. partial agonism (Emax %) and molar potency (EC₅₀) include those known to persons of skill in the art, such as the 5-HT Functional Assays described further below.

In some embodiments, also described are novel compounds and compositions, as well as methods of administering the same. In certain embodiments, the method comprises administering a serotonin 5-HT2A agonist and a serotonin 5-HT2B antagonist. Without being bound to any particular theory, in certain embodiments it has been surprisingly discovered that administering a serotonin 5-HT2A agonist and a serotonin 5-HT2B antagonist can be effective in preventing or treating one or more of the conditions described herein. In some embodiments, it has been surprisingly discovered that administering a serotonin 5-HT2A agonist and a serotonin 5-HT2B antagonist can effectively treat patients while also reducing serotonin 5-HT2B-induced cardiotoxicity (e.g., heart valve fibrosis and hypertrophy). In certain embodiments, it has also been surprisingly discovered that administering a serotonin 5-HT2B antagonist and a 5-HT2A agonist can be safely and effectively used treat patients as described herein without the patients experiencing the hallucinogenic effects that can be associated with hallucinogenic 5-HT2A agonists. In some embodiments, the 5-HT2A agonist is a full agonist. In some embodiments, the 5-HT2A agonist is a partial agonist. In some embodiments, the 5-HT2B antagonist is a full antagonist. In some embodiments, the 5-HT2B antagonist is a partial antagonist.

Exemplary serotonin 5-HT2B receptor antagonists include, but are not limited to, agomelatine, amisulpride, ariprazole, carprazine, clozapine, cyproheptadine, mCCP, sarpogrelate, lisuride, tegasurod, metadoxine, and promethazine. In certain embodiments, the 5-HT2B antagonist is not an antagonist at any of the other serotonin 5-HT type receptor subtypes, such as 5-HT1A and 5-HT2A. In certain embodiments, the 5-HT2B receptor antagonist will also be a full or partial agonist at a 5-HT1A and/or 5-HT2A receptor.

In some embodiments, the serotonin 5-HT2A agonist and 5-HT2B antagonist are administered at the same time. In some embodiments, the serotonin 5-HT2A agonist and 5-HT2B antagonist are administered at different times. In some embodiments, the serotonin 5-HT2A agonist and 5-HT2B antagonist are administered at the same time in the same composition. In some embodiments, the serotonin 5-HT1A agonist and 5-HT2B antagonist are administered sequentially. In some embodiments, the serotonin 5-HT2B antagonist is administered first, and 5-HT2A agonist is administered second. In some embodiments, the serotonin 5-HT2A agonist is administered about 30 minutes to about 12 hrs after administration of 5-HT2B antagonist, such as about 1 hr to about 6 hrs afterwards. In some embodiments, the 5-HT2A agonist is hallucinogenic. In some embodiments, the 5-HT2A agonist is non-hallucinogenic. In some embodiments, the 5-HT2A agonist is selected from compounds of Formula I, II, III, IV, V, VI, VII, VIIIa, VIIIb, IXa, IXb, Xa, Xb, XIa or XIb, such as for example compounds of Formula II(a)-II(d).

In some embodiments, also described are novel compounds and compositions, as well as methods of administering the same. In certain embodiments, the method comprises administering a serotonin 5-HT2A agonist and a serotonin 5-HT2C agonist. Without being bound to any particular theory, in certain embodiments it has been surprisingly discovered that administering a serotonin 5-HT2A agonist and a serotonin 5-HT2C agonist can be effective in preventing or treating one or more of the conditions described herein. In some embodiments, it has been surprisingly discovered that administering a serotonin 5-HT2A agonist and a serotonin 5-HT2C agonist can effectively treat patients while also reducing or eliminating the hallucinogenic “trip” typically associated with 5-HT2A agonists. In some embodiments, the 5-HT2A agonist is a full agonist. In some embodiments, the 5-HT2A agonist is a partial agonist. In some embodiments, the 5-HT2C agonist is a full agonist. In some embodiments, the 5-HT2C agonist is a partial agonist.

Exemplary serotonin 5-HT2C receptor agonists include, but are not limited to, locaserin, vabicaserin, aripiprazole, YM-348, PRX-00933, and meta-chlorophenylpiperazine. In certain embodiments, the 5-HT2C agonist is not an agonist at any of the other serotonin 5-HT type receptor subtypes, such as 5-HT1A and 5-HT2B. In certain embodiments, the 5-HT2C receptor agonist will be inactive or only a partial agonist at a 5-HT1A and/or 5-HT2B receptor.

In some embodiments, the serotonin 5-HT2A agonist and 5-HT2C agonist are administered at the same time. In some embodiments, the serotonin 5-HT2A agonist and 5-HT2C agonist are administered at different times. In some embodiments, the serotonin 5-HT2A agonist and 5-HT2C agonist are administered at the same time in the same composition. In some embodiments, the serotonin 5-HT2A agonist and 5-HT2C agonist are administered sequentially. In some embodiments, the serotonin 5-HT2C agonist is administered first, and 5-HT2A agonist is administered second. In some embodiments, the serotonin 5-HT2A agonist is administered about 30 minutes to about 12 hrs after administration of 5-HT2C agonist, such as about 1 hr to about 6 hrs afterwards. In some embodiments, the 5-HT2A agonist is hallucinogenic. In some embodiments, the 5-HT2A agonist is non-hallucinogenic. In some embodiments, the 5-HT2A agonist is selected from compounds of Formula I, II, III, IV, V, VI, VII, VIIIa, VIIIb, IXa, IXb, Xa, Xb, XIa or XIb, such as for example compounds of Formula II(a)-II(d).

In some embodiments, the 5-HT2A agonist and the 5-HT2C agonist may comprise the same compound. In some embodiments, the compounds of Formula I, II, III, IV, V, VI, VII, VIIIa, VIIIb, IXa, IXb, Xa, Xb, XIa or XIb described herein (e.g., compounds of Formula II(a)-II(d)) can act as both 5-HT2C and 5-HT2A receptor agonists. In some embodiments, the compounds described herein are full agonists for both 5-HT2A and 5-HT2C. In some embodiments, the compounds described herein act as partial agonists at 5-HT2A and full agonists at 5-HT2C. In some embodiments, the compounds described herein are partial agonists for both 5-HT2A and 5-HT2C. In some embodiments, the compounds described herein act as agonists at 5-HT2A and 5-HT2C but are only partial agonists (or inactive) at a 5-HT2B receptor.

In some embodiments, the compounds of Formula I, II, III, IV, V, VI, VII, VIIIa, VIIIb, IXa, IXb, Xa, Xb, XIa or XIb are selective agonists of 5-HT2C, wherein said compounds are more potent and/or efficacious (Emax) when compared to the other serotonin receptor subtypes (e.g., 5-HT2A and 5-HT2B). Without being bound to any particular theory, it has been surprisingly discovered that compounds of the present disclosure are orthosteric ligands of 5-HT2C that exhibit a G_{q}-mediated signaling bias and no or minimal β-arrestin recruitment or β-arrestin-mediated intracellular signaling. Applicant theorizes that this G_{q}-biased signaling mechanism results in reduced signal attenuation caused from β-arrestin recruitment, resulting in a greater therapeutic effect and fewer side effects for certain therapeutic indications (e.g., Alzheimer's psychosis, schizophrenia, addiction, obesity, etc.).

In some embodiments, the compounds of Formula I, II, III, IV, V, VI, VII, VIIIa, VIIIb, IXa, IXb, Xa, Xb, XIa or XIb are nicotinic receptor modulators. Nicotinic acetylcholine receptors, or nAChRs, are receptor polypeptides that respond to the neurotransmitter acetylcholine. Nicotinic receptors also respond to drugs such as the agonist nicotine. They are found in the central and peripheral nervous system, muscle, and many other tissues of many organisms. At the neuromuscular junction they are the primary receptor in muscle for motor nerve-muscle communication that controls muscle contraction. In the peripheral nervous system: (1) they transmit outgoing signals from the presynaptic to the postsynaptic cells within the sympathetic and parasympathetic nervous system, and (2) they are the receptors found on skeletal muscle that receive acetylcholine released to signal for muscular contraction. In the immune system, nAChRs regulate inflammatory processes and signal through distinct intracellular pathways. In insects, the cholinergic system is limited to the central nervous system. In vertebrates, nicotinic receptors are broadly classified into two subtypes based on their primary sites of expression: muscle-type nicotinic receptors and neuronal-type nicotinic receptors. The neuronal subtypes are various homomeric (all one type of subunit) or heteromeric (at least one a and one p) combinations of twelve different nicotinic receptor subunits: α₂-α₁₀ and β₂-β₄. Examples of the neuronal subtypes include: (α₄)₃(β₂)₂, (α₄)₂(β₂)₃, (α₃)₂(β₄)₃, α₄α₆β₃(β₂)₂, (α₇)₅, and many others.

In certain embodiments for compounds of Formula I, II, III, IV, V, VI, and VII, Applicant has discovered that the size and nature of alkyl groups for X and/or Y (or R₁ₐ for compounds of Formula II) can affect the metabolism of such compounds. For example, it has been theorized that compounds such as 5-MeO-Dimethyltryptamine (5-MeO-DMT) and Dimethyltryptamine (DMT) are inactive upon oral administration due to rapid metabolism of the methylamino residues by monoamine oxidase (MAO) enzymes. It has also been theorized that the oral stability of psilocin (4-OH-dimethyltryptamine), on the other hand, is due largely to intramolecular coordination (hydrogen bonding) between the 4-OH group and the dimethylamino residue, which effectively shields/inhibits rapid MAO degradation.

Without being bound to any particular scientific theory, Applicant has surprisingly found that substituting the alkyl groups X and/or Y with substituents such as deuterium and fluorine can help inhibit MAO degradation of those groups, despite the absence of a hydrogen bond donor (e.g., —OH) at the 4-position. In addition, or in the alternative, Applicant has discovered that using non-methyl alkyl groups such as ethyl or n-propyl for X and/or Y can also slow or inhibit rapid MAO metabolism upon oral administration. This, in turn, permits the preparation of orally available compounds of Formula I, II, III, IV, V, VI, VII, VIIIa, VIIIb, IXa, IXb, Xa, Xb, XIa or XIb that are highly active serotonergic drugs that do not require special formulating procedures (e.g., dosages containing MAO inhibitors), or the presence of hydrogen bond-forming donors at the 4-position that—in some cases—can negatively impact the properties of the underlying compound (e.g., reduction of 5-HT1A and/or 5-HT2A agonism).

In some embodiments, Applicant has also surprisingly discovered that alpha-deuteration of the compounds of Formula I, II, III, IV, V, VI, VII, VIIIa, VIIIb, IXa, IXb, Xa, Xb, XIa or XIb (wherein R₃ and/or R_{3′} are deuterium) and Formula II (wherein R₃ and R_{3b} are deuterium) can improve the pharmacokinetics of those compounds. Without being bound to any particular scientific theory, it is believed that the heavier deuterium isotope disrupts the enzymatic metabolism of those compounds. However, in some embodiments it may not be desirable to “over deuterate” the compound, such as further including deuterated species for residues for X and Y or deuteration at the beta position (i.e., W₂), which can further alter the compound's pharmacokinetic profiles (e.g., greatly extended half lives) in an undesirable manner. Accordingly, in some embodiments, Applicant has discovered that minimal deuteration may be used to achieve the desired pharmacokinetic outcome. For example, in some embodiments adding a single deuterium atom at the alpha position (i.e., R₃ or R_{3′}) can greatly enhance the desired pharmacokinetic profile. It is theorized that this may be due, in part, to the creation of a stereocenter at the alpha position upon deuteration that impacts the enzymes' ability to metabolize the compound (e.g., hindrance of MAO degradation and/or the ability of enzymes to oxidize the alpha position during metabolic processes).

In one embodiment, the compounds of Formula I, II, III, IV, V, VI, VII, VIIIa, VIIIb, IXa, IXb, Xa, Xb, XIa or XIb, the methods, and the pharmaceutical compositions described herein are used to modulate the activity of a neurotransmitter receptor by administering a therapeutically effective amount of a compound of Formula I, II, III, IV, V, VI, VII, VIIIa, VIIIb, IXa, IXb, Xa, Xb, XIa or XIb. Methods include the administration of a therapeutically effective amount of a compound of Formula I, II, III, IV, V, VI, VII, VIIIa, VIIIb, IXa, IXb, Xa, Xb, XIa or XIb to prevent or treat a psychological disorder such as those discussed herein. Compounds of Formula I, II, III, IV, V, VI, VII, VIIIa, VIIIb, IXa, IXb, Xa, Xb, XIa or XIb may be administered neat or as a pharmaceutical composition comprising a compound of Formula I, II, III, IV, V, VI, VII, VIIIa, VIIIb, IXa, IXb, Xa, Xb, XIa or XIb as discussed herein.

In some embodiments, the compounds of Formula I, II, III, IV, V, VI, VII, VIIIa, VIIIb, IXa, IXb, Xa, Xb, XIa or XIb may be used to prevent and/or treat a psychological disorder. The disclosure provides a method for preventing and/or treating a psychological disorder by administering to a subject in need thereof a therapeutically effective amount of a compound of Formula I, II, III, IV, V, VI, VII, VIIIa, VIIIb, IXa, IXb, Xa, Xb, XIa or XIb, including the exemplary embodiments discussed above. The psychological disorder may be chosen from depression; psychotic disorder; schizophrenia; schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome; posttraumatic stress disorder (PTSD); premenstrual dysphoric disorder (PMDD); and premenstrual syndrome (PMS).

In some embodiments, the compounds of Formula I, II, III, IV, V, VI, VII, VIIIa, VIIIb, IXa, IXb, Xa, Xb, XIa or XIb may be used to prevent and/or treat a brain disorder. The disclosure provides a method for preventing and/or treating a brain disorder by administering to a subject in need thereof a therapeutically effective amount of a compound of Formula I, II, III, IV, V, VI, VII, VIIIa, VIIIb, IXa, IXb, Xa, Xb, XIa or XIb, including the exemplary embodiments discussed above. The brain disorder may be chosen from Huntington's disease, Alzheimer's disease, dementia, and Parkinson's disease.

In some embodiments, the compounds of Formula I, II, III, IV, V, VI, VII, VIIIa, VIIIb, IXa, IXb, Xa, Xb, XIa or XIb may be used to prevent and/or treat developmental disorders, delirium, dementia, amnestic disorders and other cognitive disorders, psychiatric disorders due to a somatic condition, drug-related disorders, schizophrenia and other psychotic disorders, mood disorders, anxiety disorders, somatoform disorders, factitious disorders, dissociative disorders, eating disorders, sleep disorders, impulse control disorders, adjustment disorders, or personality disorders. The disclosure provides a method for preventing and/or treating these disorders by administering to a subject in need thereof a therapeutically effective amount of a compound of Formula I, II, III, IV, V, VI, VII, VIIIa, VIIIb, IXa, IXb, Xa, Xb, XIa or XIb, including the exemplary embodiments discussed above.

In some embodiments, the compounds of Formula I, II, III, IV, V, VI, VII, VIIIa, VIIIb, IXa, IXb, Xa, Xb, XIa or XIb may be used to prevent and/or treat inflammation and/or pain, such as, for example, inflammation and/or pain associated with inflammatory skeletal or muscular diseases or conditions. Accordingly, the disclosure relates to a method for preventing and/or treating inflammation and/or pain by administering to a subject in need thereof a therapeutically effective amount of a compound of Formula I, II, III, IV, V, VI, VII, VIIIa, VIIIb, IXa, IXb, Xa, Xb, XIa or XIb, including the exemplary embodiments discussed herein. Generally speaking, treatable “pain” includes nociceptive, neuropathic, and mix-type. A method of the disclosure may reduce or alleviate the symptoms associated with inflammation, including, but not limited to, treating localized manifestation of inflammation characterized by acute or chronic swelling, pain, redness, increased temperature, or loss of function in some cases. A method of the disclosure may reduce or alleviate the symptoms of pain regardless of the cause of the pain, including, but not limited to, reducing pain of varying severity, i.e. mild, moderate and severe pain, acute pain and chronic pain. A method of the disclosure is effective in treating joint pain, muscle pain, tendon pain, burn pain, and pain caused by inflammation such as rheumatoid arthritis. Skeletal or muscular diseases or conditions which may be treated include, but are not limited to, musculoskeletal sprains, musculoskeletal strains, tendinopathy, peripheral radiculopathy, osteoarthritis, joint degenerative disease, polymyalgia rheumatica, juvenile arthritis, gout, ankylosing spondylitis, psoriatic arthritis, systemic lupus erythematosus, costochondritis, tendonitis, bursitis, such as the common lateral epicondylitis (tennis elbow), medial epicondylitis (pitchers elbow) and trochanteric bursitis, temporomandibular joint syndrome, and fibromyalgia.

In other embodiments, the methods and compositions disclosed herein comprise regulating the activity of a neurotransmitter receptor with a formulation comprising a compound of Formula I, II, III, IV, V, VI, VII, VIIIa, VIIIb, IXa, IXb, Xa, Xb, XIa or XIb. In one embodiment, the methods and compositions disclosed herein comprise administering a first dosage formulation comprising at least one compound of Formula I, II, III, IV, V, VI, VII, VIIIa, VIIIb, IXa, IXb, Xa, Xb, XIa or XIb and a second active compound. In one embodiment, the methods disclosed herein comprise administering a first dosage formulation comprising a compound of Formula I, II, III, IV, V, VI, VII, VIIIa, VIIIb, IXa, IXb, Xa, Xb, XIa or XIb and a neurotransmitter activity modulator (e.g., a second serotonergic drug). In one embodiment, the methods disclosed herein comprise administering a first dosage formulation comprising at least one compound of Formula I, II, III, IV, V, VI, VII, VIIIa, VIIIb, IXa, IXb, Xa, Xb, XIa or XIb and a second dosage form comprising at least one cannabinoid, at least one terpene, or a second serotonergic drug.

The present disclosure relates to compositions comprising, consisting essentially of, or consisting of an effective amount of a compound of Formula I, II, III, IV, V, VI, VII, VIIIa, VIIIb, IXa, IXb, Xa, Xb, XIa or XIb and an excipient. The terms “composition” and “formulation” are used interchangeably herein. Other embodiments relate to pharmaceutical compositions comprising, consisting essentially of, or consisting of a therapeutically effective amount of a compound of Formula I, II, III, IV, V, VI, VII, VIIIa, VIIIb, IXa, IXb, Xa, Xb, XIa or XIb, including those discussed above, and a pharmaceutically acceptable excipient (also known as a pharmaceutically acceptable carrier). As discussed above, a compound of Formula I, II, III, IV, V, VI, VII, VIIIa, VIIIb, IXa, IXb, Xa, Xb, XIa or XIb may be therapeutically useful to prevent and/or treat, for example, psychological disorders, brain disorders, pain and inflammation as well as other disorders such as those discussed above.

Exemplary pharmaceutically-acceptable carriers include, but are not limited to, any pharmaceutically-acceptable material, composition or vehicle, such as a liquid or solid filler, diluent, excipient, solvent or encapsulating material, which may be involved in carrying or transporting the subject chemical from one organ, or portion of the body, to another organ, or portion of the body, e.g., oral dosage form enabling transport of compound to the stomach and/or intestinal tract. In certain embodiments, such oral dosage forms may comprise a pill, tablet, or gel cap. Each carrier is preferably “acceptable” in the sense of being compatible with the other ingredients of the formulation and not injurious to the subject. Some examples of materials which can serve as pharmaceutically-acceptable carriers include, but are not limted to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In some embodiments, the composition may further comprise one or more pharmaceutically-acceptable antioxidants. In certain embodiments, such antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In some embodiments, the compositions described herein may comprise at least one compound of Formula I, II, III, IV, V, VI, VII, VIIIa, VIIIb, IXa, IXb, Xa, Xb, XIa or XIb, and a second compound selected from at least one of a second serotonergic drug, a cannabinoid, a terpene, or an MAO inhibitor. In certain embodiments, the second compound may be derived from natural sources, such as fungi (e.g., Psilocybe mushrooms; Lion's Mane mushrooms (containing terpenes such as erinacines and hericenones)) and plants (e.g., Cannabis). Accordingly, in certain embodiments the second compound may derived or “extracted” from fungus or plant material, meaning said second compound may or may not be “purified” depending on the manner in which it was sourced and extracted.

Within the context of this disclosure, the term “purified” means separated from other compounds or materials, such as plant or fungal material, e.g., protein, chitin, cellulose, or water. In one embodiment, the term “purified” refers to a compound substantially free of other materials. In one embodiment, the term “purified” refers to a compound that is substantially free from a second compound (e.g. an enantiomeric compound of Formula I, II, III, IV, V, VI, VII, VIIIa, VIIIb, IXa, IXb, Xa, Xb, XIa or XIb exhibiting 99% enantiomeric excess after resolution). In one embodiment, the term “purified” refers to a compound substantially free from a biological material, such as mold, fungus, plant mater, or bacteria.

In one embodiment, the term “purified” refers to a compound or composition that has been crystallized. In one embodiment, the term “purified” refers to a compound or composition that has been chromatographed, for example by gas chromatography, liquid chromatography (e.g., LC, HPLC, etc.), etc. In one embodiment, the term “purified” refers to a compound or composition that has been distilled. In one embodiment, the term “purified” refers to a compound or composition that has been sublimed. In one embodiment, the term “purified” refers to a compound or composition that has been subject to two or more steps chosen from crystallization, chromatography, distillation, and sublimation.

In one embodiment, the term “purified” refers to a compound that has a purity ranging from about 80% to about 100%, meaning that the compound makes up about 80% to about 100% of the total mass of the composition. In one embodiment, the term “purified” refers to a compound that has a purity ranging from about 90% to about 100%, meaning that the compound makes up about 90% to about 100% of the total mass of the composition. In one embodiment, the term “purified” refers to a compound that has a purity ranging from about 95% to about 100%, meaning that the compound makes up about 95% to about 100% of the total mass of the composition. In one embodiment, the term “purified” refers to a compound that has a purity ranging from about 99% to about 100% pure, meaning that the compound makes up about 99% to about 100% of the total mass of the composition. In one embodiment, the term “purified” refers to a compound that has a purity ranging from about 99.9% to about 100%, meaning that the compound makes up about 99.9% to about 100% of the total mass of the composition.

As used herein, the term “particular ratio” refers to the amount of a compound in relation to the amount of another compound or compounds. In one embodiment, there is about 1:1 ratio of a 4-acetoxy-3-[2-(dimethylamino)ethyl]-benzo[b]thiophene) to 4-hydroxy-N,N-dimethyltryptamine. In one embodiment, a particular ratio of compounds is measured by the same unit, e.g., grams, kilograms, pounds, ounces, etc. In one embodiment, a particular ratio of compounds is measured in moles, i.e., molar proportions or molar ratios.

As used herein, the term “particular amount” refers to the quantity of a compound or compounds. In one embodiment, a particular amount is the combined quantity of two compounds within a sample. In one embodiment, a particular amount is measured by dry weight. In one embodiment, the particular amount has 1, 2, 3, or 4 significant figures.

Disclosed herein are compositions comprising a compound of Formula I, II, III, IV, V, VI, VII, VIIIa, VIIIb, IXa, IXb, Xa, Xb, XIa or XIb and a second compound. In one embodiment, the compositions disclosed herein comprise a molar ratio ranging from about 10:1 to about 1:10 of the compound of Formula I, II, III, IV, V, VI, VII, VIIIa, VIIIb, IXa, IXb, Xa, Xb, XIa or XIb (e.g., a 5-HT2A receptor agonist) to the second compound (e.g., a 5-HT1A receptor agonist). In one embodiment, the compositions disclosed herein comprise a molar ratio ranging from about 100:1 to about 1:100 of the compound of Formula I, II, III, IV, V, VI, VII, VIIIa, VIIIb, IXa, IXb, Xa, Xb, XIa or XIb to the second compound. In one embodiment, the compositions disclosed herein comprise a molar ratio ranging from about 1,000:1 to about 1:1,000 of the compound of Formula I, II, III, IV, V, VI, VII, VIIIa, VIIIb, IXa, IXb, Xa, Xb, XIa or XIb to the second compound. In one embodiment, the compositions disclosed herein comprise a molar ratio ranging from about 10,000:1 to about 1:10,000 of the compound of Formula I, II, III, IV, V, VI, VII, VIIIa, VIIIb, IXa, IXb, Xa, Xb, XIa or XIb to the second compound.

Within the context of this disclosure, unless otherwise specified, the serotonergic compounds (e.g., tryptamine compounds) described herein may be present in their protonated or deprotonated (salt or freebase) forms or mixtures thereof depending on the context, for example, the pH of the solution or composition. However, in certain embodiments, the serotonergic compounds described herein are lipophilic, meaning they will tend to combine with lipids and fats and can readily pass though biological membranes in the body of an animal or human (e.g., blood brain barrier). In certain embodiments, the serotonergic compound in free base form is lipophilic.

As used herein, the term “salt” refers to a neutralized ionic compound. In one embodiment, a salt is formed from the neutralization of acids and bases. In one embodiment, a salt is electrically neutral.

In one embodiment, the compositions and methods disclosed herein comprise administering a first cannabinoid. In one embodiment, a first cannabinoid is a first purified cannabinoid.

As used herein, the term “cannabinoid” refers to a compound from a class of molecules commonly found in plants of the genus cannabis and their derivatives. In one embodiment, the cannabinoid is endogenous to an animal, i.e., an endocannabinoid. In one embodiment, the cannabinoid is derived from a plant, e.g., a plant of genus cannabis, e.g., a phytocannabinoid. In one embodiment, the cannabinoid is artificially made in a lab, i.e., a synthetic cannabinoid. Many cannabinoids can be identified by the “cannabi” text in their chemical name. There are at least 113 different cannabinoids isolated from cannabis, exhibiting varied (similar and different) effects.

Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (.DELTA.8-THC), Delta-8-tetrahydrocannabinolic acid (.DELTA.8-THCA), Delta-9-tetrahydrocannabinol (THC), Delta-9-tetrahydrocannabinol-C₄ (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-metha-no-2H-1-benzoxocin-5-methanol.

In one embodiment, the term “cannabinoid” refers to a compound chosen from THC, THCA, THCV, THCVA, CBC, CBCA, CBCV, CBCVA, CBD, CBDA, CBDV, CBDVA, CBG, CBGA, CBGV, and CBGVA.

Within the context of this disclosure, the term “THC” comprises any derivative of Delta-9-tetrahydrocannabinol and/or salts thereof. In one embodiment, the compositions disclosed herein comprise THC and a compound of Formula I, II, III, IV, V, VI, VII, VIIIa, VIIIb, IXa, IXb, Xa, Xb, XIa or XIb. In one embodiment, the THC is purified THC. In one embodiment, methods disclosed herein comprise administering a composition comprising THC and a compound of Formula I, II, III, IV, V, VI, VII, VIIIa, VIIIb, IXa, IXb, Xa, Xb, XIa or XIb. In one embodiment, the THC is purified THC.

Within the context of this disclosure, the term “THCA” comprises any derivative of tetrahydrocannabinolic acid and/or salts thereof. In one embodiment, the compositions disclosed herein comprise THCA and a compound of Formula I, II, III, IV, V, VI, VII, VIIIa, VIIIb, IXa, IXb, Xa, Xb, XIa or XIb. In one embodiment, the THCA is purified THCA. In one embodiment, the methods disclosed herein comprise administering a composition comprising THCA and a compound of Formula I, II, III, IV, V, VI, VII, VIIIa, VIIIb, IXa, IXb, Xa, Xb, XIa or XIb. In one embodiment, the THCA is purified THCA.

Within the context of this disclosure, the term “THCV” comprises any derivative of Delta-9-tetrahydrocannabivarin and/or salts thereof. In one embodiment, the compositions disclosed herein comprise THCV and a compound of Formula I, II, III, IV, V, VI, VII, VIIIa, VIIIb, IXa, IXb, Xa, Xb, XIa or XIb. In one embodiment, the THCV is purified THCV. In one embodiment, the methods disclosed herein comprise administering a composition comprising THCV and a compound of Formula I, II, III, IV, V, VI, VII, VIIIa, VIIIb, IXa, IXb, Xa, Xb, XIa or XIb. In one embodiment, the THCV is purified THCV.

Within the context of this disclosure, the term “THCVA” comprises any derivative of Delta-9-tetrahydrocannabivarinic acid and/or salts thereof. In one embodiment, the compositions disclosed herein comprise THCVA and a compound of Formula I, II, III, IV, V, VI, VII, VIIIa, VIIIb, IXa, IXb, Xa, Xb, XIa or XIb. In one embodiment, the THCVA is purified THCVA. In one embodiment, the methods disclosed herein comprise administering a composition comprising THCVA and a compound of Formula I, II, III, IV, V, VI, VII, VIIIa, VIIIb, IXa, IXb, Xa, Xb, XIa or XIb. In one embodiment, the THCVA is purified THCVA.

Within the context of this disclosure, the term “CBC” comprises any derivative of Cannabichromene and/or salts thereof. In one embodiment, the compositions disclosed herein comprise CBC and a compound of Formula I, II, III, IV, V, VI, VII, VIIIa, VIIIb, IXa, IXb, Xa, Xb, XIa or XIb. In one embodiment, the CBC is purified CBC. In one embodiment, the methods disclosed herein comprise administering a composition comprising CBC and a compound of Formula I, II, III, IV, V, VI, VII, VIIIa, VIIIb, IXa, IXb, Xa, Xb, XIa or XIb. In one embodiment, the CBS is purified CBC.

Within the context of this disclosure, the term “CBCA” comprises any derivative of Cannabichromenic acid and/or salts thereof. In one embodiment, the compositions disclosed herein comprise CBCA and a compound of Formula I, II, III, IV, V, VI, VII, VIIIa, VIIIb, IXa, IXb, Xa, Xb, XIa or XIb. In one embodiment, the CBCA is purified CBCA. In one embodiment, the methods disclosed herein comprise administering a composition comprising CBCA and a compound of Formula I, II, III, IV, V, VI, VII, VIIIa, VIIIb, IXa, IXb, Xa, Xb, XIa or XIb. In one embodiment, the CBCA is purified CBCA.

Within the context of this disclosure, the term “CBCV” comprises any derivative of Cannabichromevarin and/or salts thereof. In one embodiment, the disclosed herein comprise CBCV and a compound of Formula I, II, III, IV, V, VI, VII, VIIIa, VIIIb, IXa, IXb, Xa, Xb, XIa or XIb. In one embodiment, the CBCV is purified CBCV. In one embodiment, the methods disclosed herein comprise administering a composition comprising CBCV and a compound of Formula I, II, III, IV, V, VI, VII, VIIIa, VIIIb, IXa, IXb, Xa, Xb, XIa or XIb. In one embodiment, the CBCV is purified CBCV.

Within the context of this disclosure, the term “CBCVA” comprises any derivative of Cannabichromevarinic acid and/or salts thereof. In one embodiment, the compositions disclosed herein comprise CBCVA and a compound of Formula I, II, III, IV, V, VI, VII, VIIIa, VIIIb, IXa, IXb, Xa, Xb, XIa or XIb. In one embodiment, the CBCVA is purified CBCVA. In one embodiment, the methods disclosed herein comprise administering a composition comprising CBCVA and a compound of Formula I, II, III, IV, V, VI, VII, VIIIa, VIIIb, IXa, IXb, Xa, Xb, XIa or XIb. In one embodiment, the CBCVA is purified CBCVA.

Within the context of this disclosure, the term “CBD” comprises any derivative of Cannabidiol and/or salt thereof. In one embodiment, the compositions disclosed herein comprise CBD and a compound of Formula I, II, III, IV, V, VI, VII, VIIIa, VIIIb, IXa, IXb, Xa, Xb, XIa or XIb. In one embodiment, the CBD is purified CBD. In one embodiment, the methods disclosed herein comprise administering a composition comprising CBD and a compound of Formula I, II, III, IV, V, VI, VII, VIIIa, VIIIb, IXa, IXb, Xa, Xb, XIa or XIb. In one embodiment, the CBD is purified CBD.

Within the context of this disclosure, the term “CBDA” comprises any derivative of Cannabidiolic acid and/or salts thereof. In one embodiment, the compositions disclosed herein comprise CBDA and a compound of Formula I, II, III, IV, V, VI, VII, VIIIa, VIIIb, IXa, IXb, Xa, Xb, XIa or XIb. In one embodiment, the CBDA is purified CBDA. In one embodiment, the methods disclosed herein comprise administering a composition comprising CBDA and a compound of Formula I, II, III, IV, V, VI, VII, VIIIa, VIIIb, IXa, IXb, Xa, Xb, XIa or XIb. In one embodiment, the CBDA is purified CBDA.

Within the context of this disclosure, the term “CBDV” comprises any derivative of Cannabidivarin and/or salts thereof. In one embodiment, the compositions disclosed herein comprise CBDV and a compound of Formula I, II, III, IV, V, VI, VII, VIIIa, VIIIb, IXa, IXb, Xa, Xb, XIa or XIb. In one embodiment, the CBDV is purified CBDV. In one embodiment, the methods disclosed herein comprise administering a composition comprising CBDV and a compound of Formula I, II, III, IV, V, VI, VII, VIIIa, VIIIb, IXa, IXb, Xa, Xb, XIa or XIb. In one embodiment, the CBDV is purified CBDV.

Within the context of this disclosure, the term “CBDVA” comprises any derivative of Cannabidivarinic acid and/or salts thereof. In one embodiment, the compositions disclosed herein comprise CBDVA and a compound of Formula I, II, III, IV, V, VI, VII, VIIIa, VIIIb, IXa, IXb, Xa, Xb, XIa or XIb. In one embodiment, the CBDVA is purified CBDVA. In one embodiment, the methods disclosed herein comprise administering a composition comprising CBDVA and a compound of Formula I, II, III, IV, V, VI, VII, VIIIa, VIIIb, IXa, IXb, Xa, Xb, XIa or XIb. In one embodiment, the CBDVA is purified CBDVA.

Within the context of this disclosure, the term “CBG” comprises any derivative of Cannabigerol and/or salts thereof. In one embodiment, the compositions disclosed herein comprise CBG and a compound of Formula I, II, III, IV, V, VI, VII, VIIIa, VIIIb, IXa, IXb, Xa, Xb, XIa or XIb. In one embodiment, the CBG is purified CBG. In one embodiment, the methods disclosed herein comprise administering a composition comprising CBG and a compound of Formula I, II, III, IV, V, VI, VII, VIIIa, VIIIb, IXa, IXb, Xa, Xb, XIa or XIb. In one embodiment, the CBG is purified CBG.

Within the context of this disclosure, the term “CBGA” comprises any derivative of Cannabigerolic acid and/or salts thereof. In one embodiment, the compositions disclosed herein comprise CBGA and a compound of Formula I, II, III, IV, V, VI, VII, VIIIa, VIIIb, IXa, IXb, Xa, Xb, XIa or XIb. In one embodiment, the CBGA is purified CBGA. In one embodiment, the methods disclosed herein comprise administering a composition comprising CBGA and a compound of Formula I, II, III, IV, V, VI, VII, VIIIa, VIIIb, IXa, IXb, Xa, Xb, XIa or XIb. In one embodiment, the CBGA is purified CBGA.

Within the context of this disclosure, the term “CBGV” comprises any derivative of Cannabigerovarin and/or salts thereof. In one embodiment, the compositions disclosed herein comprise CBGV and a compound of Formula I, II, III, IV, V, VI, VII, VIIIa, VIIIb, IXa, IXb, Xa, Xb, XIa or XIb. In one embodiment, the CBGV is purified CBGV. In one embodiment, the methods disclosed herein comprise administering a composition comprising CBGV and a compound of Formula I, II, III, IV, V, VI, VII, VIIIa, VIIIb, IXa, IXb, Xa, Xb, XIa or XIb. In one embodiment, the CBGV is purified CBGV.

Within the context of this disclosure, the term “CBGVA” comprises any derivative of Cannabigerovarinic acid and/or salts thereof. In one embodiment, the compositions disclosed herein comprise CBGVA and a compound of Formula I, II, III, IV, V, VI, VII, VIIIa, VIIIb, IXa, IXb, Xa, Xb, XIa or XIb. In one embodiment, the CBGVA is purified CBGVA. In one embodiment, the methods disclosed herein comprise administering a composition comprising CBGVA and a compound of Formula I, II, III, IV, V, VI, VII, VIIIa, VIIIb, IXa, IXb, Xa, Xb, XIa or XIb. In one embodiment, the CBGVA is purified CBGVA.

In one embodiment, the compositions disclosed herein comprise a particular ratio (e.g., a molar ratio) ranging from about 100:1 to about 1:100 of the compound of Formula I, II, III, IV, V, VI, VII, VIIIa, VIIIb, IXa, IXb, Xa, Xb, XIa or XIb and a purified cannabinoid. In one embodiment, the compositions disclosed herein comprise a particular ratio (e.g., a molar ratio) ranging from about 75:1 to about 1:75 of the compound of Formula I, II, III, IV, V, VI, VII, VIIIa, VIIIb, IXa, IXb, Xa, Xb, XIa or XIb and a purified cannabinoid. In one embodiment, the compositions disclosed herein comprise a particular ratio (e.g., a molar ratio) ranging from about 50:1 to about 1:50 of the compound of Formula I, II, III, IV, V, VI, VII, VIIIa, VIIIb, IXa, IXb, Xa, Xb, XIa or XIb and a purified cannabinoid. In one embodiment, the compositions disclosed herein comprise a particular ratio (e.g., a molar ratio) ranging from about 25:1 to about 1:25 of the compound of Formula I, II, III, IV, V, VI, VII, VIIIa, VIIIb, IXa, IXb, Xa, Xb, XIa or XIb and a purified cannabinoid. In one embodiment, the compositions disclosed herein comprise a particular ratio (e.g., a molar ratio) ranging from about 10:1 to about 1:10 of the compound of Formula I, II, III, IV, V, VI, VII, VIIIa, VIIIb, IXa, IXb, Xa, Xb, XIa or XIb and a purified cannabinoid. In one embodiment, the compositions disclosed herein comprise a particular ratio (e.g., a molar ratio) ranging from about 5:1 to about 1:5 of the compound of Formula I, II, III, IV, V, VI, VII, VIIIa, VIIIb, IXa, IXb, Xa, Xb, XIa or XIb and a purified cannabinoid.

In one embodiment, the compositions and methods disclosed herein comprise a compound of Formula I, II, III, IV, V, VI, VII, VIIIa, VIIIb, IXa, IXb, Xa, Xb, XIa or XIb, a first purified cannabinoid, and a second purified cannabinoid. In one embodiment, the second purified cannabinoid is chosen from THC, THCA, THCV, THCVA, CBC, CBCA, CBCV, CBCVA, CBD, CBDA, CBDV, CBDVA, CBG, CBGA, CBGV, and CBGVA.

In one embodiment, the compositions disclosed herein comprise a particular ratio (e.g., a molar ratio) ranging from about 100:1 to about 1:100 of the compound of Formula I, II, III, IV, V, VI, VII, VIIIa, VIlIb, IXa, IXb, Xa, Xb, XIa or XIb and the sum of the first purified cannabinoid and the second purified cannabinoid. In one embodiment, the compositions disclosed herein comprise a particular ratio (e.g., a molar ratio) ranging from about 75:1 to about 1:75 of a compound of Formula I, II, III, IV, V, VI, VII, VIIIa, VIIIb, IXa, IXb, Xa, Xb, XIa or XIb and the sum of the first purified cannabinoid and the second purified cannabinoid. In one embodiment, the compositions disclosed herein comprise a particular ratio (e.g., a molar ratio) ranging from about 50:1 to about 1:50 of a compound of Formula I, II, III, IV, V, VI, VII, VIIIa, VIIIb, IXa, IXb, Xa, Xb, XIa or XIb and the sum of the first purified cannabinoid and the second purified cannabinoid.

In one embodiment, the compositions disclosed herein comprise a particular ratio (e.g., a molar ratio) ranging from about 25:1 to about 1:25 of the compound of Formula I, II, III, IV, V, VI, VII, VIIIa, VIIIb, IXa, IXb, Xa, Xb, XIa or XIb and the sum of the first purified cannabinoid and the second purified cannabinoid. In one embodiment, the compositions disclosed herein comprise a particular ratio (e.g., a molar ratio) ranging from about 10:1 to about 1:10 of a compound of Formula I, II, III, IV, V, VI, VII, VIIIa, VIIIb, IXa, IXb, Xa, Xb, XIa or XIb and the sum of the first purified cannabinoid and the second purified cannabinoid. In one embodiment, the compositions disclosed herein comprise a particular ratio (e.g., a molar ratio) ranging from about 5:1 to about 1:5 of a compound of Formula I, II, III, IV, V, VI, VII, VIIIa, VIIIb, IXa, IXb, Xa, Xb, XIa or XIb and the sum of the first purified cannabinoid and the second purified cannabinoid.

In one embodiment, the compositions and methods disclosed herein comprise administering a compound of Formula I, II, III, IV, V, VI, VII, VIIIa, VIIIb, IXa, IXb, Xa, Xb, XIa or XIb and a terpene. In one embodiment, the terpene is a purified terpene.

As used herein, the term “terpene” refers to a compound belonging to a large class of compounds often biosynthesized from 5-carbon isoprene units. In one embodiment, a terpene is isolated from a plant, e.g., conifers, cannabis, basil, etc. In one embodiment, a terpene is produced by an insect, e.g., termites or swallowtail butterflies. In one embodiment, a terpene is a volatile compound. In one embodiment, a terpene produces an odor. In one embodiment, a terpene is a major component of a natural resin, e.g., turpentine produced from resin. In one embodiment, a terpene is derived biosynthetically from units of isoprene, which has the molecular formula C₅H₃. In one embodiment, the molecular formula of terpenes are multiples of (C₆H₈)ₙ, where n is the number of linked isoprene units, such as 1 to 5.

Within the context of this disclosure when a terpene is modified chemically, such as by oxidation or rearrangement of the carbon skeleton, the resulting compound is referred to as a “terpenoid.” In the relevant arts, terpenoids are sometimes referred to as isoprenoids.

In one embodiment, a terpene is the primary constituent or constituents of an essential oil from a plant and/or flower. Essential oils are used widely as fragrances in perfumery, medicine, and alternative medicines, e.g., aromatherapy.

In one embodiment, a terpene is categorized according to the number of isoprene (C₅H₃) units in the compound, for example, a monoterpene (C₁₀H₁₆), a sesquiterpene (C₁₅H₂₄), a diterpene (C₂₀H₃₂), a triterpene (C₃₀H₄₈), or a tetraterpene (C₄₀H₆₄).

Examples of terpenes within the context of this disclosure include acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, alpha-caryophyllene, beta-caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, beta-myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, and vanillin.

In one embodiment, a purified terpene is chosen from bornyl acetate, alpha-bisabolol, borneol, camphene, camphor, carene, beta-caryophyllene, cedrene, cymene, elemene, eucalyptol, eudesmol, farnesene, fenchol, geraniol, guaiacol, humulene, isoborneol, limonene, linalool, menthol, beta-myrcene, nerolidol, ocimene, phellandrene, phytol, pinene, pulegone, sabinene, terpineol, terpinolene, and valencene.

Within the context of this disclosure, the term “bornyl acetate” comprises any derivative and/or salt thereof, including any isomeric, structural and/or enantiomeric, variations thereof. In one embodiment, the compositions disclosed herein comprise bornyl acetate and a compound of Formula I, II, III, IV, V, VI, VII, VIIIa, VIIIb, IXa, IXb, Xa, Xb, XIa or XIb. In one embodiment, the bornyl acetate is purified bornyl acetate. In one embodiment, the methods disclosed herein comprise administering a composition comprising bornyl acetate and a compound of Formula I, II, III, IV, V, VI, VII, VIIIa, VIIIb, IXa, IXb, Xa, Xb, XIa or XIb. In one embodiment, the bornyl acetate is purified bornyl acetate.

Within the context of this disclosure, the term “alpha-bisabolol” comprises any derivative and/or salt thereof, including any isomeric, structural and/or enantiomeric, variations thereof. In one embodiment, the compositions disclosed herein comprise alpha-bisabolol and a compound of Formula I, II, III, IV, V, VI, VII, VIIIa, VIIIb, IXa, IXb, Xa, Xb, XIa or XIb. In one embodiment, the alpha-bisabolol is purified alpha-bisabolol. In one embodiment, the methods disclosed herein comprise administering a composition comprising alpha-bisabolol and a compound of Formula I, II, III, IV, V, VI, VII, VIIIa, VIIIb, IXa, IXb, Xa, Xb, XIa or XIb. In one embodiment, the alpha-bisabolol is purified alpha-bisabolol.

Within the context of this disclosure, the term “borneol” comprises any derivative and/or salt thereof, including any isomeric, structural, and/or enantiomeric, variations thereof. In one embodiment, the compositions disclosed herein comprise borneol and a compound of Formula I, II, III, IV, V, VI, VII, VIIIa, VIIIb, IXa, IXb, Xa, Xb, XIa or XIb. In one embodiment, the borneol is purified borneol. In one embodiment, the methods disclosed herein comprise administering a composition comprising borneol and a compound of Formula I, II, III, IV, V, VI, VII, VIIIa, VIIIb, IXa, IXb, Xa, Xb, XIa or XIb. In one embodiment, the borneol is purified borneol.

Within the context of this disclosure, the term “camphene” comprises any derivative and/or salt thereof, including any isomeric, structural and/or enantiomeric, variations thereof. In one embodiment, the compositions disclosed herein comprise camphene and a compound of Formula I, II, III, IV, V, VI, VII, VIIIa, VIIIb, IXa, IXb, Xa, Xb, XIa or XIb. In one embodiment, the camphene is purified camphene. In one embodiment, the methods disclosed herein comprise administering a composition comprising camphene and a compound of Formula I, II, III, IV, V, VI, VII, VIIIa, VIIIb, IXa, IXb, Xa, Xb, XIa or XIb. In one embodiment, the camphene is purified camphene.

Within the context of this disclosure, the term “camphor” comprises any derivative and/or salt thereof, including any isomeric, structural and/or enantiomeric, variations thereof. In one embodiment, the compositions disclosed herein comprise camphor and a compound of Formula I, II, III, IV, V, VI, VII, VIIIa, VIIIb, IXa, IXb, Xa, Xb, XIa or XIb. In one embodiment, the camphor is purified camphor. In one embodiment, the methods disclosed herein comprise administering a composition comprising camphor and a compound of Formula I, II, III, IV, V, VI, VII, VIIIa, VIlIb, IXa, IXb, Xa, Xb, XIa or XIb. In one embodiment, the camphor is purified camphor.

Within the context of this disclosure, the term “carene” comprises any derivative and/or salt thereof, including any isomeric, structural, and/or enantiomeric, variations thereof. In one embodiment, the compositions disclosed herein comprise carene and a compound of Formula I, II, III, IV, V, VI, VII, VIIIa, VIIIb, IXa, IXb, Xa, Xb, XIa or XIb. In one embodiment, the carene is purified carene. In one embodiment, the methods disclosed herein comprise administering a composition comprising carene and a compound of Formula I, II, III, IV, V, VI, VII, VIIIa, VIIIb, IXa, IXb, Xa, Xb, XIa or XIb. In one embodiment, the carene is purified carene.

Within the context of this disclosure, the term “beta-caryophyllene” comprises any derivative and/or salt thereof, including any isomeric, structural and/or enantiomeric, variations thereof. In one embodiment, the compositions disclosed herein comprise beta-caryophyllene and a compound of Formula I, II, III, IV, V, VI, VII, VIIIa, VIIIb, IXa, IXb, Xa, Xb, XIa or XIb. In one embodiment, the beta-caryophyllene is purified beta-caryophyllene. In one embodiment, the methods disclosed herein comprise administering a composition comprising beta-caryophyllene and a compound of Formula I, II, III, IV, V, VI, VII, VIIIa, VIIIb, IXa, IXb, Xa, Xb, XIa or XIb. In one embodiment, the beta-caryophyllene is purified beta-caryophyllene.

Within the context of this disclosure, the term “cedrene” comprises any derivative and/or salt thereof, including any isomeric, structural, and/or enantiomeric, variations thereof. In one embodiment, the compositions disclosed herein comprise cedrene and a compound of Formula I, II, III, IV, V, VI, VII, VIIIa, VIIIb, IXa, IXb, Xa, Xb, XIa or XIb. In one embodiment, the cedrene is purified cedrene. In one embodiment, the methods disclosed herein comprise administering a composition comprising cedrene and a compound of Formula I, II, III, IV, V, VI, VII, VIIIa, VIIIb, IXa, IXb, Xa, Xb, XIa or XIb. In one embodiment, the cedrene is purified cedrene.

Within the context of this disclosure, the term “cymene” comprises any derivative and/or salt to thereof, including any isomeric, structural and/or enantiomeric, variations thereof. In one embodiment, the compositions disclosed herein comprise cymene and a compound of Formula I, II, III, IV, V, VI, VII, VIIIa, VIIIb, IXa, IXb, Xa, Xb, XIa or XIb. In one embodiment, the cymene is purified cymene. In one embodiment, the methods disclosed herein comprise administering a composition comprising cymene and a compound of Formula I, II, III, IV, V, VI, VII, VIIIa, VIIIb, IXa, IXb, Xa, Xb, XIa or XIb. In one embodiment, the cymene is purified cymene.

Within the context of this disclosure, the term “elemene” comprises any derivative and/or salt thereof, including any isomeric, structural, and/or enantiomeric, variations thereof. In one embodiment, the compositions disclosed herein comprise elemene and a compound of Formula I, II, III, IV, V, VI, VII, VIIIa, VIIIb, IXa, IXb, Xa, Xb, XIa or XIb. In one embodiment, the elemene is purified elemene. In one embodiment, the methods disclosed herein comprise administering a composition comprising elemene and a compound of Formula I, II, III, IV, V, VI, VII, VIIIa, VIIIb, IXa, IXb, Xa, Xb, XIa or XIb. In one embodiment, the elemene is purified elemene.

Within the context of this disclosure, the term “eucalyptol” comprises any derivative and/or salt thereof, including any isomeric, structural and/or enantiomeric, variations thereof. In one embodiment, the compositions disclosed herein comprise eucalyptol and a compound of Formula I, II, III, IV, V, VI, VII, VIIIa, VIIIb, IXa, IXb, Xa, Xb, XIa or XIb. In one embodiment, the eucalyptol is purified eucalyptol. In one embodiment, the methods disclosed herein comprise administering a composition comprising eucalyptol and a compound of Formula I, II, III, IV, V, VI, VII, VIIIa, VIIIb, IXa, IXb, Xa, Xb, XIa or XIb. In one embodiment, the eucalyptol is purified eucalyptol.

Within the context of this disclosure, the term “eudesmol” comprises any derivative and/or salt thereof, including any isomeric, structural, and/or enantiomeric, variations thereof. In one embodiment, the compositions disclosed herein comprise eudesmol and a compound of Formula I, II, III, IV, V, VI, VII, VIIIa, VIIIb, IXa, IXb, Xa, Xb, XIa or XIb. In one embodiment, the eudesmol is purified eudesmol. In one embodiment, the methods disclosed herein comprise administering a composition comprising eudesmol and a compound of Formula I, II, III, IV, V, VI, VII, VIIIa, VIIIb, IXa, IXb, Xa, Xb, XIa or XIb. In one embodiment, the eudesmol is purified eudesmol.

Within the context of this disclosure, the term “farnesene” comprises any derivative and/or salt thereof, including any isomeric, structural, and/or enantiomeric, variations thereof. In one embodiment, the compositions disclosed herein comprise farnesene and a compound of Formula I, II, III, IV, V, VI, VII, VIIIa, VIIIb, IXa, IXb, Xa, Xb, XIa or XIb. In one embodiment, the farnesene is purified farnesene. In one embodiment, the methods disclosed herein comprise administering a composition comprising farnesene and a compound of Formula I, II, III, IV, V, VI, VII, VIIIa, VIIIb, IXa, IXb, Xa, Xb, XIa or XIb. In one embodiment, the farnesene is purified farnesene.

Within the context of this disclosure, the term “fenchol” comprises any derivative and/or salt thereof, including any isomeric, structural, and/or enantiomeric, variations thereof. In one embodiment, the compositions disclosed herein comprise fenchol and a compound of Formula I, II, III, IV, V, VI, VII, VIIIa, VIIIb, IXa, IXb, Xa, Xb, XIa or XIb. In one embodiment, the fenchol is purified fenchol. In one embodiment, the methods disclosed herein comprise administering a composition comprising fenchol and a compound of Formula I, II, III, IV, V, VI, VII, VIIIa, VIIIb, IXa, IXb, Xa, Xb, XIa or XIb. In one embodiment, the fenchol is purified fenchol.

Within the context of this disclosure, the term “geraniol” comprises any derivative and/or salt thereof, including any isomeric, structural and/or enantiomeric, variations thereof. In one embodiment, the compositions disclosed herein comprise geraniol and a compound of Formula I, II, III, IV, V, VI, VII, VIIIa, VIIIb, IXa, IXb, Xa, Xb, XIa or XIb. In one embodiment, the geraniol is purified geraniol. In one embodiment, the methods disclosed herein comprise administering a composition comprising geraniol and a compound of Formula I, II, III, IV, V, VI, VII, VIIIa, VIIIb, IXa, IXb, Xa, Xb, XIa or XIb. In one embodiment, the geraniol is purified geraniol.

Within the context of this disclosure, the term “guaiacol” comprises any derivative and/or salt thereof, including any isomeric, structural, and/or enantiomeric, variations thereof. In one embodiment, the compositions disclosed herein comprise guaiacol and a compound of Formula I, II, III, IV, V, VI, VII, VIIIa, VIIIb, IXa, IXb, Xa, Xb, XIa or XIb. In one embodiment, the guaiacol is purified guaiacol. In one embodiment, the methods disclosed herein comprise administering a composition comprising guaiacol and a compound of Formula I, II, III, IV, V, VI, VII, VIIIa, VIIIb, IXa, IXb, Xa, Xb, XIa or XIb. In one embodiment, the guaiacol is purified guaiacol.

Within the context of this disclosure, the term “humulene” comprises any derivative and/or salt thereof, including any isomeric, structural, and/or enantiomeric, variations thereof. In one embodiment, the compositions disclosed herein comprise humulene and a compound of Formula I, II, III, IV, V, VI, VII, VIIIa, VIIIb, IXa, IXb, Xa, Xb, XIa or XIb. In one embodiment, the humulene is purified humulene. In one embodiment, the methods disclosed herein comprise administering a composition comprising humulene and a compound of Formula I, II, III, IV, V, VI, VII, VIIIa, VIIIb, IXa, IXb, Xa, Xb, XIa or XIb. In one embodiment, the humulene is purified humulene.

Within the context of this disclosure, the term “isoborneol” comprises any derivative and/or salt thereof, including any isomeric, structural, and/or enantiomeric, variations thereof. In one embodiment, the compositions disclosed herein comprise isoborneol and a compound of Formula I, II, III, IV, V, VI, VII, VIIIa, VIIIb, IXa, IXb, Xa, Xb, XIa or XIb. In one embodiment, the isoborneol is purified isoborneol. In one embodiment, the methods disclosed herein comprise administering a composition comprising isoborneol and a compound of Formula I, II, III, IV, V, VI, VII, VIIIa, VIIIb, IXa, IXb, Xa, Xb, XIa or XIb. In one embodiment, the isoborneol purified isoborneol.

Within the context of this disclosure, the term “limonene” comprises any derivative and/or salt thereof, including any isomeric, structural and/or enantiomeric, variations thereof. In one embodiment, the compositions disclosed herein comprise limonene and a compound of Formula I, II, III, IV, V, VI, VII, VIIIa, VIIIb, IXa, IXb, Xa, Xb, XIa or XIb. In one embodiment, the limonene is purified limonene. In one embodiment, the methods disclosed herein comprise administering a composition comprising limonene and a compound of Formula I, II, III, IV, V, VI, VII, VIIIa, VIIIb, IXa, IXb, Xa, Xb, XIa or XIb. In one embodiment, the limonene is purified limonene.

Within the context of this disclosure, the term “linalool” comprises any derivative and/or salt thereof, including any isomeric, structural and/or enantiomeric, variations thereof. In one embodiment, the compositions disclosed herein comprise linalool and a compound of Formula I, II, III, IV, V, VI, VII, VIIIa, VIIIb, IXa, IXb, Xa, Xb, XIa or XIb. In one embodiment, the linalool is purified linalool. In one embodiment, the methods disclosed herein comprise administering a composition comprising linalool and a compound of Formula I, II, III, IV, V, VI, VII, VIIIa, VIIIb, IXa, IXb, Xa, Xb, XIa or XIb. In one embodiment, the linalool is purified linalool.

Within the context of this disclosure, the term “menthol” comprises any derivative and/or salt thereof, including any isomeric, structural and/or enantiomeric, variations thereof. In one embodiment, the compositions disclosed herein comprise menthol and a compound of Formula I, II, III, IV, V, VI, VII, VIIIa, VIIIb, IXa, IXb, Xa, Xb, XIa or XIb. In one embodiment, the menthol is purified menthol. In one embodiment, the methods disclosed herein comprise administering a composition comprising menthol and a compound of Formula I, II, III, IV, V, VI, VII, VIIIa, VIIIb, IXa, IXb, Xa, Xb, XIa or XIb. In one embodiment, the menthol is purified menthol.

Within the context of this disclosure, the term “beta-myrcene” comprises any derivative and/or salt thereof, including any isomeric, structural and/or enantiomeric, variations thereof. In one embodiment, the compositions disclosed herein comprise beta-myrcene and a compound of Formula I, II, III, IV, V, VI, VII, VIIIa, VIIIb, IXa, IXb, Xa, Xb, XIa or XIb. In one embodiment, the beta-myrcene is purified beta-myrcene. In one embodiment, the methods disclosed herein comprise administering a composition comprising beta-myrcene and a compound of Formula I, II, III, IV, V, VI, VII, VIIIa, VIIIb, IXa, IXb, Xa, Xb, XIa or XIb. In one embodiment, the beta-myrcene is purified beta-myrcene.

Within the context of this disclosure, the term “nerolidol” comprises any derivative and/or salt thereof, including any isomeric, structural, and/or enantiomeric, variations thereof. In one embodiment, the compositions disclosed herein comprise nerolidol and a compound of Formula I, II, III, IV, V, VI, VII, VIIIa, VIIIb, IXa, IXb, Xa, Xb, XIa or XIb. In one embodiment, the nerolidol is purified nerolidol. In one embodiment, the methods disclosed herein comprise administering a composition comprising nerolidol and a compound of Formula I, II, III, IV, V, VI, VII, VIIIa, VIIIb, IXa, IXb, Xa, Xb, XIa or XIb. In one embodiment, the nerolidol is purified nerolidol.

Within the context of this disclosure, the term “ocimene” comprises any derivative and/or salt thereof, including any isomeric, structural, and/or enantiomeric, variations thereof. In one embodiment, the compositions disclosed herein comprise ocimene and a compound of Formula I, II, III, IV, V, VI, VII, VIIIa, VIIIb, IXa, IXb, Xa, Xb, XIa or XIb. In one embodiment, the ocimene is purified ocimene. In one embodiment, the methods disclosed herein comprise administering a composition comprising ocimene and a compound of Formula I, II, III, IV, V, VI, VII, VIIIa, VIIIb, IXa, IXb, Xa, Xb, XIa or XIb. In one embodiment, the ocimene is purified ocimene.

Within the context of this disclosure, the term “phellandrene” comprises any derivative and/or salt thereof, including any isomeric, structural, and/or enantiomeric, variations thereof. In one embodiment, the compositions disclosed herein comprise phellandrene and a compound of Formula I, II, III, IV, V, VI, VII, VIIIa, VIIIb, IXa, IXb, Xa, Xb, XIa or XIb. In one embodiment, the phellandrene is purified phellandrene. In one embodiment, the methods disclosed herein comprise administering a composition comprising phellandrene and a compound of Formula I, II, III, IV, V, VI, VII, VIIIa, VIIIb, IXa, IXb, Xa, Xb, XIa or XIb. In one embodiment, the phellandrene is purified phellandrene.

Within the context of this disclosure, the term “phytol” comprises any derivative and/or salt thereof, including any isomeric, structural and/or enantiomeric, variations thereof. In one embodiment, the compositions disclosed herein comprise phytol and a compound of Formula I, II, III, IV, V, VI, VII, VIIIa, VIIIb, IXa, IXb, Xa, Xb, XIa or XIb. In one embodiment, the phytol is purified phytol. In one embodiment, the methods disclosed herein comprise administering a composition comprising phytol and a compound of Formula I, II, III, IV, V, VI, VII, VIIIa, VIIIb, IXa, IXb, Xa, Xb, XIa or XIb. In one embodiment, the phytol is purified phytol.

Within the context of this disclosure, the term “pinene” comprises any derivative and/or salt thereof, including any isomeric, structural, and/or enantiomeric, variations thereof. In one embodiment, the compositions disclosed herein comprise pinene and a compound of Formula I, II, III, IV, V, VI, VII, VIIIa, VIIIb, IXa, IXb, Xa, Xb, XIa or XIb. In one embodiment, the pinene is purified pinene. In one embodiment, the methods disclosed herein comprise administering a composition comprising pinene and a compound of Formula I, II, III, IV, V, VI, VII, VIIIa, VIIIb, IXa, IXb, Xa, Xb, XIa or XIb. In one embodiment, the pinene is purified pinene.

Within the context of this disclosure, the term “pulegone” comprises any derivative and/or salt thereof, including any isomeric, structural, and/or enantiomeric, variations thereof. In one embodiment, the compositions disclosed herein comprise pulegone and a compound of Formula I, II, III, IV, V, VI, VII, VIIIa, VIIIb, IXa, IXb, Xa, Xb, XIa or XIb. In one embodiment, the pulegone is purified pulegone. In one embodiment, the methods disclosed herein comprise administering a composition comprising pulegone and a compound of Formula I, II, III, IV, V, VI, VII, VIIIa, VIIIb, IXa, IXb, Xa, Xb, XIa or XIb. In one embodiment, the pulegone is purified pulegone.

Within the context of this disclosure, the term “sabinene” comprises any derivative and/or salt thereof, including any isomeric, structural, and/or enantiomeric, variations thereof. In one embodiment, the compositions disclosed herein comprise sabinene and a compound of Formula I, II, III, IV, V, VI, VII, VIIIa, VIIIb, IXa, IXb, Xa, Xb, XIa or XIb. In one embodiment, the sabinene is purified sabinene. In one embodiment, the methods disclosed herein comprise administering a composition comprising sabinene and a compound of Formula I, II, III, IV, V, VI, VII, VIIIa, VIIIb, IXa, IXb, Xa, Xb, XIa or XIb. In one embodiment, the sabinene is purified sabinene.

Within the context of this disclosure, the term “terpineol” comprises any derivative and/or salt thereof, including any isomeric, structural, and/or enantiomeric, variations thereof. In one embodiment, the compositions disclosed herein comprise terpineol and a compound of Formula I, II, III, IV, V, VI, VII, VIIIa, VIIIb, IXa, IXb, Xa, Xb, XIa or XIb. In one embodiment, the terpineol is purified terpineol. In one embodiment, the methods disclosed herein comprise administering a composition comprising terpineol and a compound of Formula I, II, III, IV, V, VI, VII, VIIIa, VIIIb, IXa, IXb, Xa, Xb, XIa or XIb. In one embodiment, the terpineol is purified terpineol.

Within the context of this disclosure, the term “terpinolene” comprises any derivative and/or salt thereof, including any isomeric, structural, and/or enantiomeric, variations thereof. In one embodiment, the compositions disclosed herein comprise terpinolene and a compound of Formula I, II, III, IV, V, VI, VII, VIIIa, VIIIb, IXa, IXb, Xa, Xb, XIa or XIb. In one embodiment, the terpinolene is purified terpinolene. In one embodiment, the methods disclosed herein comprise administering a composition comprising terpinolene and a compound of Formula I, II, III, IV, V, VI, VII, VIIIa, VIIIb, IXa, IXb, Xa, Xb, XIa or XIb. In one embodiment, the terpinolene is purified terpinolene.

Within the context of this disclosure, the term “valencene” comprises any derivative and/or salt thereof, including any isomeric, structural, and/or enantiomeric, variations thereof. In one embodiment, the compositions disclosed herein comprise valencene and a compound of Formula I, II, III, IV, V, VI, VII, VIIIa, VIIIb, IXa, IXb, Xa, Xb, XIa or XIb. In one embodiment, the valencene is purified valencene. In one embodiment, the methods disclosed herein comprise administering a composition comprising valencene and a compound of Formula I, II, III, IV, V, VI, VII, VIIIa, VIIIb, IXa, IXb, Xa, Xb, XIa or XIb. In one embodiment, the valencene is purified valencene.

In one embodiment, the compositions and methods disclosed herein include one or more erinacine molecules, which are optionally purified. In one embodiment, the compositions and methods disclosed herein comprise erinacine A. In one embodiment, the compositions and methods disclosed herein comprise erinacine B. In one embodiment, the compositions and methods disclosed herein comprise erinacine C. In one embodiment, the compositions and methods disclosed herein comprise erinacine D. In one embodiment, the compositions and methods disclosed herein comprise erinacine E. In one embodiment, the compositions and methods disclosed herein comprise erinacine F. In one embodiment, the compositions and methods disclosed herein comprise erinacine G. In one embodiment, the compositions and methods disclosed herein comprise erinacine H. In one embodiment, the compositions and methods disclosed herein comprise erinacine I. In one embodiment, the compositions and methods disclosed herein comprise erinacine J. In one embodiment, the compositions and methods disclosed herein comprise erinacine K. In one embodiment, the compositions and methods disclosed herein comprise erinacine P. In one embodiment, the compositions and methods disclosed herein comprise erinacine Q. In one embodiment, the compositions and methods disclosed herein comprise erinacine R. In one embodiment, the compositions and methods disclosed herein comprise erinacine S. In one embodiment, the erinacine molecule is a purified erinacine molecule. In one embodiment, the compositions and methods disclosed herein comprise one or more purified erinacine molecules and purified pyridine-3-carboxylic acid. In one embodiment, the compositions and methods disclosed herein comprise one or more purified erinacine molecules and a purified cannabinoid, such as CBD.

Within the context of this disclosure, the term “erinacine A” comprises any derivative and/or salt thereof, including any isomeric, structural and/or enantiomeric, variations thereof. In one embodiment, the compositions and methods disclosed herein comprise administering a formulation of erinacine A and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine A and a compound of Formula I, II, III, IV, V, VI, VII, VIIIa, VIIIb, IXa, IXb, Xa, Xb, XIa or XIb.

Within the context of this disclosure, the term “erinacine B” comprises any derivative and/or salt thereof, including any isomeric, structural and/or enantiomeric, variations thereof. In one embodiment, the compositions and methods disclosed herein comprise administering a formulation of erinacine B and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine B and a compound of Formula I, II, III, IV, V, VI, VII, VIIIa, VIIIb, IXa, IXb, Xa, Xb, XIa or XIb.

Within the context of this disclosure, the term “erinacine C” comprises any derivative and/or salt thereof, including any isomeric, structural and/or enantiomeric, variations thereof. In one embodiment, the compositions and methods disclosed herein comprise administering a formulation of erinacine C and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine C and a compound of Formula I, II, III, IV, V, VI, VII, VIIIa, VIIIb, IXa, IXb, Xa, Xb, XIa or XIb.

Within the context of this disclosure, the term “erinacine D” comprises any derivative and/or salt thereof, including any isomeric, structural and/or enantiomeric, variations thereof. In one embodiment, the compositions and methods disclosed herein comprise administering a formulation of erinacine D and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine D and a compound of Formula I, II, III, IV, V, VI, VII, VIIIa, VIIIb, IXa, IXb, Xa, Xb, XIa or XIb.

Within the context of this disclosure, the term “erinacine E” comprises any derivative and/or salt thereof, including any isomeric, structural and/or enantiomeric, variations thereof. In one embodiment, the compositions and methods disclosed herein comprise administering a formulation of erinacine E and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine E and a compound of Formula I, II, III, IV, V, VI, VII, VIIIa, VIIIb, IXa, IXb, Xa, Xb, XIa or XIb.

Within the context of this disclosure, the term “erinacine F” comprises any derivative and/or salt thereof, including any isomeric, structural and/or enantiomeric, variations thereof. In one embodiment, the compositions and methods disclosed herein comprise administering a formulation of erinacine F and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine F and a compound of Formula I, II, III, IV, V, VI, VII, VIIIa, VIIIb, IXa, IXb, Xa, Xb, XIa or XIb.

Within the context of this disclosure, the term “erinacine G” comprises any derivative and/or salt thereof, including any isomeric, structural and/or enantiomeric, variations thereof. In one embodiment, the compositions and methods disclosed herein comprise administering a formulation of erinacine G and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine G and a compound of Formula I, II, III, IV, V, VI, VII, VIIIa, VIIIb, IXa, IXb, Xa, Xb, XIa or XIb.

Within the context of this disclosure, the term “erinacine H” comprises any derivative and/or salt thereof, including any isomeric, structural and/or enantiomeric, variations thereof. In one embodiment, the compositions and methods disclosed herein comprise administering a formulation of erinacine H and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine H and a compound of Formula I, II, III, IV, V, VI, VII, VIIIa, VIIIb, IXa, IXb, Xa, Xb, XIa or XIb.

Within the context of this disclosure, the term “erinacine I” comprises any derivative and/or salt thereof, including any isomeric, structural and/or enantiomeric, variations thereof. In one embodiment, the compositions and methods disclosed herein comprise administering a formulation of erinacine I and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine I and a compound of Formula I, II, III, IV, V, VI, VII, VIIIa, VIIIb, IXa, IXb, Xa, Xb, XIa or XIb.

Within the context of this disclosure, the term “erinacine J” comprises any derivative and/or salt thereof, including any isomeric, structural and/or enantiomeric, variations thereof. In one embodiment, the compositions and methods disclosed herein comprise administering a formulation of erinacine J and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine J and a compound of Formula I, II, III, IV, V, VI, VII, VIIIa, VIIIb, IXa, IXb, Xa, Xb, XIa or XIb.

Within the context of this disclosure, the term “erinacine K” comprises any derivative and/or salt thereof, including any isomeric, structural and/or enantiomeric, variations thereof. In one embodiment, the compositions and methods disclosed herein comprise administering a formulation of erinacine K and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine K and a compound of Formula I, II, III, IV, V, VI, VII, VIIIa, VIIIb, IXa, IXb, Xa, Xb, XIa or XIb.

Within the context of this disclosure, the term “erinacine P” comprises any derivative and/or salt thereof, including any isomeric, structural and/or enantiomeric, variations thereof. In one embodiment, the compositions and methods disclosed herein comprise administering a formulation of erinacine P and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine P and a compound of Formula I, II, III, IV, V, VI, VII, VIIIa, VIIIb, IXa, IXb, Xa, Xb, XIa or XIb.

Within the context of this disclosure, the term “erinacine Q” comprises any derivative and/or salt thereof, including any isomeric, structural and/or enantiomeric, variations thereof. In one embodiment, the compositions and methods disclosed herein comprise administering a formulation of erinacine Q and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine Q and a compound of Formula I, II, III, IV, V, VI, VII, VIIIa, VIIIb, IXa, IXb, Xa, Xb, XIa or XIb.

Within the context of this disclosure, the term “erinacine R” comprises any derivative and/or salt thereof, including any isomeric, structural and/or enantiomeric, variations thereof. In one embodiment, the compositions and methods disclosed herein comprise administering a formulation of erinacine R and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine R and a compound of Formula I, II, III, IV, V, VI, VII, VIIIa, VIIIb, IXa, IXb, Xa, Xb, XIa or XIb.

Within the context of this disclosure, the term “erinacine S” comprises any derivative and/or salt thereof, including any isomeric, structural and/or enantiomeric, variations thereof. In one embodiment, the compositions and methods disclosed herein comprise administering a formulation of erinacine S and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified erinacine S and a compound of Formula I, II, III, IV, V, VI, VII, VIIIa, VIIIb, IXa, IXb, Xa, Xb, XIa or XIb.

The erinacine chemical structures are taken from Li I-C, Lee L-Y, Tzeng T W, et al. Neurohealth properties of Hericium erinaceus mycelia enriched with erinacines. In: Behavioural Neurology. 2018. doi:10.1155/2018/5802634

In one embodiment, the compositions and methods disclosed herein include one or more hericenone molecules, optionally purified. In one embodiment, the compositions and methods disclosed herein comprise hericenone A. In one embodiment, the compositions and methods disclosed herein comprise hericenone B. In one embodiment, the compositions and methods disclosed herein comprise hericenone C. In one embodiment, the compositions and methods disclosed herein comprise hericenone D. In one embodiment, the compositions and methods disclosed herein comprise hericenone E. In one embodiment, the compositions and methods disclosed herein comprise hericenone F. In one embodiment, the compositions and methods disclosed herein comprise hericenone G. In one embodiment, the compositions and methods disclosed herein comprise purified hericenone H.

Within the context of this disclosure, the term “hericenone A” comprises any derivative and/or salt thereof, including any isomeric, structural and/or enantiomeric, variations thereof. In one embodiment, the compositions and methods disclosed herein comprise administering a formulation of hericenone A and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone A and a compound of Formula I, II, III, IV, V, VI, VII, VIIIa, VIIIb, IXa, IXb, Xa, Xb, XIa or XIb.

Within the context of this disclosure, the term “hericenone B” comprises any derivative and/or salt thereof, including any isomeric, structural and/or enantiomeric, variations thereof. In one embodiment, the compositions and methods disclosed herein comprise administering a formulation of hericenone B and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone B and a compound of Formula I, II, III, IV, V, VI, VII, VIIIa, VIIIb, IXa, IXb, Xa, Xb, XIa or XIb.

Within the context of this disclosure, the term “hericenone C” comprises any derivative and/or salt thereof, including any isomeric, structural and/or enantiomeric, variations thereof. In one embodiment, the compositions and methods disclosed herein comprise administering a formulation of hericenone C and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone C and a compound of Formula I, II, III, IV, V, VI, VII, VIIIa, VIIIb, IXa, IXb, Xa, Xb, XIa or XIb.

Within the context of this disclosure, the term “hericenone D” comprises any derivative and/or salt thereof, including any isomeric, structural and/or enantiomeric, variations thereof. In one embodiment, the compositions and methods disclosed herein comprise administering a formulation of hericenone D and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone D and a compound of Formula I, II, III, IV, V, VI, VII, VIIIa, VIIIb, IXa, IXb, Xa, Xb, XIa or XIb.

Within the context of this disclosure, the term “hericenone E” comprises any derivative and/or salt thereof, including any isomeric, structural and/or enantiomeric, variations thereof. In one embodiment, the compositions and methods disclosed herein comprise administering a formulation of hericenone E and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone E and a compound of Formula I, II, III, IV, V, VI, VII, VIIIa, VIIIb, IXa, IXb, Xa, Xb, XIa or XIb.

Within the context of this disclosure, the term “hericenone F” comprises any derivative and/or salt thereof, including any isomeric, structural and/or enantiomeric, variations thereof. In one embodiment, the compositions and methods disclosed herein comprise administering a formulation of hericenone F and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone F and a compound of Formula I, II, III, IV, V, VI, VII, VIIIa, VIIIb, IXa, IXb, Xa, Xb, XIa or XIb.

Within the context of this disclosure, the term “hericenone G” comprises any derivative and/or salt thereof, including any isomeric, structural and/or enantiomeric, variations thereof. In one embodiment, the compositions and methods disclosed herein comprise administering a formulation of hericenone G and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone G and a compound of Formula I, II, III, IV, V, VI, VII, VIIIa, VIIIb, IXa, IXb, Xa, Xb, XIa or XIb.

Within the context of this disclosure, the term “hericenone H” comprises any derivative and/or salt thereof, including any isomeric, structural and/or enantiomeric, variations thereof. In one embodiment, the compositions and methods disclosed herein comprise administering a formulation of hericenone H and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified hericenone H and a compound of Formula I, II, III, IV, V, VI, VII, VIIIa, VIIIb, IXa, IXb, Xa, Xb, XIa or XIb.

In one embodiment, the compositions and methods disclosed herein comprise one or more purified hericenone molecules and purified pyridine-3-carboxylic acid. In one embodiment, the compositions and methods disclosed herein comprise one or more purified hericenone molecules and a purified cannabinoid, such as CBD.

Within the context of this disclosure, the term “pyridine-3-carboxylic acid” comprises any derivative and/or salt thereof, including any isomeric, structural and/or enantiomeric, variations thereof. In one embodiment, the compositions and methods disclosed herein comprise administering a formulation of pyridine-3-carboxylic acid and a compound of Formula I, II, III, IV, V, VI, VII, VIIIa, VIIIb, IXa, IXb, Xa, Xb, XIa or XIb. In one embodiment, the compositions and methods disclosed herein comprise administering a formulation of purified pyridine-3-carboxylic acid and a compound of Formula I, II, III, IV, V, VI, VII, VIIIa, VIIIb, IXa, IXb, Xa, Xb, XIa or XIb.

In one embodiment, the compositions and methods disclosed herein include one or more purified hericenone molecules and one or more purified erinacine molecules.

In one embodiment, the compositions and methods disclosed herein include one or more purified serotonergic derivatives, one or more purified hericenone molecules and one or more purified erinacine molecules.

In one embodiment, the compositions and methods disclosed herein include one or more purified serotonergic derivatives, one or more purified hericenone molecules, one or more purified erinacine molecules and one or more purified cannabinoids.

In one embodiment, the compositions and methods disclosed herein include one or more purified serotonergic derivatives, one or more purified hericenone molecules, one or more purified erinacine molecules and one or more purified pyridine-3-carboxylic acid.

In one embodiment, the compositions and methods disclosed herein include one or more compounds of Formula I, II, III, IV, V, VI, VII, VIIIa, VIIIb, IXa, IXb, Xa, Xb, XIa or XIb and one or more purified molecules attained by extracting and subsequently purifying one or more compounds from an organism chosen from Bacopa monnieri (for example, the purified molecule bacoside A3), Centella asiatica (for example, the purified molecule asiaticoside), Gingko biloba (for example, the purified molecule myricetin), Zingiber officinale (for example, the purified molecule zingerone), Ocimum sanctum (for example, the purified molecule linalool), Polygonum cuspidatum (for example, the purified molecule resveratrol), Origanum vulgare (for example, the purified molecule carvacrol), Origanum onites (for example, the purified molecule thymol), Rosmarinus officinalis (for example, the purified molecule rosmarinic acid), Rosmarinus eriocalyx (for example, the purified molecule camphor), Curcuma longa (for example, the purified molecule curcumin), Camellia sinensis (for example, the purified molecule theobromine), Lavandula spica (for example, the purified molecule caryophyllene), Scutellaria lateriflora (for example, the purified molecule baicalin), Avena sativa (for example, the purified molecule avenalin), Avena byzantina (for example, the purified molecule beta-glucan), Salvia divinorum (for example, the purified molecule salvinorin A), Banisteriopsis caapi (for example, the purified molecule harmine), Psychotria species (for example, the purified molecule dimethyltryptamine), Tabernanthe iboga (for example, the purified molecule ibogaine), Voacanga africana (for example, the purified molecule voacangine), Tabernaemontana undulata (for example, the purified molecule ibogamine), Lophophora williamsii (for example, the purified molecule mescaline), Ipomoea tricolor (for example, the purified molecule ergonovine), and Argyreia nervosa (for example, the purified molecule ergine).

In one embodiment, the compositions disclosed herein comprise a particular ratio (e.g., a molar ratio) ranging from about 100:1 to about 1:100 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 75:1 to about 1:75 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 50:1 to about 1:50 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 25:1 to about 1:25 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 10:1 to about 1:10 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 5:1 to about 1:5 of the compound of Formula I, II, III, IV, V, VI, VII, VIIIa, VIIIb, IXa, IXb, Xa, Xb, XIa or XIb and a purified terpene.

In one embodiment, the compositions and methods disclosed herein comprise a compound of Formula I, II, III, IV, V, VI, VII, VIIIa, VIIIb, IXa, IXb, Xa, Xb, XIa or XIb, a purified cannabinoid, and a purified terpene.

In one embodiment, the compositions disclosed herein comprise a particular ratio (e.g., a molar ratio) ranging from about 100:1 to about 1:100 of the compound of Formula I, II, III, IV, V, VI, VII, VIIIa, VIIIb, IXa, IXb, Xa, Xb, XIa or XIb and a purified cannabinoid and a particular ratio (e.g., a molar ratio) ranging from about 100:1 to about 1:100 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 75:1 to about 1:75 of compound of Formula I, II, III, IV, V, VI, VII, VIIIa, VIIIb, IXa, IXb, Xa, Xb, XIa or XIb and a purified cannabinoid and a particular ratio (e.g., a molar ratio) ranging from about 75:1 to about 1:75 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 50:1 to about 1:50 of the compound of Formula I, II, III, IV, V, VI, VII, VIIIa, VIlIb, IXa, IXb, Xa, Xb, XIa or XIb and a purified cannabinoid and a particular ratio (e.g., a molar ratio) ranging from about 50:1 to about 1:50 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 25:1 to about 1:25 of the compound of Formula I, II, III, IV, V, VI, VII, VIIIa, VIIIb, IXa, IXb, Xa, Xb, XIa or XIb and a purified cannabinoid and a particular ratio (e.g., a molar ratio) ranging from about 25:1 to about 1:25 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 10:1 to about 1:10 of compound of Formula I, II, III, IV, V, VI, VII, VIIIa, VIIIb, IXa, IXb, Xa, Xb, XIa or XIb and a purified cannabinoid and a particular ratio (e.g., a molar ratio) ranging from about 10:1 to about 1:10 of the compound of Formula I, II, III, IV, V, VI, VII, VIIIa, VIIIb, IXa, IXb, Xa, Xb, XIa or XIb and a purified terpene.

In one embodiment, the compositions disclosed herein comprise a particular ratio (e.g., a molar ratio) ranging from about 5:1 to about 1:5 of the compound of Formula I, II, III, IV, V, VI, VII, VIIIa, VIIIb, IXa, IXb, Xa, Xb, XIa or XIb and a purified cannabinoid and a particular ratio (e.g., a molar ratio) ranging from about 5:1 to about 1:5 of the compound of Formula I, II, III, IV, V, VI, VII, VIIIa, VIIIb, IXa, IXb, Xa, Xb, XIa or XIb and a purified terpene.

In one embodiment, a purified terpene modulates the activity of a neurotransmitter activity modulator, e.g., a compound of Formula I, II, III, IV, V, VI, VII, VIIIa, VIIIb, IXa, IXb, Xa, Xb, XIa or XIb, a serotonergic drug, an adrenergic drug, a dopaminergic drug, a psilocybin derivative, etc.

As used herein, the term “serotonergic drug” refers to a compound that binds to, blocks, or otherwise influences (e.g., via an allosteric reaction) activity at a serotonin receptor. In one embodiment, a serotonergic drug binds to a serotonin receptor. In one embodiment, a serotonergic drug indirectly affects a serotonin receptor, e.g., via interactions affecting the reactivity of other molecules at the serotonin receptor. In one embodiment, a serotonergic drug is an agonist, e.g., a compound activating a serotonin receptor. In one embodiment, a serotonergic drug is an antagonist, e.g., a compound binding but not activating a serotonin receptor, e.g., blocking a receptor. In one embodiment, a serotonergic drug is an effector molecule, e.g., a compound binding to an enzyme for allosteric regulation. In one embodiment, a serotonergic drug acts (either directly or indirectly) at more than one type of receptor (e.g., SHT, dopamine, adrenergic, acetylcholine, etc.).

In one embodiment, a serotonergic drug is an antidepressant.

In one embodiment, a serotonergic drug is an anxiolytic.

In one embodiment, a serotonergic drug is a selective serotonin reuptake inhibitor.

In one embodiment, a serotonergic drug is a selective serotonin norepinephrine reuptake inhibitor.

In some embodiments, the compounds of Formula I, II, III, IV, V, VI, VII, VIIIa, VIIIb, IXa, IXb, Xa, Xb, XIa or XIb are serotonergic drugs. In some embodiments, at least one compound of Formula I, II, III, IV, V, VI, VII, VIIIa, VIIIb, IXa, IXb, Xa, Xb, XIa or XIb is administered with a second serotonergic drug, such as one of the serotonergic drugs identified below.

Some exemplary serotonergic drugs include the following molecules: 4-hydroxy-N-methyltryptamine (aka 3[2-(methylamino)ethyl]-1H-indol-4-ol), aeruginascin (aka [3-[2-(trimethylazaniumyl)ethyl]-1H-indol-4-yl]hydrogen phosphate), baeocystin (aka [3-[2-(methylamino)ethyl]-1H-indol-4-yl]dihydrogen phosphate), bufotenidine (aka 3-[2-(trimethylazaniumypethyl]-1H-indol-5-olate), bufotenin (aka 3-[2-(dimethylamino)ethyl]-1H-indol-5-ol), ethocybin (aka [3-[2-(diethylamino)ethyl]-1H-indol-4-yl]dihydrogen phosphate), norbaeocystin (aka [3-(2-aminoethyl)-1H-indol-4-yl]dihydrogen phosphate), norpsilocin, psilocin (aka 3-[2-(dimethylamino)ethyl]-1H-indol-4-ol), psilocybin (aka [3-[2-(dimethylamino)ethyl]-1H-indol-4-yl]dihydrogen phosphate), serotonin (aka 3-(2-aminoethyl)-1H-indol-5-ol), 1 P-LSD (aka (6aR,9R)-N,N-diethyl-7-methyl-4-propanoyl-6,6a,8,9-tetrahydroindolo [4,3-fg]quinoline-9-carboxamide), ALD-52 (aka (6aR,9R)-4-acetyl-N,N-diethyl-7-methyl-6,6a,8,9-tetrahydroindolo[4,3-fg]quinoline-9-carboxamide), AL-LAD (aka (6aR,9R)-N,N-diethyl-7-prop-2-enyl-6,6a,8,9-tetrahydro-4H-indolo[4,3-fg]quinoline-9-carboxamide), BU-LAD (aka (6aR,9R)-7-butyl-N,N-diethyl-6,6a,8,9-tetrahydro-4H-indolo[4,3-fg]quinoline-9-carboxamide), DAL (aka (6aR,9R)-7-methyl-N,N-bis(prop-2-enyl)-6,6a,8,9-tetrahydro-4H-indolo[4,3-fg]quinoline-9-carboxamide), DAM-57 (aka (6aR,9R)-N,N,7-trimethyl-6,6a,8,9-tetrahydro-4H-indolo[4,3-fg]quinoline-9-carboxamide), EIPLA (aka (6aR,9R)-N-ethyl-7-methyl-N-propan-2-yl-6,6a,8,9-tetrahydro-4H-indolo[4,3-fg]quinoline-9-carboxamide), ETH-LAD (aka (6aR,9R)-N,N,7-triethyl-6,6a,8,9-tetrahydro-4H-indolo[4,3-fg]quinoline-9-carboxamide), LAE-32 (aka (6aR,9R)-N-ethyl-7-methyl-6,6a,8,9-tetrahydro-4H-indolo[4,3-fg]quinoline-9-carboxamide), LPD-824 (aka [(6aR,9R)-7-methyl-6,6a,8,9-tetrahydro-4H-indolo[4,3-fg]quinoline-9-yl]-pyrrolidin-1-ylmethanone), LSB (aka (6aR,9R)-N-butan-2-yl-7-methyl-6,6a,8,9-tetrahydro-4H-indolo[4,3-fg]quinoline-9-carboxamide), LSA (aka (6aR,9R)-7-methyl-6,6a,8,9-tetrahydro-4H-indolo[4,3-fg]quinoline-9-carboxamide), LSD-25 (aka (6aR,9R)-N,N-diethyl-7-methyl-6,6a,8,9-tetrahydro-4H-indolo[4,3-fg]quinoline-9-carboxamide), LSD-PiP (aka (7-methyl-6,6a,8,9-tetrahydro-4H-indolo[4,3-fg]quinoline-9-yl)-piperidin-1-ylmethanone), LSM-775 (aka [(6aR,9R)-7-methyl-6,6a,8,9-tetrahydro-4H-indolo[4,3-fg]quinoline-9-yl]-morpholin-4-ylmethanone), LSP (aka (6aR,9R)-7-methyl-N-pentan-3-yl-6,6a,8,9-tetrahydro-4H-indolo[4,3-fg]quinoline-9-carboxamide), LSZ (aka [(6aR,9R)-7-methyl-6,6a,8,9-tetrahydro-4H-indolo[4,3-fg]quinoline-9-yl]-[(2S,4S)-2,4-dimethylazetidin-1-yl]methanone), methergine (aka (6aR,9R)-N-(1-hydroxybutan-2-yl)-7-methyl-6,6a,8,9-tetrahydro-4H-indolo[4,3-fg]quinoline-9-carboxamide), MiPLA (aka (6aR,9R)-N,7-dimethyl-N-propan-2-yl-6,6a,8,9-tetrahydro-4H-indolo[4,3-fg]-quinoline-9-carboxamide), NDTDI, PARGY-LAD, PRO-LAD (aka (6aR,9R)-N,N-diethyl-7-propyl-6,6a,8,9-tetrahydro-4H-indolo[4,3-fg]quinoline-9-carboxamide), 2-Me-DET (aka N,N-diethyl-2-(2-methyl-1H-indol-3-yl)ethanamine), 2-Me-DMT (aka N,N-dimethyl-2-(2-methyl-1H-indol-3-yl)ethanamine), 2,alpha-DMT (aka 1-(2-methyl-1H-indol-3-yl)propan-2-amine), 4-AcO-DALT (aka [3-[2-[bis(prop-2-enyl)amino]ethyl]-1H-indol-4-yl]acetate), 4-AcO-DET (aka [3-[2-(diethylamino)ethyl]-1H-indol-4-yl]acetate), 4-AcO-DIPT (aka 3-[2-(Diisopropylamino)ethyl]-1H-indol-4-yl acetate), 4-AcO-DMT (aka [3-[2-(dimethylamino)ethyl]-1H-indol-4-yl]acetate), 4-AcO-DPT (aka [3-[2-(dipropylamino)ethyl]-1H-indol-4-yl]acetate), 4-AcO-EPT (aka 3-{2-[Ethyl(propyl)amino]ethyl}-1H-indol-4-yl acetate), 4-AcO-MET (aka [3-[2-[ethyl(methyl)amino]ethyl]-1H-indol-4-yl]acetate), 4-AcO-MIPT (aka [3-[2-[methyl(propan-2-yl)amino]ethyl]-1H-indol-4-yl]acetate), 4-AcO-MPT, 4-HO-DBT (aka 3-[2-(dibutylamino)ethyl]-1H-indol-4-ol), 4-HO-DET (aka 3-[2-(diethylamino)ethyl]-1H-indol-4-ol), 4-HO-DIPT (aka 3-[2-[di(propan-2-yl)amino]ethyl]-1H-indol-4-ol), 4-HO-DPT (aka 3-[2-(dipropylamino)ethyl]-1H-indol-4-ol), 4-HO-EPT, 4-HO-MCPT, 4-HO-MET (aka 3-[2-[ethyl(methyl)amino]ethyl]-1H-indol-4-ol), 4-HO-MIPT (aka 3-[2-[methyl(propan-2-yl)amino]ethyl]-1H-indol-4-ol), 4-HO-MPMI (aka 3-[(1-methylpyrrolidin-2-yl)methyl]-1H-indol-4-ol), 4-HO-MPT (aka 3-[2-[methyl(propyl)amino]ethyl]-1H-indol-4-ol), 4-HO-pyr-T (aka 3-(2-pyrrolidin-1-ylethyl)-1H-indol-4-ol), 4-MeO-MIPT (aka N-[2-(4-methoxy-1H-indol-3-yl)ethyl]-N-methylpropan-2-amine), 4,5-MDO-DIPT (aka N-[2-(6H-[1,3]dioxolo[4,5-e]indol-8-yl)ethyl]-N-propan-2-ylpropan-2-amine), 4,5-MDO-DMT (aka 2-(6H-[1,3]dioxolo[4,5-e]indol-8-yl)-N,N-dimethylethanamine), 5-BROMO-DMT (aka 2-(5-bromo-1H-indol-3-yl)-N,N-dimethylethanamine), 5-chloro-alpha-MT (aka 1-(5-chloro-1H-indol-3-yl)propan-2-amine), 5-fluoro-AMT (aka 1-(5-fluoro-1H-indol-3-yl)propan-2-amine), 5-MeO-AET (aka 1-(5-methoxy-1H-indol-3-yl)butan-2-amine), 5-MeO-AMT (aka 1-(5-methoxy-1H-indol-3-yl)propan-2-amine), 5-MeO-DALT (aka N-[2-(5-methoxy-1H-indol-3-yl)ethyl]-N-prop-2-enylprop-2-en-1-amine), 5-MeO-DET (aka N,N-diethyl-2-(5-methoxy-1H-indol-3-yl)ethanamine), 5-MeO-DiPT (aka N-[2-(5-methoxy-1H-indol-3-yl)ethyl]-N-propan-2-ylpropan-2-amine), 5-MeO-DMT (aka 2-(5-methoxy-1H-indol-3-yl)-N,N-dimethylethanamine), 5-MeO-DPT (aka N-[2-(5-methoxy-1H-indol-3-yl)ethyl]-N-propylpropan-1-amine), 5-MeO-EiPT (aka N-ethyl-N-[2-(5-methoxy-1H-indol-3-yl)ethyl]propan-2-amine), 5-MeO-MALT (aka N-[2-(5-Methoxy-1H-indol-3-yl)ethyl]-N-methylprop-2-en-1-amine), 5-MeO-MiPT (aka N-[2-(5-methoxy-1H-indol-3-yl)ethyl]-N-methylpropan-2-amine), 5-MeO-NMT (aka 2-(5-methoxy-1H-indol-3-yl)-N-methylethanamine hydrochloride), 5-MeO-pyr-T (aka 4-fluoro-5-methoxy-3-(2-pyrrolidin-1-ylethyl)-1H-indole), 5-MeO-TMT (aka 2-(5-methoxy-2-methyl-1H-indol-3-yl)-N,N-dimethylethanamine), 5-MeS-DMT (aka N,N-dimethyl-2-(5-methylsulfanyl-1H-indol-3-yl)ethanamine), 5,6-MDO-DIPT (aka N-[2-(5H-[1,3]dioxolo[4,5-f]indol-7-yl)ethyl]-N-propan-2-ylpropan-2-amine), 5,6-MDO-DMT (aka 2-(5H-[1,3]dioxolo[4,5-f]indol-7-yl)-N,N-dimethylethanamine), 5,6-MDO-MIPT (aka N-[2-(5H-[1,3]dioxolo[4,5-f]indol-7-yl)ethyl]-N-ethylpropan-2-amine), 5,6-MeO-MIPT (aka N-[2-(5,6-dimethoxy-1H-indol-3-yl)ethyl]-N-methylpropan-2-amine), 5,N,N-TMT (aka N,N-dimethyl-2-(5-methyl-1H-indol-3-ethanamine), 6-MeO-THH (aka 6-methoxy-1-methyl-2,3,4,9-tetrahydro-1H-pyrido[3,4-b]indole), alpha-ET (aka 1-(1H-indol-3-yl)butan-2-amine), alpha-MT (aka 1-(1H-indol-3-yl)propan-2-amine), alpha-TMT (aka 1-(1H-indol-3-yl)-N,N-dimethylpropan-2-amine), alpha,N-DMT (aka 2-(1H-indol-3-yl)-N,N-dimethylethanamine), alpha,N,O-TMS (aka 1-(5-methoxy-1H-indol-3-yl)-N-methylpropan-2-amine), alpha,O-DMS (aka 1-(5-methoxy-1H-indol-3-yl)propan-2-amine), DALT (aka N-[2-(1H-indol-3-yl)ethyl]-N-prop-2-enylprop-2-en-1-amine), DBT (aka N-butyl-N-[2-(1H-indol-3-yl)ethyl]butan-1-amine), DET (aka N,N-diethyl-2-(1H-indol-3-yl)ethanamine), DiPT (aka N-[2-(5-methoxy-1H-indol-3-yl)ethyl]-N-propan-2-ylpropan-2-amine), DMT (aka 2-(1H-indol-3-yl)-N,N-dimethylethanamine), DPT (aka N-[2-(1H-indol-3-yl)ethyl]-N-propylpropan-1-amine), EiPT (aka N-ethyl-N-[2-(1H-indol-3-yl)ethyl]propan-2-amine), Harmaline (aka 7-methoxy-1-methyl-3,4-dihydro-2H-pyrido[3,4-b]indole), Harmine (aka 7-methoxy-1-methyl-9H-pyrido[3,4-b]indole), MALT, MBT (aka 3H-1,3-benzothiazole-2-thione), Melatonin (aka N-[2-(5-methoxy-1H-indol-3-yl)ethyl]acetamide), MET (aka N-ethyl-2-(1H-indol-3-yl)-N-methylethanamine), MiPT (aka N-[2-(1H-indol-3-yl)ethyl]-N-methylpropan-2-amine), MPT (aka 3-[2-[methyl(propyl)amino]ethyl]-1H-indol-4-ol), NET (aka N-ethyl-2-(1H-indol-3-yl)ethanamine), NMT (aka 2-(1H-indol-3-yl)-N-methylethanamine), PiPT (aka N-[2-(1H-indol-3-yl)ethyl]-N-propan-2-ylpropan-1-amine), pyr-T (aka 3-(2-pyrrolidin-1-ylethyl)-1H-indole), T (aka 2-(1H-indol-3-yl)ethanamine), Tetrahydroharmine (aka 7-methoxy-1-methyl-2,3,4,9-tetrahydro-1H-pyrido[3,4-b]indole), 2-Br-4,5-MDA (aka 1-(6-bromo-1,3-benzodioxol-5-yl)propan-2-amine), 2-TIM (aka 2-(3,4-dimethoxy-2-methylsulfanylphenyl)ethanamine), 2-TOET (aka 1-(4-ethyl-5-methoxy-2-methylsulfanylphenyl)propan-2-amine), 2-TOM (aka 1-(5-methoxy-4-methyl-2-methylsulfanylphenyl)propan-2-amine), 2,4-DMA (aka 1-(2,4-dimethoxyphenyl)propan-2-amine), 2,5-DMA (aka 1-(2,5-dimethoxyphenyl)propan-2-amine), 2C-B (aka 2-(4-bromo-2,5-dimethoxyphenyl)ethanamine), 2C-C(aka 2-(4-chloro-2,5-dimethoxyphenyl)ethanamine), 2C-D (aka 2-(2,5-dimethoxy-4-methylphenyl)ethanamine), 2C-E (aka 2-(4-ethyl-2,5-dimethoxyphenyl)ethanamine), 2C—F (aka 2-(4-fluoro-2,5-dimethoxyphenyl)ethanamine), 2C-G (aka 2-(2,5-dimethoxy-3,4-dimethylpheny)ethanamine), 2C-G-3 (aka 2-(4,7-dimethoxy-2,3-dihydro-1H-inden-5-yl)ethanamine), 2C-G-4 (aka 2-(1,4-dimethoxy-5,6,7,8-tetrahydronaphthalen-2-yl)ethanamine), 2C-G-5 (aka CAS 207740-20-3), 2C-G-N(aka 2-(1,4-dimethoxynaphthalen-2-yl)ethanamine), 2C—H (aka 2-(2,5-dimethoxyphenyl)ethanamine), 2C—I (aka 2-(4-iodo-2,5-dimethoxyphenyl)ethanamine), 2C—N(aka 2-(2,5-dimethoxy-4-nitrophenyl)ethanamine), 2C—O-4 (aka 2-(2,5-dimethoxy-4-propan-2-yloxyphenyl)ethanamine), 2C—P (aka 2-(2,5-dimethoxy-4-propylphenyl)ethanamine), 2C-SE (aka 2-(2,5-dimethoxy-4-methylselanylphenyl)ethanamine), 2C-T (aka 2-(2,5-dimethoxy-4-methylsulfanylphenyl)ethanamine), 2C-T-13 (aka 2-[2,5-dimethoxy-4-(2-methoxyethylsulfanyl)phenyl]ethanamine), 2C-T-15 (aka 2-(4-cyclopropylsulfanyl-2,5-dimethoxyphenyl)ethanamine), 2C-T-17 (aka 2-(4-butan-2-ylsulfanyl-2,5-dimethoxyphenyl)ethanamine), 2C-T-2 (aka 2-(4-ethylsulfanyl-2,5-dimethoxypheny)ethanamine), 2C-T-2 (aka 2-[4-(2-fluoroethylsulfanyl)-2,5-dimethoxyphenyl]ethanamine), 2C-T-4 (aka 2-(2,5-dimethoxy-4-propan-2-ylsulfanylpheny)ethanamine), 2C-T-7 (aka 2-(2,5-dimethoxy-4-propylsulfanylphenyl)ethanamine), 2C-T-8 (aka 2-[4-(cyclopropylmethylsulfanyl)-2,5-dimethoxyphenyl]ethanamine), 2C-T-9 (aka 2-(4-butylsulfanyl-2,5-dimethoxypheny)ethanamine), 2C-TFM (aka 2-[2,5-dimethoxy-4-(trifluoromethyl)phenyl]ethanamine), 2T-MMDA-3a (aka 1-(4-methylsulfanyl-1,3-benzodioxol-5-yl)propan-2-amine), 3-T-TRIS (aka 2-(3,4-diethoxy-5-ethylsulfanylphenyl)ethanamine), 3-TASB (aka 2-(3-ethoxy-4-ethylsulfanyl-5-methoxyphenyl)ethanamine), 3-TE (aka 2-(4-ethoxy-3-methoxy-5-methylsulfanylphenyl)ethanamine), 3-TFM (aka 2-(2,4-dimethoxy-3-methylsulfanylphenyl)ethanamine), 3-TM (aka 2-(3,4-dimethoxy-5-methylsulfanylpheny)ethanamine), 3-TME (aka 2-(3-ethylsulfanyl-4,5-dimethoxyphenyl)ethanamine), 3-TSB (aka 2-(3-ethoxy-5-ethylsulfanyl-4-methoxyphenyl)ethanamine), 3,4-DMA (aka 1-(3,4-dimethoxyphenyl)propan-2-amine), 3C-BZ (aka 1-(3,5-dimethoxy-4-phenylmethoxyphenyl)propan-2-amine), 3C-E (aka 1-(4-ethoxy-3,5-dimethoxyphenyl)propan-2-amine), 4-Br-3,5-DMA (aka 1-(4-bromo-3,5-dimethoxyphenyl)propan-2-amine), 4-D (aka CAS 1020518-87-9), 4-MA (aka 1-(4-methoxyphenyl)propan-2-amine), 4-T-TRIS (aka 2-(3,5-diethoxy-4-ethylsulfanylphenyl)ethanamine), 4-TASB (aka 2-(3-ethoxy-4-ethylsulfanyl-5-methoxypheny)ethanamine), 4-TE (aka 2-(4-ethylsulfanyl-3,5-dimethoxyphenyl)ethanamine), 4-TIM (aka 2-(2,3-dimethoxy-4-methylsulfanylphenyl)ethanamine), 4-TM (aka 2-(3,5-dimethoxy-4-methylsulfanylphenyl)ethanamine), 4-TME (aka 2-(3-ethoxy-5-methoxy-4-methylsulfanylpheny)ethanamine), 4-TSB (aka 2-(3,5-diethoxy-4-methylsulfanylphenyl)ethanamine), 4T-MMDA-2 (aka 1-(5-methoxy-1,3-benzoxathiol-6-yl)propan-2-amine), 5-TASB (aka 2-(3,4-diethoxy-5-methylsulfanylphenyl)ethanamine), 5-TME (aka 2-(3-ethoxy-4-methoxy-5-methylsulfanylphenyl)ethanamine), 5-TOET (aka 1-(4-ethyl-2-methoxy-5-methylsulfanylphenyl)propan-2-amine), 5-TOM (aka 1-(2-methoxy-4-methyl-5-methylsulfanylphenyl)propan-2-amine), 25B-NBF (aka 2-(4-bromo-2,5-dimethoxyphenyl)-N-[(2-fluorophenyl)methyl]ethanamine), 25B-NBOH (aka 2-[[2-(4-bromo-2,5-dimethoxypheny)ethylaminolmethyl]phenol), 25B-NBOMe (aka 2-(4-bromo-2,5-dimethoxyphenyl)-N[(2-methoxyphenyl)methyl]ethanamine), 25C-NB3OMe (aka 2-(4-chloro-2,5-dimethoxyphenyl)-N-[((3-methoxypheny)methyl]ethanamine), 25C-NB4OMe (aka 2-(4-chloro-2,5-dimethoxyphenyl)-N-[(4-methoxyphenyl)methyl]ethanamine), 25C-NBF (aka 2-(4-chloro-2,5-dimethoxyphenyl)-N-[(2-fluorophenyl)methyl]ethanamine), 25C-NBOH (aka 2-(4-chloro-2,5-dimethoxyphenyl)ethylaminolmethyl]phenol), 25C-NBOMe (aka 2-(4-chloro-2,5-dimethoxyphenyl)-N[(2-methoxyphenyl)methyl]ethanamine), 25CN-NBOH (aka 4-[2-[(2-hydroxyphenyl)methylamino]ethyl]-2,5-dimethoxybenzonitrile), 25CN-NBOMe (aka CAS 1354632-16-8), 25D-NBOMe (aka 2-(2,5-dimethoxy-4-methylphenyl)-N-[(2-methoxyphenyl)methyl]ethanamine), 25E-NBOMe (aka 2-(4-ethyl-2,5-dimethoxyphenyl)-N4(2-methoxyphenyl)methyl]ethanamine), 25G-NBOMe (aka 2-(2,5-dimethoxy-3,4-dimethylphenyl)-N-[(2-methoxyphenyl)methyl]ethanamine), 25H-NBOMe (aka 2-(2,5-dimethoxyphenyl)-N-[(2-methoxyphenyl)methyl]ethanamine), 25H-NB34MD (aka N-(1,3-benzodioxol-5-ylmethyl)-2-(4-iodo-2,5-dimethoxyphenyl)ethanamine), 251-NB3OMe (aka 2-(4-iodo-2,5-dimethoxyphenyl)-N-[(3-methoxyphenyl)methyl]ethanamine), 251-NB4OMe (aka 2-(4-iodo-2,5-dimethoxyphenyl)-N-[(4-methoxyphenyl)methyl]ethanamine), 251-NBF (aka N-[(2-fluorophenyl)methyl]-2-(4-iodo-2,5-dimethoxyphenyl)ethanamine), 251-NBMD (aka N-(1,3-benzodioxol-4-ylmethyl)-2-(4-iodo-2,5-dimethoxyphenyl)ethanamine), 251-NBOH (aka 2-[[2-(4-iodo-2,5-dimethoxypheny)ethylamino]methyl]phenol), 251-NBOMe (aka 2-(4-iodo-2,5-dimethoxyphenyl)-N-[(2-methoxyphenyl)methyl]ethanamine), 25iP-NBOMe (aka 2-(2,5-dimethoxy-4-propan-2-ylphenyl)-N-[(2-methoxypheny)methyl]ethanamine), 25N-NBOMe (aka 2-(2,5-dimethoxy-4-nitrophenyl)-N-[(2-methoxypheny)methyl]ethanamine), 25P-NBOMe (aka 2-(2,5-dimethoxy-4-propylphenyl)-N-[(2-methoxyphenyl)methyl]ethanamine), 25TFM-NBOMe (aka 2-[2,5-dimethoxy-4-(trifluoromethyl)phenyl]-N-[(2-methoxyphenyl)methyl]ethanamine), 2CBCB-NBOMe (aka 1-[(7R)-3-bromo-2,5-dimethoxy-7-bicyclo[4.2.0]octa-1(6),2,4-trienyl]-N-[(-2-methoxypheny)methyl]methanamine), 2CBFIy-NBOMe (aka 2-(4-bromo-2,3,6,7-tetrahydrofuro[2,3-f][1]benzofuran-8-yl)-N-[(2-methoxyphenyl)methyl]thanamine), AEM (aka 1-(3,4,5-trimethoxyphenyl)butan-2-amine), AL (aka 2-(3,5-dimethoxy-4-prop-2-enoxyphenyl)ethanamine), ALEPH (aka 1-(2,5-dimethoxy-4-methylsulfanylphenyl)propan-2-amine; hydrochloride), ALEPH-2 (aka 1-(4-ethylsulfanyl-2,5-dimethoxyphenyl)propan-2-amine), ALEPH-4 (aka 1-(2,5-dimethoxy-4-propan-2-ylsulfanylphenyl)propan-2-amine), ALEPH-6 (aka 1-(2,5-dimethoxy-4-phenylsulfanylphenyl)propan-2-amine), ALEPH-7 (aka 1-(2,5-dimethoxy-4-propylsulfanylphenyl)propan-2-amine), ARIADNE (aka (2R)-1-(2,5-dimethoxy-4-methylphenyl)butan-2-amine), ASB (aka 2-(3,4-diethoxy-5-methoxyphenyl)ethanamine), B (aka 2-(4-butoxy-3,5-dimethoxyphenyl)ethanamine), BEATRICE (aka 1-(2,5-dimethoxy-4-methylphenyl)-N-methylpropan-2-amine), beta-D (aka 2,2-dideuterio-2-(3,4,5-trimethoxyphenyl)ethanamine), BIS-TOM (aka 1-[4-methyl-2,5-bis(methylsulfanyl)phenyl]propan-2-amine), bk-2C-B (aka 2-amino-1-(4-bromo-2,5-dimethoxyphenyl)ethanone), BOB (aka 2-(4-bromo-2,5-dimethoxyphenyl)-2-methoxyethanamine), BOD (aka 2-(2,5-dimethoxy-4-methylphenyl)-2-methoxyethanamine), BOH (aka 2-(1,3-benzodioxol-5-yl)-2-methoxyethanamine), BOHD (aka 2-amino-1-(2,5-dimethoxy-4-methylpheny)ethanol), BOM (aka 2-methoxy-2-(3,4,5-trimethoxyphenyl)ethanamine), bromo-dragonFLY (aka 1-(4-bromofuro[2,3-f][1]benzofuran-8-yl)propan-2-amine), butylone (aka 1-(1,3-benzodioxl-5-yl)-2-(methylamino)butan-1-one), CPM (aka 2-[4-(cyclopropylmethoxy)-3,5-dimethoxyphenyl]ethanamine), DESOXY (aka 2-(3,5-dimethoxy-4-methylpheny)ethanamine), DMCPA (aka 2-(2,5-dimethoxy-4-methylphenyl)cyclopropan-1-amine), DME (aka 2-amino-1-(3,4-dimethoxyphenyl)ethanol), DMMDA (aka 1-(4,7-dimethoxy-1,3-benzodioxol-5-yl)propan-2-amine), DMMDA-2 (aka 1-(6,7-dimethoxy-1,3-benzodioxol-5-yl)propan-2-amine), DMPEA (aka 2-(3,4-dimethoxyphenyl)ethanamine), DOAM (aka 1-(2,5-dimethoxy-4-pentylphenyl)propan-2-amine), DOB (aka 1-(4-bromo-2,5-dimethoxyphenyl)propan-2-amine), DOBU (aka 1-(4-butyl-2,5-dimethoxyphenyl)propan-2-amine), DOC (aka 1-(4-chloro-2,5-dimethoxyphenyl)propan-2-amine), DOEF (aka 1-[4-(2-fluoroethyl)-2,5-dimethoxyphenyl]propan-2-amine), DOET (aka 1-(4-ethyl-2,5-dimethoxyphenyl)propan-2-amine), DOF (aka 1-(4-fluoro-2,5-dimethoxyphenyl)propan-2-amine), DOI (aka 1-(4-iodo-2,5-dimethoxyphenyl)propan-2-amine), DOM (aka 1-(2,5-dimethoxy-4-methylphenyl)propan-2-amine), DON (aka 1-(2,5-dimethoxy-4-nitrophenyl)propan-2-amine), DOPR (aka 1-(2,5-dimethoxy-4-propylphenyl)propan-2-amine), DOTFM (aka 1-[2,5-dimethoxy-4-(trifluoromethyl)phenyl]propan-2-amine), E (aka 2-(4-ethoxy-3,5-dimethoxyphenyl)ethanamine), EBDP (aka 1-(1,3-benzodioxol-5-yl)-N-ethylpentan-2-amine), EEE (aka 1-(2,4,5-triethoxyphenyl)propan-2-amine), EEM (aka 1-(2,4-diethoxy-5-methoxyphenyl)propan-2-amine), EME (aka 1-(2,5-diethoxy-4-methoxyphenyl)propan-2-amine), EMM (aka 1-(2-ethoxy-4,5-dimethoxyphenyl)propan-2-amine), ETHYL-J (aka 1-(1,3-benzodioxol-5-yl)-N-ethylbutan-2-amine), ETHYL-K (aka 1-(1,3-benzodioxol-5-yl)-N-ethylpentan-2-amine), F-2 (aka 1-(5-methoxy-2-methyl-2,3-dihydro-1-benzofuran-6-yl)propan-2-amine), F-22 (aka 1-(5-methoxy-2,2-dimethyl-3H-1-benzofuran-6-yl)propan-2-amine), FLEA (aka N-[1-(1,3-benzodioxol-5-yl)propan-2-yl]-N-methylhydroxylamine), G-3 (aka 1-(4,7-dimethoxy-2,3-dihydro-1H-inden-5-yl)propan-2-amine), G-4 (aka 1-(1,4-dimethoxy-5,6,7,8-tetrahydronaphthalen-2-yl)propan-2-amine), G-5 (aka 3,6-dimethoxy-4-(2-aminopropyl)benzonorbornane), G-N(aka 1-(1,4-dimethoxynaphthalen-2-yl)propan-2-amine), GANESHA (aka 1-(2,5-dimethoxy-3,4-dimethylphenyl)propan-2-amine), HOT-17 (aka N-[2-(4-butan-2-ylsulfanyl-2,5-dimethoxypheny)ethyl]hydroxylamine), HOT-2 (aka N-[2-(4-ethylsulfanyl-2,5-dimethoxypheny)ethyl]hydroxylamine), HOT-7 (aka N-[2-(2,5-dimethoxy-4-propylsulfanylpheny)ethyl]hydroxylamine), IDNNA (aka 1-(4-iodo-2,5-dimethoxyphenyl)-N,N-dimethylpropan-2-amine), IM (aka 2-(2,3,4-trimethoxyphenyl)e thanamine), IP (aka 2-(3,5-dimethoxy-4-propan-2-yloxyphenyl)ethanamine), IRIS (aka 1-(5-ethoxy-2-methoxy-4-methylphenyl)propan-2-amine), J (aka 1-(1,3-benzodioxol-5-yl)butan-2-amine), jimscaline (aka [(1R)-4,5,6-trimethoxy-2,3-dihydro-1H-inden-1-yl]methanamine), LOPHOPHINE (aka 2-(7-methoxy-1,3-benzodioxol-5-yl)ethanamine), M (aka 2-(3,4,5-trimethoxypheny)ethanamine), MADAM-6 (aka N-methyl-1-(6-methyl-1,3-benzodioxol-5-yl)propan-2-amine), MAL (aka 2-[3,5-dimethoxy-4-(2-methylprop-2-enoxy)phenyl]ethanamine), MDA (aka 1-(1,3-benzodioxol-5-yl)propan-2-amine), MDAL (aka 1-(1,3-benzodioxol-5-yl)-N-prop-2-enylpropan-2-amine), MDBU (aka N-[1-(1,3-benzodioxol-5-yl)propan-2-yl]butan-1-amine), MDBZ (aka 1-(1,3-benzodioxol-5-yl)-N-benzylpropan-2-amine), MDCPM (aka 1-(3a,7a-dihydro-1,3-benzodioxol-5-yl)-N-(cyclopropylmethyl)propan-2-amine), MDDM (aka 1-(1,3-benzodioxol-5-yl)-N,N-dimethylpropan-2-amine), MDE (aka 1-(1,3-benzodioxol-5-yl)-N-ethylpropan-2-amine), MDHOET (aka 2-[1-(1,3-benzodioxol-5-yl)propan-2-ylamino]ethanol), MDIP (aka 1-(1,3-benzodioxol-5-yl)-N-propan-2-ylpropan-2-amine), MDMA (aka 1-(1,3-benzodioxol-5-yl)-N-methylpropan-2-amine), MDMC (aka 1-(2,3-dihydro-1,4-benzodioxin-6-yl)-N-methylpropan-2-amine), MDMEO (aka 1-(1,3-benzodioxol-5-yl)-N-methoxypropan-2-amine), MDMEOET (aka 1-(1,3-benzodioxol-5-yl)-N-(2-methoxyethyl)propan-2-amine), MDMP (aka 1-(1,3-benzodioxol-5-yl)-N,2-dimethylpropan-2-amine), MDOH (aka N-[1-(1,3-benzodioxol-5-yl)propan-2-yl]hydroxylamine), MDPEA (aka 2-(1,3-benzodioxol-5-yl)ethanamine), MDPH (aka 1-(1,3-benzodioxol-5-yl)-2-methylpropan-2-amine), MDPL (aka 1-(1,3-benzodioxol-5-yl)-N-prop-2-ynylpropan-2-amine), MDPR (aka 1-(1,3-benzodioxol-5-yl)-N-propylpropan-2-amine), ME (aka 2-(3-ethoxy-4,5-dimethoxyphenyl)ethanamine), MEDA (aka 1-(5-methoxy-2,3-dihydro-1,4-benzodioxin-7-yl)propan-2-amine), MEE (aka 1-(4,5-diethoxy-2-methoxyphenyl)propan-2-amine), MEM (aka 1-(4-ethoxy-2,5-dimethoxyphenyl)propan-2-amine), MEPEA (aka 2-(4-ethoxy-3-methoxyphenyl)ethanamine), META-DOB (aka 1-(5-bromo-2,4-dimethoxyphenyl)propan-2-amine), META-DOT (aka 1-(2,4-dimethoxy-5-methylsulfanylphenyl)propan-2-amine), METHYL-DMA (aka 1-(2,5-dimethoxyphenyl)-N-methylpropan-2-amine), METHYL-DOB (aka 1-(4-bromo-2,5-dimethoxyphenyl)-N-methylpropan-2-amine), METHYL-J (aka 1-(1,3-benzodioxol-5-yl)-N-methylbutan-2-amine), METHYL-K (aka 1-(1,3-benzodioxol-5-yl)-N-methylpentan-2-amine), METHYL-MA (aka 1-(4-methoxyphenyl)-N-methylpropan-2-amine), METHYL-MMDA-2 (aka 1-(6-methoxy-1,3-benzodioxol-5-yl)-N-methylpropan-2-amine), MMDA (aka 1-(7-methoxy-1,3-benzodioxol-5-yl)propan-2-amine), MMDA-2 (aka 1-(6-methoxy-1,3-benzodioxol-5-yl)propan-2-amine), MMDA-3a (aka 1-(4-methoxy-1,3-benzodioxol-5-yl)propan-2-amine), MMDA-3b (aka 1-(7-methoxy-1,3-benzodioxol-4-yl)propan-2-amine), MME (aka 1-(5-ethoxy-2,4-dimethoxyphenyl)propan-2-amine), MP (aka 2-(3,4-dimethoxy-5-propoxyphenyl)ethanamine), MPM (aka 1-(2,4-dimethoxy-5-propoxyphenyl)propan-2-amine), NBOMe-mescaline (aka N-[(2-methoxyphenyl)methyl]-2-(3,4,5-trimethoxypheny)ethanamine), ORTHO-DOT (aka 1-(4,5-dimethoxy-2-methylsulfanylphenyl)propan-2-amine), P (aka 2-(3,5-dimethoxy-4-propoxyphenyl)ethanamine), PE (aka 2-[3,5-dimethoxy-4-(2-phenylethoxy)phenyl]ethanamine), PEA (aka 2-phenylethanamine), PROPYNYL (aka 2-(3,5-dimethoxy-4-prop-2-ynoxyphenyl)ethanamine), psi-2C-T-4, psi-DOM (aka 1-(2,6-dimethoxy-4-methylphenyl)propan-2-amine), SB (aka 2-(3,5-diethoxy-4-methoxyphenyl)ethanamine), TA (aka 1-(2,3,4,5-tetramethoxyphenyl)propan-2-amine), TB (aka 2-(4-butylsulfanyl-3,5-dimethoxypheny)ethanamine), TCB-2 (aka (3-bromo-2,5-dimethoxy-7-bicyclo[4.2.0]octa-1(6), 2,4-trienyl)methanamine hydrobromide), TMA (aka 1-(3,4,5-trimethoxyphenyl)propan-2-amine), TMA-2 (aka 1-(2,4,5-trimethoxyphenyl)propan-2-amine), TMA-3 (aka 1-(2,3,4-trimethoxyphenyl)propan-2-amine), TMA-4 (aka 1-(2,3,5-trimethoxyphenyl)propan-2-amine), TMA-5 (aka 1-(2,3,6-trimethoxyphenyl)propan-2-amine), TMA-6 (aka 1-(2,4,6-trimethoxyphenyl)propan-2-amine), TMPEA (aka 2-(2,4,5-trimethoxyphenyl)ethanamine), TOMSO (aka 1-(2-methoxy-4-methyl-5-methylsulfinylphenyl)propan-2-amine), TP (aka 2-(3,5-dimethoxy-4-propylsulfanylphenyl)ethanamine), and TRIS (aka 2-(3,4,5-triethoxyphenyl)ethanamine).

In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and derivatives thereof.

In one embodiment, serotonin acts at a serotonin receptor, e.g., by acting as a ligand at a 5-HT receptor. In one embodiment, serotonin is produced by an organism for use as a neurotransmitter within that organism. In one embodiment, the compositions and methods disclosed herein increase the activity at a serotonin receptor. In one embodiment, the compositions and methods disclosed herein decrease the activity at a serotonin receptor.

As used herein, the term “serotonin receptor” refers to a collection of proteins outside a cell capable of receiving signals and activating internal signal transduction pathways causing a cellular response. In one embodiment, a serotonin receptor is found on a cell within the central nervous system of an organism. In one embodiment, a serotonin receptor is found on a cell within the peripheral nervous system of an organism. In one embodiment, serotonin is the natural ligand for a serotonin receptor. In one embodiment, a serotonin receptor modulates the release of a neurotransmitter, e.g., glutamate, gamma-Aminobutyric acid, dopamine, epinephrine (a.k.a. norepinephrine), acetylcholine, etc. In one embodiment, a serotonin receptor modulates the release of a hormone, e.g., oxytocin, prolactin, vasopressin, cortisol, corticotropin, substance P, etc.

Examples of serotonin receptors include, but are not limited to, 5-HT_{1A}, 5-HT_{1B}, 5-HT_{1D}, 5-HT_{1E}, 5-HT_{2A}, 5-HT_{2B}, 5-HT_{2C}, 5-HT₃, 5-HT₄, 5-HT_{5A}, 5-HT_{5B}, 5-HT₆, and 5-HT₇.

As used herein, the term “adrenergic drug” refers to a compound that binds, blocks, or otherwise influences (e.g., via an allosteric reaction) activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.).

In one embodiment, an adrenergic drug is an antidepressant.

In one embodiment, an adrenergic drug is a norepinephrine transporter inhibitor.

In one embodiment, an adrenergic drug is a vesicular monoamine transporter inhibitor.

In one embodiment, an adrenergic drug is chosen from adrenaline, agmatine, amoxapine, aptazapine, atomoxetine, bupropion, clonidine, doxepin, duloxetine, esmirtazpine, mianserin, mirabegron, mirtazapine, norepinephrine, phentolamine, phenylephrine, piperoxan, reserpine, ritodrine, setiptiline, tesofensine, timolol, trazodone, trimipramine, and xylazine.

In one embodiment, an adrenergic drug acts at an adrenergic receptor, e.g., by acting as a ligand at an adrenergic receptor. In one embodiment, adrenaline is produced by an organism for use as a neurotransmitter within that organism. In one embodiment, norepinephrine is produced by an organism for use as a neurotransmitter within that organism.

In one embodiment, the compositions and methods disclosed herein increase the activity at an adrenergic receptor. In one embodiment, the compositions and methods disclosed herein decrease the activity at an adrenergic receptor.

As used herein, the term “adrenergic receptor” refers to a collection of proteins outside a cell capable of receiving signals and activating internal signal transduction pathways causing a cellular response. In one embodiment, an adrenergic receptor is found on a cell within the central nervous system of an organism. In one embodiment, an adrenergic receptor is found on a cell within the sympathetic nervous system of an organism.

As used herein, the term “dopaminergic drug” refers to a compound that binds, blocks, or otherwise influences (e.g., via an allosteric reaction) activity at a dopamine receptor. In one embodiment, a dopaminergic drug binds to a dopamine receptor. In one embodiment, a dopaminergic drug indirectly affects a dopamine receptor, e.g., via interactions affecting the reactivity of other molecules at the dopamine receptor. In one embodiment, a dopaminergic drug is an agonist, e.g., a compound activating a dopamine receptor. In one embodiment, a dopaminergic drug is an antagonist, e.g., a compound binding but not activating a dopamine receptor, e.g., blocking a receptor. In one embodiment, a dopaminergic drug is an effector molecule, e.g., a compound binding to an enzyme for allosteric regulation. In one embodiment, a dopaminergic drug acts (either directly or indirectly) at more than one type of receptor (e.g., 5HT, dopamine, adrenergic, acetylcholine, etc.).

In one embodiment, a dopaminergic drug is a dopamine transporter inhibitor.

In one embodiment, a dopaminergic drug is a vesicular monoamine transporter inhibitor.

In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, and thioridazine.

In one embodiment, a dopaminergic drug acts at a dopamine receptor, e.g., by acting as a ligand at a dopamine receptor. In one embodiment, dopamine is produced by an organism for use as a neurotransmitter within that organism. In one embodiment, the compositions and methods disclosed herein increase the activity at a dopamine receptor. In one embodiment, the compositions and methods disclosed herein decrease the activity at a dopamine receptor.

As used herein, the term “dopamine receptor” refers to a collection of proteins outside a cell capable of receiving signals and activating internal signal transduction pathways causing a cellular response. In one embodiment, a dopamine receptor is found on a cell within the central nervous system of an organism.

In one embodiment, a purified terpene modulates the activity of a neurotransmitter at its native receptor, e.g., serotonin at a serotonin receptor, dopamine at a dopaminergic drug, norephedrine at an adrenergic receptor, etc.

In one embodiment, a purified terpene is active at one or more receptors, e.g., a serotonin receptor, an adrenergic receptor, a dopamine receptor, a GABAergic receptor, a glutaminergic receptor, a histaminergic receptor, a cholinergic receptor, an opioid receptor, or a glycinergic receptor.

In one embodiment, the compositions disclosed herein comprise a monoamine oxidase inhibitor.

As used herein, the term “monoamine oxidase inhibitor” refers to a molecule binding to a monoamine oxidase enzyme thereby reducing the activity of the monoamine oxidase enzyme. Within the context of this disclosure, examples of monoamine oxidase inhibitors include aurorix, deprenyl, eldepryl, emsam, humoryl, hydracarbazine, isocarboxazid, linezolid, manerix, nydrazid, phenelzine, pirazidol, procarbazine, rasagiline, and tranylcypromine. In one embodiment, monoamine oxidase catalyzes the oxidation of a monoamine, e.g., serotonin, dopamine, norepinephrine, amphetamine, adrenaline, etc.

In one embodiment, the compositions disclosed herein comprise a stabilizer. As used herein, the term “stabilizer” refers to a compound useful for preventing the degradation of an active ingredient, e.g., a compound of Formula I, II, III, IV, V, VI, VII, VIIIa, VIIIb, IXa, IXb, Xa, Xb, XIa or XIb, a psilocybin derivative, a cannabinoid, a terpene, etc. In one embodiment, a stabilizer prevents an active ingredient from degrading. In one embodiment, a stabilizer prevents a serotonergic drug from reacting with other compounds in the composition, e.g., a cannabinoid, a terpene, a base, an acid, etc. In one embodiment, a stabilizer prevents a serotonergic drug from reacting with the ambient atmosphere, e.g., heat, light, water, and/or oxygen. In one embodiment, a stabilizer comprises an antioxidant. In one embodiment, a stabilizer comprises a pH buffer.

In one embodiment, the methods and compositions disclosed herein comprise an antioxidant. As used herein, the term “antioxidant” refers to a compound and/or a composition useful for preventing oxidation. In one embodiment, an antioxidant protects an active ingredient from “free radicals”. Within the context of this disclosure, a “free radical” is an atom, molecule, or an ion with an unpaired valence electron. In one embodiment, an antioxidant is an electron donor.

In one embodiment, an antioxidant is chosen from ascorbic acid, lycopene, tocopherol, melatonin, retinol, astaxanthin, lutein, apigenin, carnosine, selenium, zinc, curcumin, and a salt or derivative thereof.

In one embodiment, an antioxidant is ascorbic acid and/or its salts or derivatives. Within the context of this disclosure, the term “ascorbic acid” comprises Vitamin C and/or a salt or derivative thereof.

In one embodiment, an antioxidant prevents the oxidation of a composition comprising one or more compounds disclosed herein, e.g., compounds of Formula I, II, III, IV, V, VI, VII, VIIIa, VIIIb, IXa, IXb, Xa, Xb, XIa or XIb, psilocybin derivatives, cannabinoids, terpenes, and/or mixtures thereof. For example, preventing the oxidation of a phenolic group attached to a psilocybin derivative.

As used herein, the term “oxidation” refers to the formal loss of electrons and/or the increase of the formal oxidation state and/or the addition of an oxygen atom or atoms. As used herein, “reduction” refers to the formal gain of electrons and/or the decrease of the formal oxidation state. Zumdahl, Steven S., et al. Chemistry, 7th. Cengage Learning, 2018.

In one embodiment, the methods and compositions disclosed herein comprise a pH buffer.

As used herein, the term “pH buffer” refers to a compound or a composition useful for maintaining the pH of a composition. In one embodiment, a pH buffer comprises a weak acid and a corresponding conjugate base. In one embodiment, a pH buffer comprises a weak base and a corresponding conjugate acid. In one embodiment, a pH buffer does not change the pH of a composition with the addition of a strong acid and/or base.

In one embodiment, a pH buffer maintains the pH of a composition around 7. In one embodiment, a pH buffer maintains the pH of a composition below about 7. In one embodiment, a pH buffer maintains the pH of a composition above about 7. In one embodiment, a pH buffer maintains the pH of a composition ranging from about 2 to about 6. In one embodiment, a pH buffer maintains the pH of a composition ranging from about 5 to about 7. In one embodiment, a pH buffer maintains the pH of a composition ranging from about 6 to about 8. In one embodiment, a pH buffer maintains the pH of a composition ranging from about 7 to about 10.

In one embodiment, a pH buffer comprises citric acid, acetic acid, monosodium phosphate, N-Cyclohexyl-2-aminoethanesulfonic acid, borate, hydrochloric acid, and/or sodium hydroxide.

In one embodiment, the methods disclosed herein comprise administering a composition comprising an acid.

As used herein, the term “acid” refers to a molecule or ion capable of donating a proton, i.e., H⁺ and/or accepting electrons. In one embodiment, an “acid” refers to a Lewis acid. In one embodiment, an “acid” refers to a Bronsted acid. In one embodiment, an acid is determined by a composition's pH. In one embodiment, a pH below 7 indicates the presence of an acid.

In one embodiment, the compositions and methods disclosed herein comprise administering a formulation comprising a base.

As used herein, the term “base” refers to a molecule or ion capable of accepting a proton, i.e., an H⁺. In one embodiment, a “base” refers to a molecule capable of donating an electron pair, i.e., a Lewis base. In one embodiment, the presence of a base is determined by a compound's pH. In one embodiment, a pH above 7 indicates the presence of a base.

In one embodiment, the compositions and methods disclosed herein comprise administering a non-water soluble composition.

In some embodiments, the compositions described herein are non-aqueous.

As used herein, the term “water soluble” refers to a compound or composition capable of dissolving in water at standard temperature and pressure. In one example, 1 g of a compound dissolves in 1 L of water. In one example, 2 g of a compound dissolves in 1 L of water. In one example, 5 g of a compound dissolves in 1 L of water. In one example, 10 g of a compound dissolves in 1 L of water. In one embodiment, a compound's solubility in water is an inherent property of a compound. In one embodiment, a compound's solubility in water is facilitated by another compound, e.g., an excipient.

In one embodiment, the compositions and methods disclosed herein comprise administering a compound of Formula I, II, III, IV, V, VI, VII, VIIIa, VIIIb, IXa, IXb, Xa, Xb, XIa or XIb present as and/or within a homogenous mixture within a dosage formulation.

In one embodiment, the compositions and methods disclosed herein comprise administering a compound of Formula I, II, III, IV, V, VI, VII, VIIIa, VIIIb, IXa, IXb, Xa, Xb, XIa or XIb and at least one second compound (e.g., serotonergic drug, cannabinoid, terpene, excipient, stabilizer, antioxidant, etc.) present as and/or within a homogenous mixture within a dosage formulation.

As used herein, the term “homogeneous mixture” refers to a solid, liquid, or gaseous composition that has two or more compounds present within one state or thing, e.g., a clear, colorless solution. In one embodiment, the homogeneous mixtures disclosed herein have the same proportion, concentration, and/or ratio of its components across different samples. In one embodiment, the components in the homogeneous mixture are in the same state of matter. In one embodiment, a homogeneous mixture comprises one or more compounds within a solution, e.g., a compound of Formula I, II, III, IV, V, VI, VII, VIIIa, VIIIb, IXa, IXb, Xa, Xb, XIa or XIb and a cannabinoid within a clear solution. In one embodiment, the compositions disclosed herein are present as a homogenous mixture, e.g., a solution with no particulates, a solution with equal concentrations across samples, a powder of similar particle size, etc.

Disclosed herein is a method of modulating activity at a neurotransmitter receptor, comprising:
administering a neurotransmitter activity modulator; and
administering a dosage formulation comprising a compound of Formula I, II, III, IV, V, VI, VII, VIIIa, VIIIb, IXa, IXb, Xa, Xb, XIa or XIb to the person in need of treatment, wherein the dosage formulation modulates activity at a neurotransmitter receptor.

As used herein, the term “modulating activity of the neurotransmitter activity modulator” refers to changing, manipulating, and/or adjusting the ability of a compound or composition to affect a neurotransmitter receptor. In one embodiment, modulating the activity of a neurotransmitter activity modulator comprises administering an agonist at a neurotransmitter receptor. In one embodiment, modulating the activity of a neurotransmitter activity modulator comprises administering an antagonist at a neurotransmitter receptor.

As used herein, the term “administering” (e.g., administering a drug) refers to dosing, treating, giving, and/or providing. In one embodiment, administering a neurotransmitter activity modulator comprises providing a neurotransmitter activity modulator to an organism (e.g., a human being) with a neurotransmitter receptor. In one embodiment, administering a neurotransmitter activity modulator comprises providing a neurotransmitter activity modulator along with a compound of Formula I, II, III, IV, V, VI, VII, VIIIa, VIIIb, IXa, IXb, Xa, Xb, XIa or XIb, e.g., a formulation having each of a neurotransmitter activity modulator and a compound of Formula I, II, III, IV, V, VI, VII, VIIIa, VIIIb, IXa, IXb, Xa, Xb, XIa or XIb in a single dosage. In one embodiment, administering a neurotransmitter activity modulator comprises applying a transdermal composition, e.g., applying a topical composition to the skin having each of a neurotransmitter activity modulator and a compound of Formula I, II, III, IV, V, VI, VII, VIIIa, VIIIb, IXa, IXb, Xa, Xb, XIa or XIb. In one embodiment, administering a neurotransmitter activity modulator comprises giving a transmucosal preparation, e.g., providing rapidly dissolving a tablet with an absorption enhancer having each of a neurotransmitter activity modulator and a compound of Formula I, II, III, IV, V, VI, VII, VIIIa, VIIIb, IXa, IXb, Xa, Xb, XIa or XIb.

In one embodiment, the methods disclosed herein comprise administering a composition by inhalation for crossing a blood-brain barrier.

As used herein, the term “neurotransmitter activity modulator” refers to a compound or composition that reacts or influences activity at a neurotransmitter receptor, e.g., a compound of Formula I, II, III, IV, V, VI, VII, VIIIa, VIIIb, IXa, IXb, Xa, Xb, XIa or XIb, a serotonergic drug, an adrenergic receptor, a dopamine receptor, a GABAergic receptor, a glutaminergic receptor, a histaminergic receptor, a cholinergic receptor, an opioid receptor, or a glycinergic receptor, etc. In one embodiment, a neurotransmitter activity modulator binds on a neurotransmitter receptor. In one embodiment, a neurotransmitter activity modulator indirectly affects a neurotransmitter receptor, e.g., via interactions affecting the reactivity of other molecules at a neurotransmitter receptor. In one embodiment, a neurotransmitter activity modulator is an agonist. In one embodiment, a neurotransmitter activity modulator is an antagonist. In one embodiment, a neurotransmitter activity modulator acts (either directly or indirectly) at more than one type of neurotransmitter receptor.

In one embodiment, a neurotransmitter activity modulator is chosen from aripiprazole, bupropion, citalopram, clomipramine, dextroamphetamine, duloxetine, escitalopram, fluoxetine, fluvoxamine, milnacipran, mirtazapine, paroxetine, quetiapine, reboxetine, risperidone, sertraline, and venlafaxine.

As used herein, the term “first dosage formulation” refers to a compound or compounds selected for the purposes of causing a reaction, effect, and/or result, e.g., causing activity at a neurotransmitter receptor, reacting with other compounds, enhancing the effects of other active ingredients, inhibiting the biosynthesis of a compound, etc., within an organism. In one embodiment, a first dosage formulation comprises a compound of Formula I, II, III, IV, V, VI, VII, VIIIa, VIIIb, IXa, IXb, Xa, Xb, XIa or XIb. In one embodiment, a first dosage formulation comprises a first purified cannabinoid. In one embodiment, a first dosage formulation comprises a first purified terpene. In one embodiment, a first dosage formulation comprises a compound of Formula I, II, III, IV, V, VI, VII, VIIIa, VIIIb, IXa, IXb, Xa, Xb, XIa or XIb and a purified serotonergic derivative. In one embodiment, a first dosage formulation comprises a compound of Formula I, II, III, IV, V, VI, VII, VIIIa, VIIIb, IXa, IXb, Xa, Xb, XIa or XIb and a first purified cannabinoid. In one embodiment, a first dosage formulation comprises a compound of Formula I, II, III, IV, V, VI, VII, VIIIa, VIIIb, IXa, IXb, Xa, Xb, XIa or XIb and a first purified terpene. In one embodiment, a first dosage formulation comprises a compound of Formula I, II, III, IV, V, VI, VII, VIIIa, VIIIb, IXa, IXb, Xa, Xb, XIa or XIb, a first purified cannabinoid, and first purified terpene. In one embodiment, a first dosage formulation comprises a compound of Formula I, II, III, IV, V, VI, VII, VIIIa, VIlIb, IXa, IXb, Xa, Xb, XIa or XIb and a neurotransmitter activity modulator.

In one embodiment, a second dosage formulation comprises a compound of Formula I, II, III, IV, V, VI, VII, VIIIa, VIIIb, IXa, IXb, Xa, Xb, XIa or XIb. In one embodiment, a second dosage formulation comprises a second compound of Formula I, II, III, IV, V, VI, VII, VIIIa, VIIIb, IXa, IXb, Xa, Xb, XIa or XIb. In one embodiment, a second dosage formulation comprises a second serotonergic drug.

In one embodiment, the methods disclosed herein comprise administering a second dosage formulation. In one embodiment, the methods disclosed herein comprise administering a third dosage formulation. In one embodiment, the methods disclosed herein comprise administering a fourth dosage formulation. In one embodiment, the methods disclosed herein comprise administering more than four dosage formulations.

In certain embodiments, the dosage formulation contains a desired amount of at least one compound of Formula I, II, III, IV, V, VI, VII, VIIIa, VIIIb, IXa, IXb, Xa, Xb, XIa or XIb. In certain embodiments, the dosage formulation contains about 0.01 to about 1,000 mg of the compound, such as about 0.1 to about 500 mg, about 0.5 to about 100 mg, or about 1 to about 50 mg. In certain embodiments, the dosage formulation is calculated to contain an amount of a compound of Formula I, II, III, IV, V, VI, VII, VIIIa, VIIIb, IXa, IXb, Xa, Xb, XIa or XIb based on mg of compound per kg of the subject (mg/kg). In certain embodiments, the mg/kg range can be about 0.001 to about 10 mg/kg, such as about 0.01 to about 5, about 0.05 to about 4, about 0.05 to about 3, about 0.05 to about 3, about 0.05 to about 2, or about 0.05 to about 1 mg/kg. In some embodiments, the compound is dosed in an amount that is less than about 1 mg/kg, such as about 0.001 to about 0.99, about 0.01 to about 0.85, about 0.05 to about 0.75, about 0.01 to about 0.50, about 0.01 to about 0.25 or about 0.01 to about 0.10 mg/kg.

In one embodiment, the methods disclosed herein comprise administering one or more active ingredients, e.g., a compound(s) of Formula I, II, III, IV, V, VI, VII, VIIIa, VIIIb, IXa, IXb, Xa, Xb, XIa or XIb, cannabinoids, terpenes, neurotransmitter activity modulators, etc., in more than two doses.

Disclosed herein is a method of treating a psychological problem in a person in need of treatment, comprising administering a compound of Formula I, II, III, IV, V, VI, VII, VIIIa, VIIIb, IXa, IXb, Xa, Xb, XIa or XIb to the person in need of treatment, wherein the compound of Formula I, II, III, IV, V, VI, VII, VIIIa, VIIIb, IXa, IXb, Xa, Xb, XIa or XIb modulates activity at a neurotransmitter receptor. In some embodiments, the method further comprises the step of identifying a person in need of treatment.

As used herein, the term “identifying a person in need of treatment” refers to analyzing, diagnosing, and/or determining whether a person requires treatment for a disease or condition. In one embodiment, identifying a person in need of treatment comprises diagnosing a person with a medical condition, e.g., a neurological condition, a chemical imbalance, a hereditary condition, etc. In one embodiment, identifying a person in need of treatment comprises performing a psychiatric evaluation. In one embodiment, identifying a person in need of treatment comprises performing a blood test. In one embodiment, identifying a person in need of treatment comprises determining whether a person has a compulsive disorder. In one embodiment, identifying a person in need of treatment comprises self-identifying as having a compulsive disorder.

In certain embodiments, the compounds and compositions described herein can be used to treat or prevent certain neurological conditions. A neurological condition, also known as neurological disorders or diseases, refer to a broad range of disorders that affect the central nervous system (brain and spinal cord), the peripheral nervous system (nerves outside the brain and spinal cord), or both. These conditions are characterized by abnormal neurological function, which may result from structural, chemical, or electrical abnormalities within the nervous system that are acquired through injury, infection, immune response, toxin or chemical exposure, or are developmental in nature. Neurological conditions can manifest with various symptoms, including impaired cognition (i.e., neurocognitive disorders), movement disorders, sensory disturbances, and changes in behavior or mood. Neurological conditions include epilepsy, stroke, brain tumors, traumatic brain injury, functional neurological disorder (FND), spinal cord injury, cerebral palsy, migraine and cluster headaches, peripheral neuropathy, Bell's Palsy, Tourette's Syndrome, post-acute sequelae SARS-CoV-2 infection (PASC), Chronic Lyme disease, Autism Spectrum Disorder etc.

As used herein, the term “psychological disorder” refers to a condition wherein a person exhibits a pattern of behavioral and/or psychological symptoms that impact multiple life areas and create distress for the person experiencing these symptoms. In one embodiment, a psychological disorder is caused by a genetic disorder. In one embodiment, a psychological disorder is caused by a biological condition, e.g., excess hormone production, a lack of activity at a neurotransmitter receptor, a lack of producing neurotransmitters, etc. In one embodiment, the neurotransmitter receptor is a serotonin receptor.

In one embodiment, the psychological problem is an anxiety disorder. In one embodiment, the psychological problem is a depressive disorder. In one embodiment, the psychological problem is a compulsive disorder. In one embodiment, the psychological problem is characterized by neurodegeneration.

As used herein, the term “anxiety disorder” refers to a state of apprehension, uncertainty, and/or fear resulting from the anticipation of an event and/or situation. An anxiety disorder can disrupt the physical and psychological functions of a person. These disruptions can cause a small hindrance to a debilitating handicap for a person's everyday life. An anxiety disorder can cause a physiological symptom, e.g., muscle tension, heart palpitations, sweating, dizziness, shortness of breath, etc. An anxiety disorder can also cause a psychological symptom, e.g., fear of dying, fear of embarrassment or humiliation, fear of an event occurring, etc.

In one embodiment, an anxiety disorder comprises acute stress disorder, anxiety due to a medical condition, generalized anxiety disorder, panic disorder, panic attack, a phobia, post-traumatic stress disorder, separation anxiety disorder, social anxiety disorder, substance-induced anxiety disorder, or selective mutism.

As used herein, the term “acute stress disorder” refers to a condition developed after exposure to one or more traumatic events. Examples of traumatic events include, but are not limited to, exposure to war, rape or sexual violence, a physical attack, a mugging, childhood physical or sexual violence, kidnapping or being taken hostage, terrorist attacks, torture, natural disasters, and/or severe accidents. In one embodiment, acute stress disorder occurs within a day of experiencing a traumatic event. In one embodiment, acute stress disorder occurs within three days of experiencing a traumatic event. In some instances, acute stress disorder occurs within a week of experiencing a traumatic event. In some instances, acute stress disorder occurs within a month of experiencing a traumatic event.

As used herein, the term “anxiety due to another medical condition” refers to a condition wherein anxiety symptoms are developed because of a physiological and psychological consequence of a non-related disease, injury, and/or illness, e.g., an endocrine disease, a cardiovascular disorder, respiratory illness, a metabolic disturbance, a neurological illness, etc.

As used herein, the term “generalized anxiety disorder” refers to a condition of persistent and excessive anxiety and worry about various domains, e.g., work, school, social settings, etc., that an individual finds difficult to control. In addition, the individual experiences physical symptoms including restlessness, alertness, and/or nervousness; being easily fatigued, difficulty concentrating or mind going blank, irritability, muscle tension, and sleep disturbance.

As used herein, the term “panic disorder” refers to a condition wherein an individual experiences recurrent and unexpected panic attacks. The individual is persistently concerned about having more panic attacks and changes his or her behavior in maladaptive ways because of these panic attacks, e.g. avoidance of exercise, unfamiliar locations, new people, etc.

As used herein, the term “panic attack” refers to an abrupt surge of intense fear or intense discomfort that reaches a peak within a short period of time, e.g., seconds, minutes, hours, etc. In some instances, a panic attack comprises a physical and/or cognitive symptom. Panic attacks may be predictable, such as in response to a typically feared object or situation. In some instances, a panic attack occurs for no apparent reason.

As used herein, the term “phobia” refers to a condition of being fearful, anxious about, or avoidant of a circumscribed object and/or situation. In some instances, a phobia comprises a fear, anxiety, or avoidance that is induced by a situation to a degree that is persistent and out of proportion to the actual risk posed. Examples of phobias include, but are not limited to, a fear or anxiety of an animal, a natural environment, an injection-injury, etc.

As used herein, the term “post-traumatic stress disorder” refers to a condition developed after experiencing and/or witnessing a traumatic event or learning that a traumatic event has happened to a loved one. In some instances, a person shows symptoms of post-traumatic stress disorder within a week of experiencing the traumatic event. In some instances, a person shows symptoms of post-traumatic stress disorder within a month of experiencing the traumatic event. In some instances, a person shows symptoms of post-traumatic stress disorder within a year of experiencing the traumatic event. In some instances, a person shows symptoms of post-traumatic stress disorder after a year or more of experiencing the traumatic event. In some instances, post-traumatic stress disorder comprises a person re-experiencing the trauma event through intrusive distressing recollections of the event, flashbacks, and/or nightmares. In some instances, a symptom of post-traumatic stress disorder comprises emotional numbness and avoidance of places, people, and activities that are reminders of the trauma. In some instances, a symptom of post-traumatic stress disorder comprises increased arousal such as difficulty sleeping and concentrating, feeling anxious, and being easily irritated and angered.

As used herein the term “neurodegeneration” refers to the progressive loss of structure or function of neurons, including but not limited to the death of neurons. Many neurodegenerative diseases—including amyotrophic lateral sclerosis, Parkinson's disease, Alzheimer's disease, and Huntington's disease—occur as a result of neurodegenerative processes. Such diseases are incurable, resulting in progressive degeneration and/or death of neuron cells. Some attempts have been made to treat such diseases and conditions using fungal and plant extracts. But those methods all suffer from a common flaw in that the fungal and/or plants extracts fail to provide consistent or reliable amounts of the therapeutic compounds on account of relying on the highly variable chemical compositions of particular naturally occurring organisms.

As used herein, the term “separation anxiety disorder” refers to a condition wherein an individual is fearful and/or anxious about separation from an attachment figure to a degree that is developmentally inappropriate. In some instances, a separation anxiety disorder comprises a fear or anxiety about harm coming to an attachment figure. In some instances, a separation anxiety disorder comprises a fear of an event leading to the loss of or separation from an attachment figure and reluctance to go away from attachment figures. In some instances, a separation anxiety disorder comprises a nightmare and/or psychical symptom of distress.

As used herein, the term “social anxiety disorder” refers to a condition wherein an individual is fearful, anxious about, or avoidant of social interactions and situations that involve the possibility of being scrutinized. These social interactions and situations include meeting unfamiliar people, situations in which the individual may be observed eating or drinking, situations in which the individual performs in front of others, etc. In some instances, a social anxiety disorder is caused by the fear of being negatively evaluated by others, by being embarrassed, humiliated, rejected, and/or offending others.

As used herein, the term “substance-induced anxiety disorder” refers to a condition wherein anxiety caused by a substance intoxication and/or a withdrawal or to a medical treatment. In some instances, a withdrawal from a substance increases anxiety.

As used herein, the term “selective mutism” refers to a condition characterized by an individual's consistent failure to speak in social situations in which there is an expectation to speak, e.g., school, a lecture, a meeting, etc., even though the individual speaks in other situations. Failure to speak has significant consequences on achievement in academics, occupational settings, and/or otherwise interferes with normal social communication.

In some instances, an anxiety disorder comprises a medical diagnosis based on the criteria and classification from the Diagnostic and Statistical Manual of Medical Disorders, 5th Ed. In some instances, an anxiety disorder comprises a medical diagnosis based on an independent medical evaluation. In some instances, an anxiety disorder comprises a medical diagnosis based on a self-evaluation.

In one embodiment, the methods and compositions disclosed herein comprise administering an anxiolytic drug.

As used herein, the term “anxiolytic drug” refers to a compound or composition that reacts or influences activity at a neurotransmitter receptor, e.g., a compound of Formula I, II, III, IV, V, VI, VII, VIIIa, VIIIb, IXa, IXb, Xa, Xb, XIa or XIb, a serotonergic drug, an adrenergic receptor, a dopamine receptor, a GABAergic receptor, a glutaminergic receptor, a histaminergic receptor, a cholinergic receptor, an opioid receptor, or a glycinergic receptor, etc. In one embodiment, an anxiolytic drug binds on a neurotransmitter receptor. In one embodiment, an anxiolytic drug indirectly affects a neurotransmitter receptor, e.g., via interactions affecting the reactivity of other molecules at a neurotransmitter receptor. In one embodiment, an anxiolytic drug is an agonist. In one embodiment, an anxiolytic drug is an antagonist. In one embodiment, an anxiolytic drug acts (either directly or indirectly) at more than one type of neurotransmitter receptor.

In one embodiment, an anxiolytic drug is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, and triazolam.

As used herein, the term “depressive disorder” refers to a condition of low mood and aversion to activity that can affect a person's thoughts, behavior, feelings, and sense of well-being lasting for a time period. In one embodiment, a depressive disorder disrupts the physical and psychological functions of a person. In one embodiment, a depressive disorder causes a physiological symptom, e.g., weight loss, aches or pains, headaches, cramps, digestive problems, etc. In one embodiment, a depressive disorder causes a psychological symptom, e.g., persistent sadness; anxiety; feelings of hopelessness and irritability; feelings of guilt, worthlessness, or helplessness; loss of interest or pleasure in hobbies and activities; difficulty concentrating, remembering, or making decisions, etc.

In one embodiment, a depressive disorder is chosen from atypical depression, bipolar disorder, catatonic depression, depressive disorder due to a medical condition, major depressive disorder, postpartum depression, premenstrual dysphoric disorder, and seasonal affective disorder.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood; loss of enjoyment in things that were once pleasurable; major changes in weight (gain or loss) or appetite; insomnia or excessive sleep almost every day; a state of physical restlessness or being rundown that is noticeable by others; daily fatigue or loss of energy; feelings of hopelessness, worthlessness, or excessive guilt almost every day; problems with concentration or making decisions almost every day; recurring thoughts of death or suicide, suicide plan, or suicide attempt.

As used herein, the term “bipolar disorder” refers to a condition that causes an individual to experience unusual shifts in mood, energy, activity levels, and the ability to carry out day-to-day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation; exaggerated sense of well-being and self-confidence; decreased need for sleep; unusual talkativeness; racing thoughts; distractibility; and poor decision-making-for example, going on buying sprees, taking sexual risks, or making foolish investments.

Exemplary symptoms of depressive episodes, low mood, include, but are not limited by, depressed mood, such as feelings of sadness, emptiness, hopelessness, or tearfulness; marked loss of interest or feeling no pleasure in all—or almost all—activities; significant weight loss, weight gain, or decrease or increase in appetite; insomnia or sleeping too much; restlessness or slowed behavior; fatigue or loss of energy; feelings of worthlessness or excessive or inappropriate guilt; decreased ability to think or concentrate, or indecisiveness; and thinking about, planning or attempting suicide.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptom(s) caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g. vitamin B12 deficiency), autoimmune conditions (e.g., lupus and rheumatoid arthritis), viral or other infections (hepatitis, mononucleosis, herpes), back pain, and certain cancers (e.g., pancreatic).

As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by two weeks. In some instances, an individual experiences periods of depression separated by years. In some instances, an individual experiences symptom of depression that are nearly always present. Major depressive disorder can negatively affect a person's personal, work, or school life, as well as sleeping, eating habits, and general health. 2-7% of adults with major depressive disorder commit suicide, and up to 60% of people who commit suicide had a major depressive disorder or another related mood disorder. Dysthymia is a subtype of major depressive disorder consisting of the same cognitive and physical problems as a major depressive disorder with less severe but longer-lasting symptoms. Exemplary symptoms of a major depressive disorder include, but are not limited to, feelings of sadness, tearfulness, emptiness or hopelessness; angry outbursts, irritability or frustration, even over small matters; loss of interest or pleasure in most or all normal activities; sleep disturbances, including insomnia or sleeping too much; tiredness and lack of energy; reduced appetite, weight loss or gain; anxiety, agitation or restlessness; slowed thinking, speaking, or body movements; feelings of worthlessness or guilt, fixating on past failures or self-blame; trouble thinking, concentrating, making decisions, and remembering things; frequent thoughts of death, suicidal thoughts, suicide attempts, or suicide; and unexplained physical problems, such as back pain or headaches.

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder include, but are not limited to, lability (e.g., mood swings), irritability or anger, depressed mood, anxiety, and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day; losing interest in activities once found enjoyable; having low energy; having problems with sleeping; experiencing changes in appetite or weight; feeling sluggish or agitated; having difficulty concentrating; feeling hopeless, worthless, or guilty; and having frequent thoughts of death or suicide.

In one embodiment, a depressive disorder comprises a medical diagnosis based on the criteria and classification from Diagnostic and Statistical Manual of Medical Disorders, 5th Ed. In one embodiment, a depressive disorder comprises a medical diagnosis based on an independent medical evaluation.

In one embodiment, the methods and compositions disclosed herein comprise administering an antidepressant.

As used herein, the term “antidepressant” refers to a compound or compounds that reacts or influences activity at a neurotransmitter receptor, e.g., a compound of Formula I, II, III, IV, V, VI, VII, VIIIa, VIIIb, IXa, IXb, Xa, Xb, XIa or XIb, a serotonergic drug, an adrenergic receptor, a dopamine receptor, a GABAergic receptor, a glutaminergic receptor, a histaminergic receptor, a cholinergic receptor, an opioid receptor, or a glycinergic receptor, etc. In one embodiment, an antidepressant binds on a neurotransmitter receptor. In one embodiment, an antidepressant indirectly affects a neurotransmitter receptor, e.g., via interactions affecting the reactivity of other molecules at a neurotransmitter receptor. In one embodiment, an antidepressant is an agonist. In one embodiment, an antidepressant is an antagonist. In one embodiment, an antidepressant acts (either directly or indirectly) at more than one type of neurotransmitter receptor.

In one embodiment, an antidepressant is chosen from bupropion, citalopram, duloxetine, escitalopram, fluoxetine, fluvoxamine, milnacipran, mirtazapine, paroxetine, reboxetine, sertraline, and venlafaxine.

Disclosed herein is a method of treating headaches and/or migraines, comprising administering a composition disclosed herein to a person in need of treatment.

Disclosed herein is a method of treating nicotine addiction, comprising administering a composition disclosed herein to a person in need of treatment.

Disclosed herein is a method of treating drug addiction, comprising administering a composition disclosed herein to a person in need of treatment.

Disclosed herein is a method of treating alcohol addiction, comprising administering a composition disclosed herein to a person in need of treatment.

In some embodiments, the methods of treatment further comprise the step of identifying a person in need of treatment.

The compositions disclosed herein are useful for the treatment of compulsive disorders in humans, a variety of intractable psychiatric disorders, chronic depression, post-traumatic stress disorder, and drug or alcohol dependency. The compositions disclosed herein are also useful within the context of meditative, spiritual, and religious practices within a variety of contexts.

As used herein, the term “compulsive disorder” refers to a condition wherein an individual has an obsession causing a feeling of anxiety, fear, apprehension, etc., and has a compulsion to perform tasks to relieve said feeling of anxiety. An obsession is a thought that recurs and persists despite the efforts of an individual to ignore or confront them. In some instances, an obsession is relatively vague involving a general sense of disarray or tension accompanied by a belief that life cannot proceed as normal while the imbalance remains. In other instances, an obsession is more intense and could be a preoccupation with the thought or image of someone close to them dying or intrusions related to relationship rightness. Other obsessions concern the possibility that someone or something other than oneself—such as God, the Devil, or disease—will harm either the person, the people or things that the person cares about. In some instances, individuals perform compulsive rituals because they inexplicably feel they have to. In some instances, individuals perform compulsive rituals to mitigate the anxiety that stems from a particular obsession. The person feels that these actions will somehow either prevent a dreaded event from occurring or will push the event from their thoughts.

In one embodiment, a compulsive disorder is chosen from addiction, body dysmorphic disorder, excoriation disorder, hoarding disorder, obsessive-compulsive disorder, and trichotillomania.

As used herein, the term “addiction” refers to a physical and/or psychological dependence on a substance, activity, and/or any other habit. In one embodiment, an addiction is caused by the altered brain chemistry of an individual in response to a stimulus, e.g., a substance releasing large amounts of serotonin, an activity releasing large amounts of adrenaline, etc. In one embodiment, an addiction is a dependence on a substance, e.g., a drug, an alcohol, nicotine, a food, etc. In one embodiment, an addiction is a dependence on an activity, e.g., gambling, eating, shopping, etc.

As used herein, the term “body dysmorphic disorder” refers to a condition characterized by the obsessive idea that some aspect of an individual's appearance is severely flawed and warrants exceptional measures to hide or fix it. Exemplary symptoms of body dysmorphic disorder includes, but are not limited to, being extremely preoccupied with a perceived flaw in appearance that to others can't be seen or appears minor; a belief that a defect in appearance makes an individual ugly or deformed; a belief that others take special notice of an individual's appearance in a negative way or mock the individual; engaging in behaviors aimed at fixing or hiding the perceived flaw that are difficult to resist or control, such as frequently checking the mirror, grooming, or skin picking; attempting to hide perceived flaws with styling, makeup, or clothes; constantly comparing one's appearance with others; always seeking reassurance about one's appearance from others; having perfectionist tendencies; seeking frequent cosmetic procedures with little satisfaction; avoiding social situations; and being so preoccupied with one's appearance that it causes major distress or problems in a person's social life, work, school, or other areas of functioning.

As used herein, the term “excoriation disorder” refers to a condition of having a repeated urge to pick at one's own skin. In some instances, an excoriation disorder causes a person to often pick their skin to the extent that damage is caused.

As used herein, the term “hoarding disorder” refers to a condition of persistent difficulty in discarding or parting with possessions, regardless of their value. Exemplary symptoms of a hoarding disorder include, but are not limited to, inability to throw away possessions; severe anxiety when attempting to discard items; great difficulty categorizing or organizing possessions; indecision about what to keep or where to put things; distress, such as feeling overwhelmed or embarrassed by possessions; suspicion of other people touching items; obsessive thoughts and actions; fear of running out of an item or of needing it in the future; checking the trash for accidentally discarded objects; and functional impairments, e.g., loss of living space, social isolation, family or marital discord, financial difficulties, health hazards, etc.

As used herein, the term “obsessive-compulsive disorder” refers to a condition in which an individual has uncontrollable, reoccurring thoughts and behaviors that he or she feels the urge to repeat over and over. In some instances, an obsessive-compulsive disorder manifests itself as an individual needing to clean in order to reduce the fear that germs, dirt, or chemicals will contaminate the individual and the individual will spend many hours washing themselves or cleaning their surroundings. In some instances, an obsessive-compulsive disorder manifests itself as an individual needing to dispel anxiety. An individual may utter a name, phrase or repeat a behavior several times. The individual knows these repetitions will not actually prevent injury, but fear of harm will occur if the repetitions are not performed. In some instances, an obsessive-compulsive disorder manifests itself as an individual needing to reduce the fear of harming oneself or others by, e.g., forgetting to lock the door or turning off appliances, developing checking rituals, etc. In some instances, an obsessive-compulsive disorder manifests itself as an individual needing to order and arrange his or her surroundings to reduce discomfort, e.g., putting objects in a certain order, arranging household items in a particular manner or in a symmetric fashion, etc. In some instances, an obsessive-compulsive disorder manifests itself as an individual needing to respond to intrusive obsessive thoughts, e.g., praying or saying phrases to reduce anxiety or prevent a dreaded future event. In some instances, obsessive-compulsive disorder is caused by another medical condition. In some instances, obsessive-compulsive disorder is caused by a substance.

As used herein, the term “trichotillomania” refers to a condition of self-induced and recurrent loss of hair, e.g., pulling one's own hair out. In some instances, trichotillomania comprises an individual pulling their hair out at one location. In some instances, trichotillomania comprises an individual pulling their hair out at multiple locations. Exemplary symptoms of trichotillomania include, but are not limited to, recurrent pulling out of one's hair resulting in noticeable hair loss; an increased sense of tension immediately before pulling out the hair or when resisting the behavior; pleasure, gratification, or relief when pulling out the hair; the disturbance is not accounted for by another mental disorder and is not due to a general medical condition (i.e., dermatological condition); repeated attempts have been made to decrease or stop hair pulling; disturbances caused significant distress or impairment in social, occupational, or other important areas of functioning; distress including feelings of loss of control, embarrassment, shame; and impairment due to avoidance of work, school, or other public situations.

In one embodiment, a compulsive disorder comprises a medical diagnosis based on the criteria and classification from Diagnostic and Statistical Manual of Medical Disorders, 5th Ed. In one embodiment, a compulsive disorder comprises a medical diagnosis based on an independent medical evaluation.

Disclosed herein is a method of treating drug addiction, comprising administering a compound described herein or a composition disclosed herein to the person in need of treatment. In certain embodiments, the drug addiction is selected from amphetamine addiction, methamphetamine addiction, opioid addiction (e.g., oxycodone, fentanyl, or heroin addiction), and cocaine addiction. In certain embodiments, the drug addiction is amphetamine addiction. In certain embodiments, the amphetamine is methamphetamine.

Disclosed herein is a method of treating alcohol addiction, comprising administering a compound described herein or a composition disclosed herein to the person in need of treatment.

As used herein, the term “addiction” refers to a physical and/or psychological dependence on a substance, activity, and/or any other habit. In one embodiment, an addiction is caused by the altered brain chemistry of an individual in response to a stimulus, e.g., a substance releasing large amounts of serotonin, an activity releasing large amounts of adrenaline, etc. In one embodiment, an addiction is a dependence on a substance, e.g., a drug, an alcohol, nicotine, a food, etc. In one embodiment, an addiction is a dependence on an activity, e.g., gambling, eating, shopping, etc.

Disclosed herein is a method of treating drug overdose, comprising administering a compound described herein or a composition disclosed herein to the person in need of treatment. In certain embodiments, the drug overdose comprises the overuse of a drug that modulates trace amine-associated receptor 1 (TAAR1). In certain embodiments, the drug comprises a TAAR1 agonist. In certain embodiments, the drug comprises amphetamine. In certain embodiments, the drug comprises methamphetamine. In certain embodiments, the drug overdose comprises an opioid overdose. In some embodiments, the opioid comprises oxycodone, heroin or fentanyl.

In certain embodiments, described herein is a method of treating or reducing the effects of methamphetamine overdose. In certain embodiments, the method comprises administering a compound of described herein or composition disclosed herein to a person in need of treatment for methamphetamine overdose. In certain embodiments, the effects of methamphetamine overdose are selected from at least one of hyperthermia, convulsions, and seizures. Without being bound to any particular scientific theory, it is believed that one or more of the compounds described may be effective at treating methamphetamine addiction, methamphetamine overdose and its associated side effects by modulation via 5-HT2C and/or 5-HT2A receptors, and/or certain nicotinic receptor subtypes (e.g., a3p4 nAChr). In some embodiments, it is believed that compounds of Formula I, II, III, IV, V, VI, VII, VIIIa, VIIIb, IXa, IXb, Xa, Xb, XIa or XIb. can effectively treat methamphetamine overdose (e.g., acute methamphetamine toxicity that results in death or negative neurological outcomes) by reducing or eliminating the side effects of methamphetamine toxicity, including (but not limited to) reducing at least one of hyperthermia, convulsions, or seizures.

As used herein, the term “overdose” refers to a condition that arises when a subject ingests, injects, applies, or otherwise internalizes an amount of a substance, such as a drug, that leads to unwanted effects. In some instances, an overdose refers to the internalization of a drug or other substance in quantities much greater than are recommended or are much greater than the amount typically used to achieve a desired effect. In some embodiments, the substance is a prescription medication, an over-the-counter medication, or an illegal substance. In some instances, the overdose is accidental. In some instances, the overdose is intentional.

In some embodiments, the compositions described herein further comprise at least one compound not acting on a serotonin receptor.

In some embodiments, the compositions described herein comprise a serotonergic drug, wherein the serotonergic drug is selected from compounds of Formula I, II, III, IV, V, VI, VII, VIIIa, VIIIb, IXa, IXb, Xa, Xb, XIa or XIb. In some embodiments, the composition comprises a single serotonergic drug. In some embodiments, the serotonergic drug consists essentially of a compound of Formula I, II, III, IV, V, VI, VII, VIIIa, VIIIb, IXa, IXb, Xa, Xb, XIa or XIb.

Although the disclosed disclosure has been described with reference to various exemplary embodiments, it is to be understood that these embodiments are merely illustrative of the principles and applications of the present disclosure. Those having skill in the art would recognize that various modifications to the exemplary embodiments may be made, without departing from the scope of the disclosure.

Where reference is made to a particular compound, it should be understood that this disclosure also contemplates salts and derivatives of that compound as well as degradation products, such as oxidized versions of explicitly disclosed molecules.

Moreover, it should be understood that various features and/or characteristics of differing embodiments herein may be combined with one another. It is, therefore, to be understood that numerous modifications may be made to the illustrative embodiments and that other arrangements may be devised without departing from the scope of the disclosure.

Furthermore, other embodiments of the disclosure will be apparent to those skilled in the art from consideration of the specification and practice of the disclosure disclosed herein. It is intended that the specification and examples be considered as exemplary only, with a scope and spirit being indicated by the claims.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, 5%, or ±1% of the indicated range, value, or structure, unless otherwise indicated.

### Synthetic Examples

### Scheme 1

Azepine compounds can be prepared in a manner consistent with those set forth below. Formation of the 7-membered ring from the primary amine starting material (where W₁=NH, S, O, or Se) are described in Mizoguchi et al., Biogenetically inspired synthesis and skeletal diversification of indole alkaloids. Nat Chem 2014, 6 (1), 57-64, which is incorporated herein by reference in its entirety:

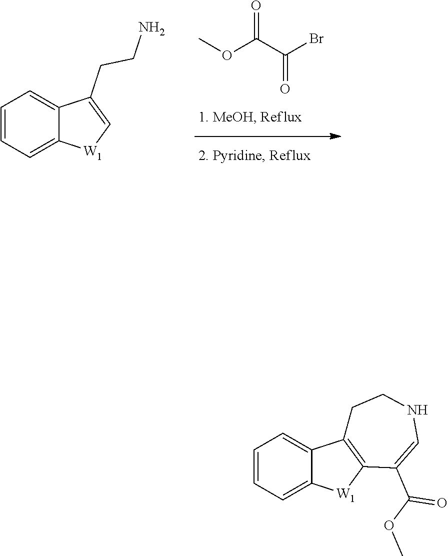

Selective reduction intermediate with NaCNBH₃: In the first step shown below, slurry of the ester in acetic acid is treated with NaBH₃CN in two portions and stirred at room temperature for 5 hr. Additional NaBH₃CN is then added. After being stirred for 1 hr to complete the reaction, the resulting mixture is slowly added conc. HCl aq. and then stirred until gas evolution is stopped. After concentration under reduced pressure, the residue is basified with concentrated aqueous solution of NH₃ at 0° C. and extracted with CH₂Cl₂, dried over Na₂SO₄, and concentrated to yield the reduced secondary amine ester, which is then carried forward for optional alkylation.

### Scheme 2

Alkylation: The crude secondary amine ester is dissolved in acetone and treated with K₂CO₃, Et₃N and alkyl, alkenyl or alkynyl bromide at r.t. for 27.5 hr. After concentration, the residue is added water and extracted with EtOAc. The organic layer is washed with water and brine, dried over Na₂SO₄, and concentrated. Purification by silica-gel column chromatography affords alkylated tertiary amine as shown below, wherein X=alkyl, alkenyl or alkynyl residue:

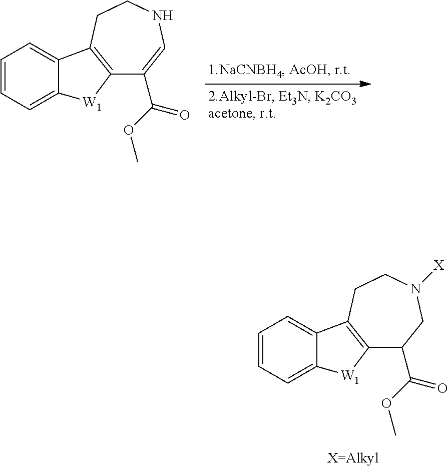

### Scheme 3

Conversion of the methyl ester residue to a carboxylic acid, followed by catalytic hydrodecarboxylation of the tertiary amine in accordance with the methods set forth in Org. Lett. Volume: 7 Issue: 11 Pages: 2157-2160 and J. Am. Chem. Soc., 2015, 137, 11340-11348 (incorporated herein by reference in its entirety) affords the desired product

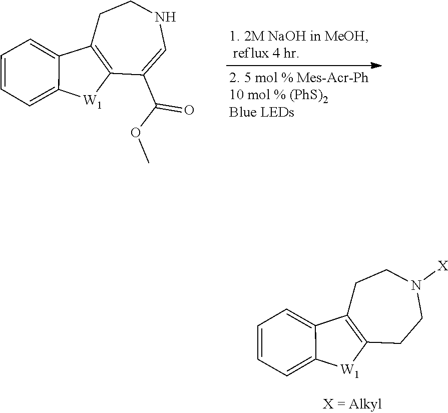

The synthetic schemes set forth above may also be used to produce the aza variants of these compounds, including aza indoles (e.g., where one or more of Z₄, Z₅, Z₆ or Z₇ of Formula II is N), by starting with the desired aza indole primary amines, many of which are commercially available:

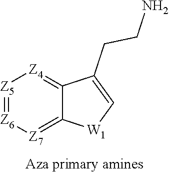

### Scheme 4

Azepine analogs according to Formulae III, IV, V, VI, VII, Vlla, VIIIb, IXa and IXb can be prepared according to the methods set forth in, e.g., International Patent Application No. PCT/US2015/062726 (WO2016/086158), Kuehne et al, J. Med. Chem. 2003, 46, 2716-2730 (doi:10.1021/jm0205620), Alleman et al., Bioorganic & Med. Chem. 2017, 27:14, 3-55-3059, and lyer et al., Nat Prod Rep. 2021 March 04; 38(2): 307-329. (doi:10.1039/d0np00033g), which are incorporated herein by reference in their entireties for all purposes. Synthetic schemes consistent with those methods are set forth below, which can be used to synthesized in an enantioselective manner from a common convergent intermediate:

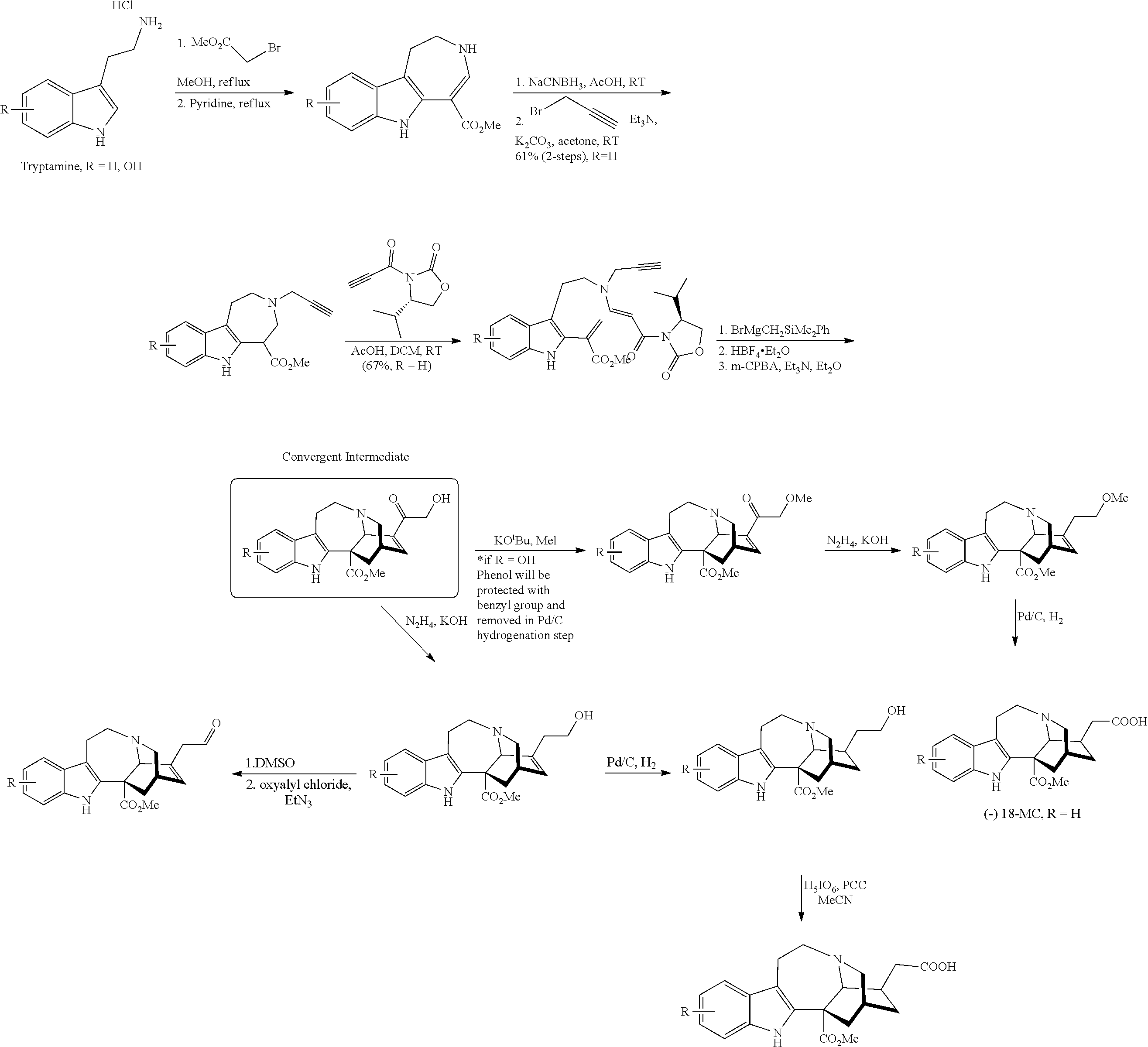

### Example 1. Production of Methyl 1-(bromomethyl)-1,2,3,4-tetrahydrobenzo[4,5]selenopheno[2,3-c]pyridine-1-carboxylate

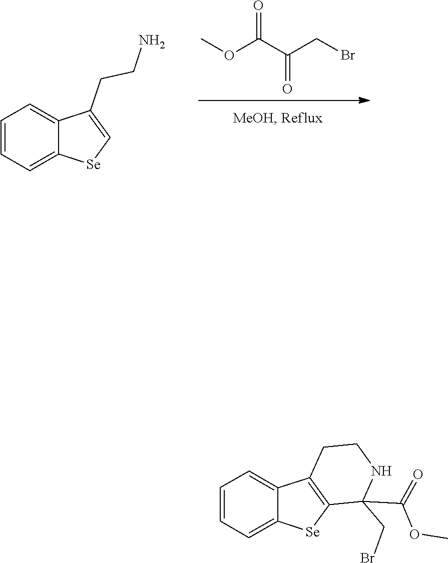

A mixture of 2-(benzo[b]selenophen-3-yl)ethan-1-amine (5 mmol), methyl bromopyruvate 13 (0.72 mL, 6 mmol), and decolorizing charcoal (0.05 g) in anhydrous MeOH (12 mL) was heated at reflux for 18 h under argon atmosphere. The resulting mixture was cooled to rt, filtered, and concentrated under reduced pressure to −2 mL and then diluted with water (15 mL). Concentrated ammonium hydroxide was added slowly until the aqueous phase became strongly basic by pH paper. The resulting mixture was filtered, and the filtrate was rinsed with 2 mL of diethyl ether. Recrystallization from acetone/water afforded methyl 1-(bromomethyl)-1,2,3,4-tetrahydrobenzo[4,5]selenopheno[2,3-c]pyridine-1-carboxylate as a yellow crystalline material. Further concentration of the mother liquor under reduced pressure yielded more material for a combined yield of 68%.

### Example 2. Production of Methyl 2,3-dihydro-1H-benzo[4,5]selenopheno[2,3-d]azepine-5-carboxylate

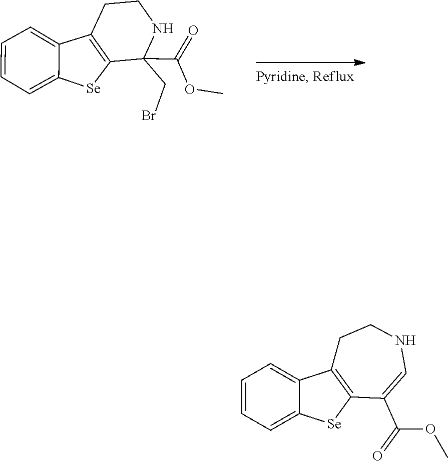 [SMILES: COC(=O)C1(CBr)NCCc2c1[se]c1ccccc21 ; COC(=O)C1=CNCCc2c1[se]c1ccccc21]

The solution of methyl 1-(bromomethyl)-1,2,3,4-tetrahydrobenzo[4,5]selenopheno[2,3-c]pyridine-1-carboxylate (0.81 mmol) in pyridine (5 mL) was refluxed under argon atmosphere for 30 min. After being cooled to rt, the solution was concentrated under reduced pressure. The residue was dissolved in dichloromethane, washed with water three times and then dried over anhydrous sodium sulfate. After filtration and removal of the solvent under reduced pressure, the crude product was purified by column chromatography on silica gel using 50% ethyl acetate in hexanes as the eluent to afford methyl 2,3-dihydro-1H-benzo[4,5]selenopheno[2,3-d]azepine-5-carboxylate as an off-white material in 77% yield.

### Example 3. Production of 2,3,4,5-Tetrahydro-1H-benzo[4,5]selenopheno[2,3-d]azepine

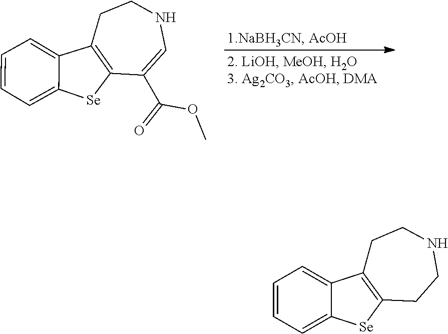 [SMILES: COC(=O)C1=CNCCc2c1[se]c1ccccc21 ; c1ccc2c3c([se]c2c1)CCNCC3]

To a stirred slurry of methyl 2,3-dihydro-1H-benzo[4,5]selenopheno[2,3-d]azepine-5-carboxylate (0.69 mmol) in glacial acetic acid (5 mL) was added sodium cyanoborohydride (0.121 g, 1.9 mmol) in small portions over a 30 minute period. Concentrated hydrochloric acid was added slowly into the reaction mixture, with cooling, until the gas evolution ceased. The reaction mixture was then concentrated under reduced pressure. The residue was poured onto ice and basified with ammonium hydroxide. The aqueous solution was then extracted thrice with dichloromethane and dried over anhydrous sodium sulfate. After filtration and removal of the solvent, the reduction adduct was obtained as an off-white material in 95% crude yield.

The crude methyl ester (0.53 mmol) was suspended in a 1:1:1 mixture of THF:MeOH:H₂O (40 mL) and to this turbid solution was added LiOH (2.7 mmol) and the resulting mixture was heated overnight at 50° C. After 18 hours the solution was cooled in an ice water bath and brought to pH ~2 by the addition of 2M HCl before diluting with water to precipitate the product as a white solid which was collected via vacuum filtration and rinsed with cold DI water. After drying overnight under reduced pressure the crude carboxylic acid was obtained in 64% yield.

The crude carboxylic acid (0.2 mmol) was charged to a vial equipped with a stir bar followed by Ag₂CO₃ (0.02 mmol), and AcOH (0.01 mmol) and DMA (0.5 M) and the vial was capped. The resulting mixture was heated in an oil bath at 100° C. for 16 h, cooled then filtered through a short plug of silica and the solvent was removed in vacuo. Purification of the crude product by flash column chromatography using 20% EtOAc in hexanes afforded 2,3,4,5-tetrahydro-1H-benzo[4,5]selenopheno[2,3-d]azepine as an off-white solid in 76% yield.

### Example 4. Production of 3-Methyl-2,3,4,5-tetrahydro-1H-benzo[4,5]selenopheno[2,3-d]azepine

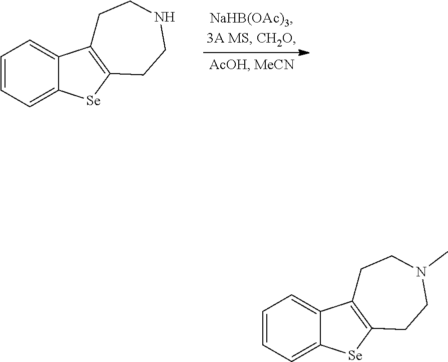

To a solution of 2,3,4,5-tetrahydro-1H-benzo[4,5]selenopheno[2,3-d]azepine (0.1 mmol) in acetonitrile (0.1 M) was added formaldehyde (1.0 mmol) followed by a few beads of activated whole 3A MS and AcOH (1 mL). After stirring at room temperature for 10 min, NaHB(OAc)₃ (>5 eq) was added at once. The reaction mixture was allowed to stir 18-24 hours at room temperature before being quenched by the addition of 1M HCl and diluted with diethyl ether. The phases were separated, and the organic phase washed with 1M HCl (×2). The combined aqueous phases were basified by the addition of solid NaOH. The aqueous phase was extracted with DCM (×3) and the combined DCM washes were dried over anhydrous sodium sulfate. After filtration, the solvent was removed in vacuo to afford 3-methyl-2,3,4,5-tetrahydro-1H-benzo[4,5]selenopheno[2,3-d]azepine in 68% yield as an off-white solid.

### Example 5. Production of 8-Methoxy-2,3,4,5-tetrahydro-1H-benzo[4,5]selenopheno[2,3-d]azepine

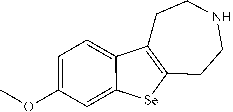 [SMILES: COc1ccc2c3c([se]c2c1)CCNCC3]

The procedures of Examples 1-3 are repeated using 2-(6-methoxybenzo[b]selenophen-3-yl)ethan-1-amine as the starting material to afford the desired target compound.

### Example 6. Production of 8-Methoxy-3-methyl-2,3,4,5-tetrahydro-1H-benzo[4,5]selenopheno[2,3-d]azepine

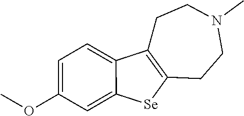

The procedure of Example 4 is repeated using the product of Example 5 as the starting material to afford the desired target compound.

### Example 7. Production of 8-Fluoro-2,3,4,5-tetrahydro-1H-benzo[4,5]selenopheno[2,3-d]azepine

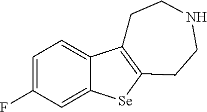 [SMILES: Fc1ccc2c3c([se]c2c1)CCNCC3]

The procedures of Examples 1-3 are repeated using 2-(6-fluorobenzo[b]selenophen-3-yl)ethan-1-amine as the starting material to afford the desired target compound.

### Example 8. Production of 8-Fluoro-3-methyl-2,3,4,5-tetrahydro-1H-benzo[4,5]selenopheno[2,3-d]azepine

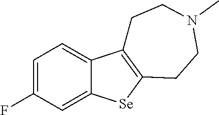 [SMILES: CN1CCc2[se]c3cc(F)ccc3c2CC1]

The procedure of Example 4 is repeated using the product of Example 7 as the starting material to afford the desired target compound.

### Example 9. Production of 8-Fluoro-7-methyl-2,3,4,5-tetrahydro-1H-benzo[4,5]selenopheno[2,3-d]azepine

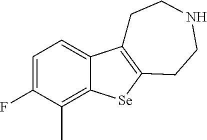 [SMILES: Cc1c(F)ccc2c3c([se]c12)CCNCC3]

The procedures of Examples 1-3 are repeated using 2-(6-fluoro-7-methylbenzo[b]selenophen-3-yl)ethan-1-amine as the starting material to afford the desired target compound.

### Example 10. Production of 8-Fluoro-3,7-dimethyl-2,3,4,5-tetrahydro-1H-benzo[4,5]selenopheno[2,3-d]azepine

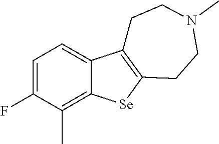 [SMILES: Cc1c(F)ccc2c3c([se]c12)CCN(C)CC3]

The procedure of Example 4 is repeated using the product of Example 9 as the starting material to afford the desired target compound.

### Example 11. Production of 8-Bromo-2,3,4,5-tetrahydro-1H-benzo[4,5]selenopheno[2,3-d]azepine

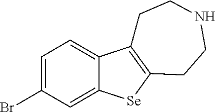 [SMILES: Brc1ccc2c3c([se]c2c1)CCNCC3]

The procedures of Examples 1-3 are repeated using 2-(6-bromobenzo[b]selenophen-3-yl)ethan-1-amine as the starting material to afford the desired target compound.

### Example 12. Production of 8-Bromo-3-methyl-2,3,4,5-tetrahydro-1H-benzo[4,5]selenopheno[2,3-d]azepine

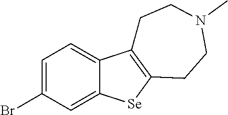 [SMILES: CN1CCc2[se]c3cc(Br)ccc3c2CC1]

The procedure of Example 4 is repeated using the product of Example 11 as the starting material to afford the desired target compound.

### Example 13. Production of Hydrofumarate Salts (Aka [1:1]Fumarate Salts)

[1:1]hydrofumarate salts of each one of the compounds are separately produced from the compounds of Examples 3-12 using the following procedure:

1 equiv of the free base product is dissolved in acetone and is added dropwise to a boiling solution of fumaric acid (1 equiv) in acetone. A precipitate forms immediately and the precipitate/acetone is stored overnight at −20° C. The solids are then filtered and washed with ice-cold acetone to yield the desired hydrofumarate salt.

### Example 14. Production of Fumarate Salts (Aka [2:1] Fumarate Salts)

[2:1] fumarate salts of each one of the compounds are separately produced from the compounds of Examples 3-12 using the following procedure:
1 equiv of the free base product is dissolved in acetone and is added dropwise to a boiling solution of fumaric acid (0.5 equiv) in acetone. A precipitate forms immediately and the precipitate/acetone is stored overnight at −20° C. The solids are then filtered and washed with ice-cold acetone to yield the desired fumarate salt.

### Biological Studies

### Head-Twitch Response (HTR) Experiments.

Dose-response studies. Dose-response studies for compounds of Formula I, II, III, IV, V, VI, VII, VIIIa, VIIIb, IXa, IXb, Xa, Xb, XIa or XIb are performed in four consecutive steps:
(a). Formulation work. A suitable (non-toxic) vehicle is identified that can be used to dissolve the compound.
(b). Pilot dose-finding study. HTR-inducing drugs typically have biphasic bell-shaped (inverted U-shaped) dose-response functions, with ascending and descending phases. To quantify the potency of a drug in a HTR dose-response study, doses covering the entire extent of the ascending phase should be included, as well as at least one dose that falls on the descending phase. A pilot dose-finding study is performed to identify a set of doses that matches those requirements. For the pilot, male C57BL/6J mice are injected with a range of doses (typically 0.3-30 mg/kg) by the IP or SC route and then behaviors are recorded in a magnetometer chamber for up to 150 minutes.
(c). Dose-response study. Groups of male C57BL/6J mice with a magnet implant are injected with vehicle or 4-5 doses of the compound (n=5-7 mice/group) by the IP or SC route and then behaviors are recorded in a magnetometer chamber for at least 30 minutes.
(d). Repeated testing. Although potency can typically be quantified based on a single dose-response study, in some instances repeated testing may be necessary. For example, the doses selected for testing may not have been ideal to calculate the median effective dose (ED50 value). If necessary, a second or third dose-response study is performed.

Analysis: The following analyses are performed for dose-response studies:

HTR Counts are Analyzed Using a 1-Way ANOVA Followed by a Post-Hoc Test (Dunnett's Test).

The median effective dose (EDso value) for the compounds (in mg/kg or moles/kg) will calculated by nonlinear regression using a gaussian or sigmoidal model. The potencies of compounds and other reference compounds can also be compared statistically using an extra-sum-of-squares F-test.

HTR counts can be binned (e.g., blocks of 1, 2, 5, or 10 minutes) and analyzed using a 2-way ANOVA (drug x time) followed by a post-hoc test (Dunnett's test or Tukey's test). 5-HT_{2A} Antagonist blockade studies. Four groups of male C57BL/6J mice with a magnet implant are pretreated SC with the selective 5-HT_{2A} antagonist M100907 (vehicle, 0.001, 0.01, or 0.1 mg/kg). Twenty minutes later, all of the animals are injected IP or SC with one dose of the compound (n=5-7 mice/group) and then behaviors are recorded in a magnetometer chamber for 30 minutes. 5-HT_{1A} Antagonist blockade studies. Four groups of male C57BL/6J mice (n=5-7 mice/group) with a magnet implant are pretreated SC with the selective 5-HT_{1A} antagonist WAY-100635 (vehicle or 1 mg/kg). Twenty minutes later, the animals are injected IP or SC with vehicle or one dose of the compound and then behaviors are recorded in a magnetometer chamber for at least 30 minutes.

Extended time-course studies. Male C57BL/6J mice with a magnet implant are injected IP or SC with up to three different treatments (n=5-6 mice/group) and then behaviors are recorded in a magnetometer chamber for up to 5 hours (the exact assessment period used will depend on the duration-of-action of the Material being tested).

Brain penetration testing. These studies are used to test whether 5-HT2A ligands that do not induce the HTR are brain penetrant in mice. Male C57BL/6J mice with a magnet implant are pretreated IP or SC with vehicle or three doses of the 5-HT2A ligand (n=5-7 mice/group); 20 minutes later, all of the mice are injected IP with 1 mg/kg (±)-DOI HCl, and then behaviors are recorded in a magnetometer chamber for 20-30 minutes.

hERG Inhibition Studies.

All experiments are conducted manually using a HEKA EPC-10 amplifier at room temperature in the whole-cell mode of the patch-clamp technique. HEK293 cells stably expressing hKv11.1 (hERG) under G418 selection can be sourced from the University of Wisconsin, Madison. Cells are cultured in DMEM containing 10% fetal bovine serum, 2 mM glutamine, 1 mM sodium pyruvate, 100 U ml-1 streptomycin, and 500 mg ml-1 penicillin, 100 μg ml-1 G418. The cell line is not authenticated or tested for mycoplasma contamination. Before experiments, cells are grown to 60-80% confluency, lifted using TrypLE, and plated onto poly-l-lysine-coated coverslips. Patch pipettes are pulled from soda lime glass (micro-haematocrit tubes) and should exhibit resistances of 2-4 MO. For the external solution, normal sodium Ringer is used (160 mM NaCl, 4.5 mM KCl, 2 mM CaCl₂, 1 mM MgCI₂, 10 mM HEPES, pH 7.4 and 290-310 mOsm). The internal solution used is potassium fluoride with ATP (160 mM KF, 2 mM MgCl2, 10 mM EGTA, 10 mM HEPES, 4 mM NaATP, pH=7.2 and 300-320 mOsm). A two-step pulse (applied every 10 s) from −80 mV initially to 40 mV for 2 s and then to −60 mV for 4 s, is used to elicit hERG currents. The percentage reduction of tail current amplitude by the compounds of Formula I, II, III, IV, V, VI, VII, VIIIa, VIIIb, IXa, IXb, Xa, Xb, XIa or XIb that are tested is determined and data are shown as mean±s.d. (n=3-4 per data point). For all experiments, solutions of the drugs are prepared fresh from 10 mM stocks in DMSO. The final DMSO concentration never exceeds 1%.

### Serotonin and Opioid Receptor Functional Assays.

Functional assay screens at 5-HT and opioid receptors are performed in parallel using the same compound dilutions and 384-well-format high-throughput assay platforms. Assays are used to assess activity at all human isoforms of the receptors, except where noted for the mouse 5-HT_{2A} receptor. Receptor constructs in pcDNA vectors are generated from the Presto-Tango GPCR library39 with minor modifications. All tested compounds of Formula I, II, III, IV, V, VI, VII, VIIIa, VIIIb, IXa, IXb, Xa, Xb, XIa or XIb are serially diluted in drug buffer (HBSS, 20 mM HEPES, pH 7.4 supplemented with 0.1% bovine serum albumin and 0.01% ascorbic acid) and dispensed into 384-well assay plates using a FLIPR Tetra automated dispenser head (Molecular Devices). Every plate includes a positive control such as 5-HT (for all 5-HT receptors), DADLE (DOR), salvinorin A (KOR), and DAMGO (MOR). For measurements of 5-HT_{2A} 5-HT_{2B}, and 5-HT₂c Gq-mediated calcium flux function, HEK Flp-In 293, T-Rex stable cell lines (Invitrogen) are loaded with Fluo-4 dye for one hour, stimulated with compounds and read for baseline (0-10 s) and peak fold-over-basal fluorescence (5 min) at 25° C. on the FLIPR Tetra system. For measurement of 5-HT₆ Gs and 5-HT₇ₐ functional assays, -mediated cAMP accumulation is detected using the split-luciferase GloSensor assay in HEKT cells measuring luminescence on a Microbeta Trilux (Perkin Elmer) with a 15 min drug incubation at 25° C. For 5-HT_{1A}, 5-HT_{1B}, 5-HT_{1F}, MOR, KOR and DOR functional assays, Gi/o, -mediated cAMP inhibition is measured using the split-luciferase GloSensor assay in HEKT cells, conducted similarly to that above, but in combination with either 0.3 μM isoproterenol (5-HT_{1A}, 5-HT_{1B}, 5-HT_{1F}) or 1 μM forskolin (MOR, KOR and DOR) to stimulate endogenous cAMP accumulation.

For measurement of 5-HT₁D, 5-HT_{1E}, 5-HT₄, and 5-HT_{5A} functional assays, β-arrestin2 recruitment is measured by the Tango assay using HTLA cells expressing tobacco etch virus (TEV) fused-p-arrestin2, as described previously with minor modifications. Cell lines are not authenticated, but they are purchased mycoplasma-free and tested for mycoplasma contamination. Data for all assays are plotted and nonlinear regression is performed using “log(agonist) vs. response” in GraphPad Prism to yield estimates of the efficacy (Emax and half-maximal effective concentration (EC₅₀)).

### Pharmacokinetic Studies.

Male and female C57/BL6J mice (12 weeks old) are administered a compound of Formula I, II, III, IV, V, VI, VII, VIIIa, VIlIlb, IXa, IXb, Xa, Xb, XIa or XIb via i.p. injection at doses of either 50 mg kg-1, 10 mg kg-1 or 1 mg kg-1. Mice are euthanized 15 min or 3 h after injection by cervical dislocation. Two males and two females are used per dose and time point. Brain and liver are collected, flash-frozen in liquid nitrogen, and stored at −80° C. until metabolomic processing. Whole brain and liver sections are lyophilized overnight to complete dryness, then homogenized with 3.2 mm diameter stainless-steel beads using a GenoGrinder for 50 s at 1,500 rpm. Ground tissue is then extracted using 225 μl cold methanol, 190 μl water, 750 μl methyl tert-butyl ether (MTBE). Seven method blanks and seven quality-control samples (pooled human serum, BiolVT) are extracted at the same time as the samples. The nonpolar fraction of MTBE is dried under vacuum and reconstituted in 60 μl of 90:10 (v/v) methanol:toluene containing 1-cyclohexyldodecanoic acid urea as an internal standard. Samples are then vortexed, sonicated and centrifuged before analysis.

For analysis of the tested compound in liver and brain, samples are randomized before injection with method blanks and quality-control samples are analyzed between every ten study samples. A six-point calibration curve is analyzed after column equilibration using blank injections, and then after all study samples. Blanks are injected after the calibration curve to ensure that none of the tested compound is retained on the column and carried over to samples. Reconstituted sample (5 μl) is injected onto a Waters Acquity UPLC CSH C₁₈ column (100 mm×2.1 mm, 1.7 μm particle size) with an Acquity UPLC CSH C₁₈ VanGuard precolumn (Waters) using a Vanquish UHPLC coupled to a TSQ Altis triple quadrupole mass spectrometer (Thermo Fisher Scientific). Mobile phase A consists of 60:40 v/v acetonitrile/water with 10 mM ammonium formate and 0.1% formic acid. Mobile phase B consists of 90:10 v/v isopropanol/acetonitrile with 10 mM ammonium formate and 0.1% formic acid. Gradients are run from 0-2 min at 15% B; 2-2.5 min 30% B; 2.5-4.5 min 48% B; 4.5-7.3 min 99% B; 7.3-10 min 15% B. The flow rate is 0.600 ml/min and the column is heated to 65° C. Mass spectrometer conditions are optimized for the target compound by direct infusion. Selected reaction monitoring is performed for the top five ions, with collision energy, source fragmentation, and radiofrequency optimized for the test compound. Data are processed with TraceFinder 4.1 (Thermo Fisher Scientific). Organ weights are recorded. The concentration in the brain is calculated using the experimentally determined number of moles of the target compound in the whole organ divided by the weight of the organ.

### 5-HT Receptor Functional Assays.

Various assays for measuring serotonin receptor activation are known to those of skill in the art, including those methods described in Olsen et al., Nat. Chem. Biol., 2020 August; 16(8):841-49, incorporated herein by reference in its entirety for all purposes. The assays described therein may be utilized to measure the functional activity of any of the serotonin receptor subtypes described herein, including 5-HT1A, 5-HT2A, 5-HT2B, and 5-HT2C. In certain embodiments, serotonin (5-hydroxytryptamine) is used as the reference compound.

### Cell Culture

HEK293T cells are maintained, passaged, and transfected in DMEM medium containing 10% FBS, 100 Units/mL penicillin, and 100 μg/mL streptomycin (Gibco-ThermoFisher, Waltham, MA) in a humidified atmosphere at 37° C. and 5% CO2. After transfection, cells are plated in DMEM containing 1% dialyzed FBS, 100 Units/mL penicillin, and 100 μg/mL streptomycin for BRET2, calcium, and GloSensor assays.

### BRET2 Assays

Cells are plated either in six-well dishes at a density of 700,000-800,000 cells/well, or 10-cm dishes at 7-8 million cells/dish. Cells are transfected 2-4 hours later, using a 1:1:1:1 DNA ratio of receptor: Gα-RLuc8:Gβ:Gγ-GFP2 (100 ng/construct for six-well dishes, 750 ng/construct for 10-cm dishes), except for the Gγ-GFP2 screen, where an ethanol co-precipitated mixture of Gβ1-4—is used at twice its normal ratio (1:1:2:1). Transit 2020 (Mirus Biosciences, Madison, WI) is used to complex the DNA at a ratio of 3 μL Transit/μg DNA, in OptiMEM (Gibco-ThermoFisher, Waltham, MA) at a concentration of 10 ng DNA/μL OptiMEM. The next day, cells are harvested from the plate using Versene (0.1M PBS+0.5 mM EDTA, pH 7.4), and plated in poly-D-lysine-coated white, clear bottom 96-well assay plates (Greiner Bio-One, Monroe, NC) at a density of 30,000-50,000 cells/well.

One day after plating in 96-well assay plates, white backings (Perkin Elmer, Waltham, MA) are applied to the plate bottoms, and growth medium is carefully aspirated and replaced immediately with 60 μL of assay buffer (1x HBSS+20 mM HEPES, pH 7.4), followed by a 10 μL addition of freshly prepared 50 μM coelenterazine 400a (Nanolight Technologies, Pinetop, AZ). After a five-minute equilibration period, cells are treated with 30 μL of drug for an additional 5 minutes. Plates are then read in an LB940 Mithras plate reader (Berthold Technologies, Oak Ridge, TN) with 395 nm (RLuc8-coelenterazine 400a) and 510 nm (GFP2) emission filters, at 1 second/well integration times. Plates are read serially six times, and measurements from the sixth read are used in all analyses. BRET2 ratios are computed as the ratio of the GFP2 emission to RLuc8 emission.

### Calcium Mobilization Assays

Cells are plated in 10-cm plates as described in the BRET2 protocol and co-transfected with receptor (1 μg) and Ga-subunit (1 μg) cDNA. The next day, cells are plated at 15,000 cells/well in poly-D-lysine coated black, clear bottom 384-well plates (Greiner Bio-One, Monroe, NC). The following day, growth medium is aspirated and replaced with 20 μL assay buffer containing 1x Fluo-4 Direct Calcium Dye (ThermoFisher Scientific, Waltham, MA) and incubated for 60 minutes at 37° C. (no CO2). Plates are brought to RT for 10 minutes in the dark before being loaded into a FLIPR Tetra@liquid-handling robot and plate reader (Molecular Devices, San Jose, CA). Baseline fluorescence measurements are taken for 10 seconds followed by robotic drug addition (10 μL) and a 60-second measurement (1 measurement/second). For antagonist assays, cells are first treated with antagonist and kept in the dark at room temperature for ten minutes before agonist addition by the FLIPR Tetra® robot. Maximal response during this time is used to calculate amplitude of the calcium transients. Measurements are analyzed as percentage of maximum signal amplitude for the construct.

Glosensor cAMP Assays

Cells are plated in 10-cm plates as previously described. Cells are transfected with plasmids encoding cDNA for the Glosensor reporter (Promega, Madison, WI), receptor, and Ga-subunit at a ratio of 2:1:1 (2 μg: 1 μg: 1 μg). The next day, cells are plated in black, clear-bottom, 384-well white plates. After aspiration of the medium on the day of the assay, cells are incubated for 60 minutes at 37° C. with 20 μL of 5 mM luciferin substrate (GoldBio, St. Louis, MO) freshly prepared in assay buffer. For Gas activity, 10 μL of drugs are added using the FLIPR Tetra@liquid-handling robot and read after 15 minutes in a Spectramax luminescence plate reader (Molecular Devices, San Jose, CA) with a 0.5 second signal integration time. For Gai activity, 10 μL of drugs are added for a 15-minute incubation period. Subsequently, 10 μL of isoproterenol (final concentration of 200 nM) are added and incubated for an additional 15-minute period before reading.

### Exemplary Embodiments

Embodiment 1: A compound of Formula II:

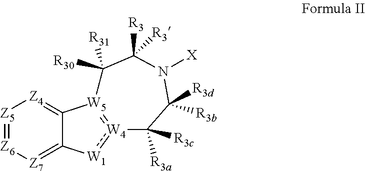

wherein
[symbol] is a single or double bond as needed to satisfy atom valences;
X is selected from hydrogen, deuterium, optionally substituted C₁-C₈ alkyl, optionally substituted C₂-C₈ alkenyl, and optionally substituted C₂-C₈ alkynyl, or X is absent and R₃, together with the carbon to which it is attached and the nitrogen associated with the N-X residue, and the atoms therebetween, are taken to form an optionally substituted 3- to 9-membered heterocyclic ring optionally including 1 to 2 additional ring heteromoieties selected from O, S, S(O), SO₂, and NR₉;
W₁ is selected from O, Se, Se(O), SeO₂, S, S(O), NR_{1′}, C(R₅₀)(R₅₁), and SO₂ when the [symbol] to which W₁ is attached is a single bond, and W₁ is selected from C(R₅₀) and N when the [symbol] to which W₁ is attached is a double bond; W₄ is selected from CR₂ and N when a [symbol] to which W₄ is attached is a single bond, and W₄ is C when a [symbol] to which W₄ is attached is a double bond;
W₅ is selected from CR₅, and N when the [symbol] to which W₅ is attached is a single bond, and W₅ is C when the [symbol] to which W₅ is attached is a double bond;
Z₄ is selected from N and CR₄;
Z₅ is selected from N and CR₅;
Z₆ is selected from N and CR₆;
Z₇ is selected from N and CR₇;
R₁ₐ is selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈; R₂, R₃, R_{3′}, R₃ₐ, R_{3b}, R_{3c}, R_{3d}, R₄, R₅, R₆, R₇, R₃₀, R₃₁, R₅₀, R₅₁, and R_{51′} are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈alkyl, optionally substituted —C₁-C₈ alkoxy, optionally substituted C₂-C₈ alkenyl, optionally substituted C₂-C₈ alkynyl, and —OSO₂R₈, or X together with the nitrogen to which is attached is taken with R₃ₐ, R_{3b}, R₃, or R_{3d} and the carbons to which they are attached, and any atoms therebetween, to form an optionally substituted 3- to 9-membered heterocyclic ring optionally including 1 to 2 additional ring heteromoieties selected from O, S, S(O), SO₂, and NR₉, or R₃ₐ or R_{3c} is taken together with R_{3d} or R_{3b}, and the carbons to which they are attached, to form an optionally substituted 3- to 8-membered cycloalkyl or heterocyclic ring optionally including 1 to 2 additional ring heteromoieties selected from O, S, S(O), SO₂, and NR₉;
R₈ is, independently for each occurrence, selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl; and
R₉ is, independently for each occurrence, is selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl; and salts, solvates, hydrates, enantiomers, polymorphs and prodrugs thereof.

Embodiment 2: The compound of Embodiment 1, wherein W₁ is NR₁a.

Embodiment 3: The compound according to any one of the preceding Embodiments, wherein R_{3b} is hydrogen.

Embodiment 4: The compound according to any one of the preceding Embodiments, wherein R₁ₐ is unsubstituted C₁-C₈ alkyl, unsubstituted C₂-C₈ alkenyl, or unsubstituted C₂-C₈ alkynyl.

Embodiment 5: The compound according to Embodiment 4, wherein R₁ₐ is methyl.

Embodiment 6: The compound according to Embodiment 4, wherein R₁ₐ is ethyl.

Embodiment 7: The compound according to Embodiment 4, wherein R₁ₐ is n-propyl.

Embodiment 8: The compound according to Embodiment 4, wherein R₁ₐ is isopropyl.

Embodiment 9: The compound according to any one of Embodiments 1-3, wherein R₁ₐ is hydrogen.

Embodiment 10: The compound according to Embodiment 1 or 3, wherein W₁ is S or Se.

Embodiment 11: The compound according to Embodiment 1 or 3, wherein W₁ is S.

Embodiment 12: The compound according to Embodiment 1 or 3, wherein W₁ is O.

Embodiment 13: The compound according to Embodiment 1 or 3, wherein W₁ is Se.

Embodiment 14: The compound according to any one of the preceding embodiments, wherein the [symbol] between W₄ and W₅ is a double bond.

Embodiment 15: The compound according to any one of the preceding Embodiments, wherein at least one of Z₄, Z₅, Z₆ or Z₇ is N.

Embodiment 16: The compound according to any one of the preceding Embodiments, wherein Z₆ is CR₆.

Embodiment 17: The compound according to any one of the preceding Embodiments, wherein Z₇ is CR₇.

Embodiment 18: The compound according to any one of the preceding Embodiments, wherein R₃ₐ is —C(O)OR₈.

Embodiment 19: The compound according to Embodiment 1 or 16, wherein R₆ is selected from halo, hydroxyl, —C(O)OR₈, C₁-C₈ alkoxy, hydrogen, and deuterium.

Embodiment 20: The compound according to Embodiment 19, wherein R₆ is hydroxyl.

Embodiment 21: The compound according to Embodiment 19, wherein R₆ is —OC(O)R₈.

Embodiment 22: The compound according to Embodiment 19, wherein R₆ is deuterium.

Embodiment 23: The compound according to Embodiment 19, wherein R₆ is —C(O)OR₈.

Embodiment 24: The compound according to any one of Embodiments 1-16 and 18-23, wherein Z₇ is N.

Embodiment 25: The compound according to any one of the preceding Embodiments, wherein R₁ₐ is selected from hydrogen and unsubstituted C₁-C₄ alkyl.

Embodiment 26: The compound according to Embodiment 25, wherein R₁ₐ is selected from methyl, ethyl and propyl.

Embodiment 27: The compound according to any one of the preceding Embodiments, wherein R₄ and R₅ are independently selected from hydrogen, deuterium, halo, hydroxyl, —C₁-C₈ alkoxy, and —OC(O)R₈.

Embodiment 28: The compound according to any of the preceding Embodiments, wherein R₄ is selected hydrogen, deuterium, halo, hydroxyl, —C₁-C₈ alkoxy, and —C(O)OR₈.

Embodiment 29: The compound according to Embodiment 28, wherein R₄ is deuterium.

Embodiment 30: The compound according to Embodiment 28, wherein R₄ is hydroxyl.

Embodiment 31: The compound according to Embodiment 28, wherein R₄ is methoxy.

Embodiment 32: The compound according to any one of the preceding Embodiments, wherein R₃ and R₃, are each independently selected from hydrogen, deuterium, hydroxyl, and —OC(O)R₈.

Embodiment 33: The compound according to Embodiment 32, wherein R₃ is hydroxyl and R_{3′} is hydrogen.

Embodiment 34: The compound according to Embodiment 32, wherein R_{3′} is hydroxyl and R₃ is hydrogen.

Embodiment 35: The compound according to Embodiment 32, wherein R₃ and R₃, are both deuterium.

Embodiment 36: The compound according to any of the preceding Embodiments, wherein R₃ₐ or R_{3c} is taken together with R_{3d} or R_{3b}, and the carbons to which they are attached, to form an optionally substituted 3- to 8-membered cycloalkyl or heterocyclic ring optionally including 1 to 2 additional ring heteromoieties selected from O, S, S(O), SO₂, and NR₉.

Embodiment 37: The compound according to Embodiment 36, wherein X is absent and R_{3c} is taken with the carbon to which it is attached and the nitrogen associated with the N-X residue, and any atoms in between, to form an optionally substituted 3- to 9-membered polycyclic heterocycle optionally including 1 to 2 additional ring heteromoieties selected from O, S, S(O), SO₂, and NR₉.

Embodiment 38: A compound of Formula VIIIa or Formula VIIIb:

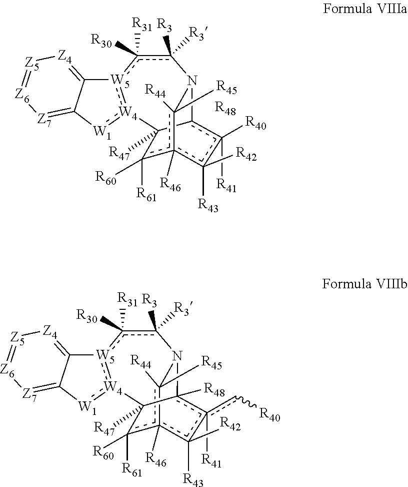

wherein
[symbol] is a single or double bond as needed to satisfy atom valences;
W₁ is selected from O, Se, Se(O), SeO₂, S, S(O), NR₁ₐ, C(R₅₀)(R₅₁), and SO₂ when the [symbol] to which W₁ is attached is a single bond, and W₁ is selected from C(R₅₀) and N when the [symbol] to which W₁ is attached is a double bond;
W₄ is selected from CR₂ and N when a [symbol] to which W₄ is attached is a single bond, and W₄ is C when a [symbol] to which W₄ is attached is a double bond;
W₅ is selected from CR₅ᵥ and N when both of the [symbol] groups to which W₅ is attached are single bonds, and W₅ is C when one of the [symbol] groups to which W₅ is attached is a double bond;
Z₄ is selected from N and CR₄;
Z₅ is selected from N and CR₅;
Z₆ is selected from N and CR₆;
Z₇ is selected from N and CR₇;
R₁ₐ is selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈;
R₂, R₃, R_{3′}, R₄, R₅, R₆, R₇, R₃₀, R₃₁, R₄₄, R₄₅, R₄₆, R₄₈, R₅₀, R₅₁, R_{51′}, R₆₀ and R₆₁ are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl and —OSO₂R₈; R₄₀, R₄₁, R₄₂, R₄₃, and R₄₇ are each independently selected from hydrogen, deuterium, —N(R₉)₂, —C(O)R₈, —OC(O)OR₈, —C(O)OR₈, —OC(O)R₈, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl, optionally substituted heteroaryl, optionally substituted aryl, optionally substituted cycloalkyl and optionally substituted heterocyclyl, or R₄₀ is taken together with R₄₁ or R₄₂, together with the carbons to which they are attached, to form an optionally substituted 3- to 9-membered heterocyclic ring optionally including 1 to 2 additional ring heteromoieties selected from O, S, S(O), SO₂, and NR₉, or R₄₁ is taken together with R₄₃, together with the carbons to which they are attached, to form an optionally substituted 3- to 9-membered heterocyclic ring optionally including 1 to 2 additional ring heteromoieties selected from O, S, S(O), SO₂, and NR₉;
R₈ is, independently for each occurrence, selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl;
R₉ is, independently for each occurrence, selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl;
and further wherein when the [symbol] adjacent to R₃ is a double bond, R₃₁ and R₃, are absent and the [symbol] adjacent to R₃₀ and W₅ is a single bond; when the [symbol] adjacent to R₄₄ is a double bond, R₄₅ and R₄₆ are absent; when the [symbol] adjacent to R₆₀ is a double bond, R₆₁ and R₄₆ are absent; when the [symbol] adjacent to R₄₂ and R₄₆ is a double bond, R₄₆ and R₄₃ are absent; when the [symbol] adjacent to R₄₁ and R₄₈ is a double bond, R₄₁ and R₄₈ are absent; when the [symbol] adjacent to R₄₂ and R₄₁ is a double bond, R₄₁ and R₄₃ are absent; when the [symbol] adjacent to R₄₀ in Formula VIIlb is a double bond, R₄₁ is absent; and when the [symbol] adjacent to R₃₀ and W₅ is a double bond, then W₅ is C and the [symbol] between W₅ and W₄ is a single bond,
and salts, solvates, hydrates, enantiomers, polymorphs and prodrugs thereof.

Embodiment 39: The compound according to Embodiment 38, wherein R₄₇ is —C(O)OR₈.

Embodiment 40: The compound according to any of Embodiments 38-39, wherein R₄₀ is optionally substituted C₁-C₈ alkyl.

Embodiment 41: The compound according to any of Embodiment 40, wherein R₄₀ is C₁-C₈ alkyl substituted with at least one of hydroxyl, C₁-C₈ alkoxy, or —OC(O)R₈.

Embodiment 42: The compound according to any one of Embodiments 38-41, wherein the compound comprises at least one deuterium.

Embodiment 43: The compound according to Embodiment 42, wherein at least one of R₄, R₅, R₆ and R₇ is deuterium.

Embodiment 44: The compound according to Embodiment 42 or 43, wherein R₄₇ is —C(O)OR₈, and R₈ is —CD₃.

Embodiment 45: The compound according to any one of Embodiments 42-44, wherein R₄₀ is a C₁-C₈ alkyl substituted with —OCD₃.

Embodiment 46: The compound according to any one of Embodiments 42-45, wherein R₅ is —OC(O)R₈.

Embodiment 47: The compound according to any one of Embodiments 42-46, wherein R₆ is —OC(O)R₈.

Embodiment 48: The compound according to any one of the preceding Embodiments, wherein the compound comprises a [1:1] salt.

Embodiment 49: The compound according to any one of Embodiments 1-47, wherein the compound comprises a [2:1] salt.

Embodiment 50: The compound according to Embodiment 49, wherein the [2:1] salt comprises an oxalate salt or a fumarate salt.

Embodiment 51: The compound according to any one of the preceding Embodiments, wherein the compound is crystalline.

Embodiment 52: A composition comprising, consisting essentially of, or consisting of a compound according to any one of Embodiments 1-51 and an excipient.

Embodiment 53: The composition according to Embodiment 52, wherein the composition is a pharmaceutical composition comprising, consisting essentially of, or consisting of a therapeutically effective amount of a compound according to any one of Embodiments 1-51 and a pharmaceutically acceptable excipient.

Embodiment 54: A composition comprising, consisting essentially of, or consisting of a first component: a compound according to any one of Embodiments 1-51; a second component selected from (a) a serotonergic drug, (b) a cannabinoid and (c) a terpene; and a pharmaceutically acceptable excipient.

Embodiment 55: The composition according to Embodiment 54, wherein the second component is purified.

Embodiment 56: A method of preventing or treating a psychological disorder comprising administering to a subject in need thereof a therapeutically effective amount of a compound according to any one of Embodiments 1-51 or a composition according to any one of Embodiments 52-55.

Embodiment 57: A method of preventing or treating an addiction, comprising administering to a subject in need thereof a therapeutically effective amount of a compound according to any one of Embodiments 1-51 or a composition according to any one of Embodiments 52-55.

Embodiment 58: The method of Embodiment 57, wherein the addiction comprises cocaine use, methamphetamine use, nicotine use, or opioid use.

Embodiment 59: The compound of Embodiment 38, wherein the compound of Formula VIIIa is selected from compounds of Formula Xa or Formula Xb:

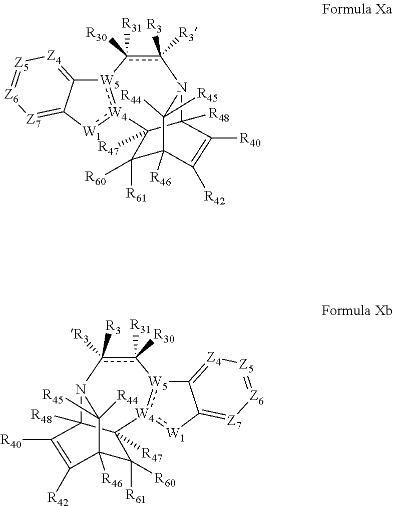

and salts, solvates, hydrates, enantiomers, polymorphs and prodrugs thereof.

Embodiment 60: The compound of Embodiment 59, wherein R₄₀ is a residue selected from

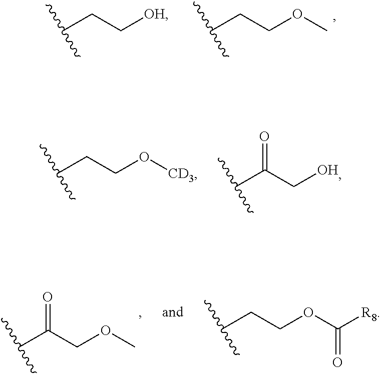

Embodiment 61: The compound of Embodiment 59 or 60, wherein R₃, R_{3′}, R₃₀, R₃₁, R₄₂, R₄₄, R₄₅, R₄₆, R₄₈, R₆₀ and R₆₁ are selected from hydrogen and deuterium.

Embodiment 62: The compound of any one of Embodiments 59-61, wherein the [symbol] to which W₄ and W₅ are attached is a double bound, and wherein W₄ and W₅ are both C.

Embodiment 63: The compound of any one of Embodiments 59-62, wherein the [symbol] to which W₁ is attached is a single bond, and wherein W₁ is selected from O, Se, S, and NR₁ₐ.

Embodiment 64: The compound of any one of Embodiments 59-63, wherein W₁ is NR₁ₐand R₁ₐ is hydrogen.

Embodiment 65: The compound of any one of Embodiments 59-64, wherein Z₄ is CR₄; Z₅ is CR₅; Z₆ is CR₆; and Z₇ is CR₇.

Embodiment 66: The compound of any one of Embodiments 59-65, wherein the compound is selected from:

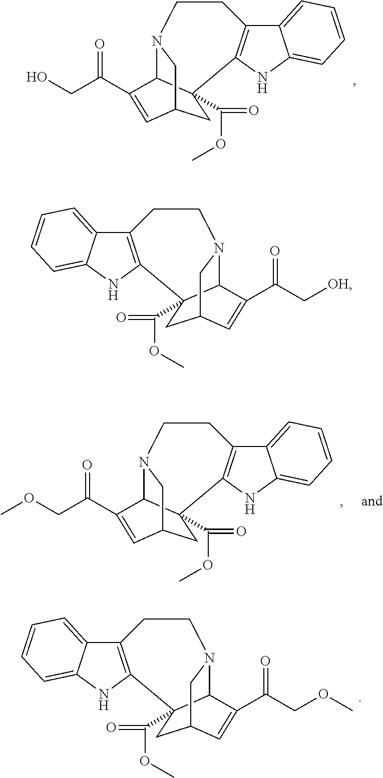

Embodiment 67: The compound of any one of Embodiments 59-65, wherein the compound is selected from:

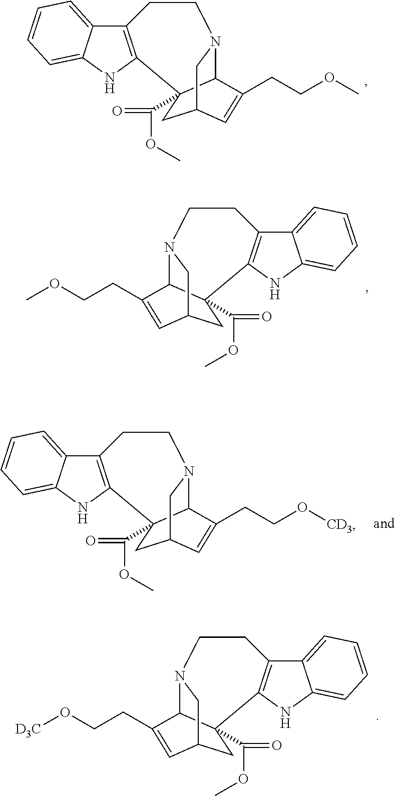

Embodiment 68: The compound of any one of Embodiments 59-65, wherein the compound is selected from:

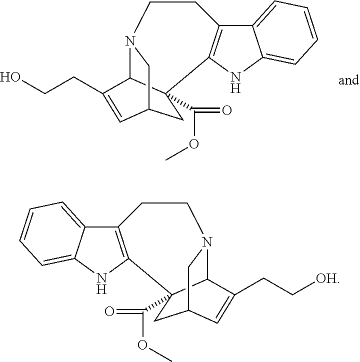 [SMILES: COC(=O)[C@]12CC3C=C(CCO)C1N(CCc1c2[nH]c2ccccc12)C3 ; COC(=O)[C@@]12CC3C=C(CCO)C1N(CCc1c2[nH]c2ccccc12)C3]

Embodiment 69: The compound of any one of Embodiments 1-47 and 59-68, wherein the compound is solid at a temperature of 22° C., humidity of 50% and pressure of 1 atm.

Embodiment 70: The compound of any one of Embodiments 1-51 and 59-68, wherein the compound has a purity of at least 95.0 wt. %.

Embodiment 71: The compound of any one of Embodiments 1-51 and 59-68, wherein the compound has a purity of at least 98.5 wt. %.

Embodiment 72: The compound of any one of Embodiments 1-51 and 59-68, wherein the compound has a purity of at least 99.0 wt. %.

Embodiment 73: The compound of any one of Embodiments 1-51 and 59-68, wherein the compound has a purity of at least 99.5 wt. %.

Embodiment 74: The compound of any one of Embodiments 1-51 and 59-68, wherein the compound has a purity of at least 99.0 wt. %.

Embodiment 75: The compound of any one of Embodiments 59-74, wherein the compound comprises a salt form.

Embodiment 76: The compound of any one of Embodiments 59-75, wherein the compound is an HCl salt.

Embodiment 77: The compound of any one of Embodiments 59-76, wherein the compound is crystalline.

Embodiment 78: A composition comprising a compound of any one of Embodiments 59-77 and a pharmaceutically acceptable carrier.

Embodiment 79: A method of treating or preventing substance use disorder in an individual, comprising administering an effective amount of a compound or composition according to any one of Embodiments 59-79.

Embodiment 80: The method of Embodiment 79, wherein the compound or composition is administered orally.

Embodiment 81: The method of any one of Embodiments 79-80, wherein the individual is a mammal.

Embodiment 82: The method of any one of Embodiments 79-81, wherein the individual is a human.

Embodiment 83: The method of any one of Embodiments 79-81, wherein the substance is selected from at least one of cocaine, nicotine, opiates, alcohol, morphine, fentanyl, and methamphetamine.

Embodiment 84: The compound of Embodiment 39, wherein the compound is selected from:

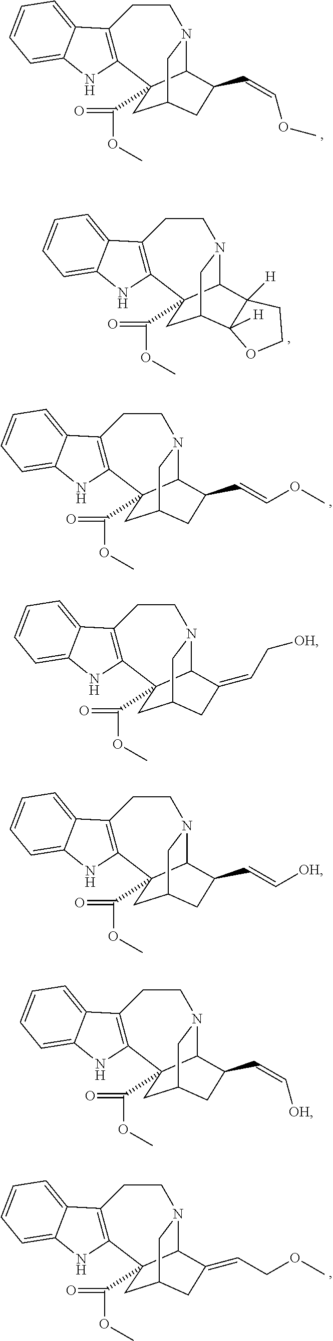

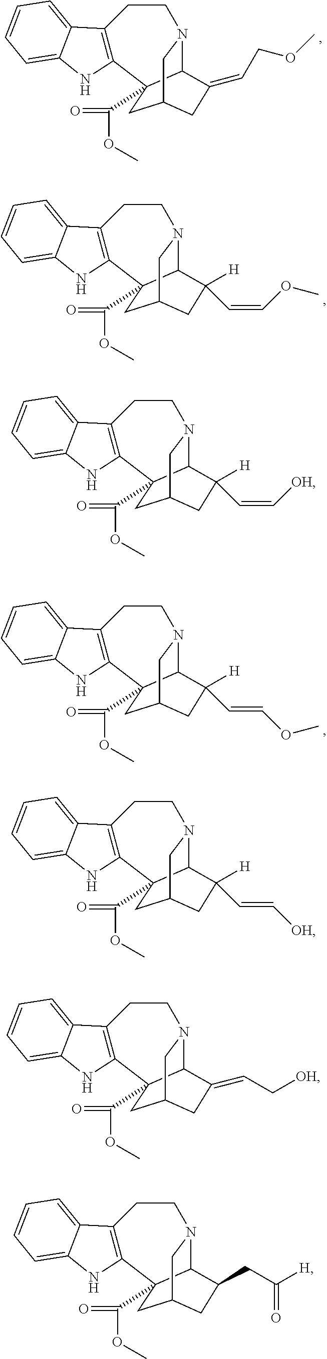

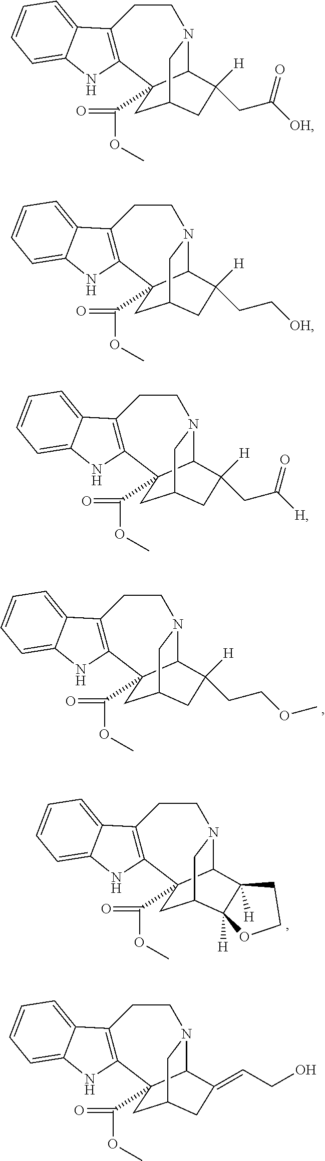

and salts, solvates, hydrates, enantiomers, polymorphs and prodrugs thereof.

Embodiment 85: The compound of Embodiment 39, wherein the compound is selected from:

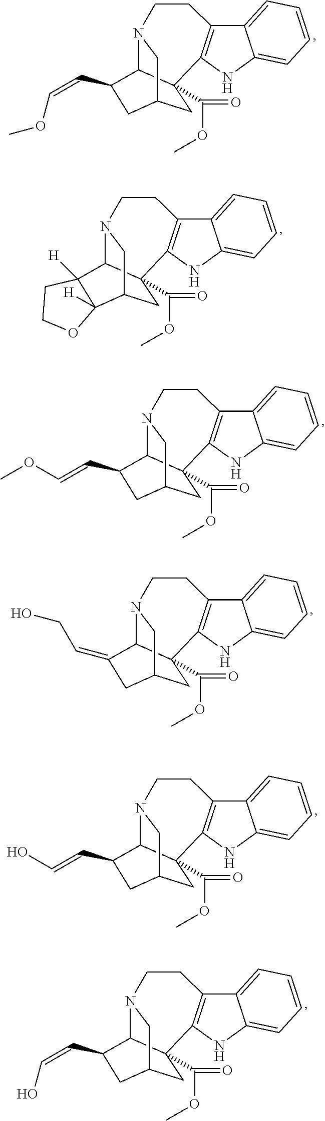

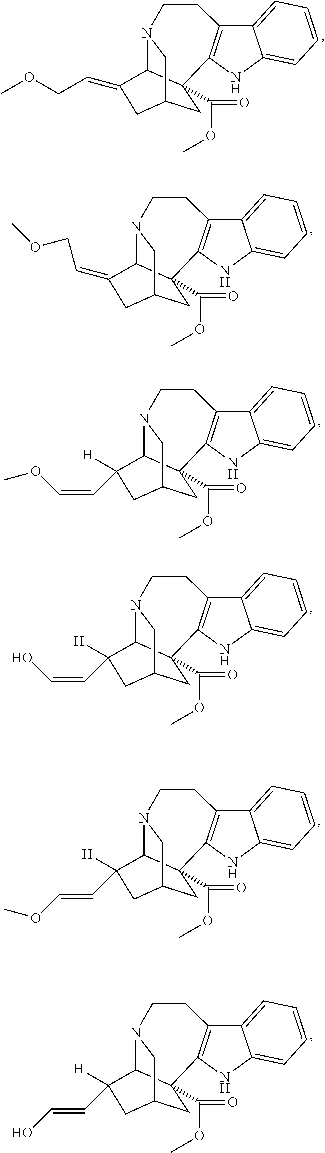

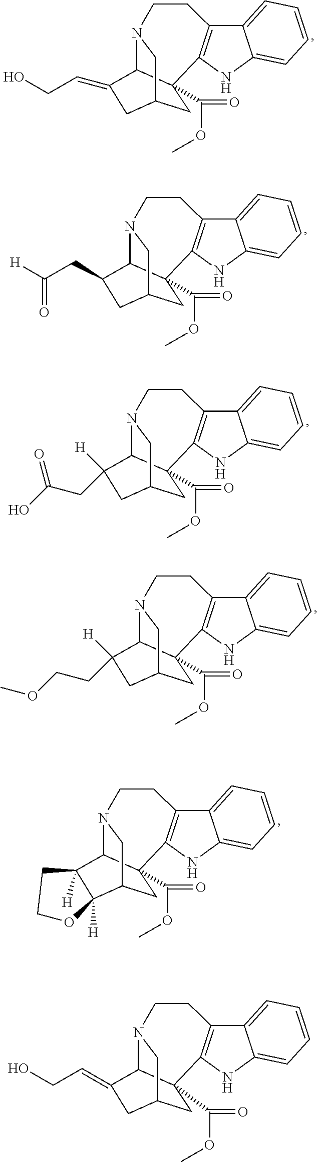

and salts, solvates, hydrates, enantiomers, polymorphs and prodrugs thereof.

Embodiment 86: The compound of Embodiment 39, wherein the compound is selected from:

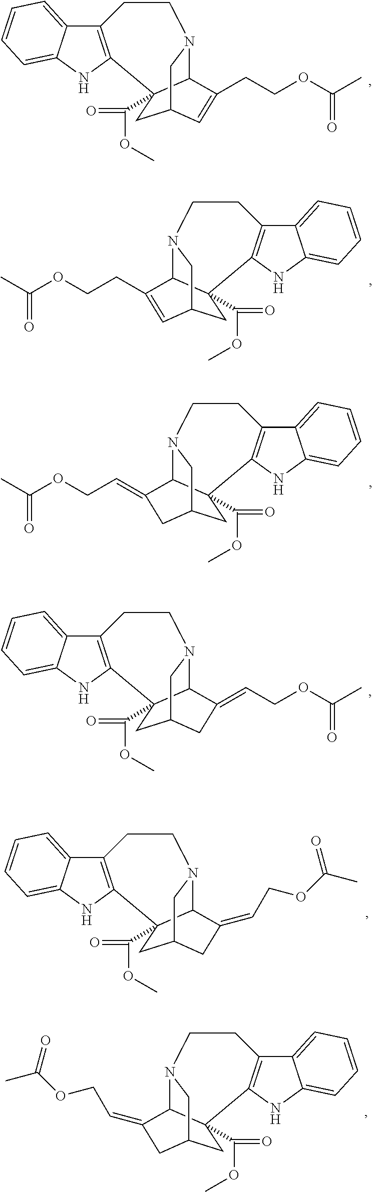

and salts, solvates, hydrates, enantiomers, polymorphs and prodrugs thereof.

Embodiment 87: The compound according to any one of Embodiments 84-86, wherein the compound is in salt form.

Embodiment 88: The compound of Embodiment 87, wherein the compound is in HCl salt form.

Embodiment 89: The compound according to any one of Embodiments 84-88, wherein the compound is crystalline.

Embodiment 90: A composition comprising a compound of any one of Embodiments 84-89 and a pharmaceutically acceptable excipient.

Embodiment 91: A method of preventing or treating a psychological disorder comprising administering to a subject in need thereof a therapeutically effective amount of a compound according to any one of Embodiments 84-86 or a composition according to Embodiment 88.

Embodiment 92: A method of preventing or treating a substance use disorder, comprising administering to a subject in need thereof a therapeutically effective amount of a compound according to any one of Embodiments 84-89 or a composition according to Embodiment 90.

Embodiment 93: The method of Embodiment 92, wherein the substance is selected from at least one of cocaine, nicotine, opiates, alcohol, morphine, fentanyl and methamphetamine.

Embodiment 94: The compound of any one of Embodiments 59-65, wherein the compound is selected from:

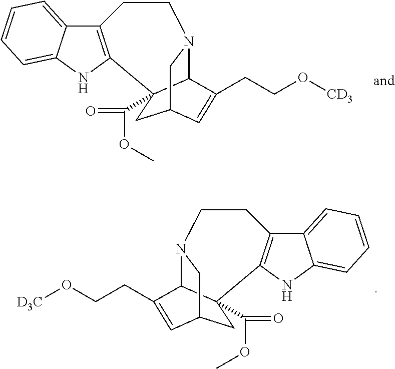 [SMILES: [2H]C([2H])([2H])OCCC1=CC2CN3CCc4c([nH]c5ccccc45)[C@](C(=O)OC)(C2)C13 ; [2H]C([2H])([2H])OCCC1=CC2CN3CCc4c([nH]c5ccccc45)[C@@](C(=O)OC)(C2)C13]

Embodiment 95: The compound of any one of Embodiments 59-65, wherein the compound is selected from:

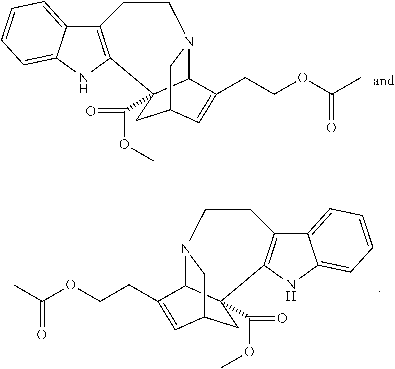

Finally, it is noted that, as used in this specification and the appended claims, the singular forms “a,” “an,” and “the,” include plural referents unless expressly and unequivocally limited to one referent and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. A compound of Formula III: 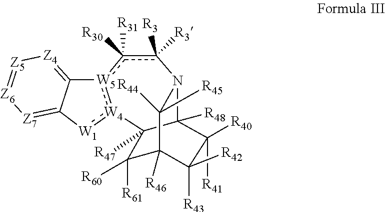
wherein [symbol]
is a single or double bond as needed to satisfy atom valences;
W₁ is NR₁ₐ when the [symbol] to which W₁ is attached is a single bond, or W₁ is N when the [symbol] to which W₁ is attached is a double bond;
W₄ is C and one of the [symbol] to which W₄ is attached is a double bond;
W₅ is C and one of the [symbol] groups to which W₅ is attached is a double bond, wherein when the [symbol] adjacent to R₃ is a double bond then R₃₁ and R₃, are absent, and wherein when the [symbol] adjacent to R₃₀ and W₅ is a double bond then R₃₁ is absent and the [symbol] between W₅ and W₄ is a single bond;
Z₄ is CR₄;
Z₅ is CR₅;
Z₆ is CR₆;
Z₇ is CR₇;
R₁ₐ is selected from hydrogen, deuterium, optionally substituted C₁-C₈ alkyl, optionally substituted C₂-C₈ alkenyl, optionally substituted C₂-C₈ alkynyl, —C(O)R₈, —C(O)OR₈, —P(O)(OR₉)₂, —C(O)N(R₉)₂, —SOR₈, and —SO₂R₈;
R₃, R_{3′}, R₄, R₅, R₆, R₇, R₃₀, R₄₄, R₄₅, R₄₆, R₄₈, R₅₀, R₅₁, R_{51′}, R₆₀, and R₆₁ are each independently selected from hydrogen, deuterium, —N(R₉)₂, —C(O)R₈, —OC(O)OR₈, —C(O)OR₈, —OC(O)R₃, —SR₉, —P(O)(OR₉)₂, —C(O)N(R₉)₂, halo, hydroxyl, optionally substituted C₁-C₈ alkyl, optionally substituted —C₁-C₈ alkoxy, optionally substituted C₂-C₈ alkenyl, optionally substituted C₂-C₈ alkynyl and —OSO₂R₃;
R₄₁, R₄₂, and R₄₃ are each independently selected from hydrogen, deuterium, —N(R₉)₂, —C(O)R₃, —OC(O)OR₃, —C(O)OR₃, —OC(O)R₃, —SR₉, —P(O)(OR₉)₂, —C(O)N(R₉)₂, —OSO₂R₃, halo, hydroxyl, optionally substituted C₁-C₈ alkyl, optionally substituted —C₁-C₈ alkoxy, optionally substituted C₂-C₈ alkenyl and optionally substituted C₂-C₈ alkynyl, or R₄₀ is taken together with R₄₁ or R₄₂, together with the carbons to which they are attached, to form an optionally substituted 3- to 9-membered heterocyclic ring optionally including 1 to 2 additional ring heteromoieties selected from O, S, S(O), SO₂, and NR₉, or R₄₁ is taken together with R₄₃, together with the carbons to which they are attached, to form an optionally substituted 3- to 9-membered heterocyclic ring optionally including 1 to 2 additional ring heteromoieties selected from O, S, S(O), SO₂, and NR₉;
R₄₀ is a substituted C₁-C₈ alkyl;
R₄₇ is —C(O)OR₈;
R₈ is, independently for each occurrence, selected from hydrogen, deuterium, —N(R₉)₂, optionally substituted C₁-C₈ alkyl, optionally substituted C₂-C₈ alkenyl, optionally substituted C₂-C₈ alkynyl, optionally substituted heteroaryl, and optionally substituted aryl;
R₉ is, independently for each occurrence, selected from hydrogen, deuterium, optionally substituted C₁-C₈ alkyl, optionally substituted C₂-C₈ alkenyl, optionally substituted C₂-C₈ alkynyl, optionally substituted heteroaryl, and optionally substituted aryl,
and salts, solvates, hydrates, enantiomers, polymorphs and prodrugs thereof.

2. The compound of claim 1, wherein R₃₁ and R₃, are absent and the [symbol] adjacent to R₃ is a double bond.

3. The compound of claim 2, wherein R₃ and R₃₀ are each independently selected from hydrogen and deuterium.

4. The compound of claim 3, wherein R₄₁, R₄₂, R₄₄, R₄₅, R₄₆, R₄₈, R₆₀ and R₆₁ are each hydrogen.

5. The compound of claim 4, wherein R₄₀ is C₁—C alkyl substituted with at least one of hydroxyl or an optionally substituted C₁-C₈ alkoxy.

6. The compound of claim 5, wherein R₄₀ is C₁-C₈ alkyl substituted with —OCD₃.

7. The compound of claim 1, wherein R₃₁ is absent and the [symbol] adjacent to R₃₀ and W₅ is a double bond and the [symbol] between W₅ and W₄ is a single bond, and wherein the [symbol] between R₃ and R₃₀ is a single bond.

8. The compound of claim 7, wherein R₄₀ is C₁-C₈ alkyl substituted with at least one of hydroxyl or an optionally substituted C₁-C₈ alkoxy.

9. The compound of claim 8, wherein R₄₀ is C₁-C₈ alkyl substituted with —OCD₃.

10. The compound of claim 9, wherein R₄₀ is an ethyl group substituted with —OCD₃.

11. The compound of claim 9, wherein the compound of Formula III is selected from: 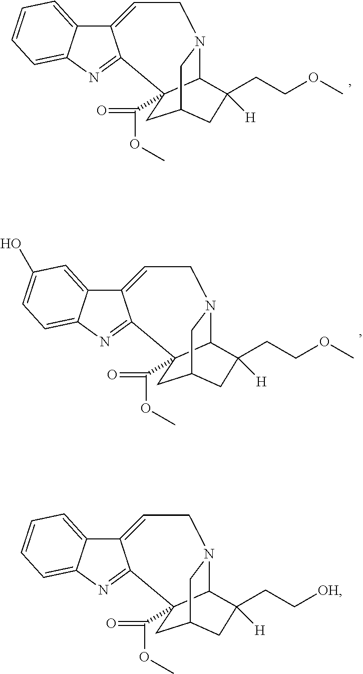 [SMILES: COCCC1CC2CN3CC=C4C(=Nc5ccccc54)[C@](C(=O)OC)(C2)C13 ; COCCC1CC2CN3CC=C4C(=Nc5ccc(O)cc54)[C@](C(=O)OC)(C2)C13 ; COC(=O)[C@@]12CC3CC(CCO)C1N(CC=C1C2=Nc2ccccc21)C3]
and salts, solvates, hydrates, enantiomers, polymorphs and prodrugs thereof.

12. The compound of claim 9, wherein the compound of Formula III is selected from: 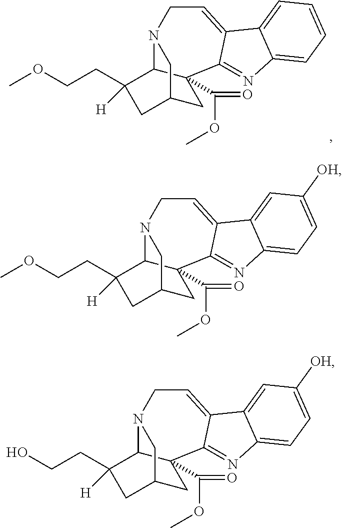 [SMILES: COCCC1CC2CN3CC=C4C(=Nc5ccccc54)[C@@](C(=O)OC)(C2)C13 ; COCCC1CC2CN3CC=C4C(=Nc5ccc(O)cc54)[C@@](C(=O)OC)(C2)C13 ; COC(=O)[C@]12CC3CC(CCO)C1N(CC=C1C2=Nc2ccc(O)cc21)C3]
and salts, solvates, hydrates, enantiomers, polymorphs and prodrugs thereof.

13. The compound of claim 9, wherein R₄, R₅, R₆ and R₇ are hydrogen.

14. The compound of claim 13, wherein R₃ is methyl.

15. The compound of claim 14, wherein R₄₀ is C₁-C₈ alkyl substituted with —OCD₃.

16. The compound of claim 5, wherein R₄, R₅, R₆ and R₇ are hydrogen.

17. The compound of claim 16, wherein R₃ is methyl.

18. The compound of claim 17, wherein R₄₀ is C₁-C₈ alkyl substituted with —OCD₃.

19. A method of preventing or treating a substance use disorder, comprising administering to a subject in need thereof a therapeutically effective amount of a compound of claim 1.

20. The method of claim 19, wherein the substance is selected from at least one of cocaine, nicotine, opiates, alcohol, morphine, fentanyl or methamphetamine.
